(19) United States
(12) Patent Application Publication
(10) Pub. No.: US 2024/0116952 A1
(43) Pub. Date: Apr. 11, 2024

(54) KRAS INHIBITOR AND PHARMACEUTICAL USES THEREOF

(71) Applicant: RISEN (SUZHOU) PHARMA TECH CO., LTD., Suzhou (CN)

(72) Inventors: Jiasheng LU, Suzhou (CN); Xiang JI, Suzhou (CN); Xianchao DU, Suzhou (CN); Yanpeng WU, Suzhou (CN); Xiaolin HE, Suzhou (CN); Guangwei REN, Suzhou (CN); Lina CHU, Suzhou (CN); Chuanhao HUANG, Suzhou (CN); Xingwu ZHU, Suzhou (CN); Yuhua ZHANG, Suzhou (CN); Jian GE, Suzhou (CN); Tianlun ZHOU, Suzhou (CN); Xiangsheng YE, Suzhou (CN); Xianqi KONG, Dollard-des-Ormeaux (CA); Dawei CHEN, Suzhou (CN)

(21) Appl. No.: 18/463,439

(22) Filed: Sep. 8, 2023

(30) Foreign Application Priority Data
Sep. 9, 2022 (CN) 202211101617.3

(51) Int. Cl. C07D 519/00; A61K 45/06; A61P 35/00; C07F 9/6561

(52) U.S. Cl. CPC C07D 519/00; A61K 45/06; A61P 35/00; C07F 9/6561

(57) ABSTRACT

The disclosure relates to KRAS^{G12D} inhibitor compounds having the structure of Formula (A) or Formula (B), pharmaceutical compositions thereof, and methods of use thereof for inhibiting, treating, and/or preventing KRAS^{G12D} mutation-associated diseases, disorders and conditions.

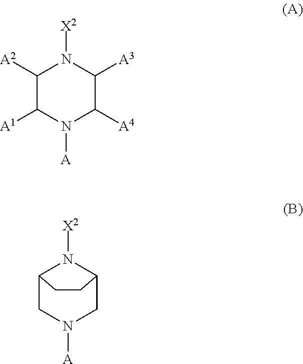

### CROSS-REFERENCE TO RELATED APPLICATIONS

This application claims the benefit of priority from Chinese patent application no. CN202211101617.3, filed on Sep. 9, 2022, which is incorporated by reference herein in its entirety.

### FIELD

The present disclosure relates to KRAS inhibitors and pharmaceutically acceptable salts, esters, hydrates, solvates and stereoisomers thereof, as well as pharmaceutical compositions thereof and methods of use for inhibiting, treating and/or preventing KRAS^{G12D} mutation-associated diseases.

### BACKGROUND

The Kirsten Rat Sarcoma Viral Oncogene Homolog (K-Ras) gene belongs to the Ras family of oncogenes and is one of the most common gene mutations in human cancers. Its encoded protein (KRAS) is part of the RAS/MAPK signal transduction pathway which regulates cell growth and differentiation. KRAS is a small GTPase, a class of enzymes which convert the nucleotide guanosine triphosphate (GTP) into guanosine diphosphate (GDP). It is turned on (activated) by binding to GTP and turned off (inactivated) by converting the GTP to GDP. In this way KRAS acts as a molecular on/off switch. In most cells, KRAS is inactivated. When activated, it can activate several downstream signaling pathways including the MAPK signal transduction pathway, the PI3K signal transduction pathway and the Ral-GEFs signal transduction pathway. These signal transduction pathways play an important role in promoting cell survival, proliferation, and cytokine release, thus affecting tumor occurrence and development.

In human cancers, K-Ras gene mutations occur in nearly 90% of pancreatic cancers, approximately 30-40% of colon cancers, approximately 17% of endometrial cancers, and approximately 15-20% of lung cancers (mostly non-small cell lung cancer, NSCLC). K-Ras gene mutations also occur in bile duct cancers, cervical cancers, bladder cancers, liver cancers, and breast cancers, as well as leukemias. K-Ras gene mutations are thus found at high rates in many different types of cancer.

Most K-Ras missense mutations occur in Codon 12, which results in changing the glycine at position 12 (G12) to another amino acid. Among these mutations, G12C, G12D, G12R and G12V are the most common KRAS mutations in patients. For instance, KRAS^{G12D} and KRAS^{G12V} mutations are found in approximately 90% of pancreatic cancers, wherein as KRAS^{G12D} is the most common KRAS mutation in colon cancer. KRAS^{G12C} mutant protein has gained significant attention recently as a prominent target for research. In 2021, The U.S. Food and Drug Administration (FDA) approved sotorasib as the first KRAS^{G12C} blocking drug for the treatment of adult patients with NSCLC. The KRAS^{G12C} inhibitor adagrasib was also approved by the U.S. FDA in 2022 for treatment of NSCLC. However, existing KRAS inhibitors face significant limitations. One of the biggest obstacles to KRAS inhibitor treatment is the emergence of drug resistance. While the biological basis of acquired drug resistance is not well understood, it has been suggested that several factors may play a role, including cellular heterogeneity in tumors; the activation of wild-type RAS by multiple receptor tyrosine kinases (RTKs) rather than a single RTK; and secondary gene mutations (see, e.g., Liu et al., Cancer Gene Therapy 2022, 29:875-878).

Unfortunately, there are very few reports on inhibitor compounds involving KRAS^{G12D} primarily due to the chemical challenge of effectively binding to the specific amino acid residues at the mutation site. Some KRAS^{G12D} inhibitors have been disclosed in International (PCT) Application Publication Nos. WO2021041671 and WO2021106231. However, their clinical use is limited. There is a need for new KRAS^{G12D} inhibitors with higher activity and improved therapeutic effect for clinical use.

### SUMMARY

The present disclosure relates to KRAS^{G12D} inhibitor compounds, compositions thereof, and methods of use thereof for inhibiting, treating or preventing a a KRAS-G12D-associated disease, disorder or condition such as hyperproliferative disorder. Specifically, the disclosure provides KRAS^{G12D} inhibitor compounds having the structure shown in Formula (A), as well as a pharmaceutically acceptable salts, esters, hydrates, solvates or stereoisomers thereof. As reported hereinbelow, inhibitor compounds of the disclosure demonstrate favorable anti-tumor activity and are useful therapeutically for treatment or prevention of KRAS^{G12D}-associated cancers and tumors and related conditions.

In a first broad aspect, there are provided compounds of Formula (A) and pharmaceutically acceptable salts, esters, hydrates, solvates or stereoisomers thereof:

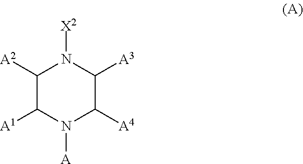

where: X² hydrogen, substituted or unsubstituted alkyl, substituted or unsubstituted heteroalkyl, substituted or unsubstituted acyl (including saturated or unsaturated aliphatic acyl and aroyl), one or more amino acid residues, substituted or unsubstituted oligopeptide (including dipeptide, tripeptide, or tetrapeptide) residues, phosphoryl, phosphonyl, aminophosphonyl, sulfonyl, thioacyl, substituted or unsubstituted benzyl, substituted or unsubstituted alkoxycarbonyl, substituted or unsubstituted aminocarbonyl, substituted or unsubstituted mercaptothiocarbonyl, substituted or unsubstituted alkylthio (including thiocarbonyl), substituted or unsubstituted ester alkyl, substituted or unsubstituted benzyloxycarbonyl, glucoside, glucuronide, or cholic acid substituent;
A refers to an organic group containing a cyclic structure, including a monocyclic ring, a bicyclic ring, a fused ring, a bridged ring, a spiro ring, a heterocyclic ring, an aromatic ring, an aromatic heterocyclic ring, an aliphatic ring, or any combination thereof, wherein the cyclic structure contains two or more substituent groups; and
A¹, A², A³ and A⁴ groups are independently hydrogen or C₁-C₆ short-chain hydrocarbyl; or, alternatively, one or two of the A¹, A², A³ and A⁴ groups can connect to the piperazine ring and form a bridged ring, fused ring, or spiro ring and the remaining (unconnected) A¹, A², A³ and/or A⁴ groups are independently hydrogen or C₁-C₆ short-chain hydrocarbyl.

In some such embodiments, A¹ and A² are connected to the piperazine ring and form a bridged ring, fused ring, or spiro ring, and A³ and A⁴ are independently hydrogen or C₁-C₆ short-chain hydrocarbyl. In other embodiments, A³ and A⁴ are connected to the piperazine ring and form a bridged ring, fused ring, or spiro ring, and A¹ and A² are independently hydrogen or C₁-C₆ short-chain hydrocarbyl. In some such embodiments, A² and A³ are connected to the piperazine ring and form a bridged ring, fused ring, or spiro ring, and A¹ and A⁴ are independently hydrogen or C₁-C₆ short-chain hydrocarbyl. In some such embodiments, A¹ and A⁴ are connected to the piperazine ring and form a bridged ring, fused ring, or spiro ring, and A² and A³ are independently hydrogen or C₁-C₆ short-chain hydrocarbyl.

In some such embodiments, A² and A³ are connected to the piperazine ring and form a bridged ring, fused ring, or spiro ring, and A¹ and A⁴ are hydrogen. A² and A³ are connected to the piperazine ring and form a bridged ring, and A¹ and A⁴ are independently hydrogen.

In some embodiments, the compound represented by Formula (A) is a compound represented by Formula (B) or a pharmaceutically acceptable salt, ester, hydrate, solvate, or stereoisomer thereof:

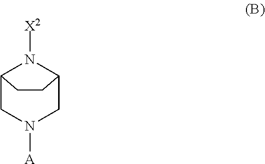

where: X² is hydrogen, substituted or unsubstituted alkyl, substituted or unsubstituted heteroalkyl, substituted or unsubstituted acyl (including saturated or unsaturated aliphatic acyl and aroyl), one or more amino acid residues, substituted or unsubstituted oligopeptide (including dipeptide, tripeptide, or tetrapeptide) residues, phosphoryl, phosphonyl, aminophosphonyl, sulfonyl, thioacyl, substituted or unsubstituted benzyl, substituted or unsubstituted alkoxycarbonyl, substituted or unsubstituted aminocarbonyl, substituted or unsubstituted mercaptothiocarbonyl, substituted or unsubstituted alkylthio (including thiocarbonyl), substituted or unsubstituted ester alkyl, substituted or unsubstituted benzyloxycarbonyl, glucoside, glucuronide, or cholic acid substituent; and
A refers to an organic group containing a cyclic structure, including a monocyclic ring, a bicyclic ring, a fused ring, a bridged ring, a spiro ring, a heterocyclic ring, an aromatic ring, a heteroaromatic ring, an aliphatic ring, and any combination thereof, wherein the cyclic structure contains two or more substituent groups.

In some embodiments of Formula (A) and Formula (B), X² is hydrogen, C₁-C₂₀ saturated or unsaturated alkoxycarbonyl, C₁-C₂₀ saturated or unsaturated alkyl acyl, 6- to 15-membered (hetero) arylcarbonyl, 4- to 15-membered (hetero) cycloalkylcarbonyl, C₁-C₂₀ alkylthio,

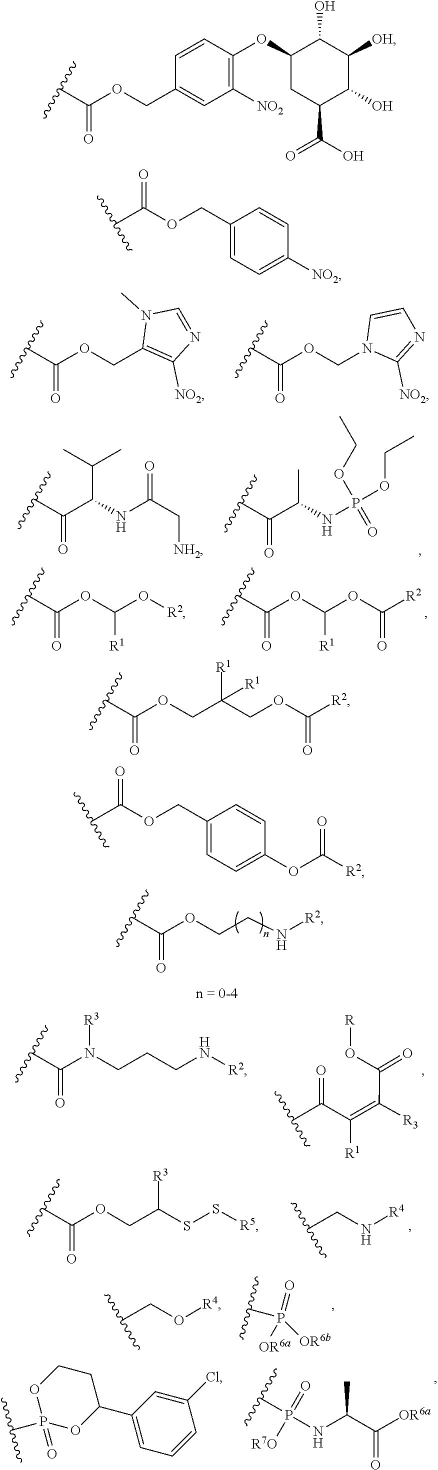

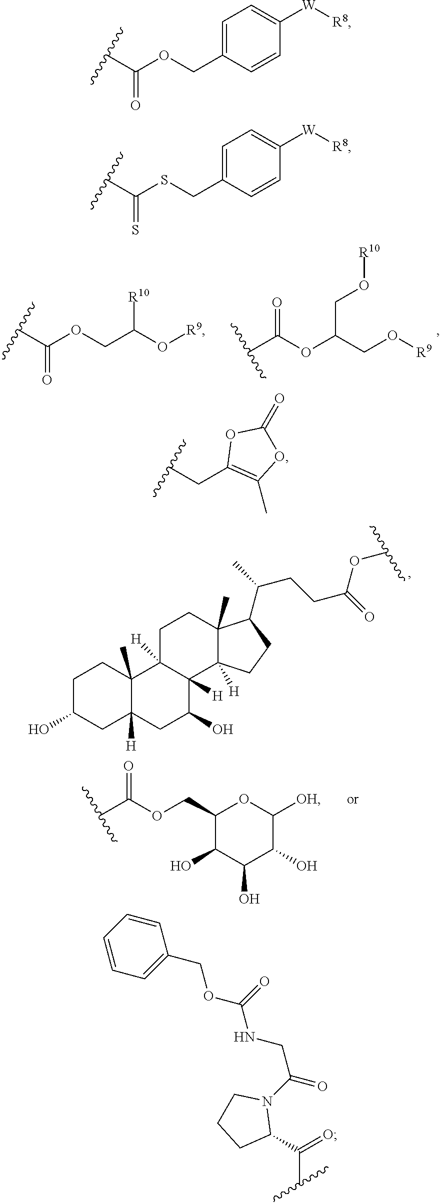

wherein, R¹ is hydrogen, methyl, ethyl, propyl, isopropyl, C₃-C₆ cycloalkyl, or aryl; R² is hydrogen, C₁-C₂₀ saturated or unsaturated alkyl, C₁-C₂₀ saturated or unsaturated alkyl acyl, heteroazanyl, aryl hydrocarbyl, heterocyclic aromatic hydrocarbyl, C₃-C₈ carbocyclic or heterocyclic hydrocarbyl, fused ring, naphthalene ring, bridged ring hydrocarbyl, one or more amino acid residues,

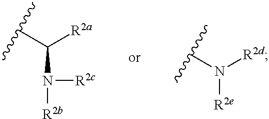

wherein, R^{2a}, R^{2b}, R^{2c}, R^{2d} and R^{2e} are independently hydrogen, C₁-C₆ substituted or unsubstituted alkyl or C₁-C₆ substituted or unsubstituted hydrocarbyl; R³ is hydrogen, methyl, ethyl or propyl; R⁴ is hydrogen, C₂-C₂₀ alkyl, isopropyl, isobutyl, aryl hydrocarbyl, carbocyclic hydrocarbyl, heterocyclic hydrocarbyl, or C₂-C₂₀ alkanoyloxy; R⁵ is ethyl with a 2-position substituted, wherein the substituents at the 2-position are amino, alkoxycarbonyl, alkanoyloxy, or acyloxy derived from amino acid; R^{6a} and R^{6b} are independently hydrogen, C₁-C₂₀ hydrocarbyl, C₁-C₂₀ cyclic hydrocarbyl, aryl, or

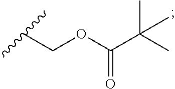 [SMILES: *COC(=O)C(C)(C)C]

R⁷ is C₁-C₆ alkyl or substituted or unsubstituted aryl; R⁸ is C₂-C₂₀ substituted or unsubstituted saturated or unsaturated alkanoyl or saturated or unsaturated alkoxycarbonyl; R⁹ is lower alkyl, substituted or unsubstituted benzyl, substituted or unsubstituted imidazol-5-methyl, oligoglycolyl (—[CH₂CH₂O]ₙCH₃, where n is an integer from 0 to 4), or C₂-C₂₀ saturated or unsaturated alkanoyl; and R¹⁰ is hydrogen, C₁-C₆ alkoxy, C₂-C₂₀ saturated or unsaturated alkanoyl, C₂-C₂₀ substituted or unsubstituted saturated or unsaturated alkanoyl, or saturated or unsaturated alkoxycarbonyl.

In some embodiments of Formula (A) and (B), X² is

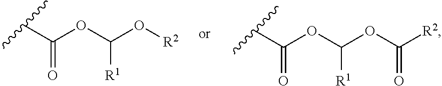

wherein R¹ is methyl, and R² is C₁-C₂₀ saturated or unsaturated alkyl or C₁-C₂₀ saturated or unsaturated alkyl acyl. In some such embodiments, R² is C₁-C₆ saturated or unsaturated alkyl or C₁-C₆ saturated or unsaturated alkyl acyl. In some such embodiments, R² is C₁-C₆ saturated alkyl or C₁-C₆ saturated alkyl acyl. In some such embodiments, R² is C₃ alkyl or C₃ alkyl acyl.

In some embodiments, the compound represented by Formula (A) or Formula (B) is a compound represented by Formula (I) or a pharmaceutically acceptable salt, ester, hydrate, solvate or stereoisomer thereof:

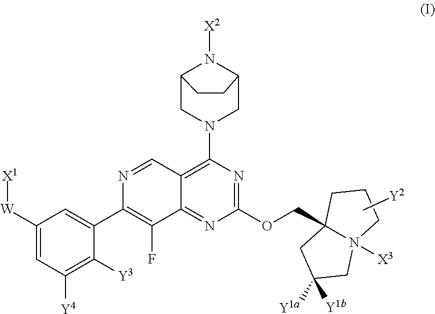

where: W is oxygen (O), sulfur (S) or nitrogen (NH); and
X¹ and X² are independently hydrogen, substituted or unsubstituted alkyl, substituted or unsubstituted heteroalkyl, substituted or unsubstituted acyl (including saturated or unsaturated aliphatic acyl and aroyl), one or more amino acid residues, substituted or unsubstituted oligopeptide (including dipeptide, tripeptide, tetrapeptide) residues, phosphoryl, phosphonyl, aminophosphonyl, sulfonyl, thioacyl, substituted or unsubstituted benzyl, substituted or unsubstituted alkoxycarbonyl, substituted or unsubstituted aminocarbonyl, substituted or unsubstituted mercaptothiocarbonyl, substituted or unsubstituted alkylthio (including thiocarbonyl), substituted or unsubstituted ester alkyl, substituted or unsubstituted benzyloxycarbonyl, glucoside, glucuronide, or cholic acid substituent;
In some embodiments of compounds of Formula (I), X¹ and X² are independently substituted or unsubstituted alkyl, substituted or unsubstituted acyl, substituted or unsubstituted heteroalkyl, or cholic acid substituent, and X³ is

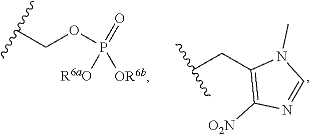

or lone pair electrons, wherein, when X³ is lone pair electrons, X¹ and X² cannot both be hydrogen, and when X³ is

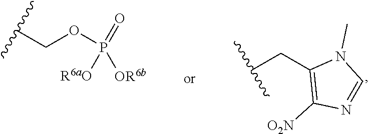

an N atom links to X³ and forms a quaternary ammonium ion with a positive charge which further forms an inner salt with an intramolecular negative ion or a salt with an additional acid molecule (including but not limited to hydrohalic acid salt), wherein R^{6a} and R^{6b} are independently hydrogen, C₁-C₂₀ hydrocarbyl (optionally, C₁-C₆ hydrocarbyl), C₁-C₂₀ cyclohydrocarbyl (optionally, C₁-C₆ cyclohydrocarbyl), or

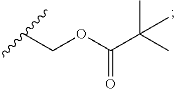 [SMILES: *COC(=O)C(C)(C)C]

Y^{1a} and Y^{1b} are independently hydrogen, halogen (including F, Cl, or Br), hydroxy, amino, amine, hydroxymethyl, alkoxy, or acyloxy;

Y² is hydrogen, halogen, hydroxy, amino, amine, hydroxymethyl, alkoxy, acyloxy, or lower (e.g., C₁-C₆, C₁, C₂, C₃, C₄, C₅, or C₆) hydrocarbyl; and

Y³ and Y⁴ are independently hydrogen (H), halogen, halomethyl (including monohalomethyl, dihalomethyl, and trihalomethyl); or, Y³ and Y⁴ are connected to the benzene ring structure and form a substituted or unsubstituted benzo fused ring (including but not limited to a naphthalene ring structure).

In some embodiments of compounds of Formula (I), X¹ and X² are independently hydrogen, C₁-C₂₀ saturated or unsaturated alkoxycarbonyl (optionally, C₁-C₄, C₁, C₂, C₃, or C₄ alkoxycarbonyl), C₁-C₂₀ saturated or unsaturated alkyl acyl (optionally, C₁-C₆, C₁, C₂, C₃, C₄, C₅, or C₆ alkylacyl), 6- to 15-membered (hetero) arylcarbonyl (for example and without limitation, phenylcarbonyl, naphthylcarbonyl, pyridylcarbonyl), 4- to 15-membered (hetero) cycloalkylcarbonyl (for example and without limitation, cyclohexylcarbonyl, tetrahydropyranylcarbonyl), C₁-C₂₀ alkylthio (optionally, C₁-C₆, C₁, C₂, C₃, C₄, C₅, or C₆ alkylthio),

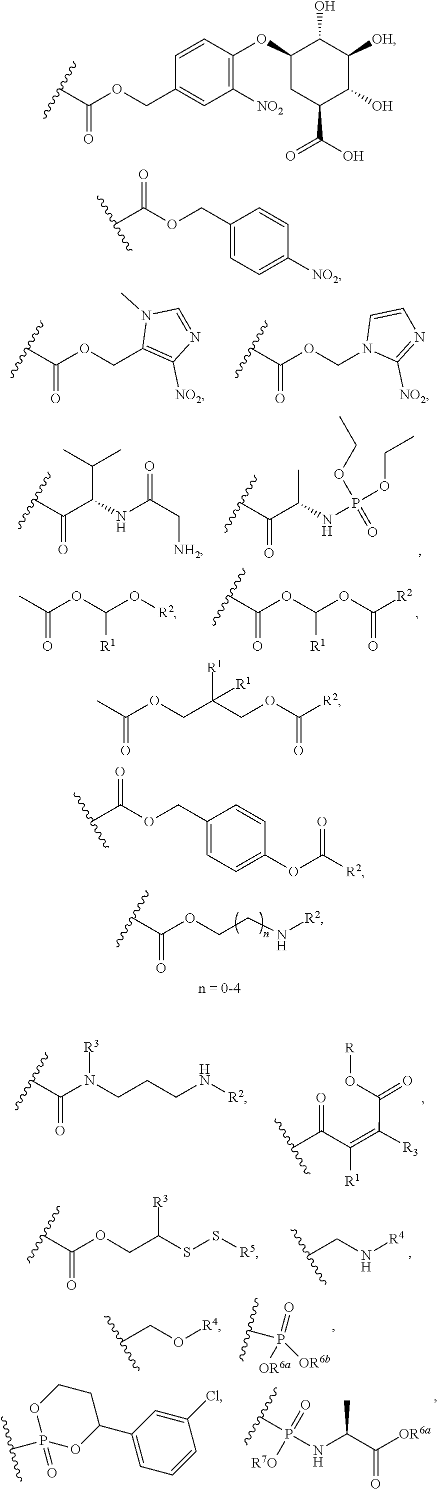

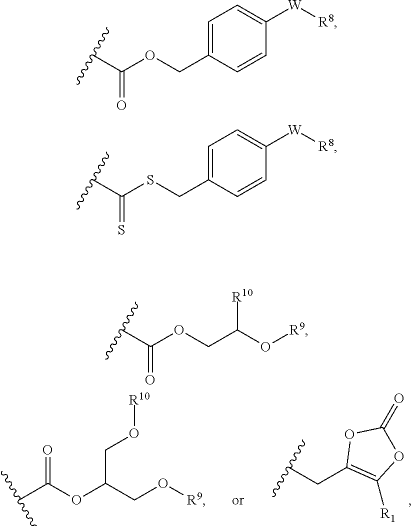

wherein: R¹ is hydrogen, methyl, ethyl, propyl, isopropyl, C₃-C₆ cycloalkyl (optionally, C₃, C₄, C₅, or C₆ cycloalkyl), aryl (for example and without limitation, substituted or unsubstituted phenyl, substituted or unsubstituted naphthyl);

R² is hydrogen, C₁-C₂₀ saturated or unsaturated alkyl (optionally, lower aliphatic hydrocarbyl, i.e., C₁-C₆, C₁, C₂, C₃, C₄, C₅, or C₆ aliphatic hydrocarbyl), C₁-C₂₀ saturated or unsaturated alkyl acyl (optionally, lower aliphatic acyl, i.e., C₁-C₆, C₁, C₂, C₃, C₄, C₅, or C₆ acyl), heteroazanyl, aryl hydrocarbyl, heterocyclic aromatic hydrocarbyl, C₃-C₈ carbocyclic or heterocyclic hydrocarbyl (optionally, C₅, C₆, C₇, or C₈ carbocyclic or heterocyclic hydrocarbyl), fused ring, naphthalene ring, bridged ring hydrocarbyl, or one or more amino acid residue(s), wherein the amino acid residue(s) may be natural or unnatural and hydrogen (H) at any position on the amino acid residue(s) may be substituted or unsubstituted. In some such embodiments of compounds of Formula (I), R² is

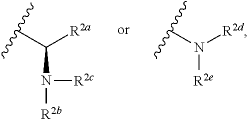

wherein: R^{2a}, R^{2b}, R^{2c}, R^{2d} and R^{2e} are independently hydrogen, C₁-C₆ substituted or unsubstituted alkyl (optionally, C₁, C₂, C₃, C₄, C₅, or C₆), C₁-C₆ substituted or unsubstituted hydrocarbyl (optionally, C₁, C₂, C₃, C₄, C₅, or C₆). In some embodiments, R^{2a} is hydrogen, methyl, ethyl, propyl, isopropyl, 2-isobutyl, 3-isobutyl, or phenylmethyl; R^{2b} and R^{2c} are independently hydrogen, C₁-C₆ substituted or unsubstituted alkyl (optionally, C₁, C₂, C₃, C₄, C₅, or C₆), C₁-C₆ substituted or unsubstituted hydrocarbyl (optionally, C₁, C₂, C₃, C₄, C₅, or C₆); and R^{2d} and R^{2e} are independently hydrogen or C₁-C₆ alkyl (optionally, C₁, C₂, C₃, C₄, C₅, or C₆ alkyl);

R³ is hydrogen, methyl, ethyl or propyl;

R⁴ is hydrogen, C₂-C₂₀ alkyl, isopropyl, isobutyl, aryl hydrocarbyl, carbocyclic or heterocyclic hydrocarbyl (optionally, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀), C₂-C₂₀ alkanoyloxy (optionally, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀);

R⁵ is ethyl with a 2-position substituted, wherein substituents at the 2-position include but are not limited to amino, alkoxycarbonyl, alkanoyloxy, and acyloxy derived from amino acid;

R^{6a} and R^{6b} are independently hydrogen, C₁-C₂₀ hydrocarbyl (optionally, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀), or C₁-C₂₀ cyclic hydrocarbyl (optionally, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀), aryl,

or

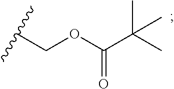 [SMILES: *COC(=O)C(C)(C)C]

R⁷ is lower alkyl (e.g., C₁-C₆, C₁, C₂, C₃, C₄, C₅, or C₆ alkyl) or substituted or unsubstituted aryl;

R⁸ is C₂-C₂₀ substituted or unsubstituted saturated or unsaturated alkanoyl or saturated or C₂-C₂₀ substituted or unsubstituted unsaturated alkoxycarbonyl;

R⁹ is lower alkyl (e.g., C₁-C₆, C₁, C₂, C₃, C₄, C₅, or C₆ alkyl), substituted or unsubstituted benzyl, substituted or unsubstituted imidazol-5-methyl, oligoglycolyl (—[CH₂CH₂O]ₙCH₃, wherein n is an integer from 0 to 4, or 1, 2, 3, or 4), C₂-C₂₀ saturated or unsaturated alkanoyl (optionally, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀);

R¹⁰ is hydrogen, C₁-C₆ alkoxy (optionally, C₁, C₂, C₃, C₄, C₅, or C₆), C₂-C₂₀ saturated or unsaturated alkanoyl (optionally, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀), C₂-C₂₀ substituted or unsubstituted saturated or unsaturated alkanoyl (optionally, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀), or C₂-C₂₀ substituted or unsubstituted saturated or unsaturated alkoxycarbonyl (optionally, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀); and

n is an integer of 0 to 4, optionally 1, 2, 3, or 4.

In some embodiments of compounds of Formula (I), X¹ and X² are independently

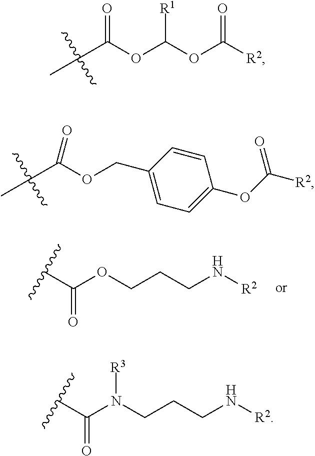

In some embodiments of compounds of Formula (I), X¹ and X² are independently hydrogen,

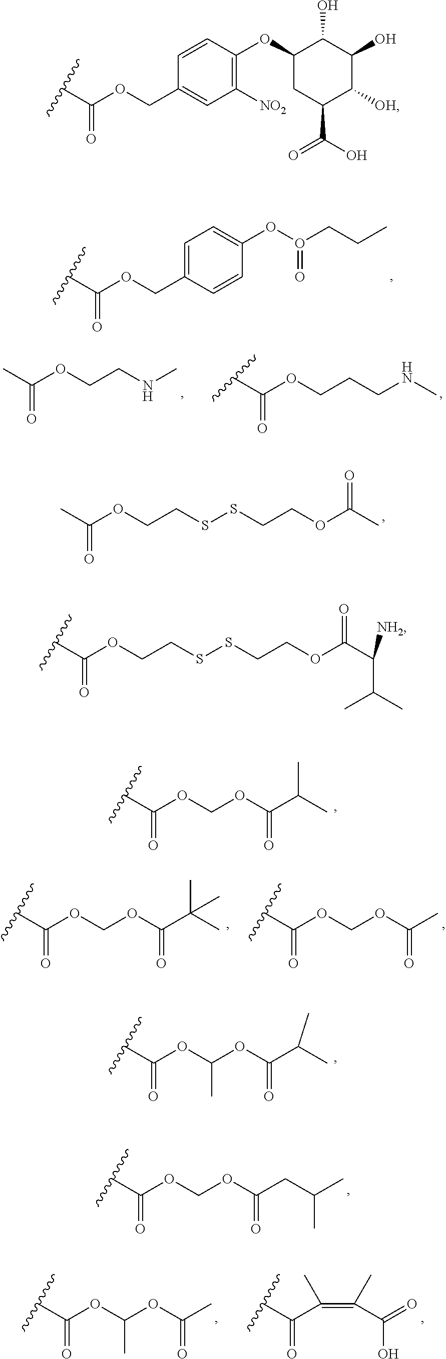

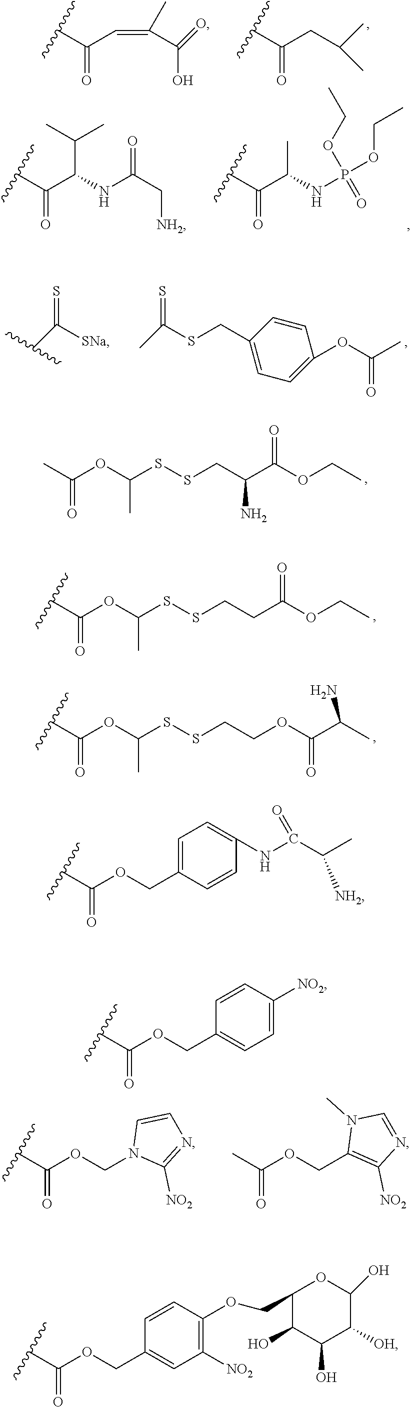

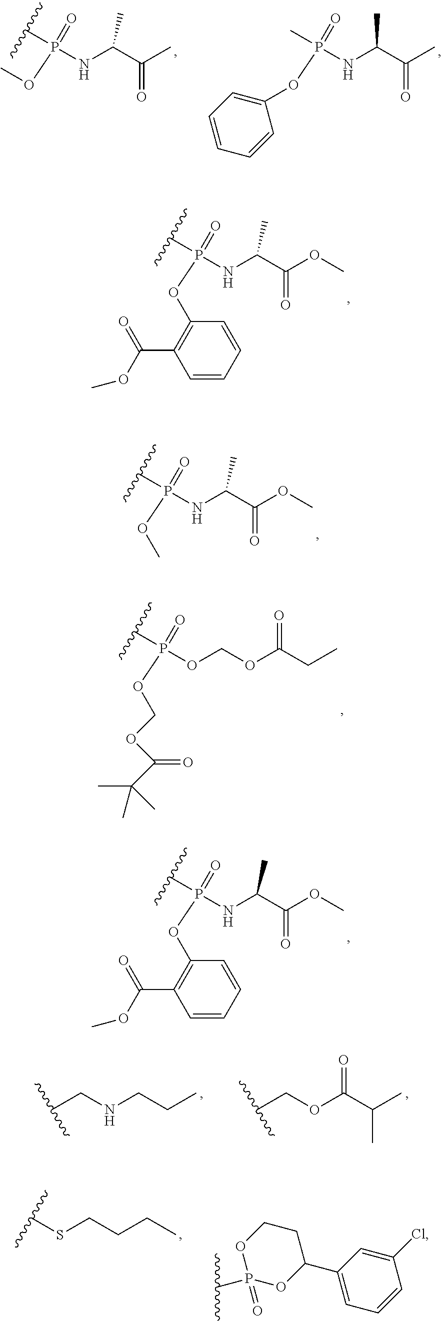

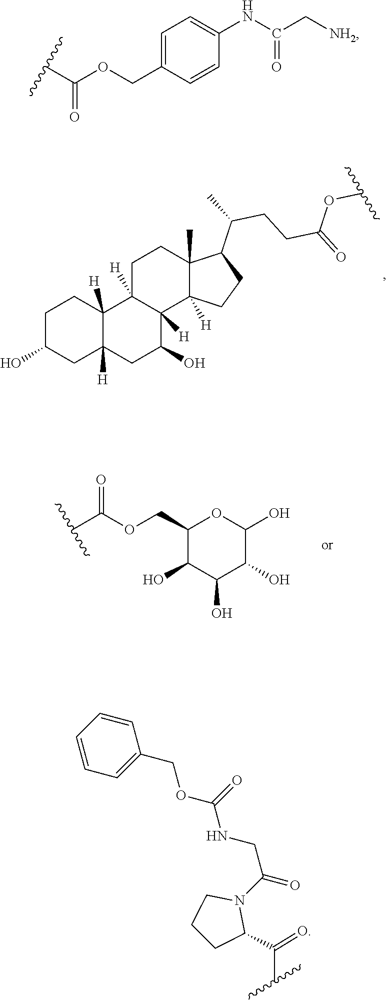 [SMILES: *C(=O)OCc1ccc(NC(=O)CN)cc1 ; *OC(=O)CC[C@@H](C)[C@H]1CC[C@H]2[C@H]3[C@H](CC[C@]12C)[C@H]1CC[C@@H](O)C[C@H]1C[C@@H]3O ; *C(=O)OC[C@H]1OC(O)[C@H](O)[C@@H](O)[C@H]1O ; *C(=O)[C@@H]1CCCN1C(=O)CNC(=O)OCc1ccccc1]

In some embodiments of compounds of Formula (I), X¹ and X² are independently

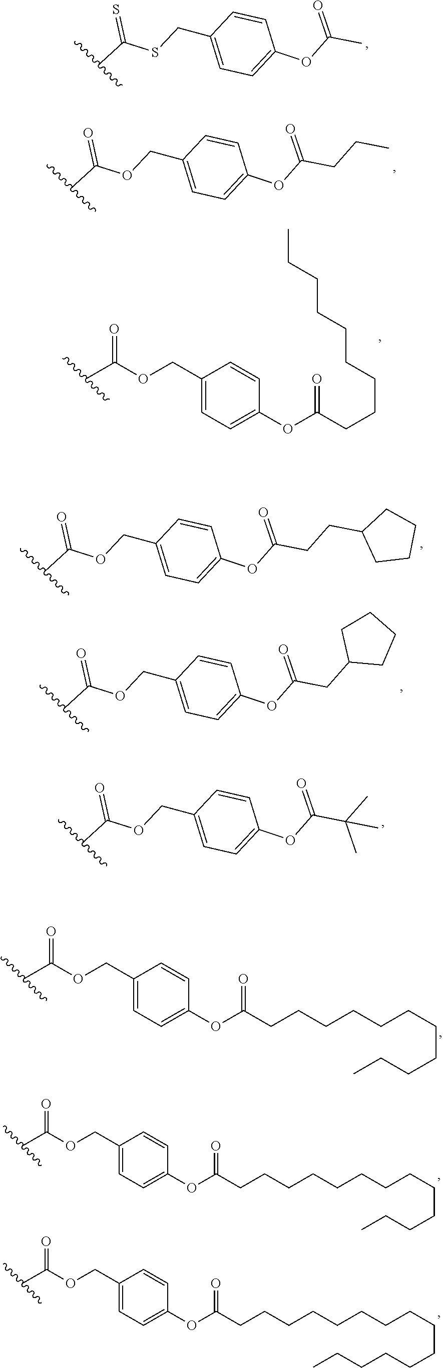

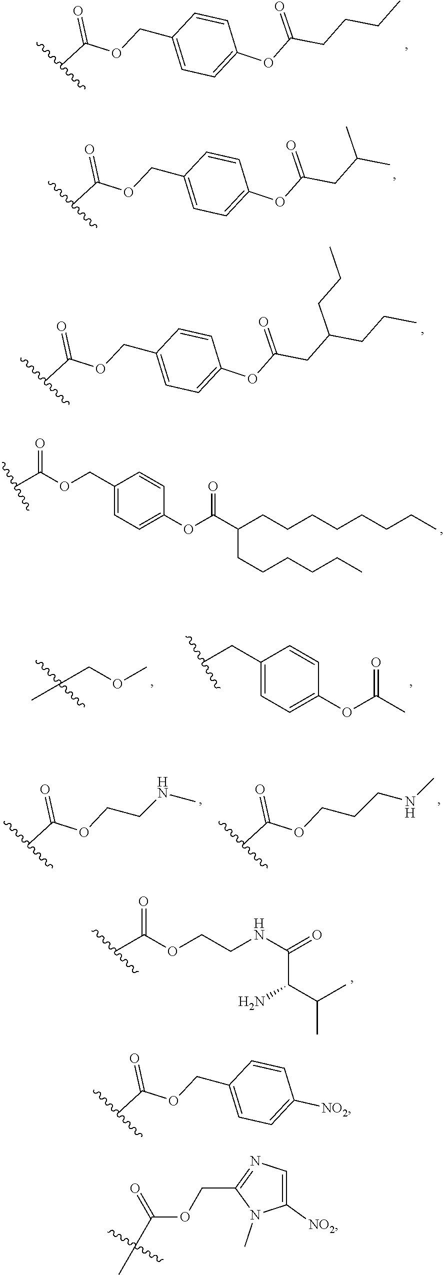

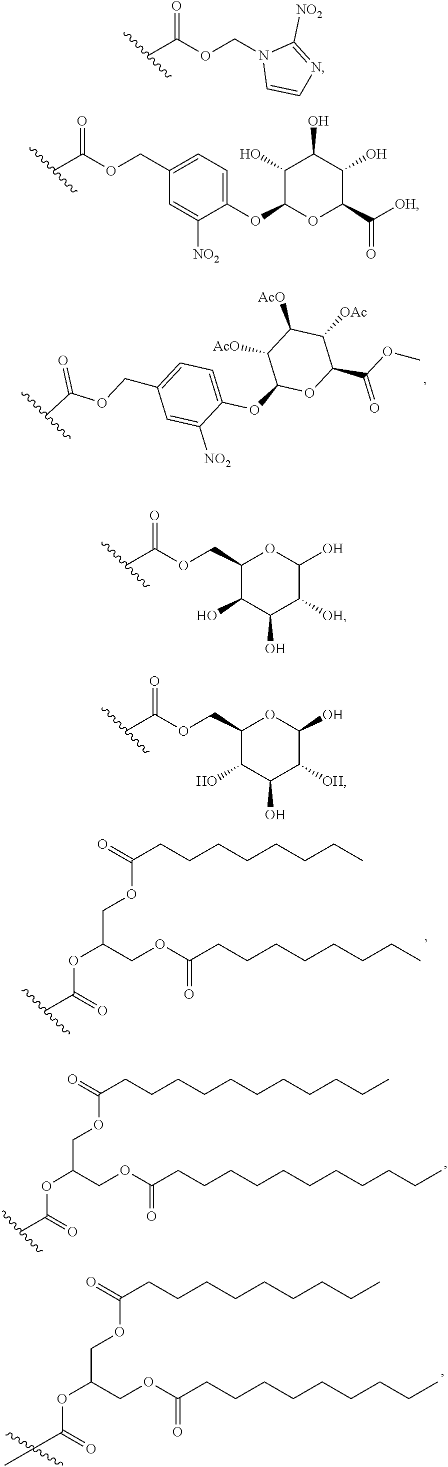

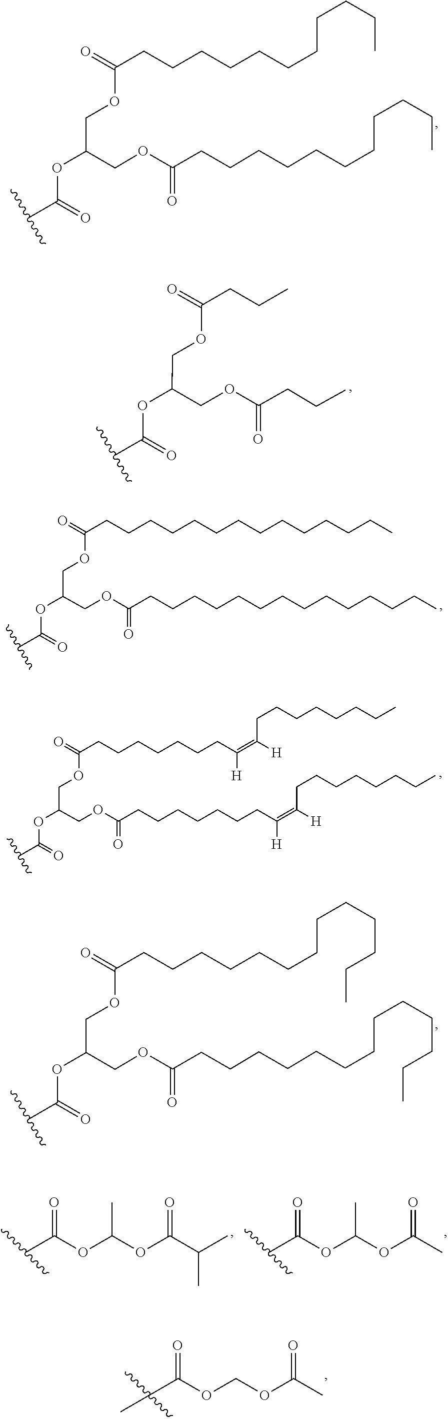

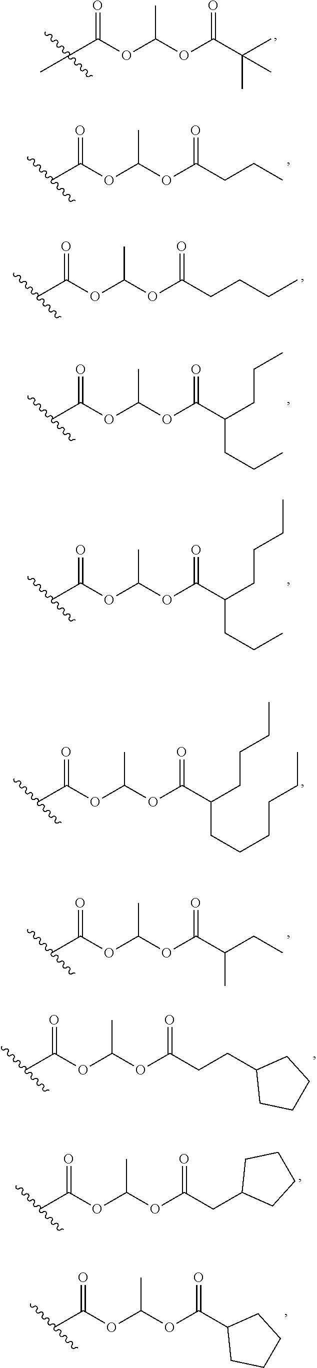

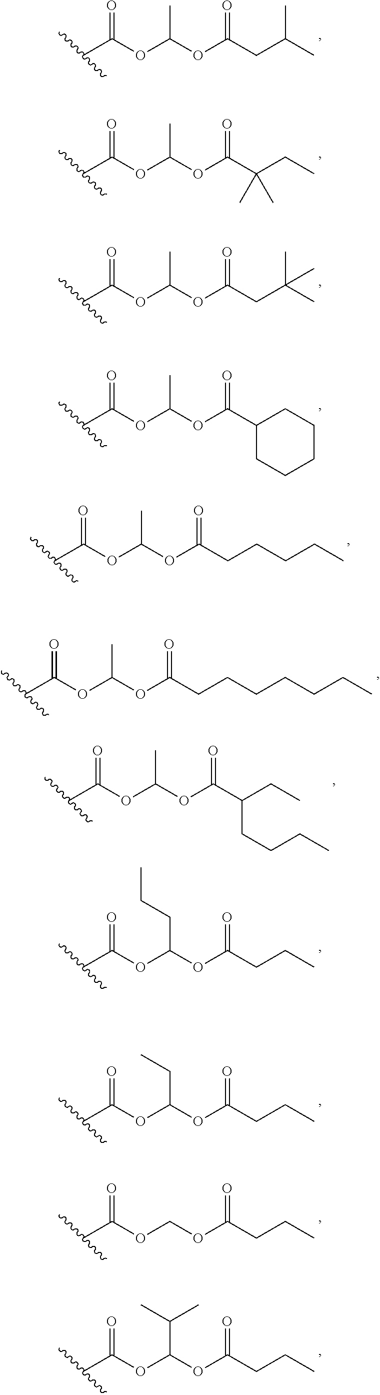

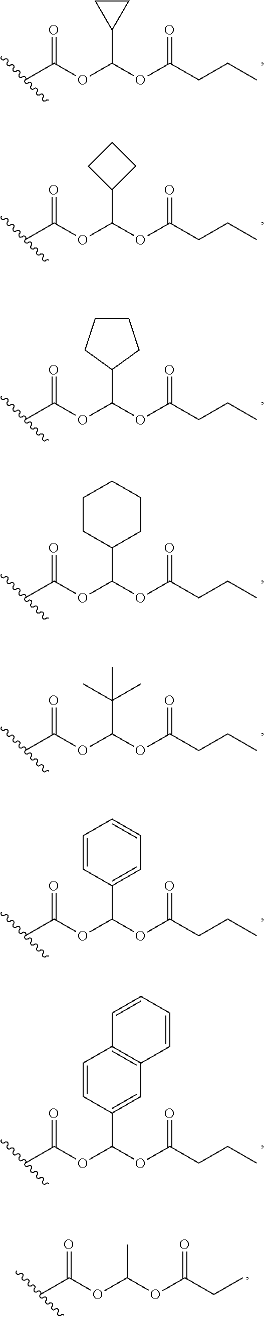

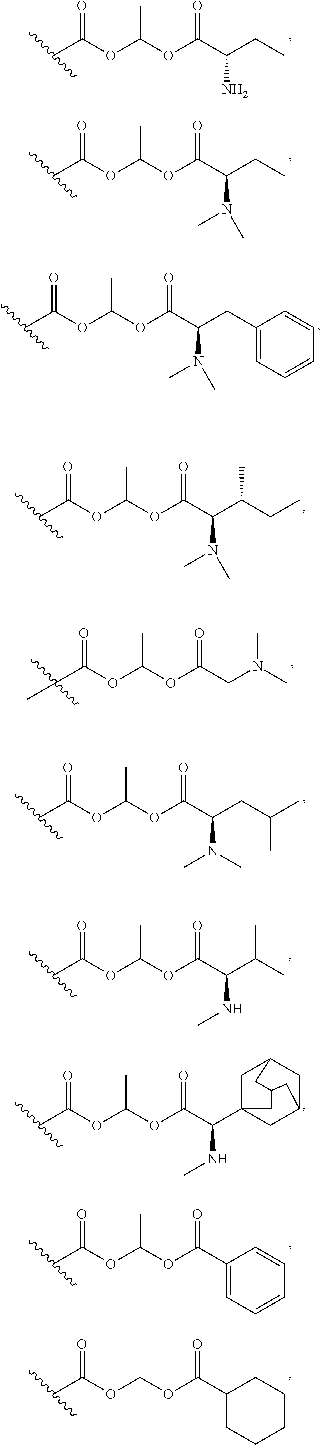

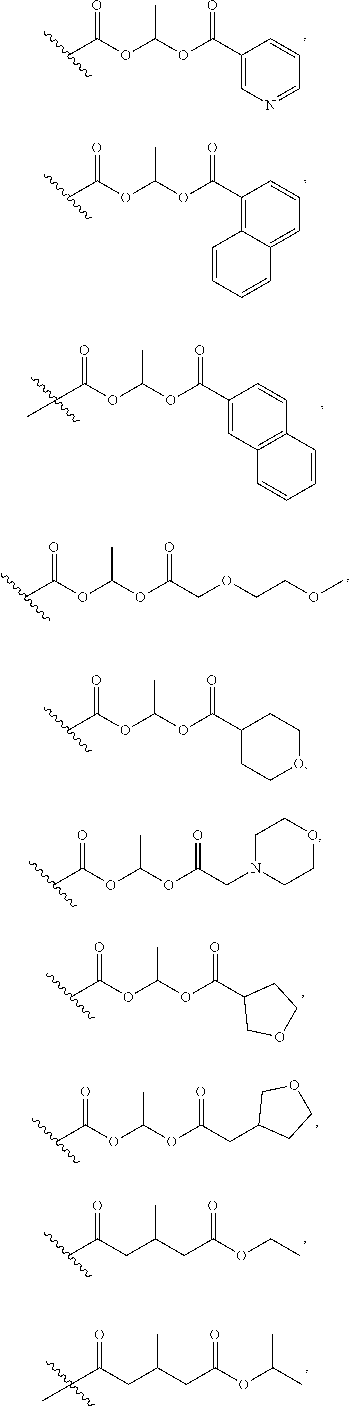

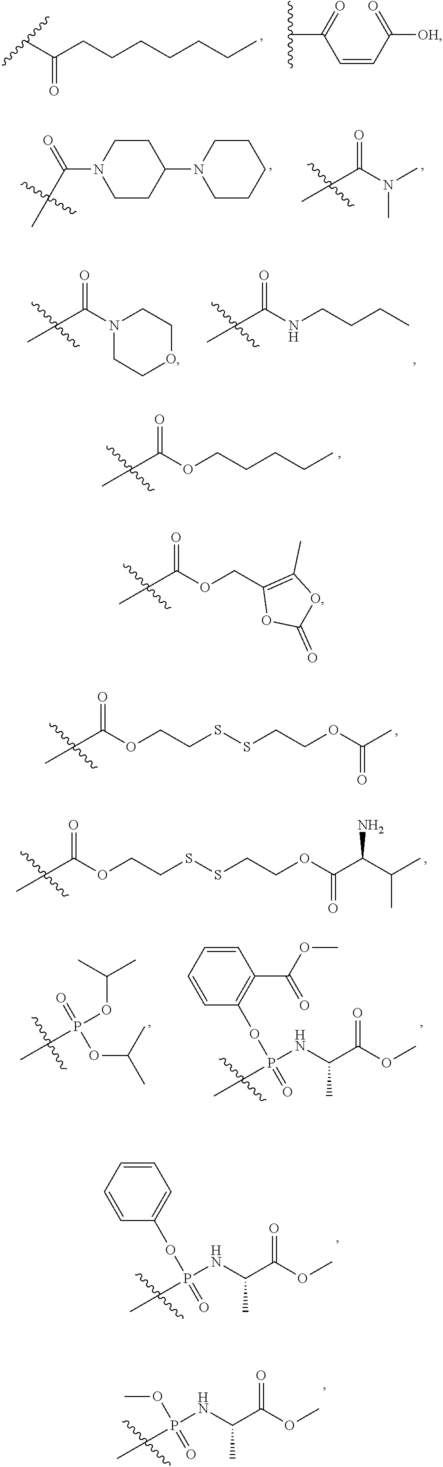

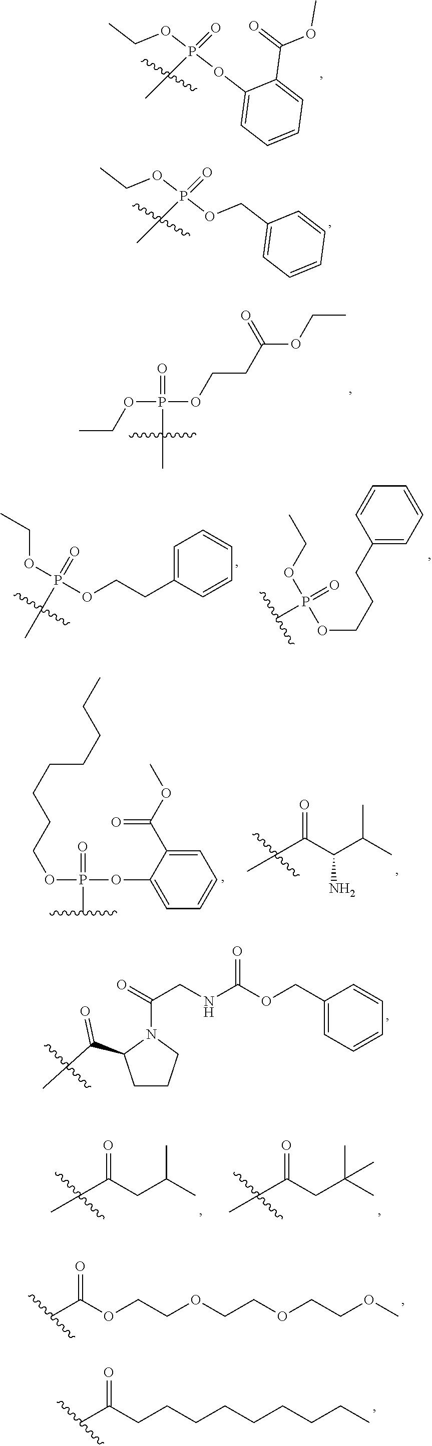

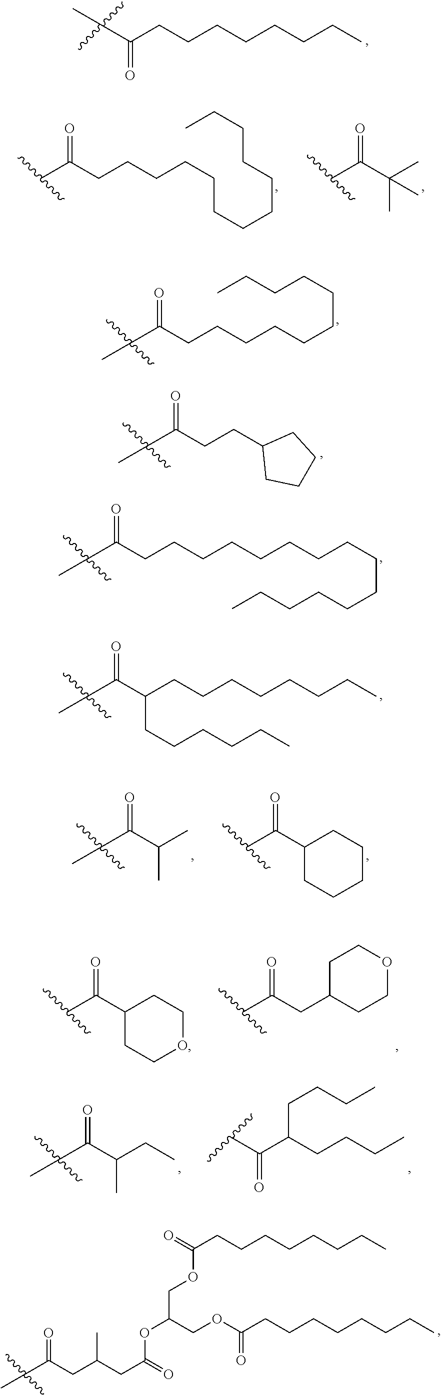

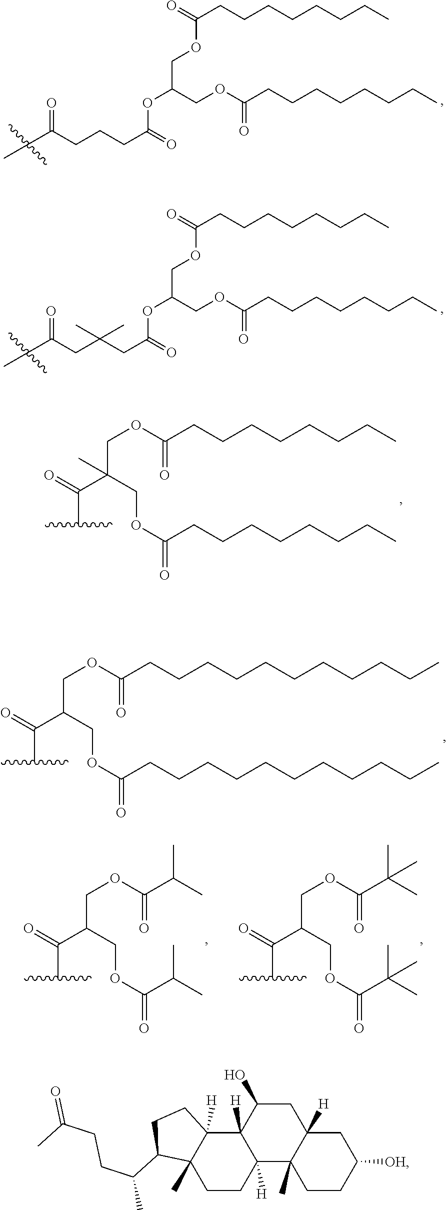

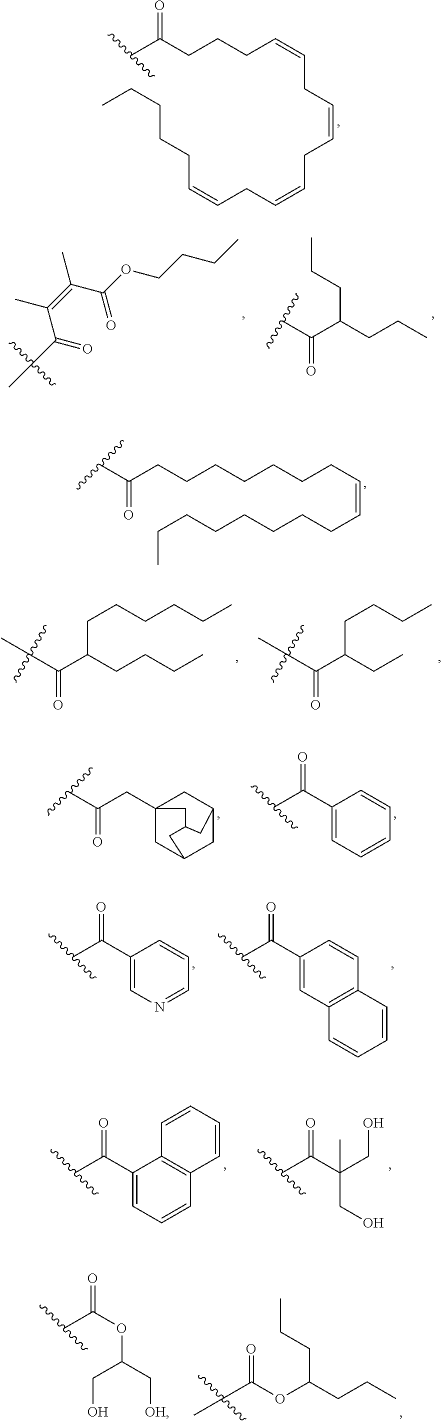

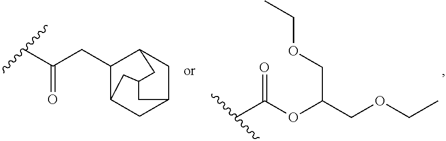 [SMILES: *C(=O)CC1C2CC3CC(C2)CC1C3 ; *C(=O)OC(COCC)COCC]

In some embodiments of compounds of Formula (I), X¹ and X² are independently hydrogen or

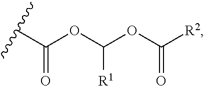

wherein R¹ is methyl, propyl, isopropyl, or cyclohexyl, and R² is C₁-C₂₀ saturated or unsaturated alkyl (optionally, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀), pyridyl, phenyl, or naphthyl, etc. In some such embodiments, R¹ is methyl. In some such embodiments, R² is

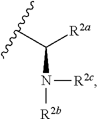

wherein R^{2a} is hydrogen, methyl, ethyl, propyl, isopropyl, 2-isobutyl, 3-isobutyl, or aryl, and R^{2b} and R^{2c} are independently hydrogen, C₁-C₆ substituted or unsubstituted alkyl, or C₁-C₆ substituted or unsubstituted hydrocarbyl.

In some embodiments of compounds of Formula (I), X¹ is hydrogen and X² is

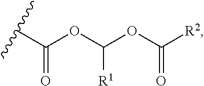

wherein R¹ is methyl, propyl, isopropyl, or cyclohexyl, and R² is C₁-C₂₀ saturated or unsaturated alkyl (optionally, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀), pyridyl, phenyl, or naphthyl, etc. In some such embodiments, R¹ is methyl. In some such embodiments, R² is

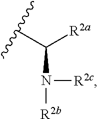

wherein R^{2a} is hydrogen, methyl, ethyl, propyl, isopropyl, 2-isobutyl, 3-isobutyl, or aryl, and R^{2b} and R^{2c} are independently hydrogen, C₁-C₆ substituted or unsubstituted alkyl, or C₁-C₆ substituted or unsubstituted hydrocarbyl.

In some embodiments of compounds of Formula (I), X¹ is C₁-C₂₀ saturated or unsaturated alkyl, C₁-C₂₀ saturated or unsaturated acyl, C₁-C₂₀ saturated or unsaturated pyridyl, C₁-C₂₀ saturated or unsaturated phenyl, or C₁-C₂₀ saturated or unsaturated naphthyl, and X² is hydrogen.

In some embodiments of compounds of Formula (I), X² or X¹ is

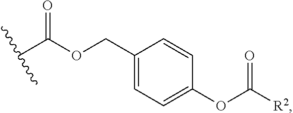

wherein R² is C₁-C₂₀ saturated or unsaturated alkyl (optionally, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀).

In some embodiments of compounds of Formula (I), Y^{1a}, Y^{1b} and Y² are independently hydrogen or halogen (including F, Cl, or Br). In some embodiments of compounds of Formula (I), Y^{1a}, Y^{1b} and Y² are independently hydrogen or F.

In some embodiments of compounds of Formula (I), X³ is

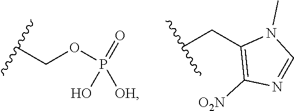 [SMILES: *COP(=O)(O)O ; *Cc1c([N+](=O)[O-])ncn1C]

or lone pair electrons.

In some embodiments of compounds of Formula (I), Y³ and Y⁴ are independently H, Cl, or CF₃, or Y³ and Y⁴ are connected to phenyl ring structures and form one or more substituted or unsubstituted naphthalene ring (for example and without limitation

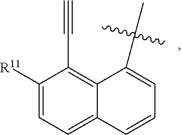

wherein R¹¹ is hydrogen, halogen (including F, Cl, or Br), hydroxyl, substituted hydroxyl, or lower alkyl, and Y⁴ is hydrogen, halogen, hydroxyl, substituted hydroxyl, or lower alkyl.

In some embodiments of compounds of Formula (I), W is oxygen (O).

In some embodiments of compounds of Formula (I), X¹ is hydrogen (H).

In some embodiments of compounds of Formula (I), X¹ is

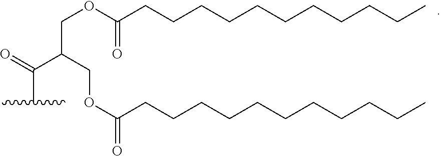 [SMILES: *C(=O)C(COC(=O)CCCCCCCCCCC)COC(=O)CCCCCCCCCCC]

In some embodiments of compounds of Formula (I), X² is

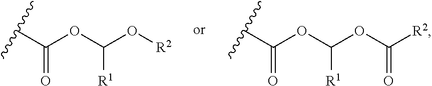

wherein R¹ is methyl, and R² is C₁-C₂₀ saturated or unsaturated alkyl or C₁-C₂₀ saturated or unsaturated alkyl acyl. In some such embodiments, R² is C₁-C₆ saturated or unsaturated alkyl or C₁-C₆ saturated or unsaturated alkyl acyl. In some such embodiments, R² is C₁-C₆ saturated alkyl or C₁-C₆ saturated alkyl acyl, e.g., C₁, C₂, C₃, C₄, C₅ or C₆ alkyl or acyl.

In some embodiments of compounds of Formula (I), X¹ is

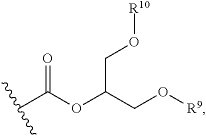

wherein R⁹ is lower alkyl, substituted or unsubstituted benzyl, substituted or unsubstituted imidazol-5-methyl, oligoglycolyl (—[CH₂CH₂O]ₙCH₃, where n is an integer from 0 to 4), or C₂-C₂₀ saturated or unsaturated alkanoyl; and R¹⁰ is hydrogen, C₁-C₆ alkoxy, C₂-C₂₀ saturated or unsaturated alkanoyl, C₂-C₂₀ substituted or unsubstituted saturated or unsaturated alkanoyl, or saturated or unsaturated alkoxycarbonyl. In some such embodiments, R⁹ and R¹⁰ are independently C₂-C₂₀ substituted or unsubstituted saturated or unsaturated alkanoyl, e.g., C₂-C₂₀ unsubstituted saturated or unsaturated alkanoyl, e.g., C₂-C₂₀ unsubstituted, saturated alkanoyl. In some such embodiments, W is O.

In some embodiments of compounds of Formula (I), the halogen is fluorine (F).

In some embodiments of compounds of Formula (I), W is O and X¹ is hydrogen (H).

In some embodiments of compounds of Formula (I), W is O and X¹ is

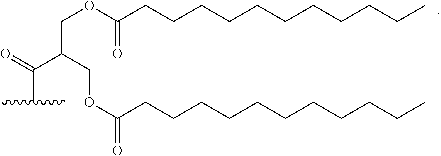 [SMILES: *C(=O)C(COC(=O)CCCCCCCCCCC)COC(=O)CCCCCCCCCCC]

In some embodiments, the compound of the disclosure is a compound represented by Formula (II), (IIA), or (III), or a pharmaceutically acceptable salt, ester, hydrate, solvate, or stereoisomer thereof:

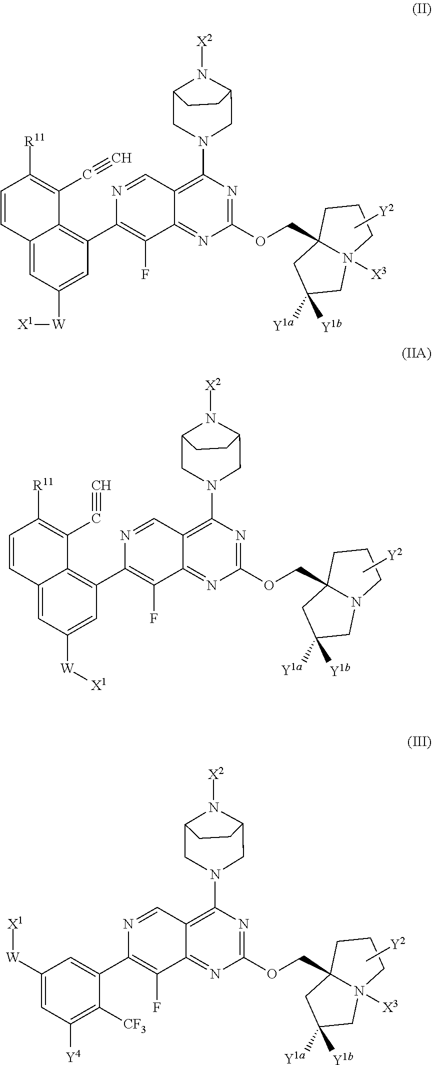

where: R¹¹ is hydrogen, halogen, hydroxy, substituted hydroxy, or lower alkyl and Y⁴ is hydrogen, halogen, hydroxyl, substituted hydroxyl, or lower alkyl; and the other substituents are as defined above.

In some embodiments of compounds of Formula (II), (IIA) or (III), W is oxygen (O). In some such embodiments, X¹ is hydrogen.

In some embodiments of compounds of Formula (II), (IIA) or (III), X¹ is hydrogen.

In some embodiments of compounds of Formula (II), (IIA) or (III), R¹¹ is hydrogen.

In some embodiments of compounds of Formula (II), (IIA) or (III), R¹¹ is halogen (including F, Cl or Br). In some such embodiments, R¹¹ is F.

In some embodiments of compounds of Formula (II), (IIA) or (III), Y² is hydrogen, halogen, hydroxy, amino, amine, hydroxymethyl, alkoxy, acyloxy, or C₁-C₆ hydrocarbyl. In some such embodiments, Y² is halogen (including F, Cl or Br). In some such embodiments, Y² is F.

In some embodiments of compounds of Formula (II), (IIA) or (III), Y^{1a} and Y^{1b} are independently hydrogen, halogen (including F, Cl, or Br), hydroxy, amino, amine, hydroxymethyl, alkoxy, or acyloxy. In some such embodiments, Y^{1a} and Y^{1b} are both hydrogen.

In some embodiments of compounds of Formula (II), (IIA) or (III), X³ is

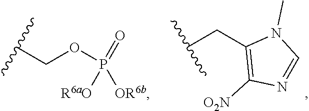

or lone pair electrons. In one embodiment, X³ is lone pair electrons.

In some embodiments of compounds of Formula (II), (IIA) or (III), X² is

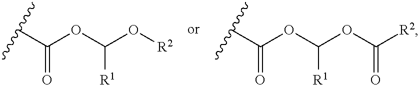

wherein R¹ is methyl, and R² is C₁-C₂₀ saturated or unsaturated alkyl or C₁-C₂₀ saturated or unsaturated alkyl acyl. In some such embodiments, R² is C₁-C₆ saturated or unsaturated alkyl or C₁-C₆ saturated or unsaturated alkyl acyl. In some such embodiments, R² is C₁-C₆ saturated alkyl or C₁-C₆ saturated alkyl acyl, e.g., C₁, C₂, C₃, C₄, C₅ or C₆ alkyl or acyl.

In some embodiments of compounds of Formula (II) or (IIA), W is oxygen and R¹¹ is hydrogen.

In some embodiments of compounds of Formula (II) or (IIA), W is oxygen and R¹¹ is fluorine.

In some embodiments of compounds of Formula (III), W is oxygen and Y⁴ is chlorine.

In some embodiments of compounds of Formula (III), W is NH and Y⁴ is hydrogen.

In some embodiments of compounds of Formula (III), W is NH and Y⁴ is chlorine.

In some such embodiments of compounds of Formula (II), (IIA) or Formula (III), Y^{1b} and Y² are both hydrogen.

In some embodiments, the compound of the disclosure is a compound represented by Formula (IV), (V), (VI) or (VII), or a pharmaceutically acceptable salt, ester, hydrate, solvate, or stereoisomer thereof:

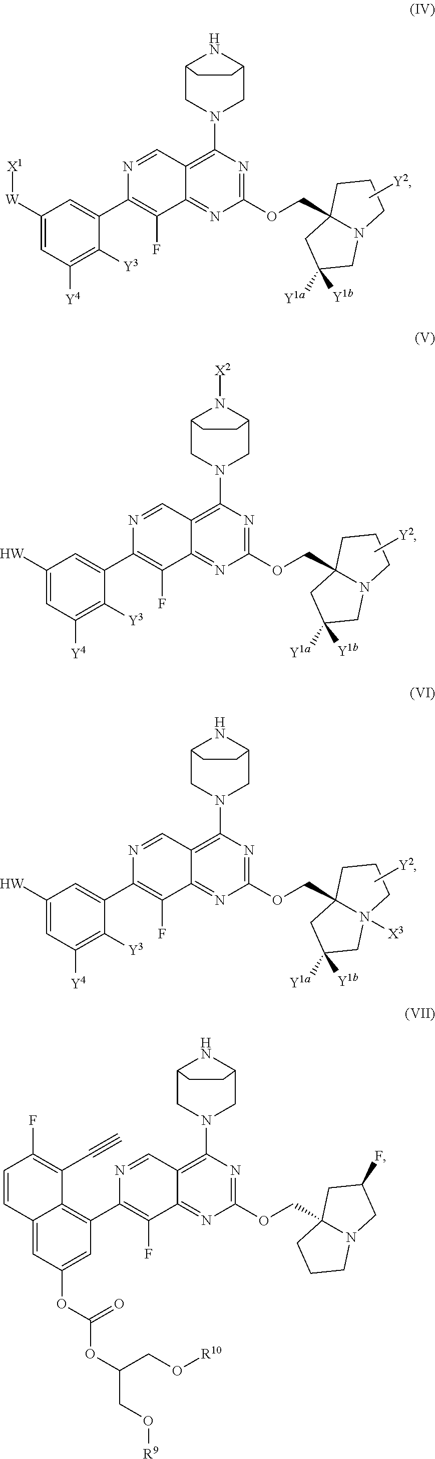

where the substitutents are as defined above.

In some embodiments of compounds of Formulae (IV)-(VII), R⁹ and R¹⁰ are independently C₂-C₂₀ alkanoyl (optionally, C₂-C₅, C₆-C₉, C₁₀-C₁₅, C₁₆-C₂₀, C₆, C₇, C₈, C₉, C₁₀, C₁₁, C₁₂, C₁₃, C₁₄, C₁₅, C₁₆, C₁₇, C₁₈, C₁₉, or C₂₀).

In some embodiments of compounds of Formulae (IV)-(VII), W is oxygen (O).

In some embodiments of compounds of Formulae (IV)-(VII), R⁹ is lower alkyl, substituted or unsubstituted benzyl, substituted or unsubstituted imidazol-5-methyl, oligoglycolyl (—[CH₂CH₂O]ₙCH₃, where n is an integer from 0 to 4), or C₂-C₂₀ saturated or unsaturated alkanoyl; and R¹⁰ is hydrogen, C₁-C₆ alkoxy, C₂-C₂₀ saturated or unsaturated alkanoyl, C₂-C₂₀ substituted or unsubstituted saturated or unsaturated alkanoyl, or saturated or unsaturated alkoxycarbonyl. In some such embodiments, R⁹ and R¹⁰ are independently C₂-C₂₀ substituted or unsubstituted saturated or unsaturated alkanoyl, e.g., C₂-C₂₀ unsubstituted saturated or unsaturated alkanoyl, e.g., C₂-C₂₀ unsubstituted, saturated alkanoyl.

In some embodiments of compounds of Formula (VII), W is O, and R⁹ and R¹⁰ are C₂-C₂₀ alkanoyl, e.g., C₂, C₃, C₄, C₅, C₆, C₇, C₈, C₉, C₁₀, C₁₁, C₁₂, C₁₃, C₁₄, Cis, C₁₆, C₁₇, C₁₈, C₁₉, or C₂₀ alkanoyl.

In some embodiments, the compounds of the disclosure are A1-, A2-, or A3-based derivatives, wherein A1, A2, and A3 are base compounds; or pharmaceutically acceptable salts, esters, hydrates, solvates, or stereoisomers thereof. The chemical structures of Base Compounds A1-A3 are shown in Table 1. The compounds of the disclosure may be those represented by the corresponding structures shown, or may be pharmaceutically acceptable salts, esters, hydrates, solvates, or stereoisomers thereof.

TABLE 1
Structures of base compounds A1, A2 and A3

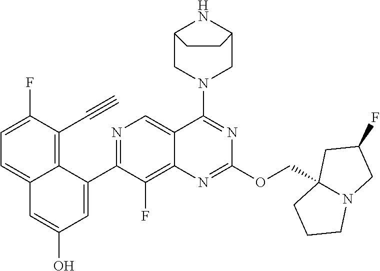 [SMILES: C#Cc1c(F)ccc2cc(O)cc(-c3ncc4c(N5CC6CCC(C5)N6)nc(OC[C@@]56CCCN5C[C@H](F)C6)nc4c3F)c12]

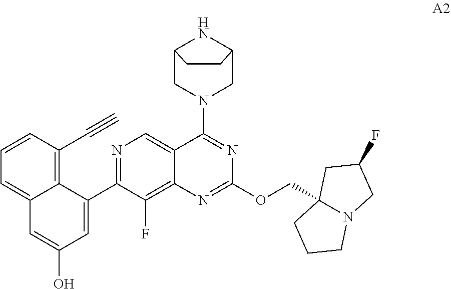 [SMILES: C#Cc1cccc2cc(O)cc(-c3ncc4c(N5CC6CCC(C5)N6)nc(OC[C@@]56CCCN5C[C@H](F)C6)nc4c3F)c12]

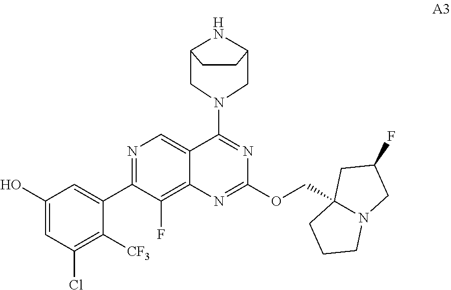 [SMILES: Oc1cc(Cl)c(C(F)(F)F)c(-c2ncc3c(N4CC5CCC(C4)N5)nc(OC[C@@]45CCCN4C[C@H](F)C5)nc3c2F)c1]

In some embodiments, the compound of the disclosure is a compound shown in Table 2 or Table 2a, or a pharmaceutically acceptable salt, ester, hydrate, solvate, or stereoisomer thereof.

TABLE 2
Structures of exemplary compounds in accordance with certain embodiments
of the disclosure.

| Cpd | |
|---|---|
| No. | Structure |
| 1 | 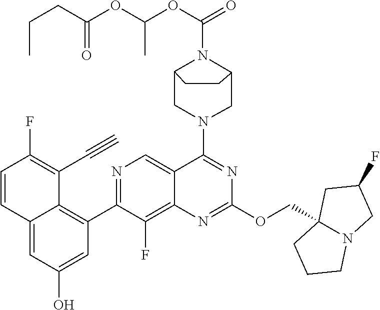 [SMILES: C#Cc1c(F)ccc2cc(O)cc(-c3ncc4c(N5CC6CCC(C5)N6C(=O)OC(C)OC(=O)CCC)nc(OC[C@@]56CCCN5C[C@H](F)C6)nc4c3F)c12] |
| 2 | 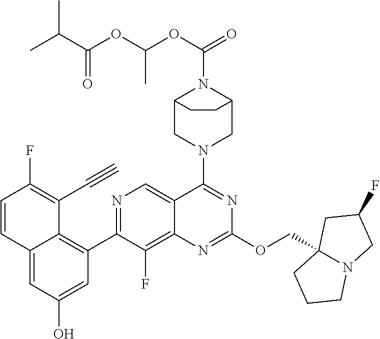 [SMILES: C#Cc1c(F)ccc2cc(O)cc(-c3ncc4c(N5CC6CCC(C5)N6C(=O)OC(C)OC(=O)C(C)C)nc(OC[C@@]56CCCN5C[C@H](F)C6)nc4c3F)c12] |
| 3 | 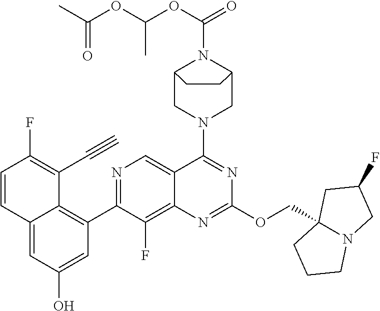 [SMILES: C#Cc1c(F)ccc2cc(O)cc(-c3ncc4c(N5CC6CCC(C5)N6C(=O)OC(C)OC(C)=O)nc(OC[C@@]56CCCN5C[C@H](F)C6)nc4c3F)c12] |
| 4 | 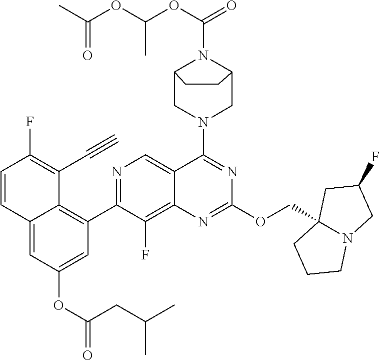 [SMILES: C#Cc1c(F)ccc2cc(OC(=O)CC(C)C)cc(-c3ncc4c(N5CC6CCC(C5)N6C(=O)OC(C)OC(C)=O)nc(OC[C@@]56CCCN5C[C@H](F)C6)nc4c3F)c12] |
| 5 | 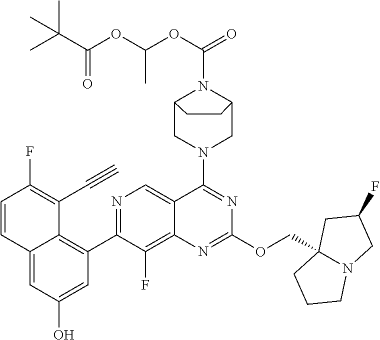 [SMILES: C#Cc1c(F)ccc2cc(O)cc(-c3ncc4c(N5CC6CCC(C5)N6C(=O)OC(C)OC(=O)C(C)(C)C)nc(OC[C@@]56CCCN5C[C@H](F)C6)nc4c3F)c12] |
| 6 | 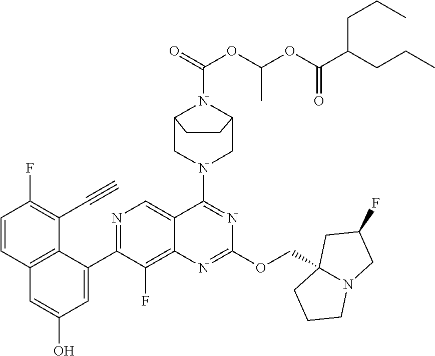 [SMILES: C#Cc1c(F)ccc2cc(O)cc(-c3ncc4c(N5CC6CCC(C5)N6C(=O)OC(C)OC(=O)C(CCC)CCC)nc(OC[C@@]56CCCN5C[C@H](F)C6)nc4c3F)c12] |
| 7 | 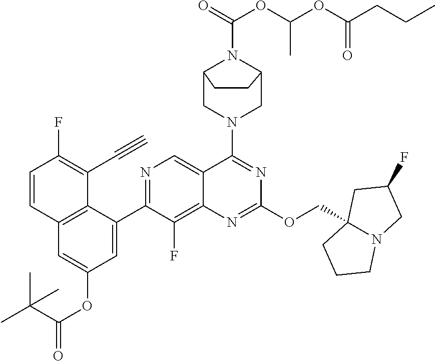 [SMILES: C#Cc1c(F)ccc2cc(OC(=O)C(C)(C)C)cc(-c3ncc4c(N5CC6CCC(C5)N6C(=O)OC(C)OC(=O)CCC)nc(OC[C@@]56CCCN5C[C@H](F)C6)nc4c3F)c12] |
| 8 | 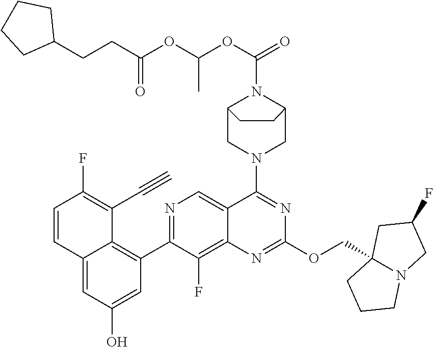 [SMILES: C#Cc1c(F)ccc2cc(O)cc(-c3ncc4c(N5CC6CCC(C5)N6C(=O)OC(C)OC(=O)CCC5CCCC5)nc(OC[C@@]56CCCN5C[C@H](F)C6)nc4c3F)c12] |
| 9 | 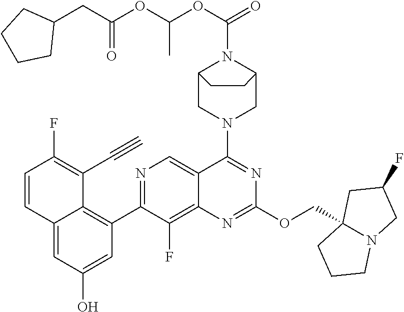 [SMILES: C#Cc1c(F)ccc2cc(O)cc(-c3ncc4c(N5CC6CCC(C5)N6C(=O)OC(C)OC(=O)CC5CCCC5)nc(OC[C@@]56CCCN5C[C@H](F)C6)nc4c3F)c12] |
| 10 | 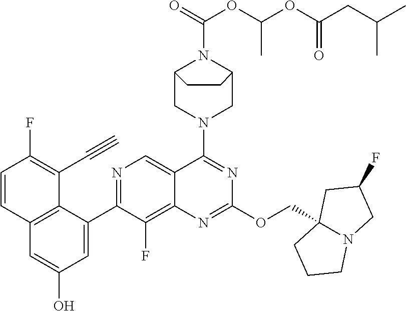 [SMILES: C#Cc1c(F)ccc2cc(O)cc(-c3ncc4c(N5CC6CCC(C5)N6C(=O)OC(C)OC(=O)CC(C)C)nc(OC[C@@]56CCCN5C[C@H](F)C6)nc4c3F)c12] |
| 11 | 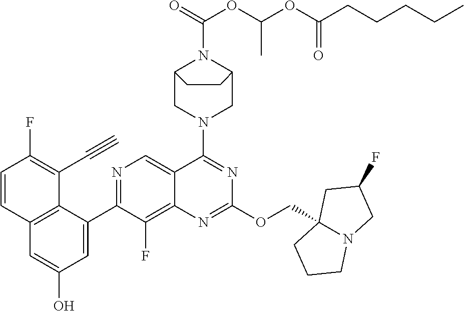 [SMILES: C#Cc1c(F)ccc2cc(O)cc(-c3ncc4c(N5CC6CCC(C5)N6C(=O)OC(C)OC(=O)CCCCC)nc(OC[C@@]56CCCN5C[C@H](F)C6)nc4c3F)c12] |
| 12 | 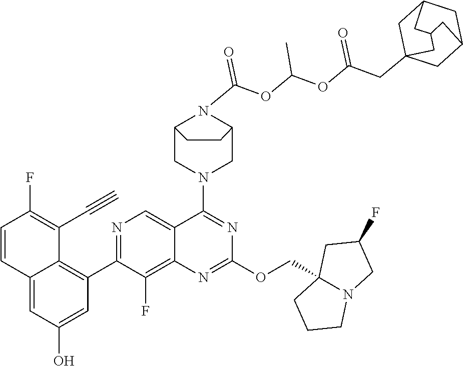 [SMILES: C#Cc1c(F)ccc2cc(O)cc(-c3ncc4c(N5CC6CCC(C5)N6C(=O)OC(C)OC(=O)CC56CC7CC(CC(C7)C5)C6)nc(OC[C@@]56CCCN5C[C@H](F)C6)nc4c3F)c12] |
| 13 | 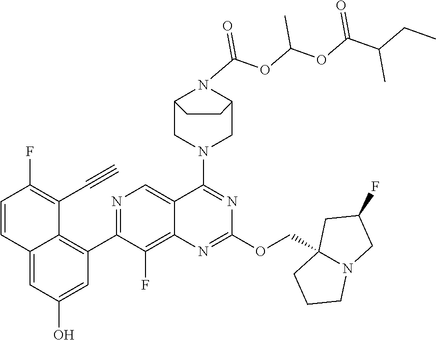 [SMILES: C#Cc1c(F)ccc2cc(O)cc(-c3ncc4c(N5CC6CCC(C5)N6C(=O)OC(C)OC(=O)C(C)CC)nc(OC[C@@]56CCCN5C[C@H](F)C6)nc4c3F)c12] |
| 14 | 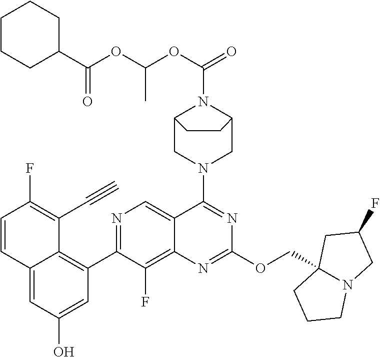 [SMILES: C#Cc1c(F)ccc2cc(O)cc(-c3ncc4c(N5CC6CCC(C5)N6C(=O)OC(C)OC(=O)C5CCCCC5)nc(OC[C@@]56CCCN5C[C@H](F)C6)nc4c3F)c12] |
| 15 | 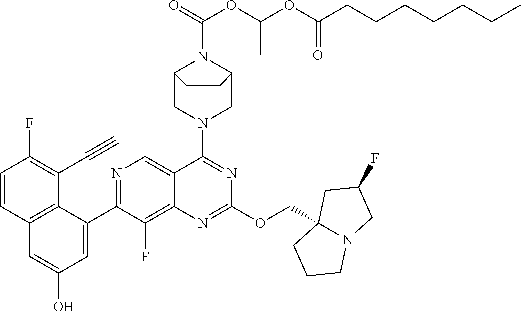 [SMILES: C#Cc1c(F)ccc2cc(O)cc(-c3ncc4c(N5CC6CCC(C5)N6C(=O)OC(C)OC(=O)CCCCCCC)nc(OC[C@@]56CCCN5C[C@H](F)C6)nc4c3F)c12] |
| 16 | 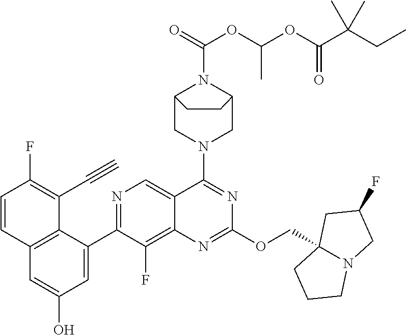 [SMILES: C#Cc1c(F)ccc2cc(O)cc(-c3ncc4c(N5CC6CCC(C5)N6C(=O)OC(C)OC(=O)C(C)(C)CC)nc(OC[C@@]56CCCN5C[C@H](F)C6)nc4c3F)c12] |
| 17 | 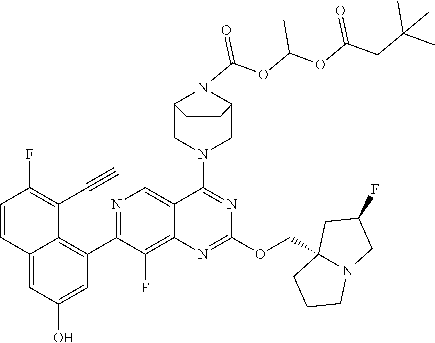 [SMILES: C#Cc1c(F)ccc2cc(O)cc(-c3ncc4c(N5CC6CCC(C5)N6C(=O)OC(C)OC(=O)CC(C)(C)C)nc(OC[C@@]56CCCN5C[C@H](F)C6)nc4c3F)c12] |
| 18 | 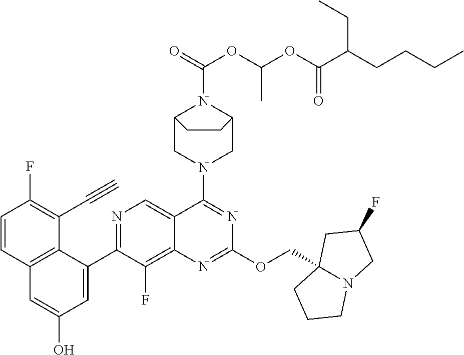 [SMILES: C#Cc1c(F)ccc2cc(O)cc(-c3ncc4c(N5CC6CCC(C5)N6C(=O)OC(C)OC(=O)C(CC)CCCC)nc(OC[C@@]56CCCN5C[C@H](F)C6)nc4c3F)c12] |
| 19 | 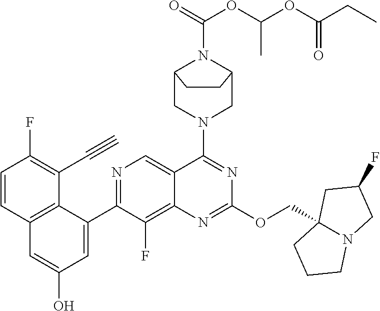 [SMILES: C#Cc1c(F)ccc2cc(O)cc(-c3ncc4c(N5CC6CCC(C5)N6C(=O)OC(C)OC(=O)CC)nc(OC[C@@]56CCCN5C[C@H](F)C6)nc4c3F)c12] |
| 20 | 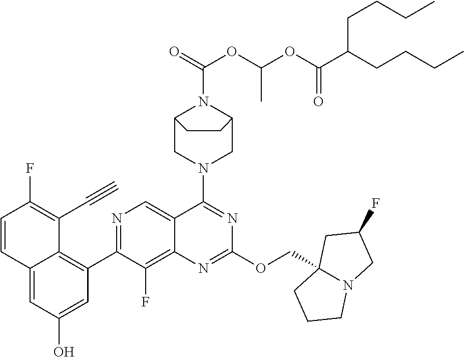 [SMILES: C#Cc1c(F)ccc2cc(O)cc(-c3ncc4c(N5CC6CCC(C5)N6C(=O)OC(C)OC(=O)C(CCCC)CCCC)nc(OC[C@@]56CCCN5C[C@H](F)C6)nc4c3F)c12] |
| 21 | 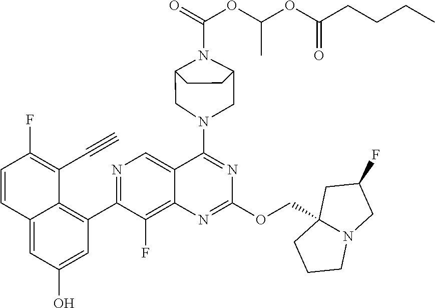 [SMILES: C#Cc1c(F)ccc2cc(O)cc(-c3ncc4c(N5CC6CCC(C5)N6C(=O)OC(C)OC(=O)CCCC)nc(OC[C@@]56CCCN5C[C@H](F)C6)nc4c3F)c12] |
| 22 | 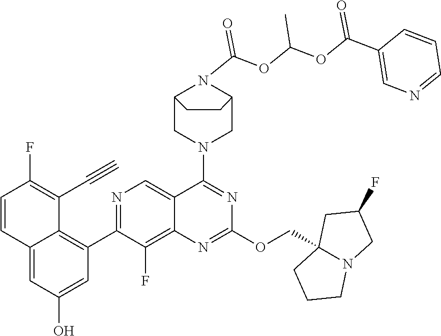 [SMILES: C#Cc1c(F)ccc2cc(O)cc(-c3ncc4c(N5CC6CCC(C5)N6C(=O)OC(C)OC(=O)c5cccnc5)nc(OC[C@@]56CCCN5C[C@H](F)C6)nc4c3F)c12] |
| 23 | 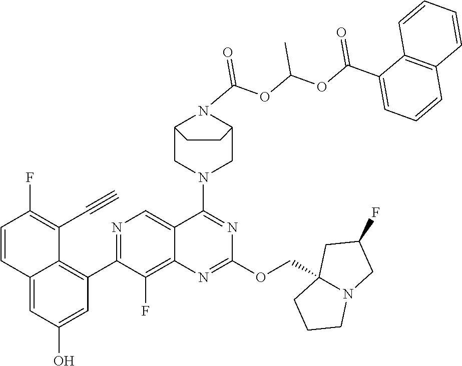 [SMILES: C#Cc1c(F)ccc2cc(O)cc(-c3ncc4c(N5CC6CCC(C5)N6C(=O)OC(C)OC(=O)c5cccc6ccccc56)nc(OC[C@@]56CCCN5C[C@H](F)C6)nc4c3F)c12] |
| 24 | 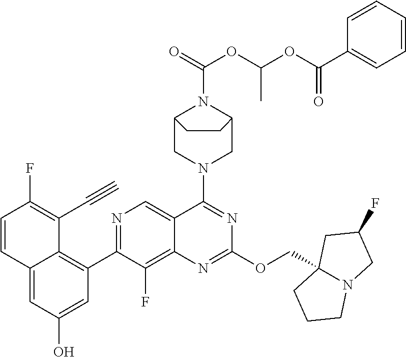 [SMILES: C#Cc1c(F)ccc2cc(O)cc(-c3ncc4c(N5CC6CCC(C5)N6C(=O)OC(C)OC(=O)c5ccccc5)nc(OC[C@@]56CCCN5C[C@H](F)C6)nc4c3F)c12] |
| 25 | 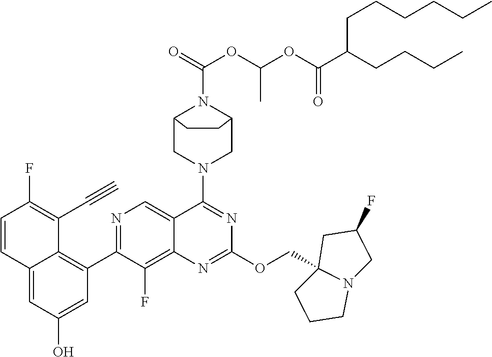 [SMILES: C#Cc1c(F)ccc2cc(O)cc(-c3ncc4c(N5CC6CCC(C5)N6C(=O)OC(C)OC(=O)C(CCCC)CCCCCC)nc(OC[C@@]56CCCN5C[C@H](F)C6)nc4c3F)c12] |
| 26 | 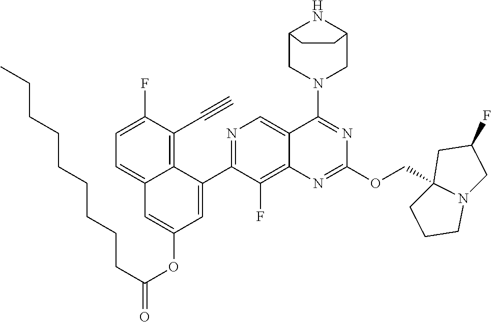 [SMILES: C#Cc1c(F)ccc2cc(OC(=O)CCCCCCCCC)cc(-c3ncc4c(N5CC6CCC(C5)N6)nc(OC[C@@]56CCCN5C[C@H](F)C6)nc4c3F)c12] |
| 27 | 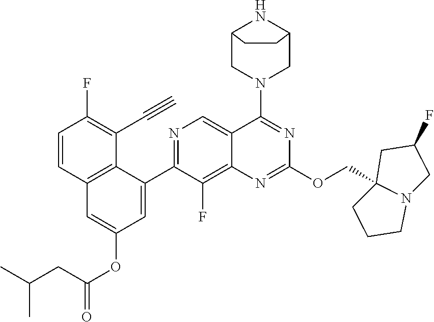 [SMILES: C#Cc1c(F)ccc2cc(OC(=O)CC(C)C)cc(-c3ncc4c(N5CC6CCC(C5)N6)nc(OC[C@@]56CCCN5C[C@H](F)C6)nc4c3F)c12] |
| 28 | 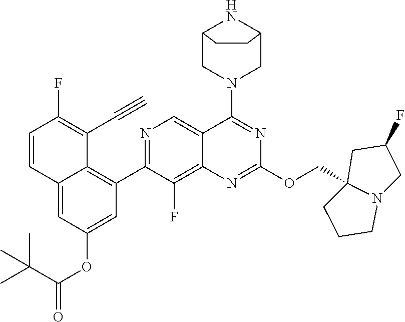 [SMILES: C#Cc1c(F)ccc2cc(OC(=O)C(C)(C)C)cc(-c3ncc4c(N5CC6CCC(C5)N6)nc(OC[C@@]56CCCN5C[C@H](F)C6)nc4c3F)c12] |
| 29 | 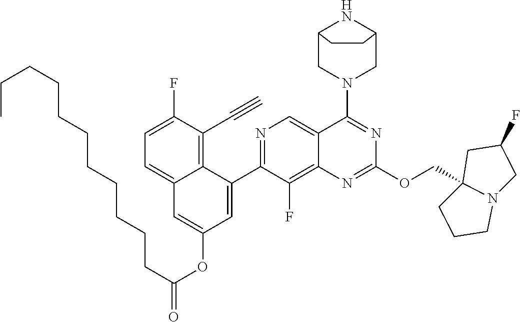 [SMILES: C#Cc1c(F)ccc2cc(OC(=O)CCCCCCCCCCC)cc(-c3ncc4c(N5CC6CCC(C5)N6)nc(OC[C@@]56CCCN5C[C@H](F)C6)nc4c3F)c12] |
| 30 | 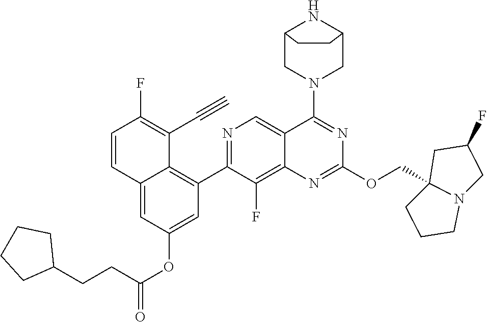 [SMILES: C#Cc1c(F)ccc2cc(OC(=O)CCC3CCCC3)cc(-c3ncc4c(N5CC6CCC(C5)N6)nc(OC[C@@]56CCCN5C[C@H](F)C6)nc4c3F)c12] |
| 31 | 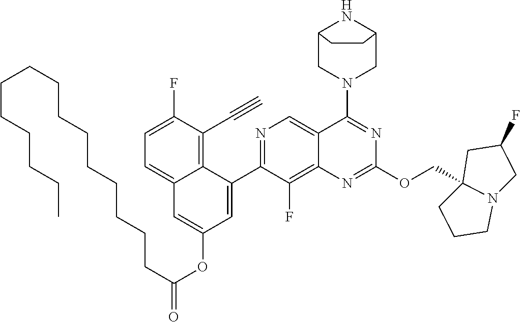 [SMILES: C#Cc1c(F)ccc2cc(OC(=O)CCCCCCCCCCCCCCC)cc(-c3ncc4c(N5CC6CCC(C5)N6)nc(OC[C@@]56CCCN5C[C@H](F)C6)nc4c3F)c12] |
| 32 | 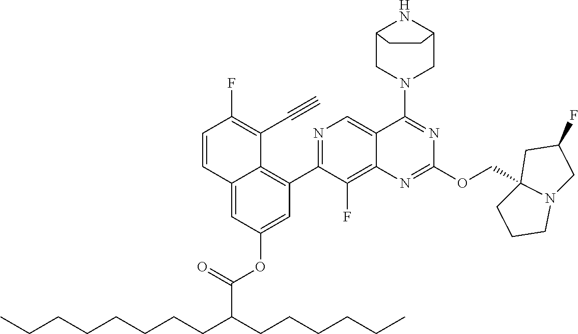 [SMILES: C#Cc1c(F)ccc2cc(OC(=O)C(CCCCCC)CCCCCCCC)cc(-c3ncc4c(N5CC6CCC(C5)N6)nc(OC[C@@]56CCCN5C[C@H](F)C6)nc4c3F)c12] |
| 33 | 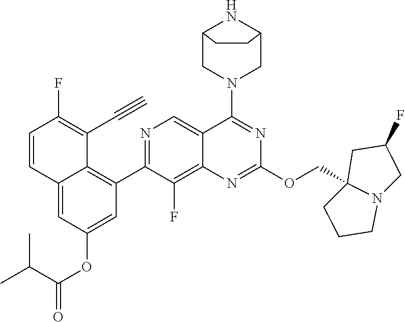 [SMILES: C#Cc1c(F)ccc2cc(OC(=O)C(C)C)cc(-c3ncc4c(N5CC6CCC(C5)N6)nc(OC[C@@]56CCCN5C[C@H](F)C6)nc4c3F)c12] |
| 34 | 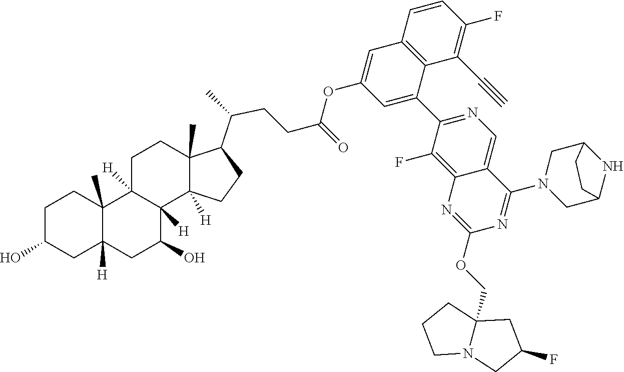 [SMILES: C#Cc1c(F)ccc2cc(OC(=O)CC[C@@H](C)[C@H]3CC[C@H]4[C@@H]5[C@@H](O)C[C@@H]6C[C@H](O)CC[C@]6(C)[C@H]5CC[C@]34C)cc(-c3ncc4c(N5CC6CCC(C5)N6)nc(OC[C@@]56CCCN5C[C@H](F)C6)nc4c3F)c12] |
| 35 | 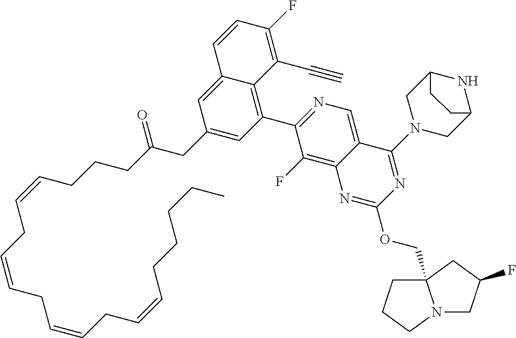 [SMILES: C#Cc1c(F)ccc2cc(CC(=O)CCC/C=C\C/C=C\C/C=C\C/C=C\CCCCC)cc(-c3ncc4c(N5CC6CCC(C5)N6)nc(OC[C@@]56CCCN5C[C@H](F)C6)nc4c3F)c12] |
| 36 | 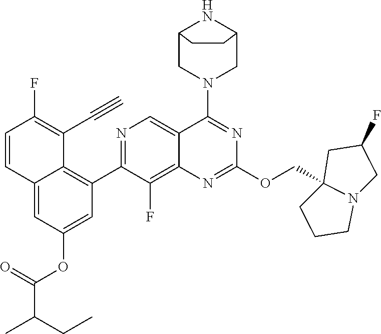 [SMILES: C#Cc1c(F)ccc2cc(OC(=O)C(C)CC)cc(-c3ncc4c(N5CC6CCC(C5)N6)nc(OC[C@@]56CCCN5C[C@H](F)C6)nc4c3F)c12] |
| 37 | 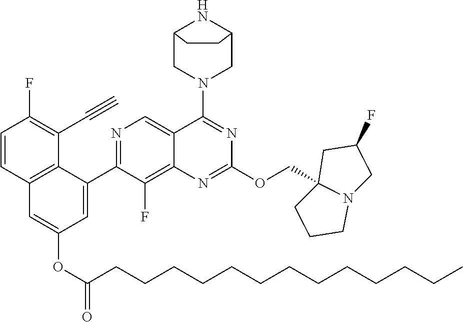 [SMILES: C#Cc1c(F)ccc2cc(OC(=O)CCCCCCCCCCCCC)cc(-c3ncc4c(N5CC6CCC(C5)N6)nc(OC[C@@]56CCCN5C[C@H](F)C6)nc4c3F)c12] |
| 38 | 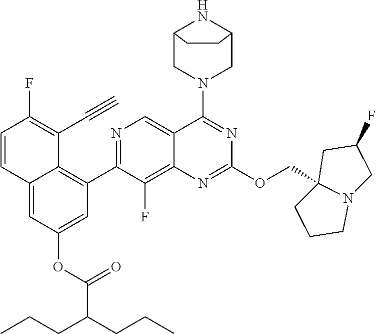 [SMILES: C#Cc1c(F)ccc2cc(OC(=O)C(CCC)CCC)cc(-c3ncc4c(N5CC6CCC(C5)N6)nc(OC[C@@]56CCCN5C[C@H](F)C6)nc4c3F)c12] |
| 39 | 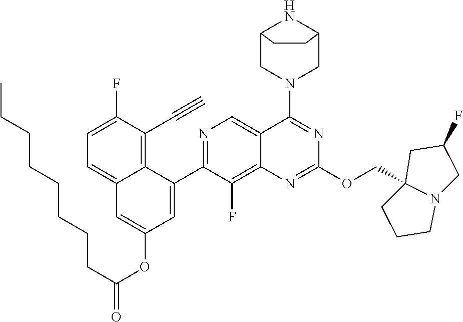 [SMILES: C#Cc1c(F)ccc2cc(OC(=O)CCCCCCCC)cc(-c3ncc4c(N5CC6CCC(C5)N6)nc(OC[C@@]56CCCN5C[C@H](F)C6)nc4c3F)c12] |
| 40 | 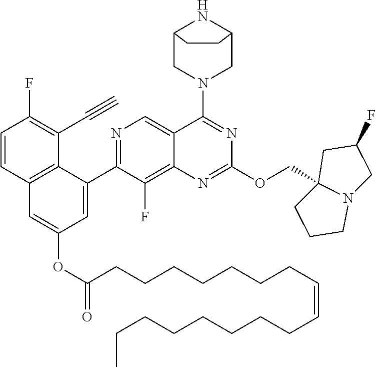 [SMILES: C#Cc1c(F)ccc2cc(OC(=O)CCCCCCC/C=C\CCCCCCCC)cc(-c3ncc4c(N5CC6CCC(C5)N6)nc(OC[C@@]56CCCN5C[C@H](F)C6)nc4c3F)c12] |
| 41 | 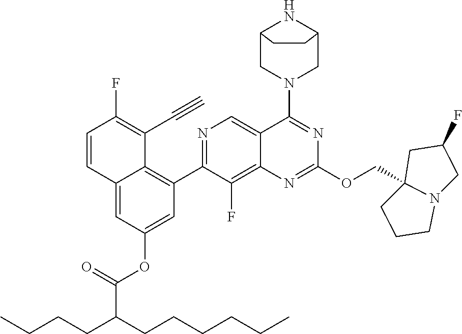 [SMILES: C#Cc1c(F)ccc2cc(OC(=O)C(CCCC)CCCCCC)cc(-c3ncc4c(N5CC6CCC(C5)N6)nc(OC[C@@]56CCCN5C[C@H](F)C6)nc4c3F)c12] |
| 42 | 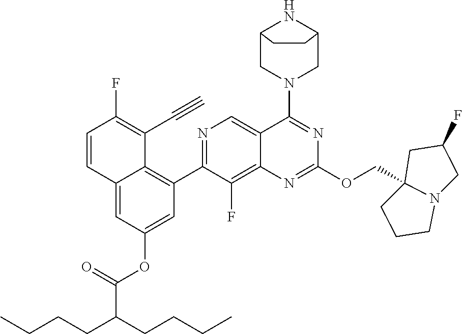 [SMILES: C#Cc1c(F)ccc2cc(OC(=O)C(CCCC)CCCC)cc(-c3ncc4c(N5CC6CCC(C5)N6)nc(OC[C@@]56CCCN5C[C@H](F)C6)nc4c3F)c12] |
| 43 | 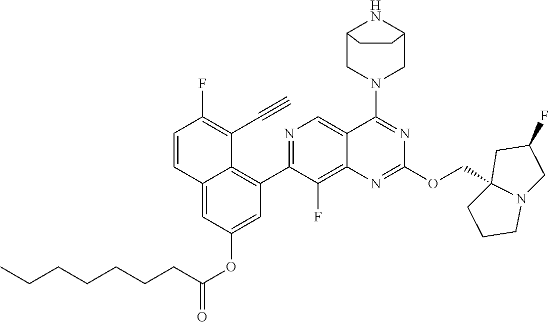 [SMILES: C#Cc1c(F)ccc2cc(OC(=O)CCCCCCC)cc(-c3ncc4c(N5CC6CCC(C5)N6)nc(OC[C@@]56CCCN5C[C@H](F)C6)nc4c3F)c12] |
| 44 | 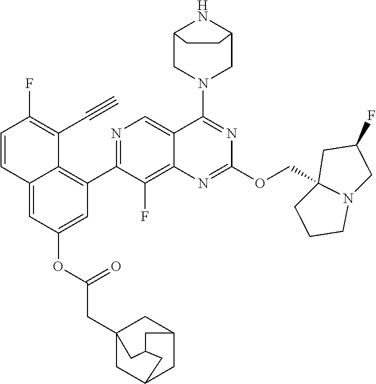 [SMILES: C#Cc1c(F)ccc2cc(OC(=O)CC34CC5CC(CC(C5)C3)C4)cc(-c3ncc4c(N5CC6CCC(C5)N6)nc(OC[C@@]56CCCN5C[C@H](F)C6)nc4c3F)c12] |
| 45 | 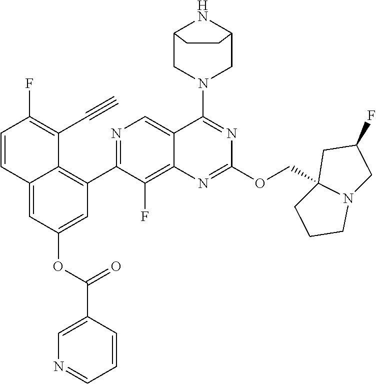 [SMILES: C#Cc1c(F)ccc2cc(OC(=O)c3cccnc3)cc(-c3ncc4c(N5CC6CCC(C5)N6)nc(OC[C@@]56CCCN5C[C@H](F)C6)nc4c3F)c12] |
| 46 | 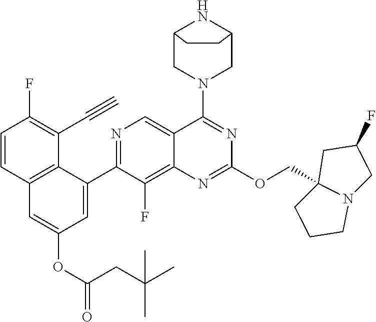 [SMILES: C#Cc1c(F)ccc2cc(OC(=O)CC(C)(C)C)cc(-c3ncc4c(N5CC6CCC(C5)N6)nc(OC[C@@]56CCCN5C[C@H](F)C6)nc4c3F)c12] |
| 47 | 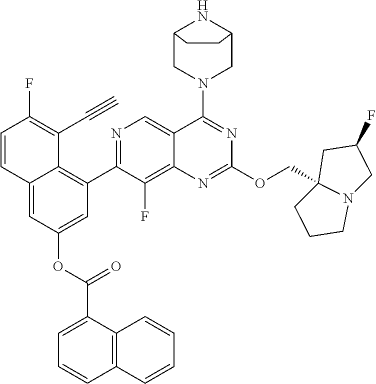 [SMILES: C#Cc1c(F)ccc2cc(OC(=O)c3cccc4ccccc34)cc(-c3ncc4c(N5CC6CCC(C5)N6)nc(OC[C@@]56CCCN5C[C@H](F)C6)nc4c3F)c12] |
| 48 | 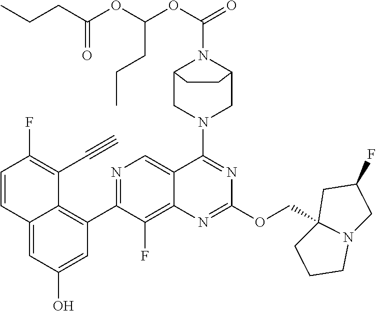 [SMILES: C#Cc1c(F)ccc2cc(O)cc(-c3ncc4c(N5CC6CCC(C5)N6C(=O)OC(CCC)OC(=O)CCC)nc(OC[C@@]56CCCN5C[C@H](F)C6)nc4c3F)c12] |
| 49 | 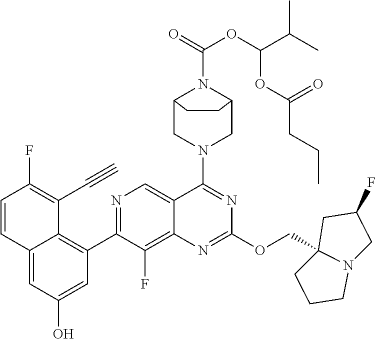 [SMILES: C#Cc1c(F)ccc2cc(O)cc(-c3ncc4c(N5CC6CCC(C5)N6C(=O)OC(OC(=O)CCC)C(C)C)nc(OC[C@@]56CCCN5C[C@H](F)C6)nc4c3F)c12] |
| 50 | 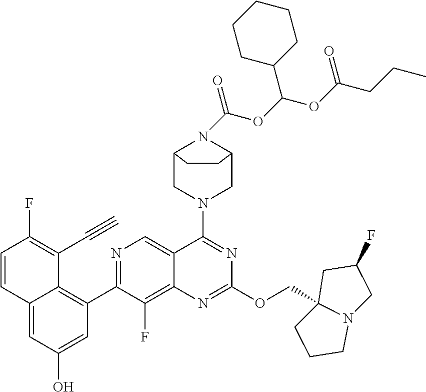 [SMILES: C#Cc1c(F)ccc2cc(O)cc(-c3ncc4c(N5CC6CCC(C5)N6C(=O)OC(OC(=O)CCC)C5CCCCC5)nc(OC[C@@]56CCCN5C[C@H](F)C6)nc4c3F)c12] |
| 51 | 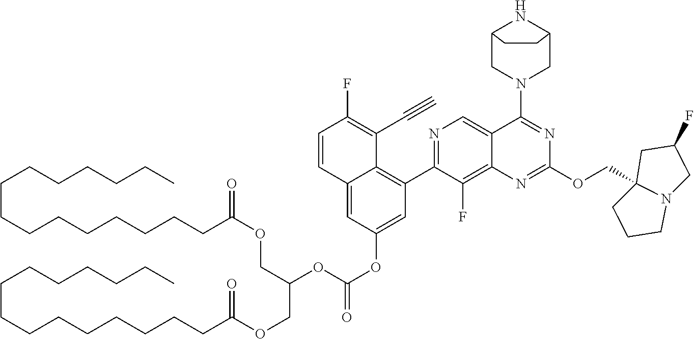 [SMILES: C#Cc1c(F)ccc2cc(OC(=O)OC(COC(=O)CCCCCCCCCCCCCCC)COC(=O)CCCCCCCCCCCCCCC)cc(-c3ncc4c(N5CC6CCC(C5)N6)nc(OC[C@@]56CCCN5C[C@H](F)C6)nc4c3F)c12] |
| 52 | 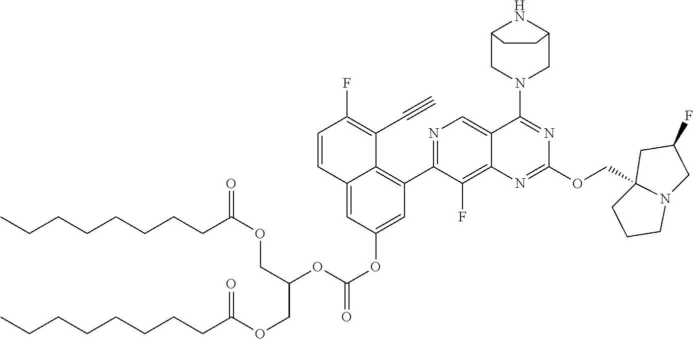 [SMILES: C#Cc1c(F)ccc2cc(OC(=O)OC(COC(=O)CCCCCCCC)COC(=O)CCCCCCCC)cc(-c3ncc4c(N5CC6CCC(C5)N6)nc(OC[C@@]56CCCN5C[C@H](F)C6)nc4c3F)c12] |
| 53 | 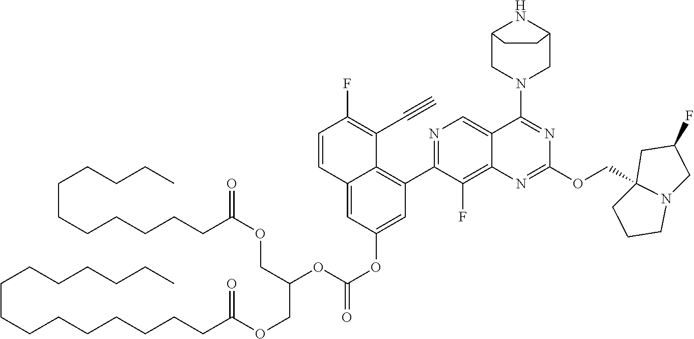 [SMILES: C#Cc1c(F)ccc2cc(OC(=O)OC(COC(=O)CCCCCCCCCCC)COC(=O)CCCCCCCCCCCCCCC)cc(-c3ncc4c(N5CC6CCC(C5)N6)nc(OC[C@@]56CCCN5C[C@H](F)C6)nc4c3F)c12] |
| 54 | 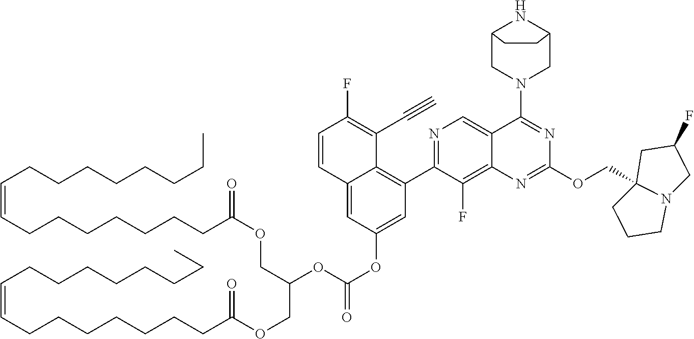 [SMILES: C#Cc1c(F)ccc2cc(OC(=O)OC(COC(=O)CCCCCCC/C=C\CCCCCCCC)COC(=O)CCCCCCC/C=C\CCCCCCCC)cc(-c3ncc4c(N5CC6CCC(C5)N6)nc(OC[C@@]56CCCN5C[C@H](F)C6)nc4c3F)c12] |
| 55 | 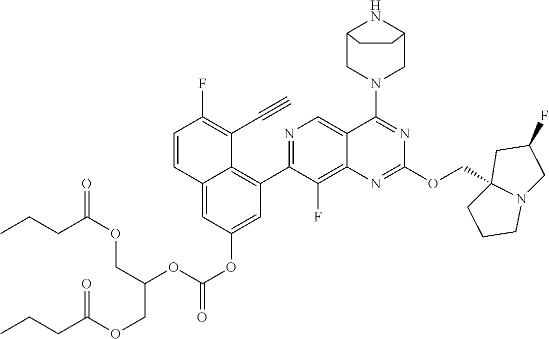 [SMILES: C#Cc1c(F)ccc2cc(OC(=O)OC(COC(=O)CCC)COC(=O)CCC)cc(-c3ncc4c(N5CC6CCC(C5)N6)nc(OC[C@@]56CCCN5C[C@H](F)C6)nc4c3F)c12] |
| 56 | 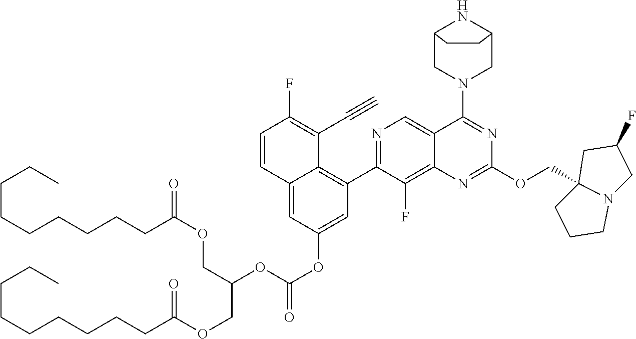 [SMILES: C#Cc1c(F)ccc2cc(OC(=O)OC(COC(=O)CCCCCCCCC)COC(=O)CCCCCCCCC)cc(-c3ncc4c(N5CC6CCC(C5)N6)nc(OC[C@@]56CCCN5C[C@H](F)C6)nc4c3F)c12] |
| 57 | 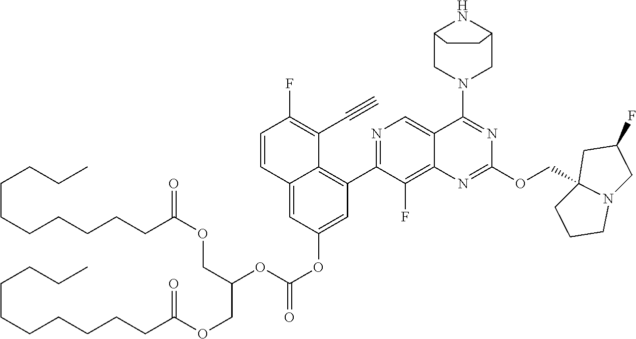 [SMILES: C#Cc1c(F)ccc2cc(OC(=O)OC(COC(=O)CCCCCCCCCC)COC(=O)CCCCCCCCCC)cc(-c3ncc4c(N5CC6CCC(C5)N6)nc(OC[C@@]56CCCN5C[C@H](F)C6)nc4c3F)c12] |
| 58 | 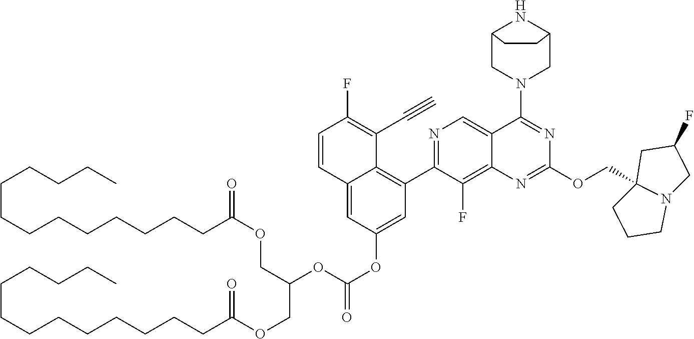 [SMILES: C#Cc1c(F)ccc2cc(OC(=O)OC(COC(=O)CCCCCCCCCCCCC)COC(=O)CCCCCCCCCCCCC)cc(-c3ncc4c(N5CC6CCC(C5)N6)nc(OC[C@@]56CCCN5C[C@H](F)C6)nc4c3F)c12] |
| 59 | 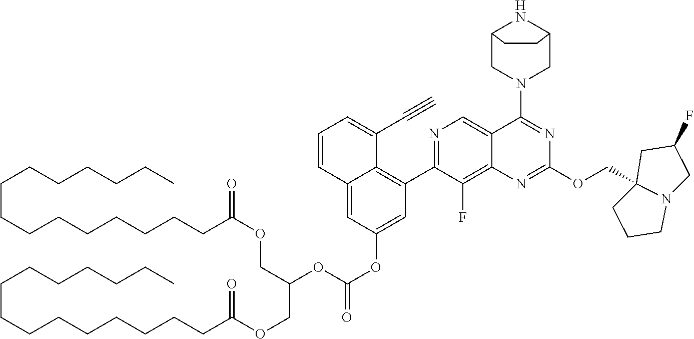 [SMILES: C#Cc1cccc2cc(OC(=O)OC(COC(=O)CCCCCCCCCCCCCCC)COC(=O)CCCCCCCCCCCCCCC)cc(-c3ncc4c(N5CC6CCC(C5)N6)nc(OC[C@@]56CCCN5C[C@H](F)C6)nc4c3F)c12] |
| 60 | 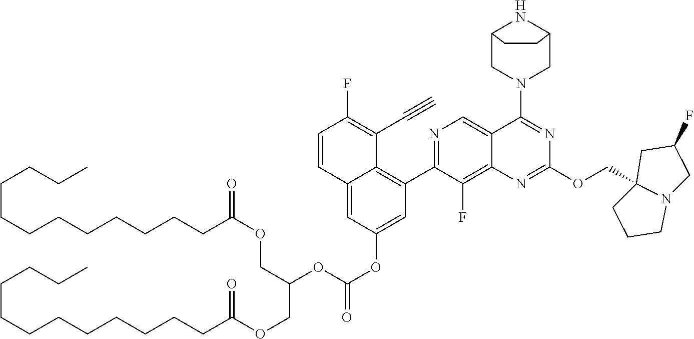 [SMILES: C#Cc1c(F)ccc2cc(OC(=O)OC(COC(=O)CCCCCCCCCCCC)COC(=O)CCCCCCCCCCCC)cc(-c3ncc4c(N5CC6CCC(C5)N6)nc(OC[C@@]56CCCN5C[C@H](F)C6)nc4c3F)c12] |
| 61 | 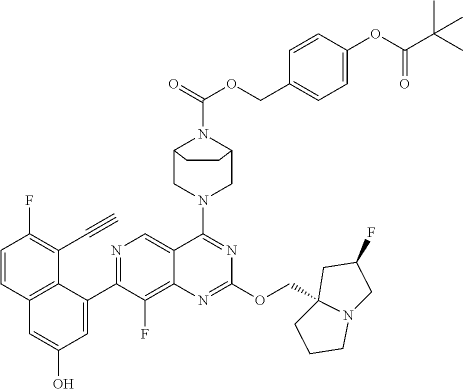 [SMILES: C#Cc1c(F)ccc2cc(O)cc(-c3ncc4c(N5CC6CCC(C5)N6C(=O)OCc5ccc(OC(=O)C(C)(C)C)cc5)nc(OC[C@@]56CCCN5C[C@H](F)C6)nc4c3F)c12] |
| 62 | 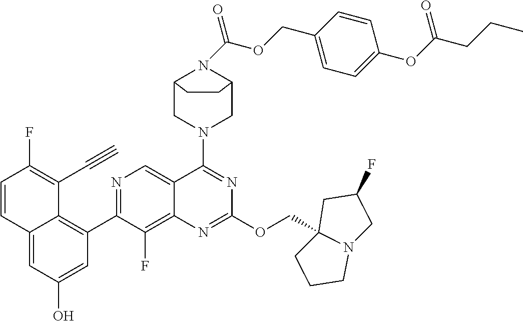 [SMILES: C#Cc1c(F)ccc2cc(O)cc(-c3ncc4c(N5CC6CCC(C5)N6C(=O)OCc5ccc(OC(=O)CCC)cc5)nc(OC[C@@]56CCCN5C[C@H](F)C6)nc4c3F)c12] |
| 63 | 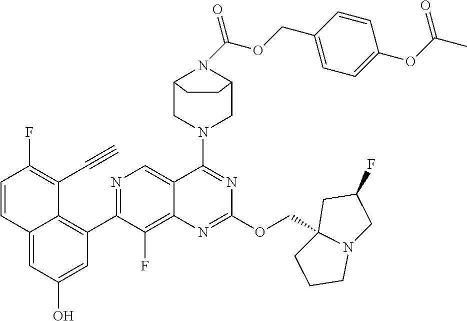 [SMILES: C#Cc1c(F)ccc2cc(O)cc(-c3ncc4c(N5CC6CCC(C5)N6C(=O)OCc5ccc(OC(C)=O)cc5)nc(OC[C@@]56CCCN5C[C@H](F)C6)nc4c3F)c12] |
| 64 | 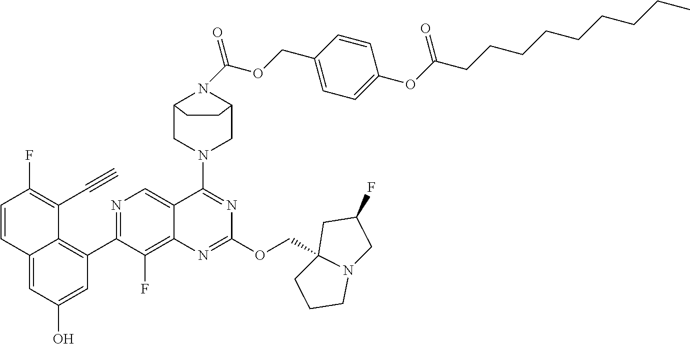 [SMILES: C#Cc1c(F)ccc2cc(O)cc(-c3ncc4c(N5CC6CCC(C5)N6C(=O)OCc5ccc(OC(=O)CCCCCCCCC)cc5)nc(OC[C@@]56CCCN5C[C@H](F)C6)nc4c3F)c12] |
| 65 | 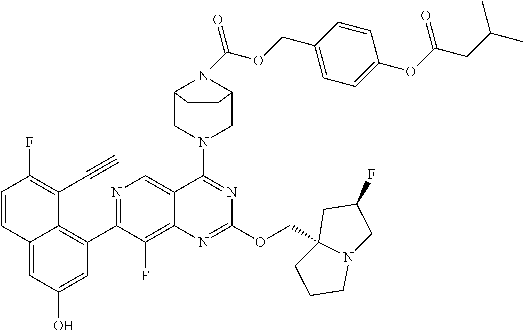 [SMILES: C#Cc1c(F)ccc2cc(O)cc(-c3ncc4c(N5CC6CCC(C5)N6C(=O)OCc5ccc(OC(=O)CC(C)C)cc5)nc(OC[C@@]56CCCN5C[C@H](F)C6)nc4c3F)c12] |
| 66 | 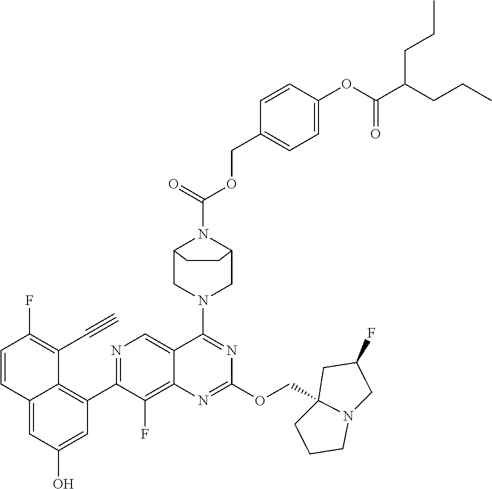 [SMILES: C#Cc1c(F)ccc2cc(O)cc(-c3ncc4c(N5CC6CCC(C5)N6C(=O)OCc5ccc(OC(=O)C(CCC)CCC)cc5)nc(OC[C@@]56CCCN5C[C@H](F)C6)nc4c3F)c12] |
| 67 | 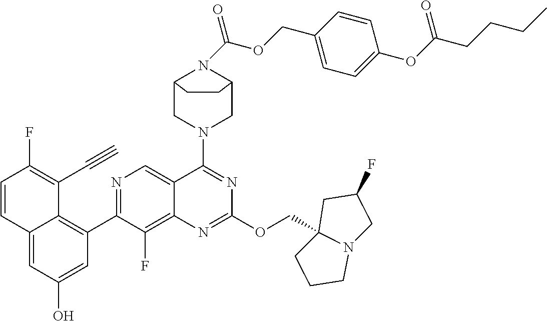 [SMILES: C#Cc1c(F)ccc2cc(O)cc(-c3ncc4c(N5CC6CCC(C5)N6C(=O)OCc5ccc(OC(=O)CCCC)cc5)nc(OC[C@@]56CCCN5C[C@H](F)C6)nc4c3F)c12] |
| 68 | 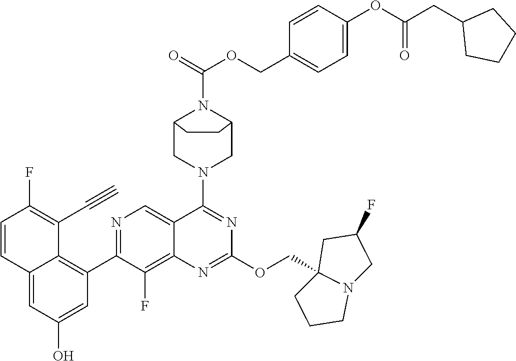 [SMILES: C#Cc1c(F)ccc2cc(O)cc(-c3ncc4c(N5CC6CCC(C5)N6C(=O)OCc5ccc(OC(=O)CC6CCCC6)cc5)nc(OC[C@@]56CCCN5C[C@H](F)C6)nc4c3F)c12] |
| 69 | 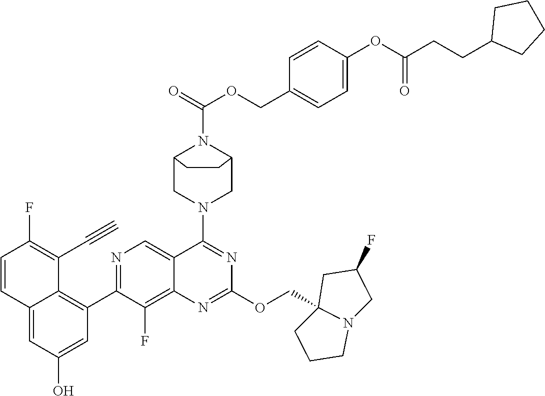 [SMILES: C#Cc1c(F)ccc2cc(O)cc(-c3ncc4c(N5CC6CCC(C5)N6C(=O)OCc5ccc(OC(=O)CCC6CCCC6)cc5)nc(OC[C@@]56CCCN5C[C@H](F)C6)nc4c3F)c12] |
| 70 | 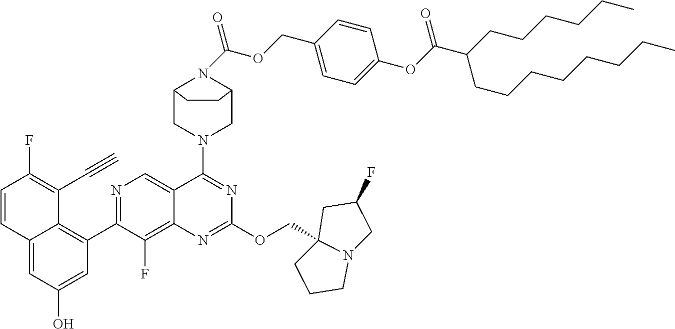 [SMILES: C#Cc1c(F)ccc2cc(O)cc(-c3ncc4c(N5CC6CCC(C5)N6C(=O)OCc5ccc(OC(=O)C(CCCCCC)CCCCCCCC)cc5)nc(OC[C@@]56CCCN5C[C@H](F)C6)nc4c3F)c12] |
| 71 | 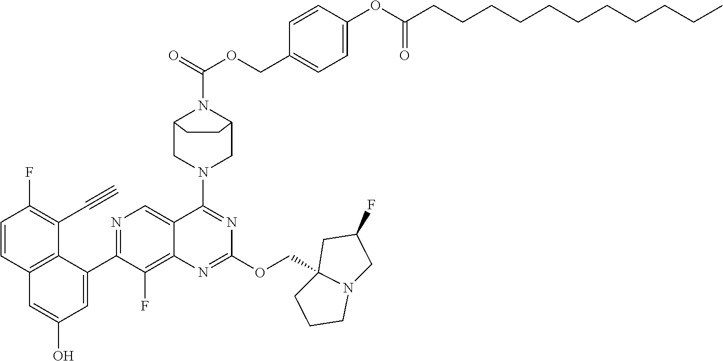 [SMILES: C#Cc1c(F)ccc2cc(O)cc(-c3ncc4c(N5CC6CCC(C5)N6C(=O)OCc5ccc(OC(=O)CCCCCCCCCCC)cc5)nc(OC[C@@]56CCCN5C[C@H](F)C6)nc4c3F)c12] |
| 72 | 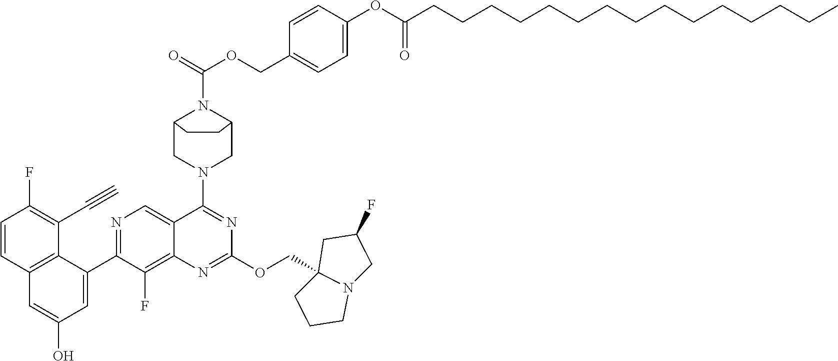 [SMILES: C#Cc1c(F)ccc2cc(O)cc(-c3ncc4c(N5CC6CCC(C5)N6C(=O)OCc5ccc(OC(=O)CCCCCCCCCCCCCCC)cc5)nc(OC[C@@]56CCCN5C[C@H](F)C6)nc4c3F)c12] |
| 73 | 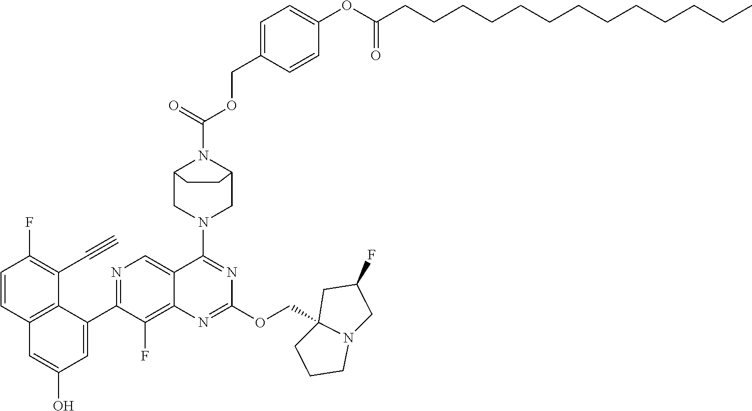 [SMILES: C#Cc1c(F)ccc2cc(O)cc(-c3ncc4c(N5CC6CCC(C5)N6C(=O)OCc5ccc(OC(=O)CCCCCCCCCCCCC)cc5)nc(OC[C@@]56CCCN5C[C@H](F)C6)nc4c3F)c12] |
| 74 | 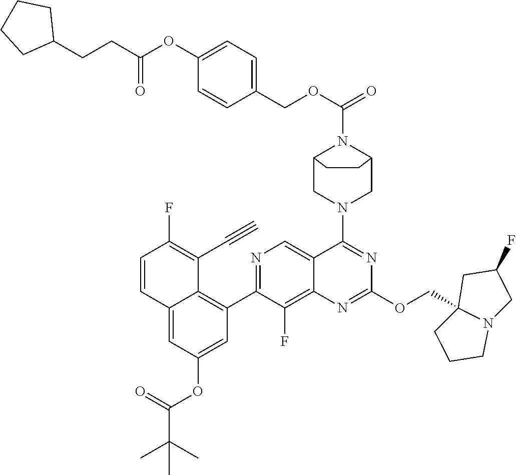 [SMILES: C#Cc1c(F)ccc2cc(OC(=O)C(C)(C)C)cc(-c3ncc4c(N5CC6CCC(C5)N6C(=O)OCc5ccc(OC(=O)CCC6CCCC6)cc5)nc(OC[C@@]56CCCN5C[C@H](F)C6)nc4c3F)c12] |
| 75 | 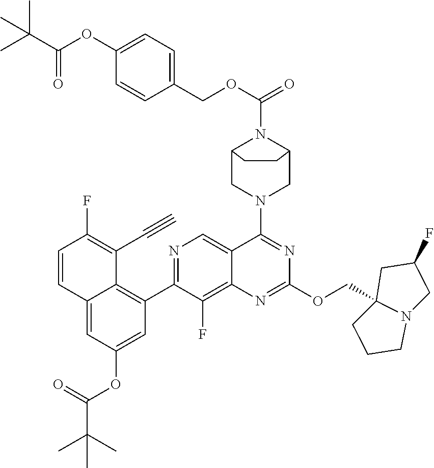 [SMILES: C#Cc1c(F)ccc2cc(OC(=O)C(C)(C)C)cc(-c3ncc4c(N5CC6CCC(C5)N6C(=O)OCc5ccc(OC(=O)C(C)(C)C)cc5)nc(OC[C@@]56CCCN5C[C@H](F)C6)nc4c3F)c12] |
| 76 | 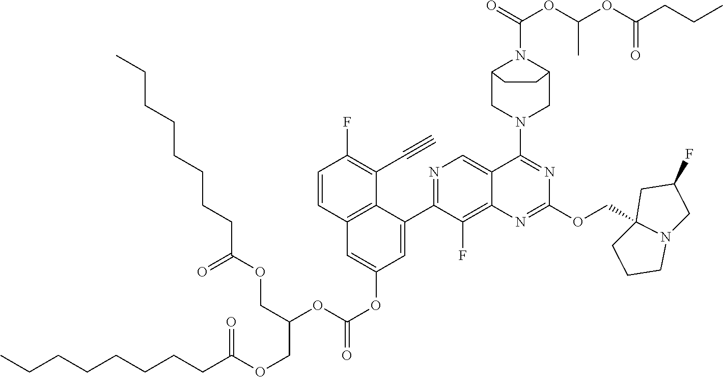 [SMILES: C#Cc1c(F)ccc2cc(OC(=O)OC(COC(=O)CCCCCCCC)COC(=O)CCCCCCCC)cc(-c3ncc4c(N5CC6CCC(C5)N6C(=O)OC(C)OC(=O)CCC)nc(OC[C@@]56CCCN5C[C@H](F)C6)nc4c3F)c12] |
| 77 | 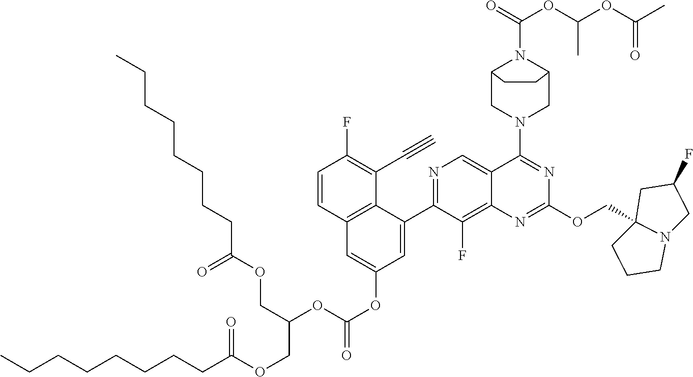 [SMILES: C#Cc1c(F)ccc2cc(OC(=O)OC(COC(=O)CCCCCCCC)COC(=O)CCCCCCCC)cc(-c3ncc4c(N5CC6CCC(C5)N6C(=O)OC(C)OC(C)=O)nc(OC[C@@]56CCCN5C[C@H](F)C6)nc4c3F)c12] |
| 78 | 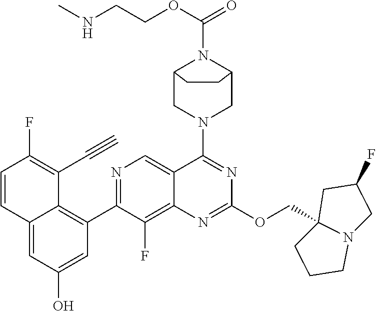 [SMILES: C#Cc1c(F)ccc2cc(O)cc(-c3ncc4c(N5CC6CCC(C5)N6C(=O)OCCNC)nc(OC[C@@]56CCCN5C[C@H](F)C6)nc4c3F)c12] |
| 79 | 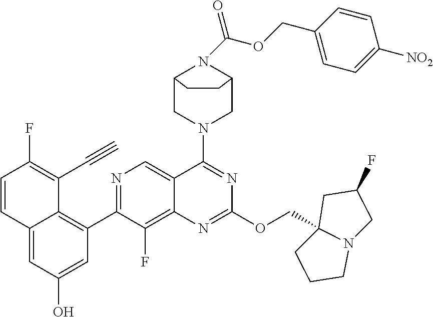 [SMILES: C#Cc1c(F)ccc2cc(O)cc(-c3ncc4c(N5CC6CCC(C5)N6C(=O)OCc5ccc([N+](=O)[O-])cc5)nc(OC[C@@]56CCCN5C[C@H](F)C6)nc4c3F)c12] |
| 80 | 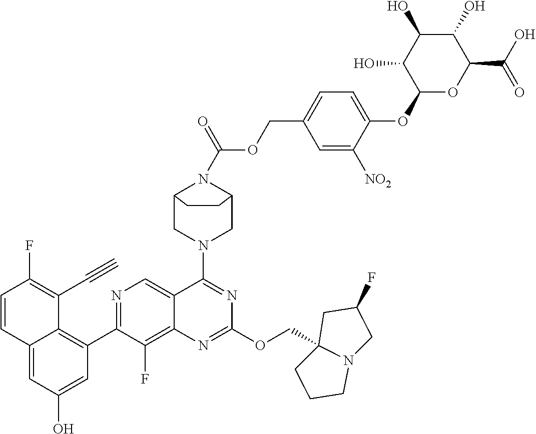 [SMILES: C#Cc1c(F)ccc2cc(O)cc(-c3ncc4c(N5CC6CCC(C5)N6C(=O)OCc5ccc(O[C@@H]6O[C@H](C(=O)O)[C@@H](O)[C@H](O)[C@H]6O)c([N+](=O)[O-])c5)nc(OC[C@@]56CCCN5C[C@H](F)C6)nc4c3F)c12] |
| 81 | 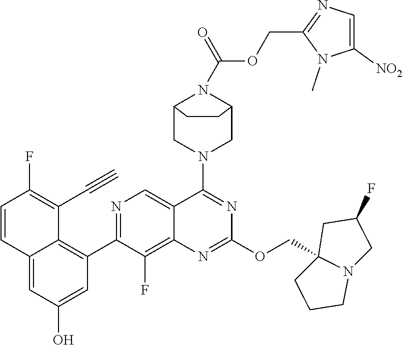 [SMILES: C#Cc1c(F)ccc2cc(O)cc(-c3ncc4c(N5CC6CCC(C5)N6C(=O)OCc5ncc([N+](=O)[O-])n5C)nc(OC[C@@]56CCCN5C[C@H](F)C6)nc4c3F)c12] |
| 82 | 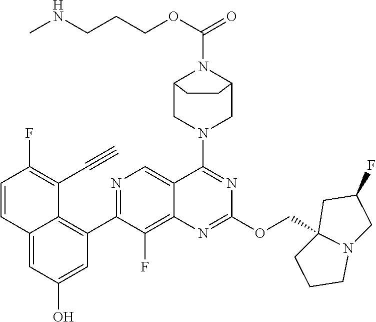 [SMILES: C#Cc1c(F)ccc2cc(O)cc(-c3ncc4c(N5CC6CCC(C5)N6C(=O)OCCCNC)nc(OC[C@@]56CCCN5C[C@H](F)C6)nc4c3F)c12] |
| 83 | 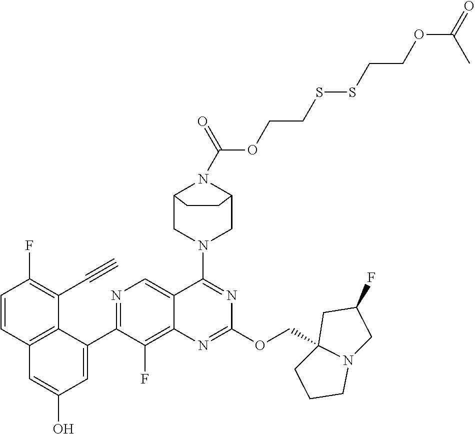 [SMILES: C#Cc1c(F)ccc2cc(O)cc(-c3ncc4c(N5CC6CCC(C5)N6C(=O)OCCSSCCOC(C)=O)nc(OC[C@@]56CCCN5C[C@H](F)C6)nc4c3F)c12] |
| 84 | 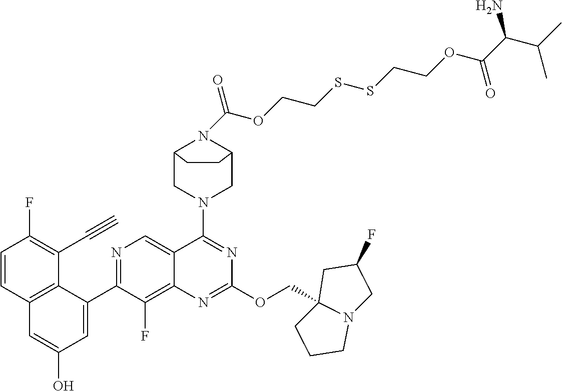 [SMILES: C#Cc1c(F)ccc2cc(O)cc(-c3ncc4c(N5CC6CCC(C5)N6C(=O)OCCSSCCOC(=O)[C@@H](N)C(C)C)nc(OC[C@@]56CCCN5C[C@H](F)C6)nc4c3F)c12] |
| 85 | 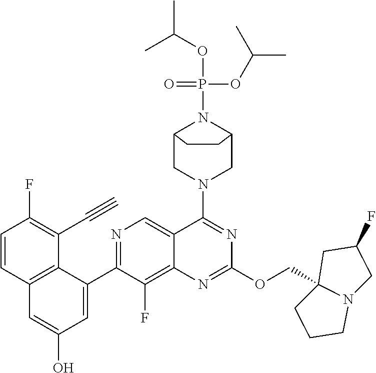 [SMILES: C#Cc1c(F)ccc2cc(O)cc(-c3ncc4c(N5CC6CCC(C5)N6P(=O)(OC(C)C)OC(C)C)nc(OC[C@@]56CCCN5C[C@H](F)C6)nc4c3F)c12] |
| 86 | 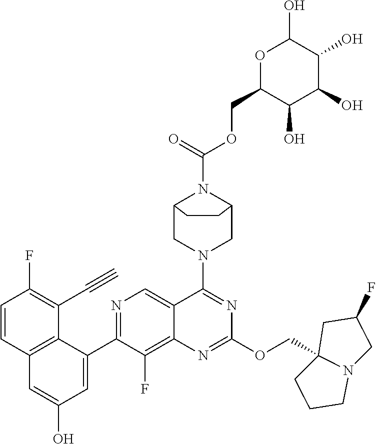 [SMILES: C#Cc1c(F)ccc2cc(O)cc(-c3ncc4c(N5CC6CCC(C5)N6C(=O)OC[C@H]5OC(O)[C@H](O)[C@@H](O)[C@H]5O)nc(OC[C@@]56CCCN5C[C@H](F)C6)nc4c3F)c12] |
| 87 | 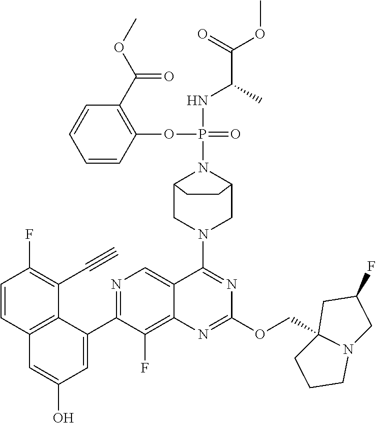 [SMILES: C#Cc1c(F)ccc2cc(O)cc(-c3ncc4c(N5CC6CCC(C5)N6P(=O)(N[C@@H](C)C(=O)OC)Oc5ccccc5C(=O)OC)nc(OC[C@@]56CCCN5C[C@H](F)C6)nc4c3F)c12] |
| 88 | 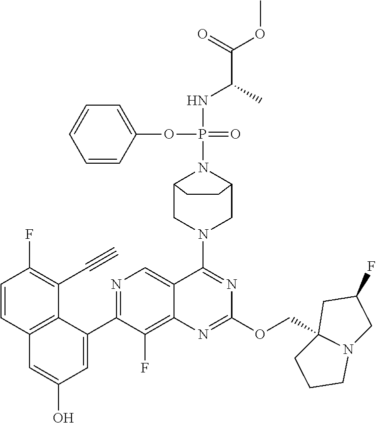 [SMILES: C#Cc1c(F)ccc2cc(O)cc(-c3ncc4c(N5CC6CCC(C5)N6P(=O)(N[C@@H](C)C(=O)OC)Oc5ccccc5)nc(OC[C@@]56CCCN5C[C@H](F)C6)nc4c3F)c12] |
| 89 | 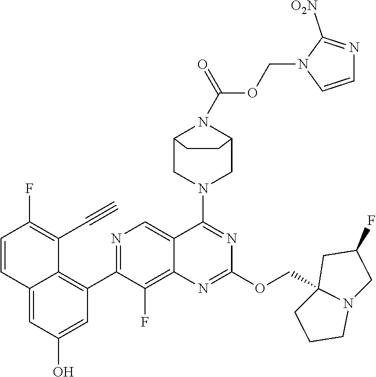 [SMILES: C#Cc1c(F)ccc2cc(O)cc(-c3ncc4c(N5CC6CCC(C5)N6C(=O)OCn5ccnc5[N+](=O)[O-])nc(OC[C@@]56CCCN5C[C@H](F)C6)nc4c3F)c12] |
| 90 | 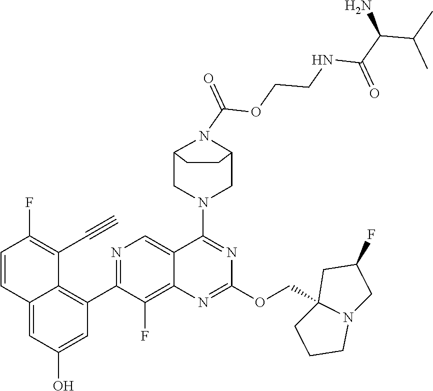 [SMILES: C#Cc1c(F)ccc2cc(O)cc(-c3ncc4c(N5CC6CCC(C5)N6C(=O)OCCNC(=O)[C@@H](N)C(C)C)nc(OC[C@@]56CCCN5C[C@H](F)C6)nc4c3F)c12] |
| 91 | 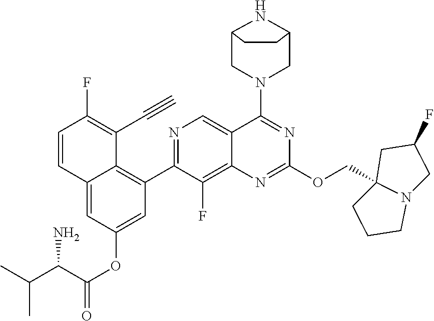 [SMILES: C#Cc1c(F)ccc2cc(OC(=O)[C@@H](N)C(C)C)cc(-c3ncc4c(N5CC6CCC(C5)N6)nc(OC[C@@]56CCCN5C[C@H](F)C6)nc4c3F)c12] |
| 92 | 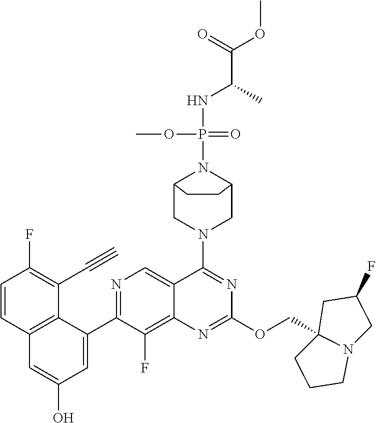 [SMILES: C#Cc1c(F)ccc2cc(O)cc(-c3ncc4c(N5CC6CCC(C5)N6P(=O)(N[C@@H](C)C(=O)OC)OC)nc(OC[C@@]56CCCN5C[C@H](F)C6)nc4c3F)c12] |
| 93 | 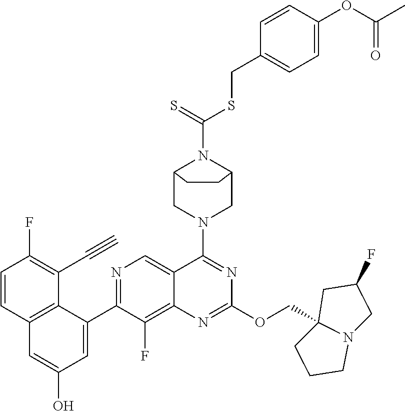 [SMILES: C#Cc1c(F)ccc2cc(O)cc(-c3ncc4c(N5CC6CCC(C5)N6C(=S)SCc5ccc(OC(C)=O)cc5)nc(OC[C@@]56CCCN5C[C@H](F)C6)nc4c3F)c12] |
| 94 | 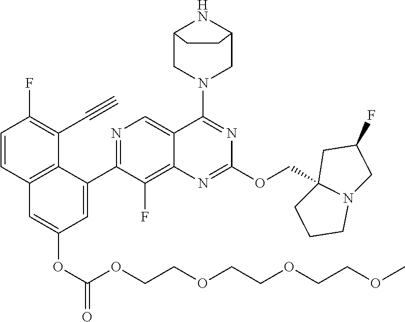 [SMILES: C#Cc1c(F)ccc2cc(OC(=O)OCCOCCOCCOC)cc(-c3ncc4c(N5CC6CCC(C5)N6)nc(OC[C@@]56CCCN5C[C@H](F)C6)nc4c3F)c12] |
| 95 | 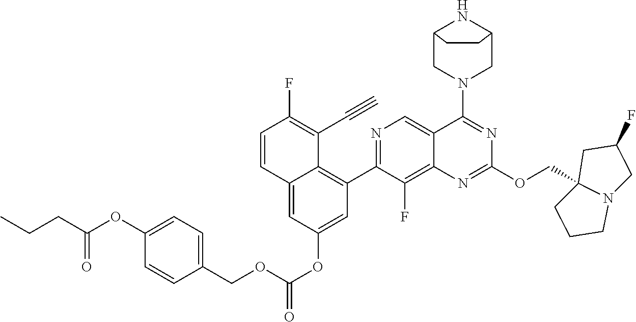 [SMILES: C#Cc1c(F)ccc2cc(OC(=O)OCc3ccc(OC(=O)CCC)cc3)cc(-c3ncc4c(N5CC6CCC(C5)N6)nc(OC[C@@]56CCCN5C[C@H](F)C6)nc4c3F)c12] |
| 96 | 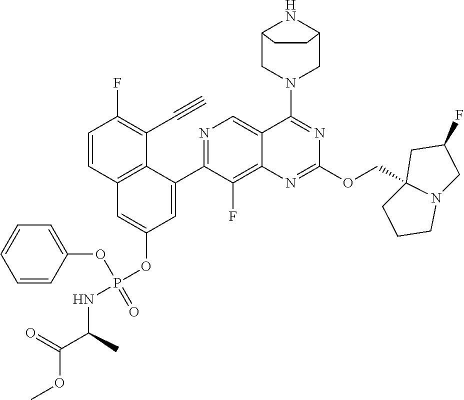 [SMILES: C#Cc1c(F)ccc2cc(OP(=O)(N[C@@H](C)C(=O)OC)Oc3ccccc3)cc(-c3ncc4c(N5CC6CCC(C5)N6)nc(OC[C@@]56CCCN5C[C@H](F)C6)nc4c3F)c12] |
| 97 | 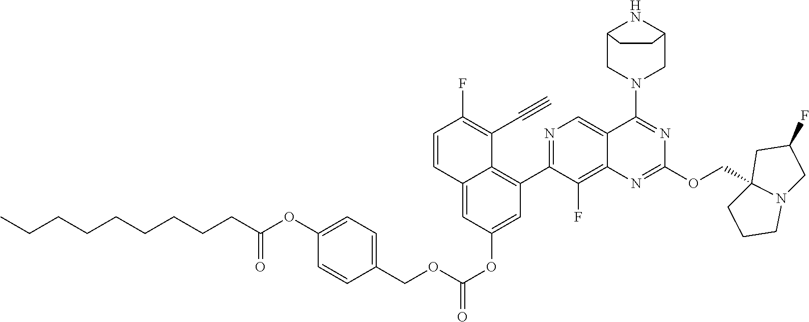 [SMILES: C#Cc1c(F)ccc2cc(OC(=O)OCc3ccc(OC(=O)CCCCCCCCC)cc3)cc(-c3ncc4c(N5CC6CCC(C5)N6)nc(OC[C@@]56CCCN5C[C@H](F)C6)nc4c3F)c12] |
| 98 | 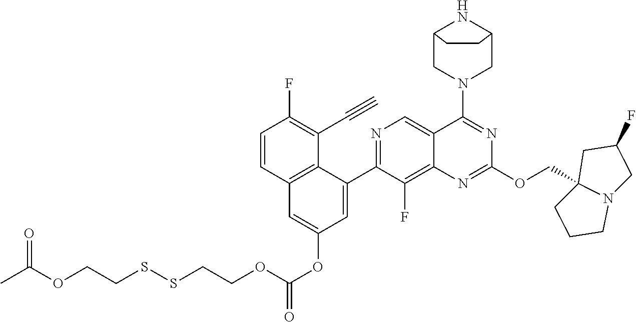 [SMILES: C#Cc1c(F)ccc2cc(OC(=O)OCCSSCCOC(C)=O)cc(-c3ncc4c(N5CC6CCC(C5)N6)nc(OC[C@@]56CCCN5C[C@H](F)C6)nc4c3F)c12] |
| 99 | 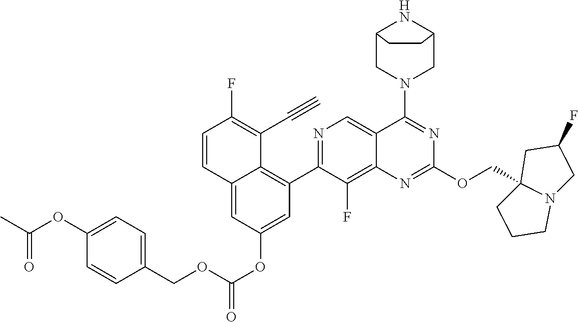 [SMILES: C#Cc1c(F)ccc2cc(OC(=O)OCc3ccc(OC(C)=O)cc3)cc(-c3ncc4c(N5CC6CCC(C5)N6)nc(OC[C@@]56CCCN5C[C@H](F)C6)nc4c3F)c12] |
| 100 | 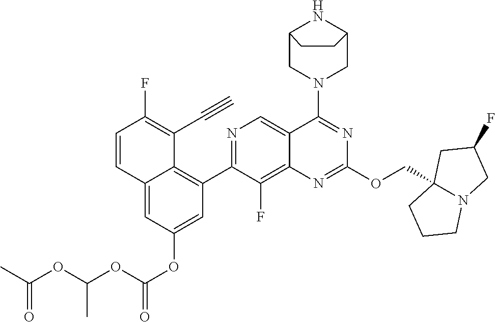 [SMILES: C#Cc1c(F)ccc2cc(OC(=O)OC(C)OC(C)=O)cc(-c3ncc4c(N5CC6CCC(C5)N6)nc(OC[C@@]56CCCN5C[C@H](F)C6)nc4c3F)c12] |
| 101 | 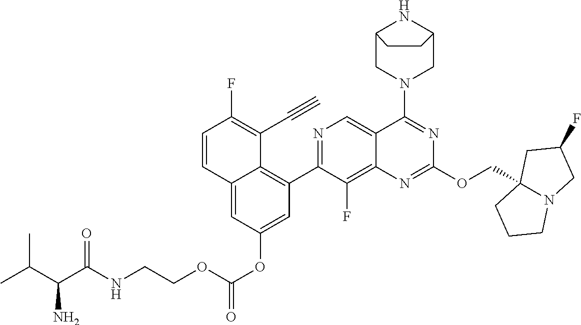 [SMILES: C#Cc1c(F)ccc2cc(OC(=O)OCCNC(=O)[C@@H](N)C(C)C)cc(-c3ncc4c(N5CC6CCC(C5)N6)nc(OC[C@@]56CCCN5C[C@H](F)C6)nc4c3F)c12] |
| 102 | 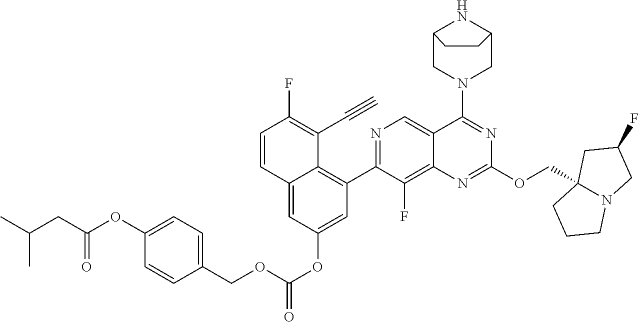 [SMILES: C#Cc1c(F)ccc2cc(OC(=O)OCc3ccc(OC(=O)CC(C)C)cc3)cc(-c3ncc4c(N5CC6CCC(C5)N6)nc(OC[C@@]56CCCN5C[C@H](F)C6)nc4c3F)c12] |
| 103 | 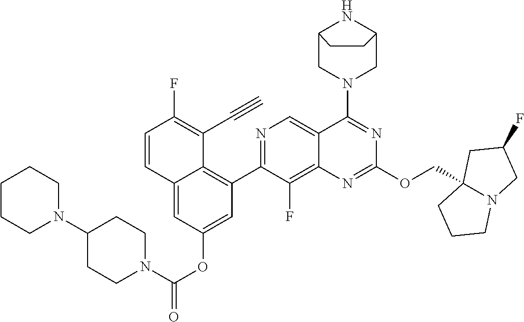 [SMILES: C#Cc1c(F)ccc2cc(OC(=O)N3CCC(N4CCCCC4)CC3)cc(-c3ncc4c(N5CC6CCC(C5)N6)nc(OC[C@@]56CCCN5C[C@H](F)C6)nc4c3F)c12] |
| 104 | 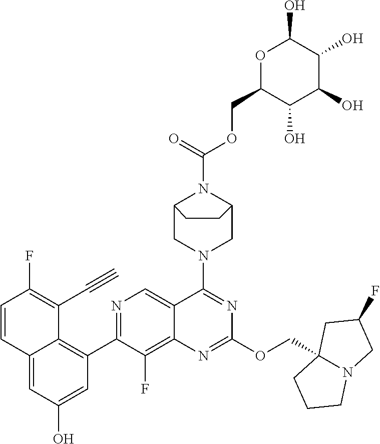 [SMILES: C#Cc1c(F)ccc2cc(O)cc(-c3ncc4c(N5CC6CCC(C5)N6C(=O)OC[C@H]5O[C@@H](O)[C@H](O)[C@@H](O)[C@@H]5O)nc(OC[C@@]56CCCN5C[C@H](F)C6)nc4c3F)c12] |
| 105 | 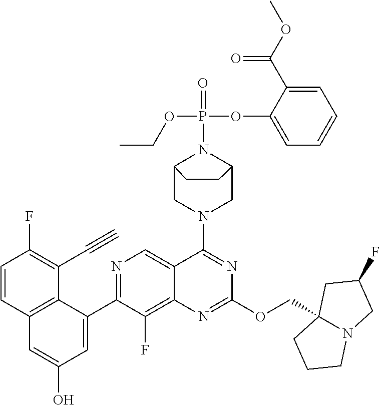 [SMILES: C#Cc1c(F)ccc2cc(O)cc(-c3ncc4c(N5CC6CCC(C5)N6P(=O)(OCC)Oc5ccccc5C(=O)OC)nc(OC[C@@]56CCCN5C[C@H](F)C6)nc4c3F)c12] |
| 106 | 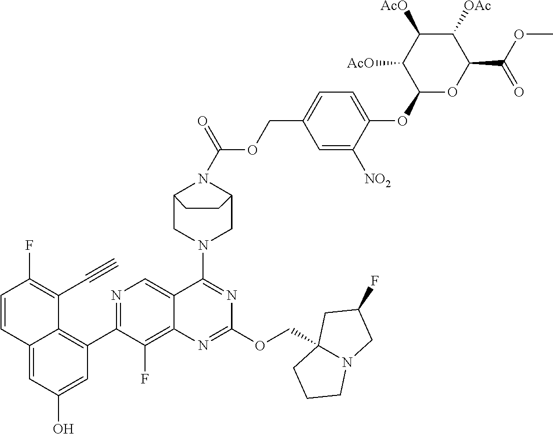 [SMILES: C#Cc1c(F)ccc2cc(O)cc(-c3ncc4c(N5CC6CCC(C5)N6C(=O)OCc5ccc(O[C@@H]6O[C@H](C(=O)OC)[C@@H](OC(C)=O)[C@H](OC(C)=O)[C@H]6OC(C)=O)c([N+](=O)[O-])c5)nc(OC[C@@]56CCCN5C[C@H](F)C6)nc4c3F)c12] |
| 107 | 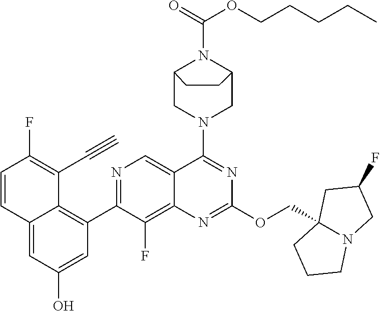 [SMILES: C#Cc1c(F)ccc2cc(O)cc(-c3ncc4c(N5CC6CCC(C5)N6C(=O)OCCCCC)nc(OC[C@@]56CCCN5C[C@H](F)C6)nc4c3F)c12] |
| 108 | 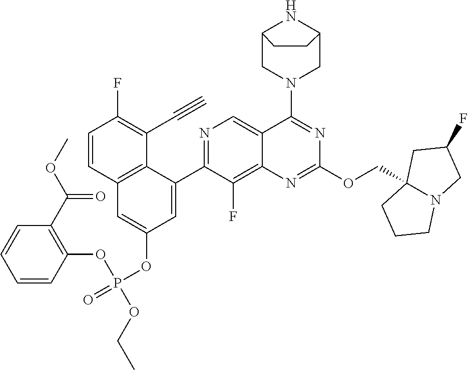 [SMILES: C#Cc1c(F)ccc2cc(OP(=O)(OCC)Oc3ccccc3C(=O)OC)cc(-c3ncc4c(N5CC6CCC(C5)N6)nc(OC[C@@]56CCCN5C[C@H](F)C6)nc4c3F)c12] |
| 109 | 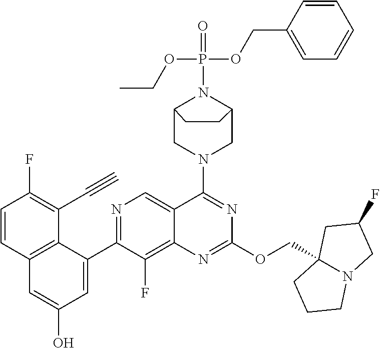 [SMILES: C#Cc1c(F)ccc2cc(O)cc(-c3ncc4c(N5CC6CCC(C5)N6P(=O)(OCC)OCc5ccccc5)nc(OC[C@@]56CCCN5C[C@H](F)C6)nc4c3F)c12] |
| 110 | 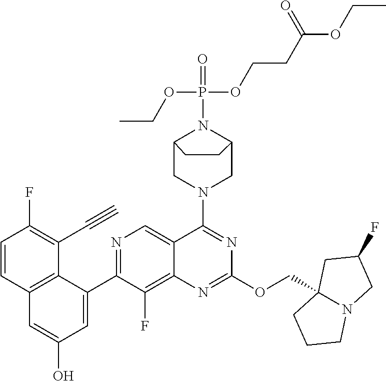 [SMILES: C#Cc1c(F)ccc2cc(O)cc(-c3ncc4c(N5CC6CCC(C5)N6P(=O)(OCC)OCCC(=O)OCC)nc(OC[C@@]56CCCN5C[C@H](F)C6)nc4c3F)c12] |
| 111 | 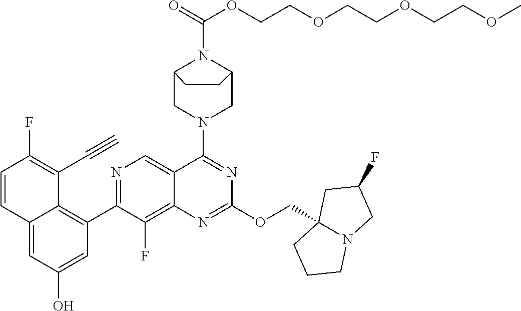 [SMILES: C#Cc1c(F)ccc2cc(O)cc(-c3ncc4c(N5CC6CCC(C5)N6C(=O)OCCOCCOCCOC)nc(OC[C@@]56CCCN5C[C@H](F)C6)nc4c3F)c12] |
| 112 | 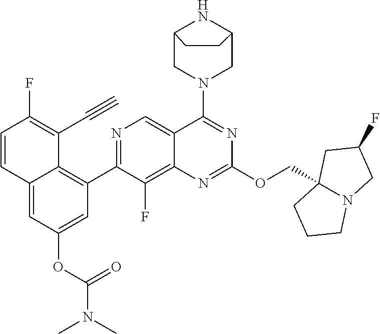 [SMILES: C#Cc1c(F)ccc2cc(OC(=O)N(C)C)cc(-c3ncc4c(N5CC6CCC(C5)N6)nc(OC[C@@]56CCCN5C[C@H](F)C6)nc4c3F)c12] |
| 113 | 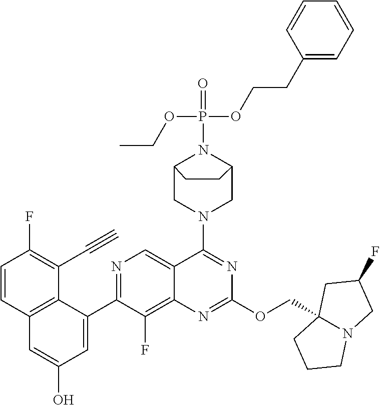 [SMILES: C#Cc1c(F)ccc2cc(O)cc(-c3ncc4c(N5CC6CCC(C5)N6P(=O)(OCC)OCCc5ccccc5)nc(OC[C@@]56CCCN5C[C@H](F)C6)nc4c3F)c12] |
| 114 | 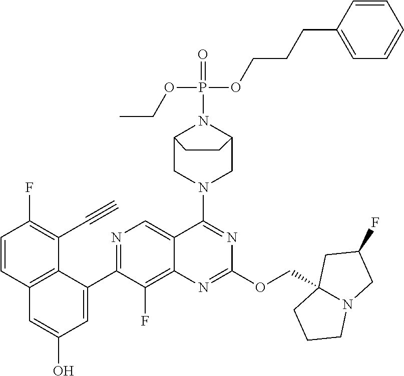 [SMILES: C#Cc1c(F)ccc2cc(O)cc(-c3ncc4c(N5CC6CCC(C5)N6P(=O)(OCC)OCCCc5ccccc5)nc(OC[C@@]56CCCN5C[C@H](F)C6)nc4c3F)c12] |
| 115 | 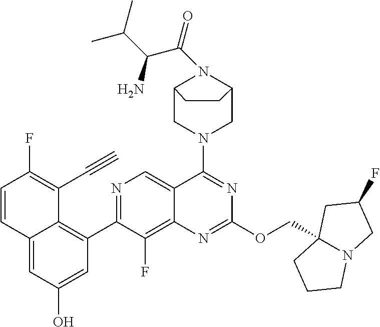 [SMILES: C#Cc1c(F)ccc2cc(O)cc(-c3ncc4c(N5CC6CCC(C5)N6C(=O)[C@@H](N)C(C)C)nc(OC[C@@]56CCCN5C[C@H](F)C6)nc4c3F)c12] |
| 116 | 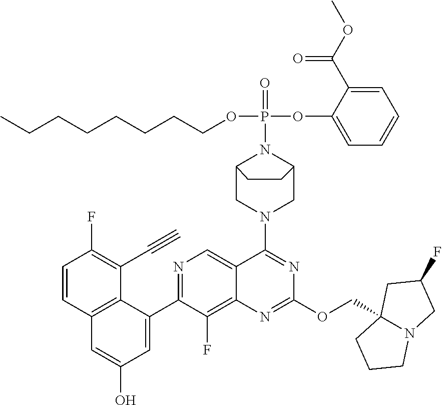 [SMILES: C#Cc1c(F)ccc2cc(O)cc(-c3ncc4c(N5CC6CCC(C5)N6P(=O)(OCCCCCCCC)Oc5ccccc5C(=O)OC)nc(OC[C@@]56CCCN5C[C@H](F)C6)nc4c3F)c12] |
| 117 | 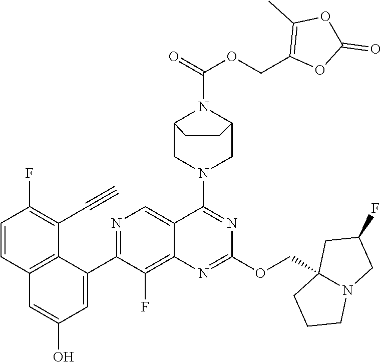 [SMILES: C#Cc1c(F)ccc2cc(O)cc(-c3ncc4c(N5CC6CCC(C5)N6C(=O)OCc5oc(=O)oc5C)nc(OC[C@@]56CCCN5C[C@H](F)C6)nc4c3F)c12] |
| 118 | 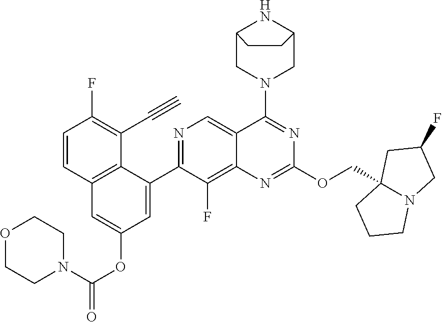 [SMILES: C#Cc1c(F)ccc2cc(OC(=O)N3CCOCC3)cc(-c3ncc4c(N5CC6CCC(C5)N6)nc(OC[C@@]56CCCN5C[C@H](F)C6)nc4c3F)c12] |
| 119 | 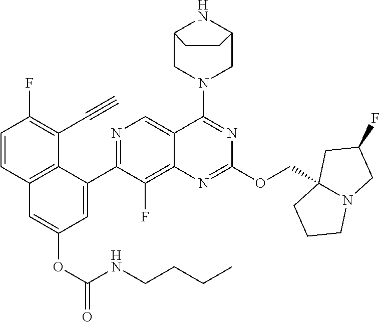 [SMILES: C#Cc1c(F)ccc2cc(OC(=O)NCCCC)cc(-c3ncc4c(N5CC6CCC(C5)N6)nc(OC[C@@]56CCCN5C[C@H](F)C6)nc4c3F)c12] |
| 120 | 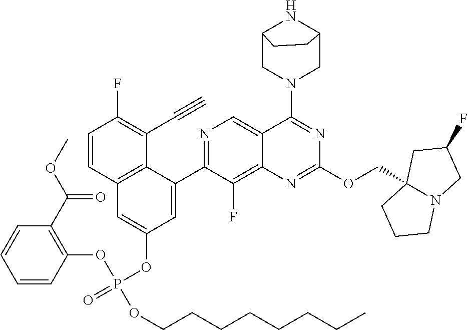 [SMILES: C#Cc1c(F)ccc2cc(OP(=O)(OCCCCCCCC)Oc3ccccc3C(=O)OC)cc(-c3ncc4c(N5CC6CCC(C5)N6)nc(OC[C@@]56CCCN5C[C@H](F)C6)nc4c3F)c12] |
| 121 | 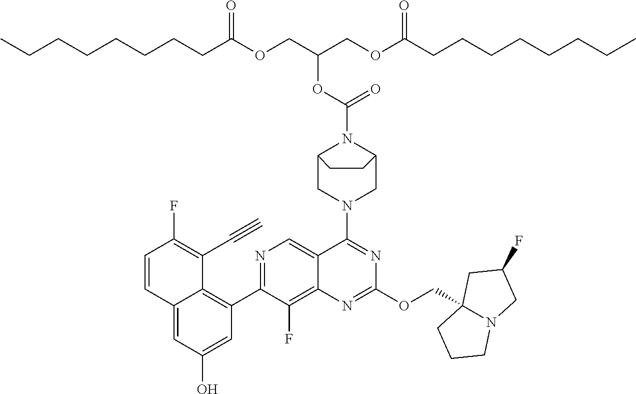 [SMILES: C#Cc1c(F)ccc2cc(O)cc(-c3ncc4c(N5CC6CCC(C5)N6C(=O)OC(COC(=O)CCCCCCCC)COC(=O)CCCCCCCC)nc(OC[C@@]56CCCN5C[C@H](F)C6)nc4c3F)c12] |
| 122 | 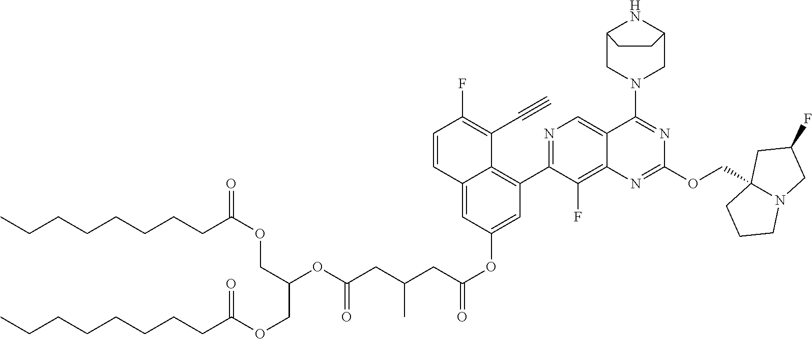 [SMILES: C#Cc1c(F)ccc2cc(OC(=O)CC(C)CC(=O)OC(COC(=O)CCCCCCCC)COC(=O)CCCCCCCC)cc(-c3ncc4c(N5CC6CCC(C5)N6)nc(OC[C@@]56CCCN5C[C@H](F)C6)nc4c3F)c12] |
| 123 | 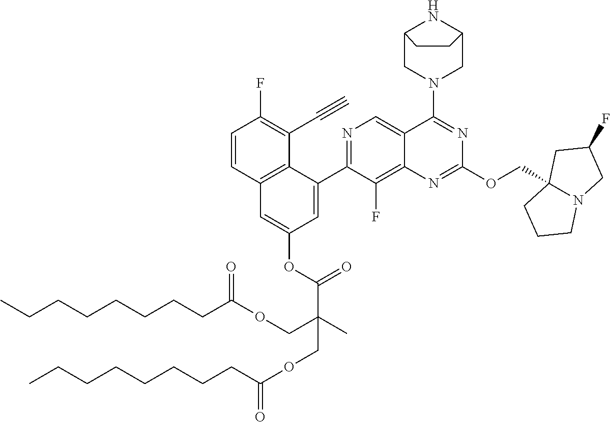 [SMILES: C#Cc1c(F)ccc2cc(OC(=O)C(C)(COC(=O)CCCCCCCC)COC(=O)CCCCCCCC)cc(-c3ncc4c(N5CC6CCC(C5)N6)nc(OC[C@@]56CCCN5C[C@H](F)C6)nc4c3F)c12] |
| 124 | 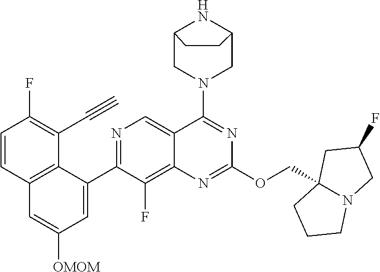 |
| 125 | 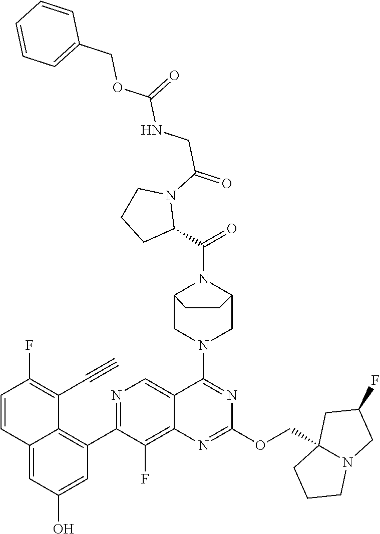 [SMILES: C#Cc1c(F)ccc2cc(O)cc(-c3ncc4c(N5CC6CCC(C5)N6C(=O)[C@@H]5CCCN5C(=O)CNC(=O)OCc5ccccc5)nc(OC[C@@]56CCCN5C[C@H](F)C6)nc4c3F)c12] |
| 126 | 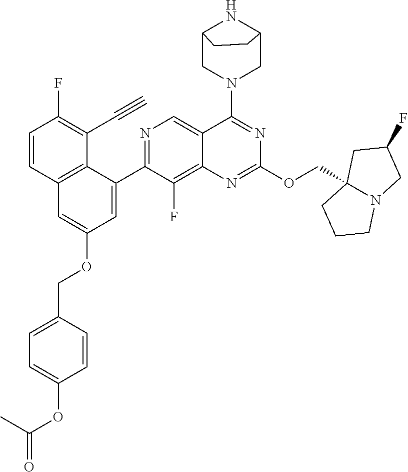 [SMILES: C#Cc1c(F)ccc2cc(OCc3ccc(OC(C)=O)cc3)cc(-c3ncc4c(N5CC6CCC(C5)N6)nc(OC[C@@]56CCCN5C[C@H](F)C6)nc4c3F)c12] |
| 127 | 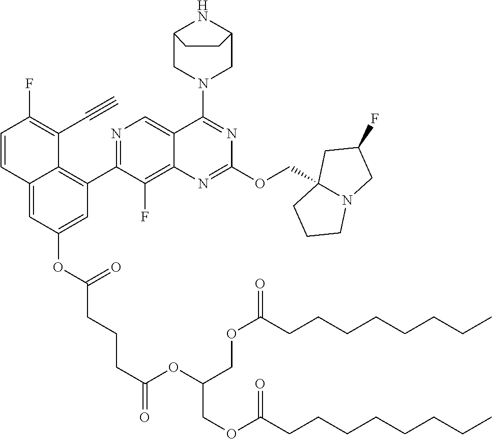 [SMILES: C#Cc1c(F)ccc2cc(OC(=O)CCCC(=O)OC(COC(=O)CCCCCCCC)COC(=O)CCCCCCCC)cc(-c3ncc4c(N5CC6CCC(C5)N6)nc(OC[C@@]56CCCN5C[C@H](F)C6)nc4c3F)c12] |
| 128 | 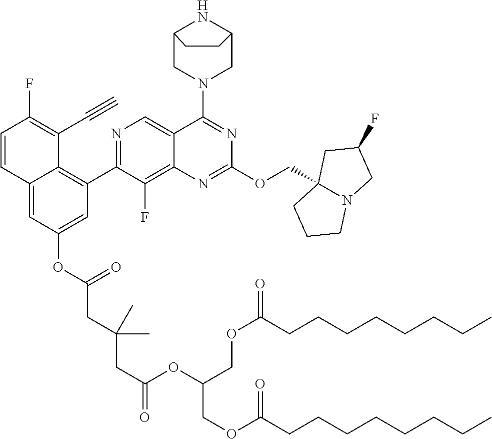 [SMILES: C#Cc1c(F)ccc2cc(OC(=O)CC(C)(C)CC(=O)OC(COC(=O)CCCCCCCC)COC(=O)CCCCCCCC)cc(-c3ncc4c(N5CC6CCC(C5)N6)nc(OC[C@@]56CCCN5C[C@H](F)C6)nc4c3F)c12] |
| 129 | 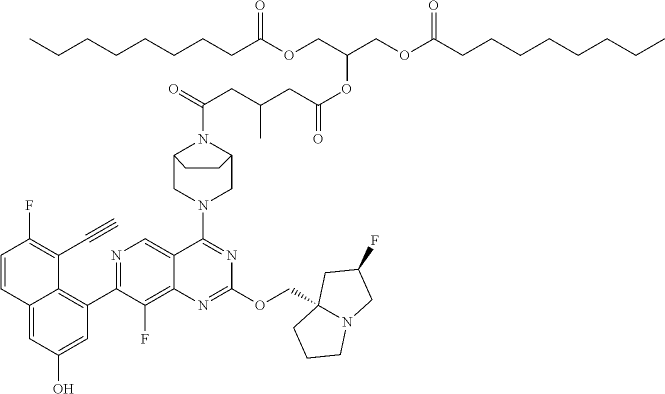 [SMILES: C#Cc1c(F)ccc2cc(O)cc(-c3ncc4c(N5CC6CCC(C5)N6C(=O)CC(C)CC(=O)OC(COC(=O)CCCCCCCC)COC(=O)CCCCCCCC)nc(OC[C@@]56CCCN5C[C@H](F)C6)nc4c3F)c12] |
| 130 | 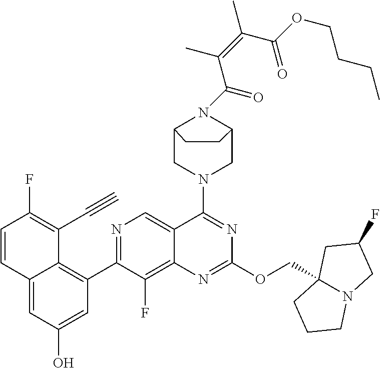 [SMILES: C#Cc1c(F)ccc2cc(O)cc(-c3ncc4c(N5CC6CCC(C5)N6C(=O)/C(C)=C(/C)C(=O)OCCCC)nc(OC[C@@]56CCCN5C[C@H](F)C6)nc4c3F)c12] |
| 131 | 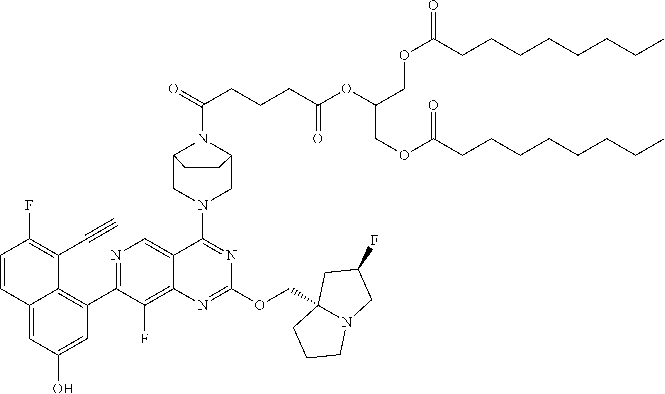 [SMILES: C#Cc1c(F)ccc2cc(O)cc(-c3ncc4c(N5CC6CCC(C5)N6C(=O)CCCC(=O)OC(COC(=O)CCCCCCCC)COC(=O)CCCCCCCC)nc(OC[C@@]56CCCN5C[C@H](F)C6)nc4c3F)c12] |
| 132 | 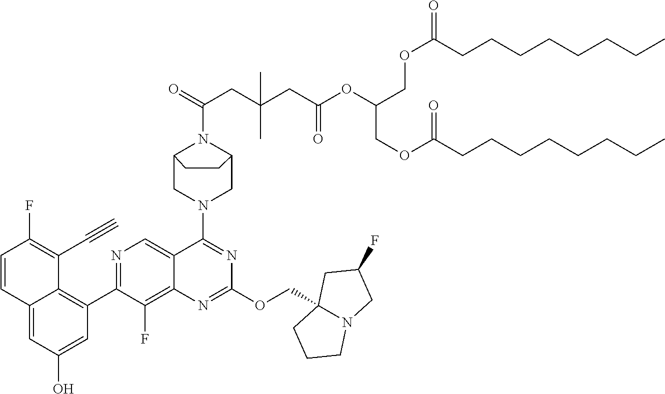 [SMILES: C#Cc1c(F)ccc2cc(O)cc(-c3ncc4c(N5CC6CCC(C5)N6C(=O)CC(C)(C)CC(=O)OC(COC(=O)CCCCCCCC)COC(=O)CCCCCCCC)nc(OC[C@@]56CCCN5C[C@H](F)C6)nc4c3F)c12] |
| 133 | 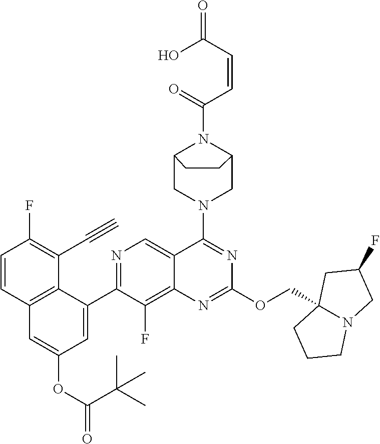 [SMILES: C#Cc1c(F)ccc2cc(OC(=O)C(C)(C)C)cc(-c3ncc4c(N5CC6CCC(C5)N6C(=O)/C=C\C(=O)O)nc(OC[C@@]56CCCN5C[C@H](F)C6)nc4c3F)c12] |
| 134 | 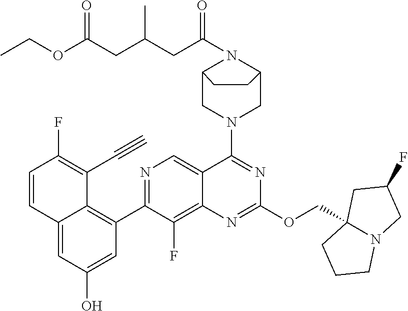 [SMILES: C#Cc1c(F)ccc2cc(O)cc(-c3ncc4c(N5CC6CCC(C5)N6C(=O)CC(C)CC(=O)OCC)nc(OC[C@@]56CCCN5C[C@H](F)C6)nc4c3F)c12] |
| 135 | 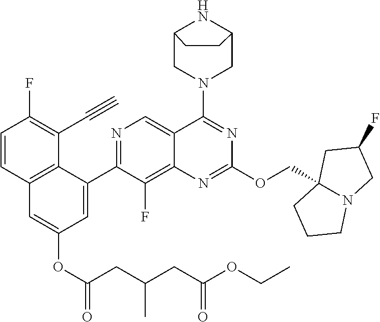 [SMILES: C#Cc1c(F)ccc2cc(OC(=O)CC(C)CC(=O)OCC)cc(-c3ncc4c(N5CC6CCC(C5)N6)nc(OC[C@@]56CCCN5C[C@H](F)C6)nc4c3F)c12] |
| 136 | 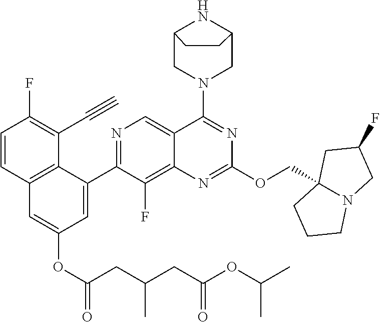 [SMILES: C#Cc1c(F)ccc2cc(OC(=O)CC(C)CC(=O)OC(C)C)cc(-c3ncc4c(N5CC6CCC(C5)N6)nc(OC[C@@]56CCCN5C[C@H](F)C6)nc4c3F)c12] |
| 137 | 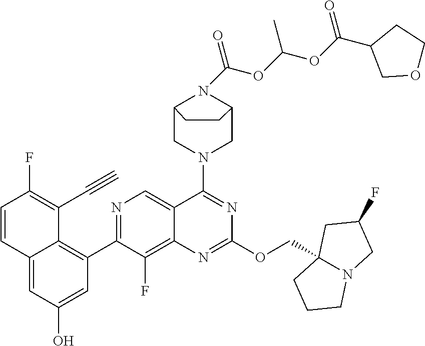 [SMILES: C#Cc1c(F)ccc2cc(O)cc(-c3ncc4c(N5CC6CCC(C5)N6C(=O)OC(C)OC(=O)C5CCOC5)nc(OC[C@@]56CCCN5C[C@H](F)C6)nc4c3F)c12] |
| 138 | 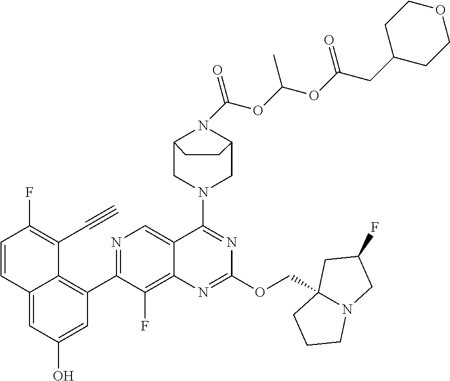 [SMILES: C#Cc1c(F)ccc2cc(O)cc(-c3ncc4c(N5CC6CCC(C5)N6C(=O)OC(C)OC(=O)CC5CCOCC5)nc(OC[C@@]56CCCN5C[C@H](F)C6)nc4c3F)c12] |
| 139 | 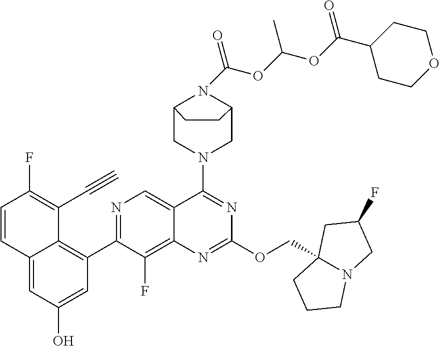 [SMILES: C#Cc1c(F)ccc2cc(O)cc(-c3ncc4c(N5CC6CCC(C5)N6C(=O)OC(C)OC(=O)C5CCOCC5)nc(OC[C@@]56CCCN5C[C@H](F)C6)nc4c3F)c12] |
| 140 | 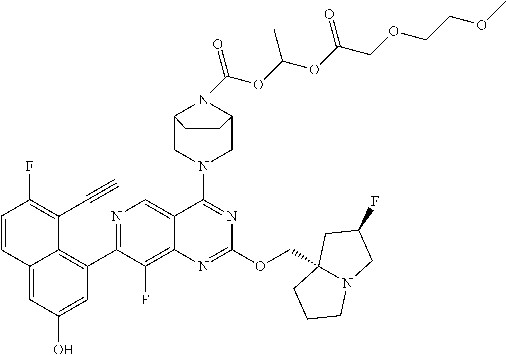 [SMILES: C#Cc1c(F)ccc2cc(O)cc(-c3ncc4c(N5CC6CCC(C5)N6C(=O)OC(C)OC(=O)COCCOC)nc(OC[C@@]56CCCN5C[C@H](F)C6)nc4c3F)c12] |
| 141 | 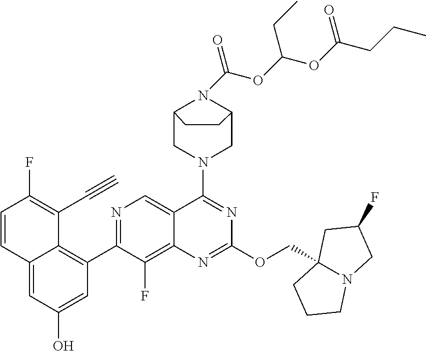 [SMILES: C#Cc1c(F)ccc2cc(O)cc(-c3ncc4c(N5CC6CCC(C5)N6C(=O)OC(CC)OC(=O)CCC)nc(OC[C@@]56CCCN5C[C@H](F)C6)nc4c3F)c12] |
| 142 | 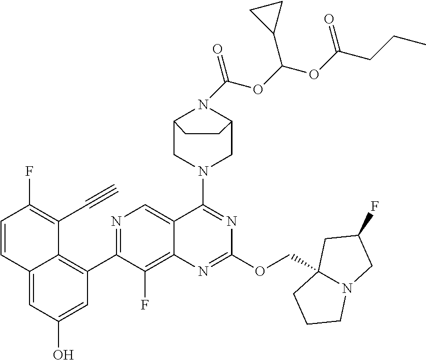 [SMILES: C#Cc1c(F)ccc2cc(O)cc(-c3ncc4c(N5CC6CCC(C5)N6C(=O)OC(OC(=O)CCC)C5CC5)nc(OC[C@@]56CCCN5C[C@H](F)C6)nc4c3F)c12] |
| 143 | 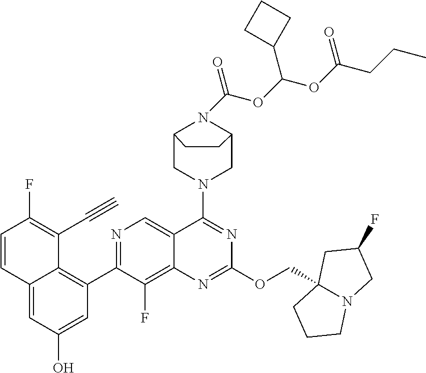 [SMILES: C#Cc1c(F)ccc2cc(O)cc(-c3ncc4c(N5CC6CCC(C5)N6C(=O)OC(OC(=O)CCC)C5CCC5)nc(OC[C@@]56CCCN5C[C@H](F)C6)nc4c3F)c12] |
| 144 | 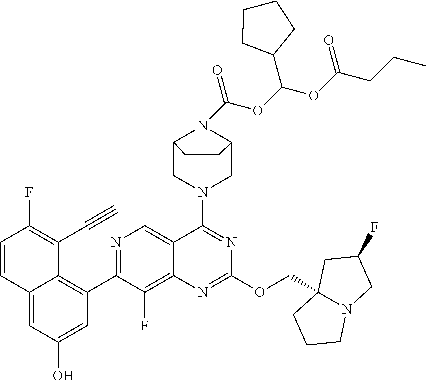 [SMILES: C#Cc1c(F)ccc2cc(O)cc(-c3ncc4c(N5CC6CCC(C5)N6C(=O)OC(OC(=O)CCC)C5CCCC5)nc(OC[C@@]56CCCN5C[C@H](F)C6)nc4c3F)c12] |
| 145 | 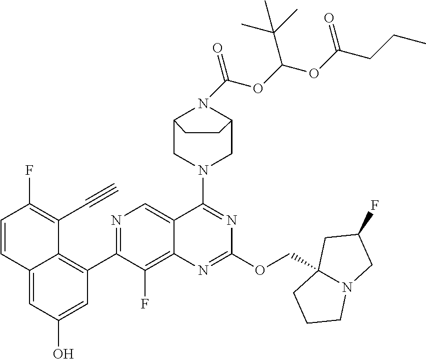 [SMILES: C#Cc1c(F)ccc2cc(O)cc(-c3ncc4c(N5CC6CCC(C5)N6C(=O)OC(OC(=O)CCC)C(C)(C)C)nc(OC[C@@]56CCCN5C[C@H](F)C6)nc4c3F)c12] |
| 146 | 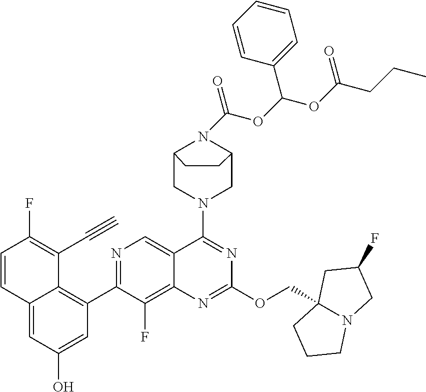 [SMILES: C#Cc1c(F)ccc2cc(O)cc(-c3ncc4c(N5CC6CCC(C5)N6C(=O)OC(OC(=O)CCC)c5ccccc5)nc(OC[C@@]56CCCN5C[C@H](F)C6)nc4c3F)c12] |
| 147 | 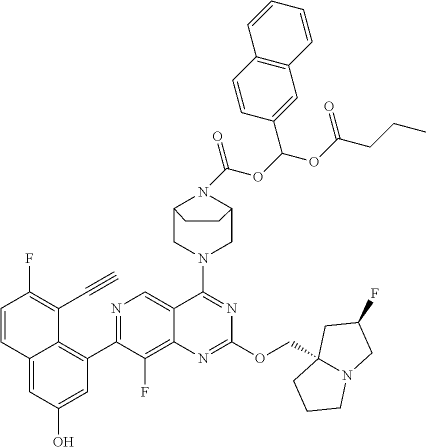 [SMILES: C#Cc1c(F)ccc2cc(O)cc(-c3ncc4c(N5CC6CCC(C5)N6C(=O)OC(OC(=O)CCC)c5ccc6ccccc6c5)nc(OC[C@@]56CCCN5C[C@H](F)C6)nc4c3F)c12] |
| 148 | 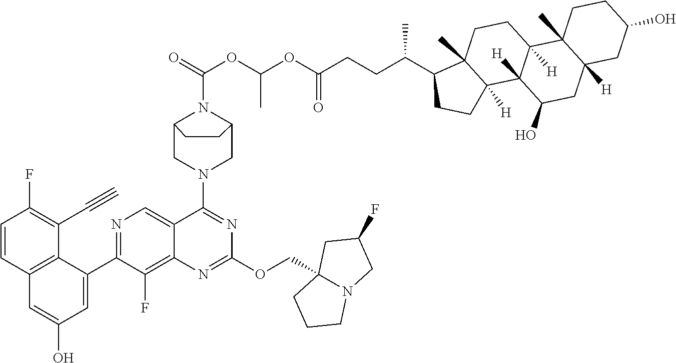 |
| 149 | 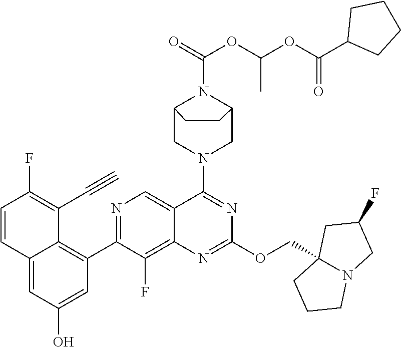 [SMILES: C#Cc1c(F)ccc2cc(O)cc(-c3ncc4c(N5CC6CCC(C5)N6C(=O)OC(C)OC(=O)C5CCCC5)nc(OC[C@@]56CCCN5C[C@H](F)C6)nc4c3F)c12] |
| 150 | 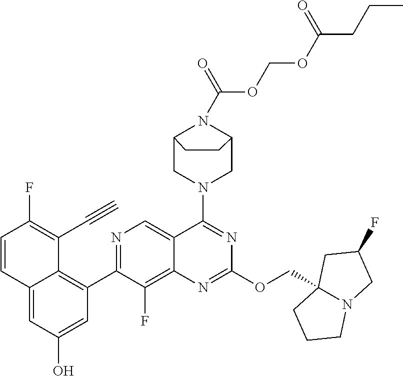 [SMILES: C#Cc1c(F)ccc2cc(O)cc(-c3ncc4c(N5CC6CCC(C5)N6C(=O)OCOC(=O)CCC)nc(OC[C@@]56CCCN5C[C@H](F)C6)nc4c3F)c12] |
| 151 | 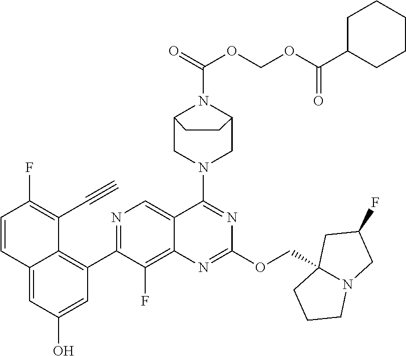 [SMILES: C#Cc1c(F)ccc2cc(O)cc(-c3ncc4c(N5CC6CCC(C5)N6C(=O)OCOC(=O)C5CCCCC5)nc(OC[C@@]56CCCN5C[C@H](F)C6)nc4c3F)c12] |
| 152 | 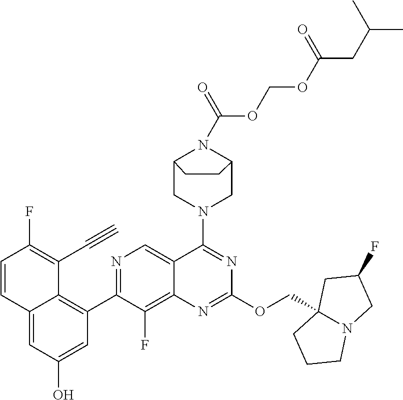 [SMILES: C#Cc1c(F)ccc2cc(O)cc(-c3ncc4c(N5CC6CCC(C5)N6C(=O)OCOC(=O)CC(C)C)nc(OC[C@@]56CCCN5C[C@H](F)C6)nc4c3F)c12] |
| 153 | 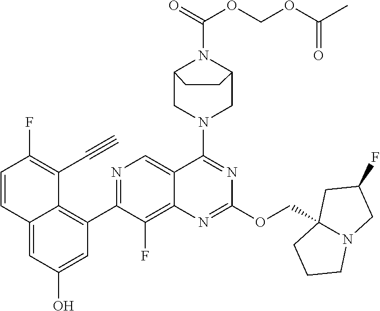 [SMILES: C#Cc1c(F)ccc2cc(O)cc(-c3ncc4c(N5CC6CCC(C5)N6C(=O)OCOC(C)=O)nc(OC[C@@]56CCCN5C[C@H](F)C6)nc4c3F)c12] |
| 154 | 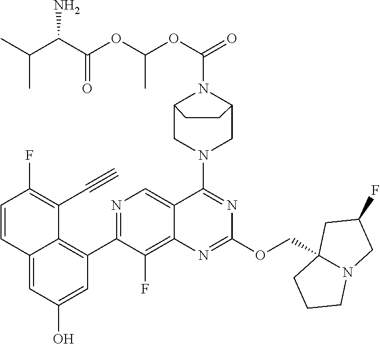 [SMILES: C#Cc1c(F)ccc2cc(O)cc(-c3ncc4c(N5CC6CCC(C5)N6C(=O)OC(C)OC(=O)[C@@H](N)C(C)C)nc(OC[C@@]56CCCN5C[C@H](F)C6)nc4c3F)c12] |
| 155 | 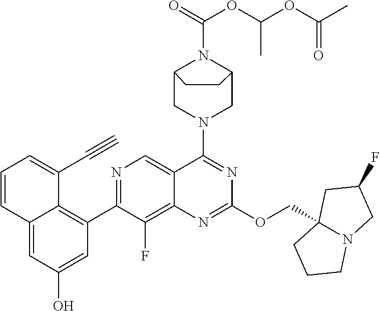 [SMILES: C#Cc1cccc2cc(O)cc(-c3ncc4c(N5CC6CCC(C5)N6C(=O)OC(C)OC(C)=O)nc(OC[C@@]56CCCN5C[C@H](F)C6)nc4c3F)c12] |
| 156 | 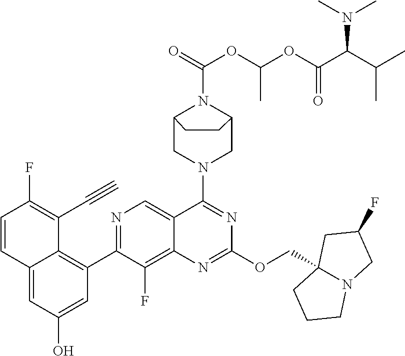 [SMILES: C#Cc1c(F)ccc2cc(O)cc(-c3ncc4c(N5CC6CCC(C5)N6C(=O)OC(C)OC(=O)[C@H](C(C)C)N(C)C)nc(OC[C@@]56CCCN5C[C@H](F)C6)nc4c3F)c12] |
| 157 | 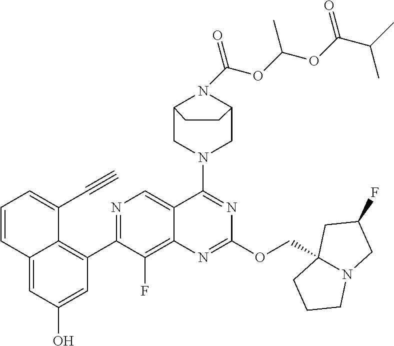 [SMILES: C#Cc1cccc2cc(O)cc(-c3ncc4c(N5CC6CCC(C5)N6C(=O)OC(C)OC(=O)C(C)C)nc(OC[C@@]56CCCN5C[C@H](F)C6)nc4c3F)c12] |
| 158 | 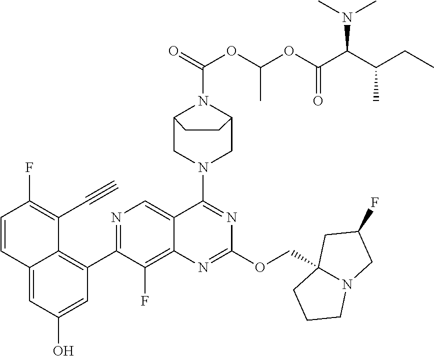 [SMILES: C#Cc1c(F)ccc2cc(O)cc(-c3ncc4c(N5CC6CCC(C5)N6C(=O)OC(C)OC(=O)[C@H]([C@@H](C)CC)N(C)C)nc(OC[C@@]56CCCN5C[C@H](F)C6)nc4c3F)c12] |
| 159 | 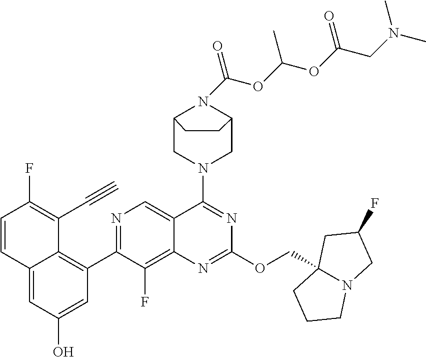 [SMILES: C#Cc1c(F)ccc2cc(O)cc(-c3ncc4c(N5CC6CCC(C5)N6C(=O)OC(C)OC(=O)CN(C)C)nc(OC[C@@]56CCCN5C[C@H](F)C6)nc4c3F)c12] |
| 160 | 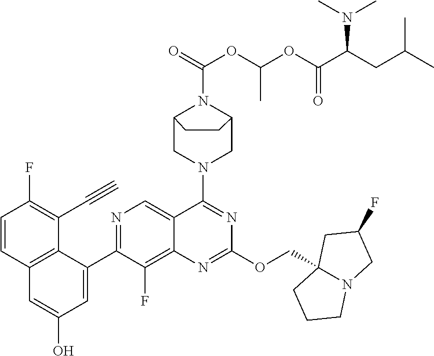 [SMILES: C#Cc1c(F)ccc2cc(O)cc(-c3ncc4c(N5CC6CCC(C5)N6C(=O)OC(C)OC(=O)[C@H](CC(C)C)N(C)C)nc(OC[C@@]56CCCN5C[C@H](F)C6)nc4c3F)c12] |
| 161 | 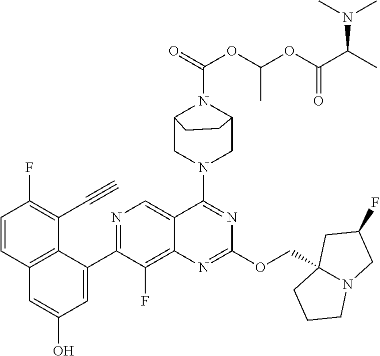 [SMILES: C#Cc1c(F)ccc2cc(O)cc(-c3ncc4c(N5CC6CCC(C5)N6C(=O)OC(C)OC(=O)[C@H](C)N(C)C)nc(OC[C@@]56CCCN5C[C@H](F)C6)nc4c3F)c12] |
| 162 | 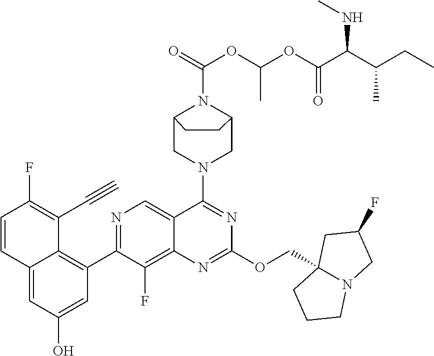 [SMILES: C#Cc1c(F)ccc2cc(O)cc(-c3ncc4c(N5CC6CCC(C5)N6C(=O)OC(C)OC(=O)[C@@H](NC)[C@@H](C)CC)nc(OC[C@@]56CCCN5C[C@H](F)C6)nc4c3F)c12] |
| 163 | 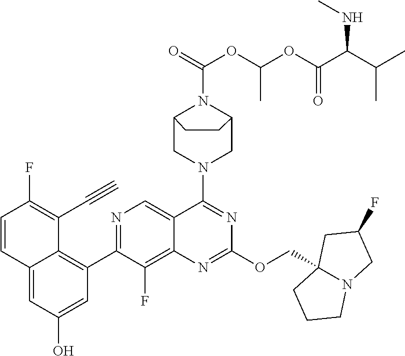 [SMILES: C#Cc1c(F)ccc2cc(O)cc(-c3ncc4c(N5CC6CCC(C5)N6C(=O)OC(C)OC(=O)[C@@H](NC)C(C)C)nc(OC[C@@]56CCCN5C[C@H](F)C6)nc4c3F)c12] |
| 164 | 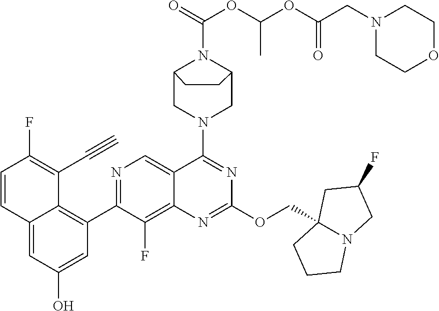 [SMILES: C#Cc1c(F)ccc2cc(O)cc(-c3ncc4c(N5CC6CCC(C5)N6C(=O)OC(C)OC(=O)CN5CCOCC5)nc(OC[C@@]56CCCN5C[C@H](F)C6)nc4c3F)c12] |
| 165 | 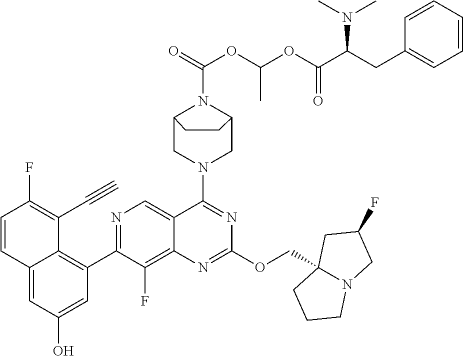 [SMILES: C#Cc1c(F)ccc2cc(O)cc(-c3ncc4c(N5CC6CCC(C5)N6C(=O)OC(C)OC(=O)[C@H](Cc5ccccc5)N(C)C)nc(OC[C@@]56CCCN5C[C@H](F)C6)nc4c3F)c12] |
| 166 | 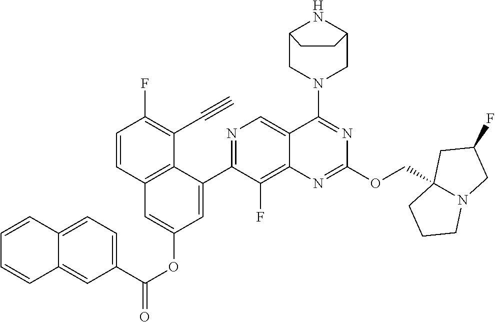 [SMILES: C#Cc1c(F)ccc2cc(OC(=O)c3ccc4ccccc4c3)cc(-c3ncc4c(N5CC6CCC(C5)N6)nc(OC[C@@]56CCCN5C[C@H](F)C6)nc4c3F)c12] |
| 167 | 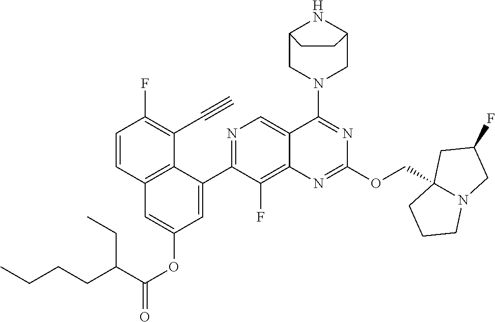 [SMILES: C#Cc1c(F)ccc2cc(OC(=O)C(CC)CCCC)cc(-c3ncc4c(N5CC6CCC(C5)N6)nc(OC[C@@]56CCCN5C[C@H](F)C6)nc4c3F)c12] |
| 168 | 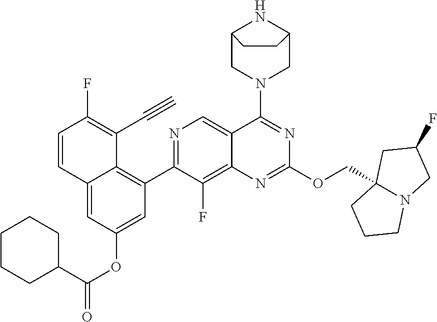 [SMILES: C#Cc1c(F)ccc2cc(OC(=O)C3CCCCC3)cc(-c3ncc4c(N5CC6CCC(C5)N6)nc(OC[C@@]56CCCN5C[C@H](F)C6)nc4c3F)c12] |
| 169 | 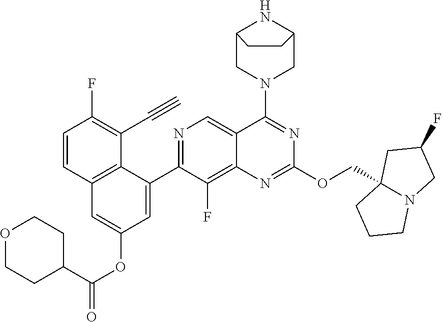 [SMILES: C#Cc1c(F)ccc2cc(OC(=O)C3CCOCC3)cc(-c3ncc4c(N5CC6CCC(C5)N6)nc(OC[C@@]56CCCN5C[C@H](F)C6)nc4c3F)c12] |
| 170 | 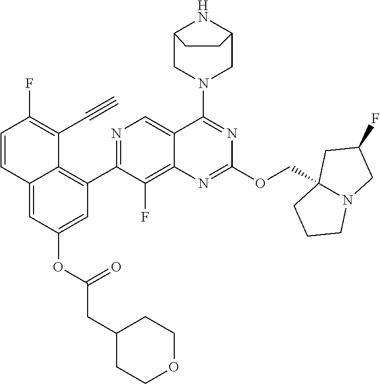 [SMILES: C#Cc1c(F)ccc2cc(OC(=O)CC3CCOCC3)cc(-c3ncc4c(N5CC6CCC(C5)N6)nc(OC[C@@]56CCCN5C[C@H](F)C6)nc4c3F)c12] |
| 171 | 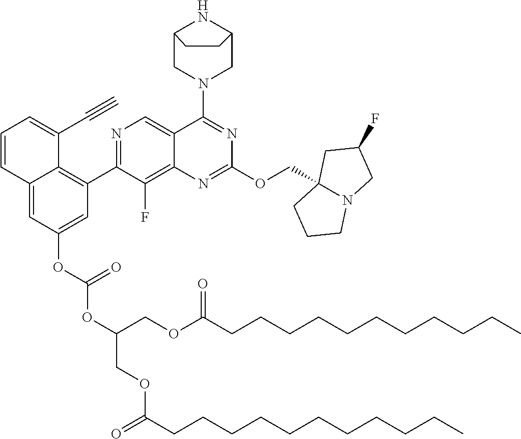 [SMILES: C#Cc1cccc2cc(OC(=O)OC(COC(=O)CCCCCCCCCCC)COC(=O)CCCCCCCCCCC)cc(-c3ncc4c(N5CC6CCC(C5)N6)nc(OC[C@@]56CCCN5C[C@H](F)C6)nc4c3F)c12] |
| 172 | 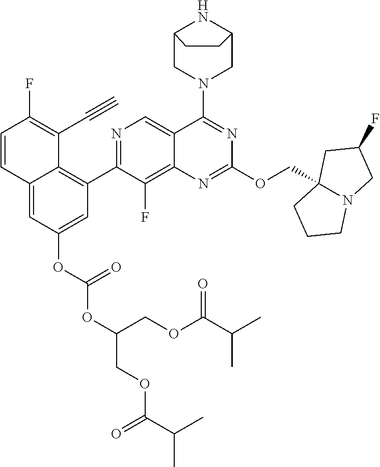 [SMILES: C#Cc1c(F)ccc2cc(OC(=O)OC(COC(=O)C(C)C)COC(=O)C(C)C)cc(-c3ncc4c(N5CC6CCC(C5)N6)nc(OC[C@@]56CCCN5C[C@H](F)C6)nc4c3F)c12] |
| 173 | 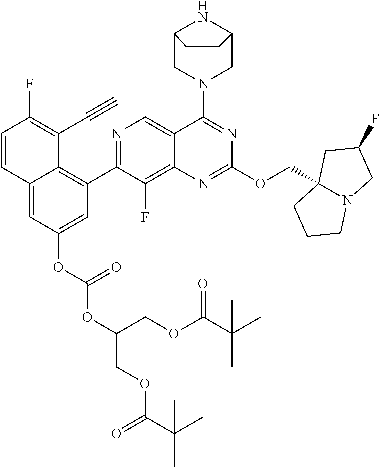 [SMILES: C#Cc1c(F)ccc2cc(OC(=O)OC(COC(=O)C(C)(C)C)COC(=O)C(C)(C)C)cc(-c3ncc4c(N5CC6CCC(C5)N6)nc(OC[C@@]56CCCN5C[C@H](F)C6)nc4c3F)c12] |
| 174 | 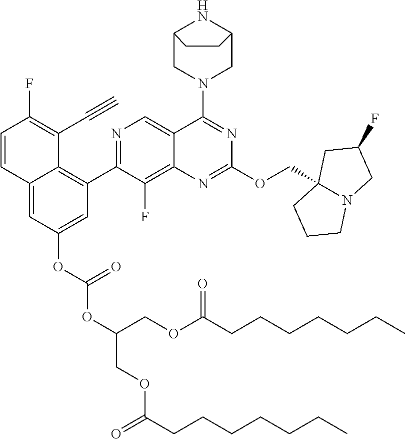 [SMILES: C#Cc1c(F)ccc2cc(OC(=O)OC(COC(=O)CCCCCCC)COC(=O)CCCCCCC)cc(-c3ncc4c(N5CC6CCC(C5)N6)nc(OC[C@@]56CCCN5C[C@H](F)C6)nc4c3F)c12] |
| 175 | 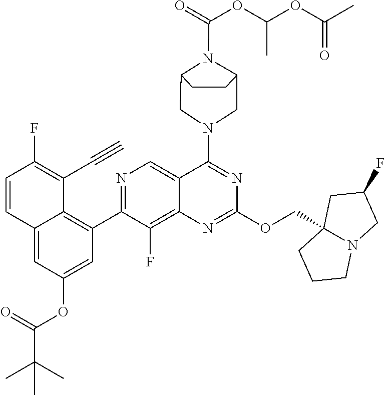 [SMILES: C#Cc1c(F)ccc2cc(OC(=O)C(C)(C)C)cc(-c3ncc4c(N5CC6CCC(C5)N6C(=O)OC(C)OC(C)=O)nc(OC[C@@]56CCCN5C[C@H](F)C6)nc4c3F)c12] |
| 176 | 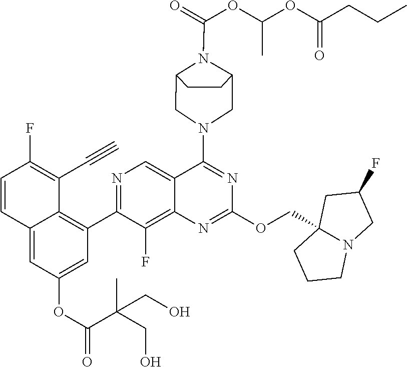 [SMILES: C#Cc1c(F)ccc2cc(OC(=O)C(C)(CO)CO)cc(-c3ncc4c(N5CC6CCC(C5)N6C(=O)OC(C)OC(=O)CCC)nc(OC[C@@]56CCCN5C[C@H](F)C6)nc4c3F)c12] |
| 177 | 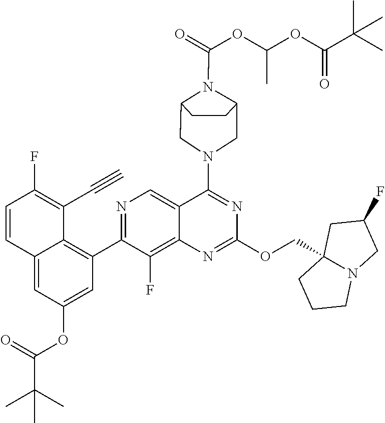 [SMILES: C#Cc1c(F)ccc2cc(OC(=O)C(C)(C)C)cc(-c3ncc4c(N5CC6CCC(C5)N6C(=O)OC(C)OC(=O)C(C)(C)C)nc(OC[C@@]56CCCN5C[C@H](F)C6)nc4c3F)c12] |
| 178 | 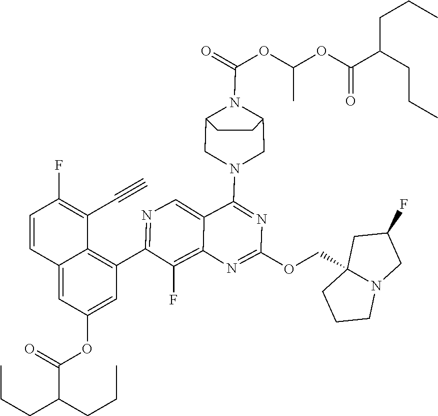 [SMILES: C#Cc1c(F)ccc2cc(OC(=O)C(CCC)CCC)cc(-c3ncc4c(N5CC6CCC(C5)N6C(=O)OC(C)OC(=O)C(CCC)CCC)nc(OC[C@@]56CCCN5C[C@H](F)C6)nc4c3F)c12] |
| 179 | 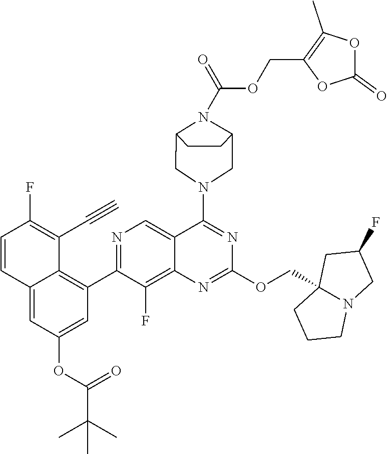 [SMILES: C#Cc1c(F)ccc2cc(OC(=O)C(C)(C)C)cc(-c3ncc4c(N5CC6CCC(C5)N6C(=O)OCc5oc(=O)oc5C)nc(OC[C@@]56CCCN5C[C@H](F)C6)nc4c3F)c12] |
| 180 | 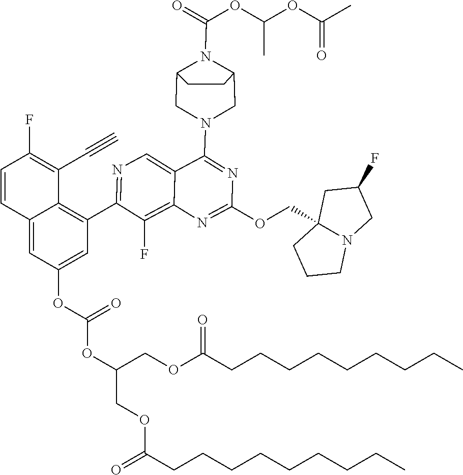 [SMILES: C#Cc1c(F)ccc2cc(OC(=O)OC(COC(=O)CCCCCCCCC)COC(=O)CCCCCCCCC)cc(-c3ncc4c(N5CC6CCC(C5)N6C(=O)OC(C)OC(C)=O)nc(OC[C@@]56CCCN5C[C@H](F)C6)nc4c3F)c12] |
| 181 | 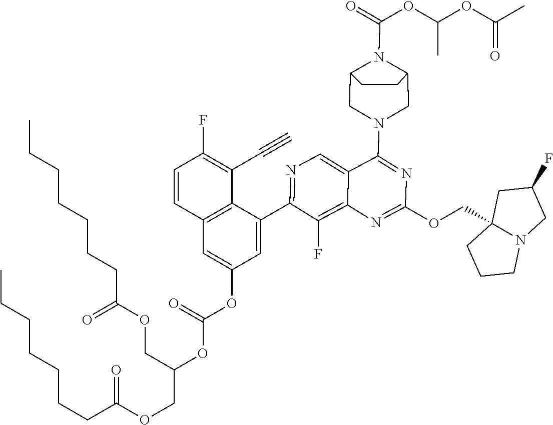 [SMILES: C#Cc1c(F)ccc2cc(OC(=O)OC(COC(=O)CCCCCCC)COC(=O)CCCCCCC)cc(-c3ncc4c(N5CC6CCC(C5)N6C(=O)OC(C)OC(C)=O)nc(OC[C@@]56CCCN5C[C@H](F)C6)nc4c3F)c12] |
| 182 | 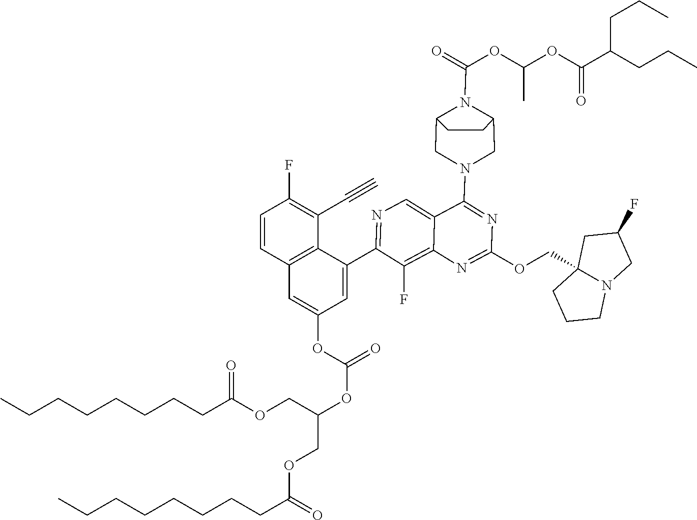 [SMILES: C#Cc1c(F)ccc2cc(OC(=O)OC(COC(=O)CCCCCCCC)COC(=O)CCCCCCCC)cc(-c3ncc4c(N5CC6CCC(C5)N6C(=O)OC(C)OC(=O)C(CCC)CCC)nc(OC[C@@]56CCCN5C[C@H](F)C6)nc4c3F)c12] |
| 183 | 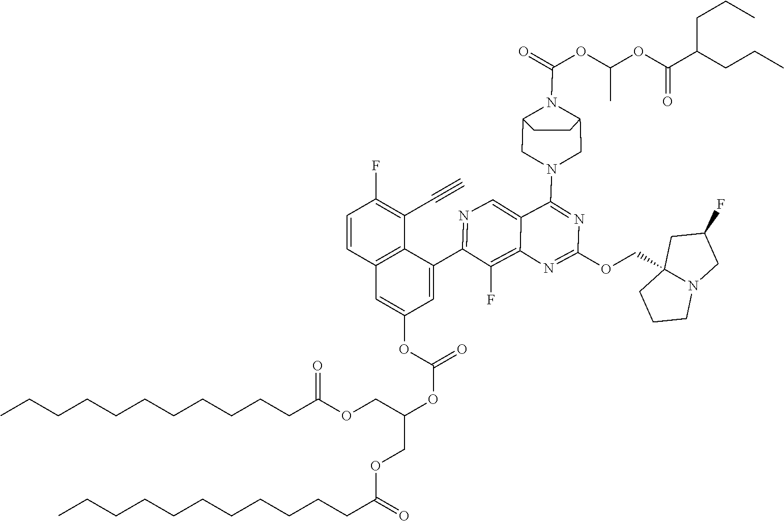 [SMILES: C#Cc1c(F)ccc2cc(OC(=O)OC(COC(=O)CCCCCCCCCCC)COC(=O)CCCCCCCCCCC)cc(-c3ncc4c(N5CC6CCC(C5)N6C(=O)OC(C)OC(=O)C(CCC)CCC)nc(OC[C@@]56CCCN5C[C@H](F)C6)nc4c3F)c12] |
| 184 | 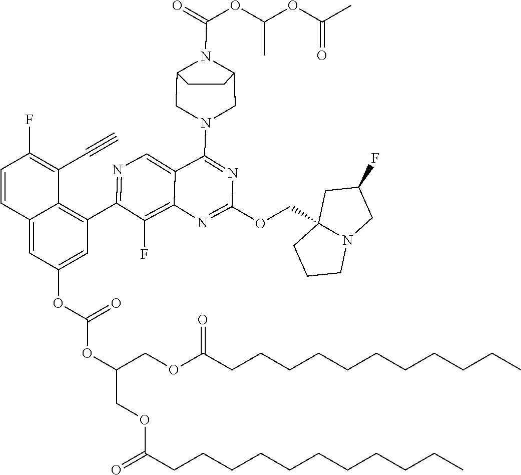 [SMILES: C#Cc1c(F)ccc2cc(OC(=O)OC(COC(=O)CCCCCCCCCCC)COC(=O)CCCCCCCCCCC)cc(-c3ncc4c(N5CC6CCC(C5)N6C(=O)OC(C)OC(C)=O)nc(OC[C@@]56CCCN5C[C@H](F)C6)nc4c3F)c12] |
| 185 | 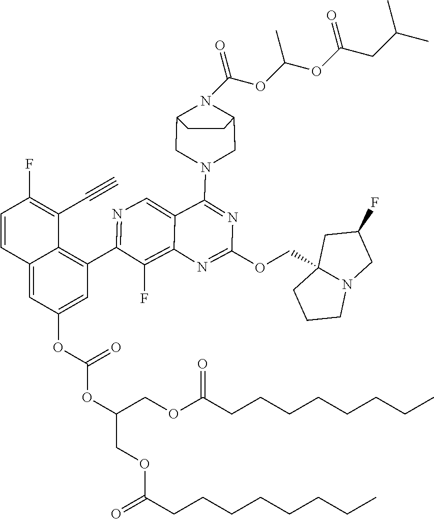 [SMILES: C#Cc1c(F)ccc2cc(OC(=O)OC(COC(=O)CCCCCCCC)COC(=O)CCCCCCCC)cc(-c3ncc4c(N5CC6CCC(C5)N6C(=O)OC(C)OC(=O)CC(C)C)nc(OC[C@@]56CCCN5C[C@H](F)C6)nc4c3F)c12] |
| 186 | 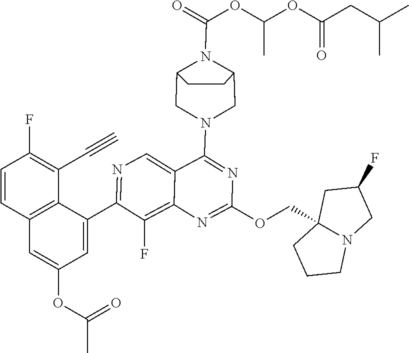 [SMILES: C#Cc1c(F)ccc2cc(OC(C)=O)cc(-c3ncc4c(N5CC6CCC(C5)N6C(=O)OC(C)OC(=O)CC(C)C)nc(OC[C@@]56CCCN5C[C@H](F)C6)nc4c3F)c12] |
| 187 | 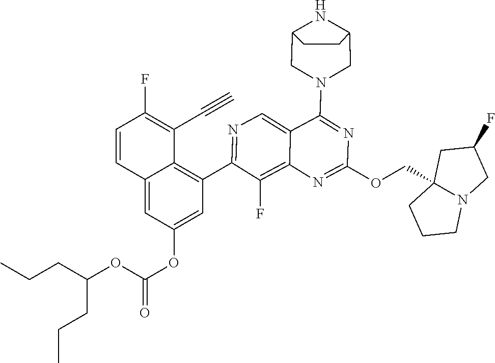 [SMILES: C#Cc1c(F)ccc2cc(OC(=O)OC(CCC)CCC)cc(-c3ncc4c(N5CC6CCC(C5)N6)nc(OC[C@@]56CCCN5C[C@H](F)C6)nc4c3F)c12] |
| 188 | 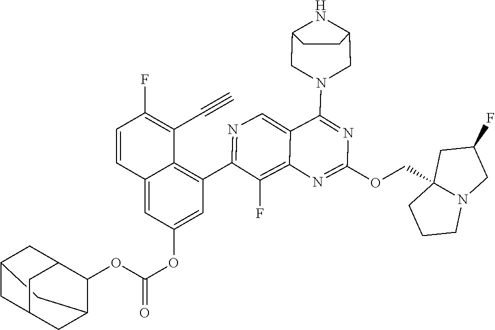 [SMILES: C#Cc1c(F)ccc2cc(OC(=O)OC3C4CC5CC(C4)CC3C5)cc(-c3ncc4c(N5CC6CCC(C5)N6)nc(OC[C@@]56CCCN5C[C@H](F)C6)nc4c3F)c12] |
| 189 | 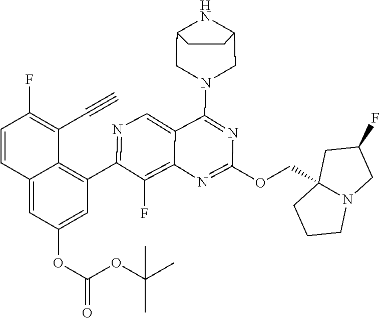 [SMILES: C#Cc1c(F)ccc2cc(OC(=O)OC(C)(C)C)cc(-c3ncc4c(N5CC6CCC(C5)N6)nc(OC[C@@]56CCCN5C[C@H](F)C6)nc4c3F)c12] |
| 190 | 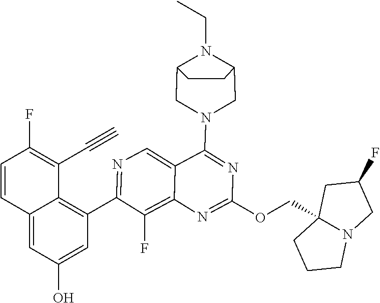 [SMILES: C#Cc1c(F)ccc2cc(O)cc(-c3ncc4c(N5CC6CCC(C5)N6CC)nc(OC[C@@]56CCCN5C[C@H](F)C6)nc4c3F)c12] |
| 191 | 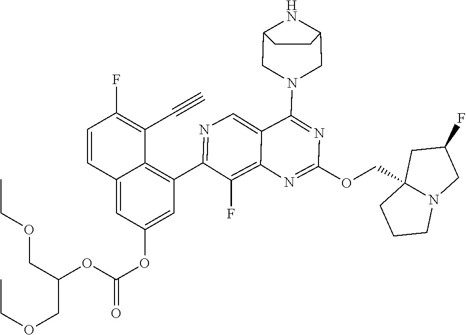 [SMILES: C#Cc1c(F)ccc2cc(OC(=O)OC(COCC)COCC)cc(-c3ncc4c(N5CC6CCC(C5)N6)nc(OC[C@@]56CCCN5C[C@H](F)C6)nc4c3F)c12] |
| 192 | 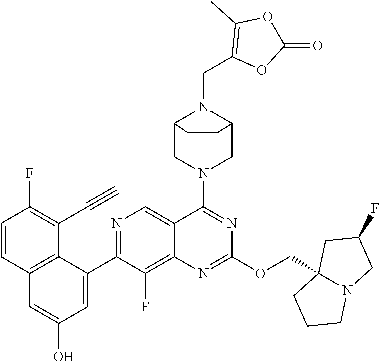 [SMILES: C#Cc1c(F)ccc2cc(O)cc(-c3ncc4c(N5CC6CCC(C5)N6Cc5oc(=O)oc5C)nc(OC[C@@]56CCCN5C[C@H](F)C6)nc4c3F)c12] |
| 193 | 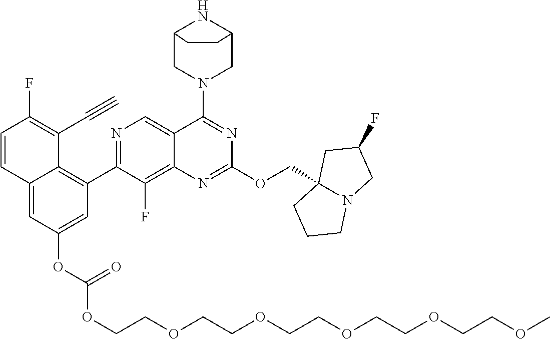 [SMILES: C#Cc1c(F)ccc2cc(OC(=O)OCCOCCOCCOCCOCCOC)cc(-c3ncc4c(N5CC6CCC(C5)N6)nc(OC[C@@]56CCCN5C[C@H](F)C6)nc4c3F)c12] |
| 194 | 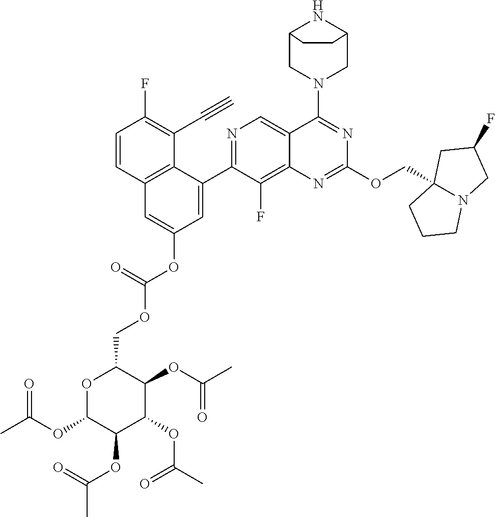 [SMILES: C#Cc1c(F)ccc2cc(OC(=O)OC[C@H]3O[C@@H](OC(C)=O)[C@H](OC(C)=O)[C@@H](OC(C)=O)[C@@H]3OC(C)=O)cc(-c3ncc4c(N5CC6CCC(C5)N6)nc(OC[C@@]56CCCN5C[C@H](F)C6)nc4c3F)c12] |
| 195 | 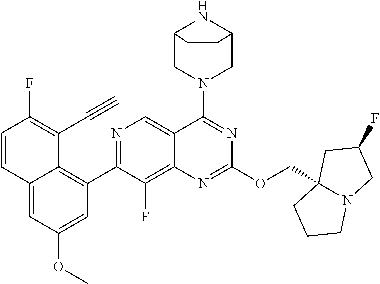 [SMILES: C#Cc1c(F)ccc2cc(OC)cc(-c3ncc4c(N5CC6CCC(C5)N6)nc(OC[C@@]56CCCN5C[C@H](F)C6)nc4c3F)c12] |
| 196 | 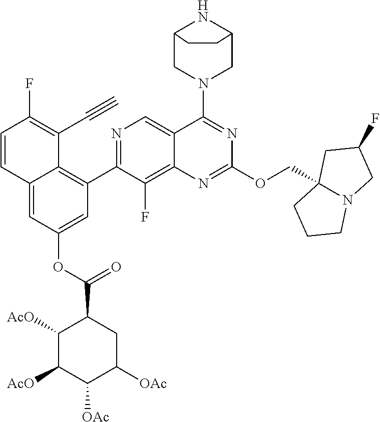 [SMILES: C#Cc1c(F)ccc2cc(OC(=O)[C@H]3CC(OC(C)=O)[C@H](OC(C)=O)[C@@H](OC(C)=O)[C@@H]3OC(C)=O)cc(-c3ncc4c(N5CC6CCC(C5)N6)nc(OC[C@@]56CCCN5C[C@H](F)C6)nc4c3F)c12] |
| 197 | 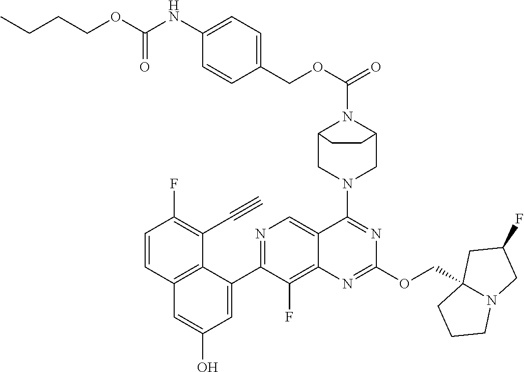 [SMILES: C#Cc1c(F)ccc2cc(O)cc(-c3ncc4c(N5CC6CCC(C5)N6C(=O)OCc5ccc(NC(=O)OCCCC)cc5)nc(OC[C@@]56CCCN5C[C@H](F)C6)nc4c3F)c12] |
| 198 | 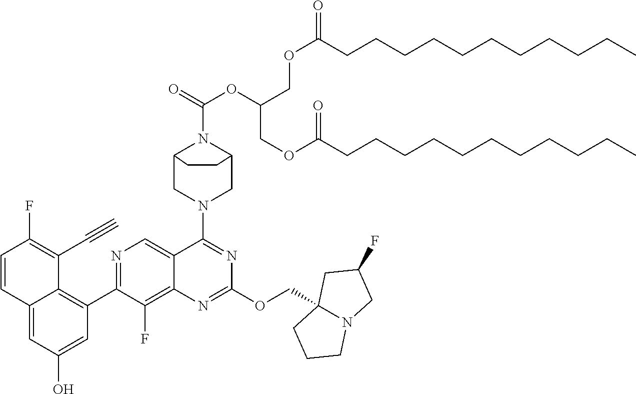 [SMILES: C#Cc1c(F)ccc2cc(O)cc(-c3ncc4c(N5CC6CCC(C5)N6C(=O)OC(COC(=O)CCCCCCCCCCC)COC(=O)CCCCCCCCCCC)nc(OC[C@@]56CCCN5C[C@H](F)C6)nc4c3F)c12] |
| 199 | 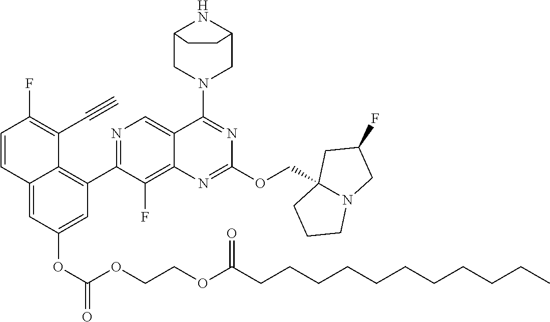 [SMILES: C#Cc1c(F)ccc2cc(OC(=O)OCCOC(=O)CCCCCCCCCCC)cc(-c3ncc4c(N5CC6CCC(C5)N6)nc(OC[C@@]56CCCN5C[C@H](F)C6)nc4c3F)c12] |
| 200 | 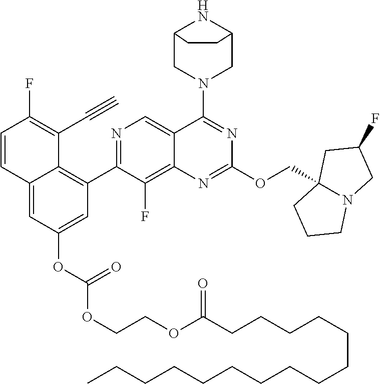 [SMILES: C#Cc1c(F)ccc2cc(OC(=O)OCCOC(=O)CCCCCCCCCCCCCCC)cc(-c3ncc4c(N5CC6CCC(C5)N6)nc(OC[C@@]56CCCN5C[C@H](F)C6)nc4c3F)c12] |

TABLE 2a
Structures of exemplary compounds in accordance with certain
embodiments of the disclosure.

| Cpd | |
|---|---|
| No. | Structure |
| 1a | 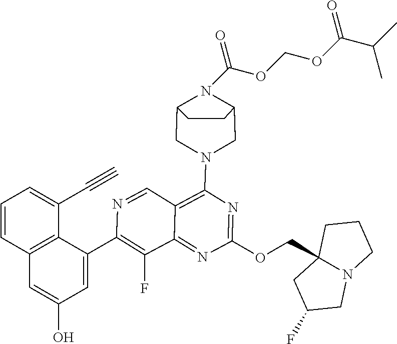 [SMILES: C#Cc1cccc2cc(O)cc(-c3ncc4c(N5CC6CCC(C5)N6C(=O)OCOC(=O)C(C)C)nc(OC[C@@]56CCCN5C[C@H](F)C6)nc4c3F)c12] |
| 2a | 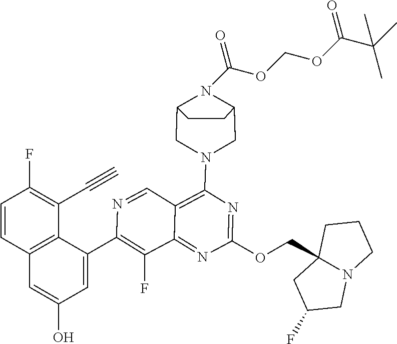 [SMILES: C#Cc1c(F)ccc2cc(O)cc(-c3ncc4c(N5CC6CCC(C5)N6C(=O)OCOC(=O)C(C)(C)C)nc(OC[C@@]56CCCN5C[C@H](F)C6)nc4c3F)c12] |
| 3a | 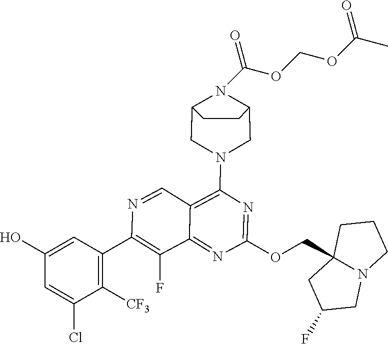 [SMILES: CC(=O)OCOC(=O)N1C2CCC1CN(c1nc(OC[C@@]34CCCN3C[C@H](F)C4)nc3c(F)c(-c4cc(O)cc(Cl)c4C(F)(F)F)ncc13)C2] |
| 4a | 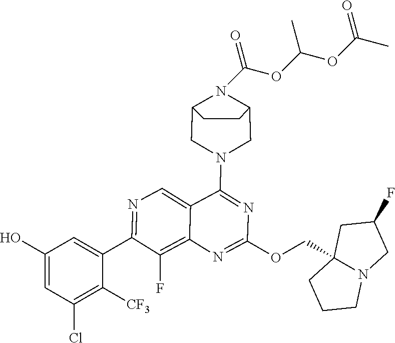 [SMILES: CC(=O)OC(C)OC(=O)N1C2CCC1CN(c1nc(OC[C@@]34CCCN3C[C@H](F)C4)nc3c(F)c(-c4cc(O)cc(Cl)c4C(F)(F)F)ncc13)C2] |
| 5a | 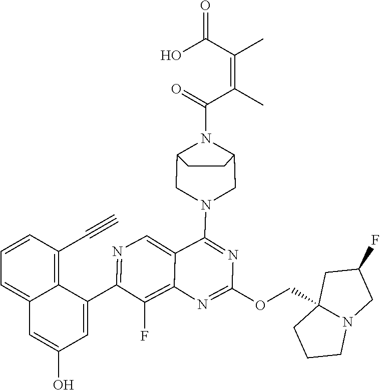 [SMILES: C#Cc1cccc2cc(O)cc(-c3ncc4c(N5CC6CCC(C5)N6C(=O)/C(C)=C(/C)C(=O)O)nc(OC[C@@]56CCCN5C[C@H](F)C6)nc4c3F)c12] |
| 6a | 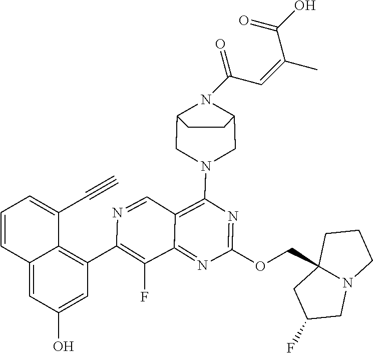 [SMILES: C#Cc1cccc2cc(O)cc(-c3ncc4c(N5CC6CCC(C5)N6C(=O)/C=C(/C)C(=O)O)nc(OC[C@@]56CCCN5C[C@H](F)C6)nc4c3F)c12] |
| 7a | 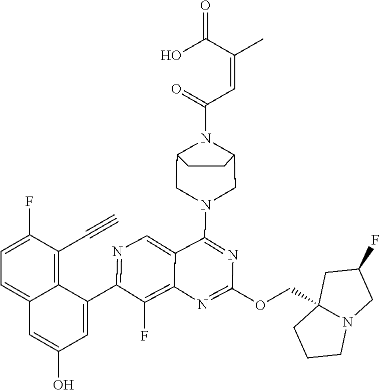 [SMILES: C#Cc1c(F)ccc2cc(O)cc(-c3ncc4c(N5CC6CCC(C5)N6C(=O)/C=C(/C)C(=O)O)nc(OC[C@@]56CCCN5C[C@H](F)C6)nc4c3F)c12] |
| 8a | 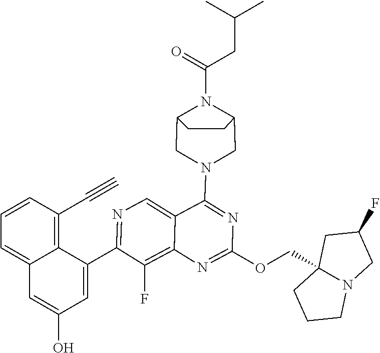 [SMILES: C#Cc1cccc2cc(O)cc(-c3ncc4c(N5CC6CCC(C5)N6C(=O)CC(C)C)nc(OC[C@@]56CCCN5C[C@H](F)C6)nc4c3F)c12] |
| 9a | 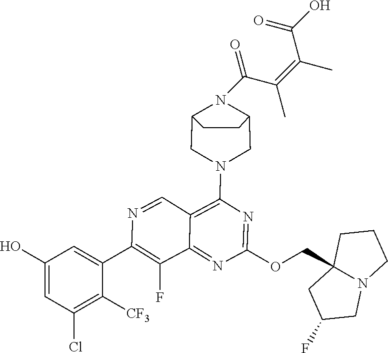 [SMILES: C/C(C(=O)O)=C(\C)C(=O)N1C2CCC1CN(c1nc(OC[C@@]34CCCN3C[C@H](F)C4)nc3c(F)c(-c4cc(O)cc(Cl)c4C(F)(F)F)ncc13)C2] |
| 10a | 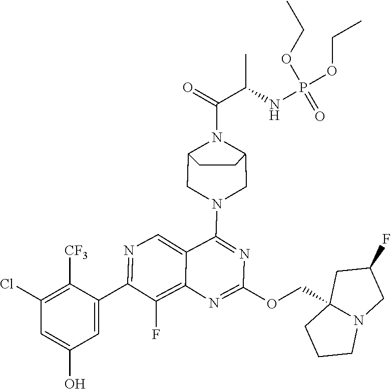 [SMILES: CCOP(=O)(N[C@@H](C)C(=O)N1C2CCC1CN(c1nc(OC[C@@]34CCCN3C[C@H](F)C4)nc3c(F)c(-c4cc(O)cc(Cl)c4C(F)(F)F)ncc13)C2)OCC] |
| 11a | 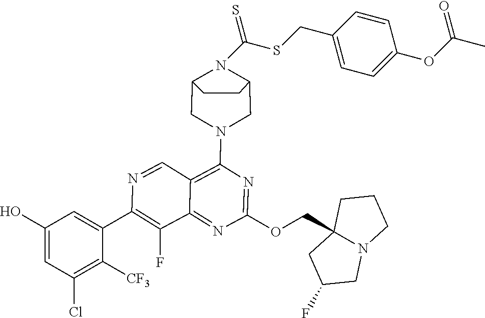 [SMILES: CC(=O)Oc1ccc(CSC(=S)N2C3CCC2CN(c2nc(OC[C@@]45CCCN4C[C@H](F)C5)nc4c(F)c(-c5cc(O)cc(Cl)c5C(F)(F)F)ncc24)C3)cc1] |
| 12a | 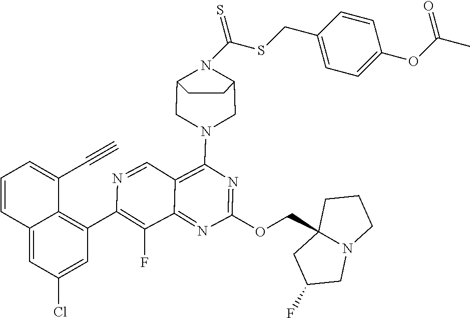 [SMILES: C#Cc1cccc2cc(Cl)cc(-c3ncc4c(N5CC6CCC(C5)N6C(=S)SCc5ccc(OC(C)=O)cc5)nc(OC[C@@]56CCCN5C[C@H](F)C6)nc4c3F)c12] |
| 13a | 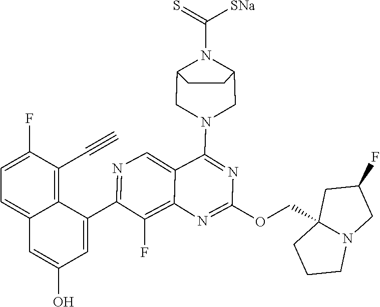 |
| 14a | 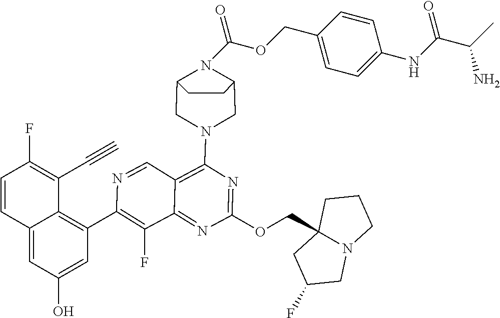 [SMILES: C#Cc1c(F)ccc2cc(O)cc(-c3ncc4c(N5CC6CCC(C5)N6C(=O)OCc5ccc(NC(=O)[C@H](C)N)cc5)nc(OC[C@@]56CCCN5C[C@H](F)C6)nc4c3F)c12] |
| 15a | 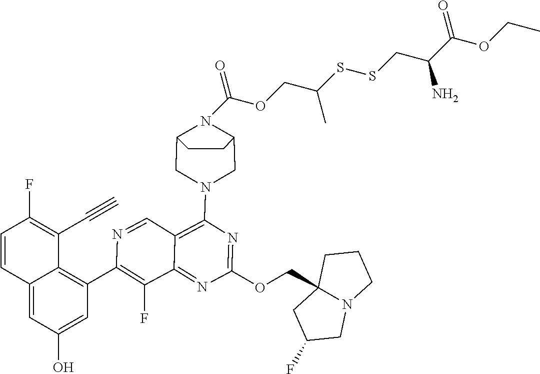 [SMILES: C#Cc1c(F)ccc2cc(O)cc(-c3ncc4c(N5CC6CCC(C5)N6C(=O)OCC(C)SSC[C@H](N)C(=O)OCC)nc(OC[C@@]56CCCN5C[C@H](F)C6)nc4c3F)c12] |
| 16a | 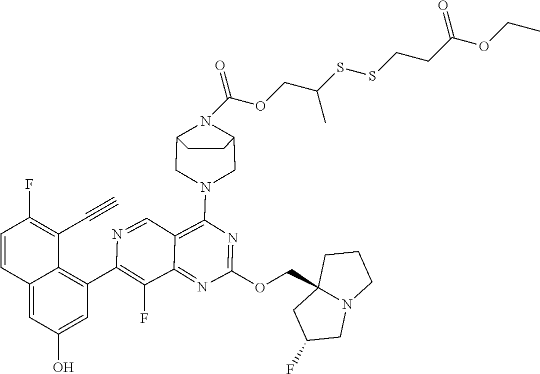 [SMILES: C#Cc1c(F)ccc2cc(O)cc(-c3ncc4c(N5CC6CCC(C5)N6C(=O)OCC(C)SSCCC(=O)OCC)nc(OC[C@@]56CCCN5C[C@H](F)C6)nc4c3F)c12] |
| 17a | 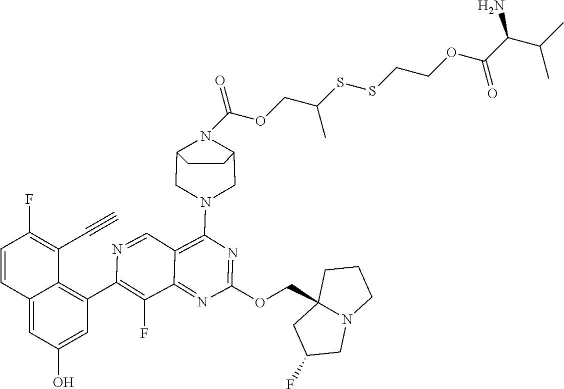 [SMILES: C#Cc1c(F)ccc2cc(O)cc(-c3ncc4c(N5CC6CCC(C5)N6C(=O)OCC(C)SSCCOC(=O)[C@@H](N)C(C)C)nc(OC[C@@]56CCCN5C[C@H](F)C6)nc4c3F)c12] |
| 18a | 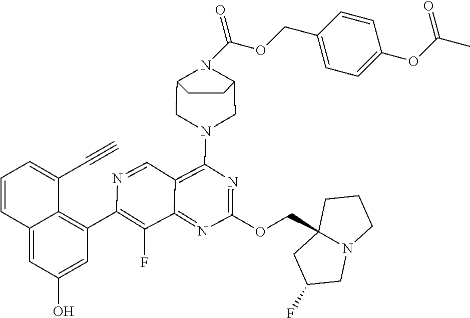 [SMILES: C#Cc1cccc2cc(O)cc(-c3ncc4c(N5CC6CCC(C5)N6C(=O)OCc5ccc(OC(C)=O)cc5)nc(OC[C@@]56CCCN5C[C@H](F)C6)nc4c3F)c12] |
| 19a | 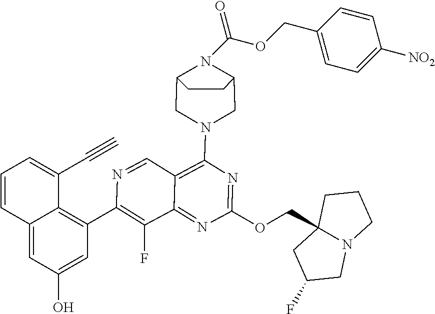 [SMILES: C#Cc1cccc2cc(O)cc(-c3ncc4c(N5CC6CCC(C5)N6C(=O)OCc5ccc([N+](=O)[O-])cc5)nc(OC[C@@]56CCCN5C[C@H](F)C6)nc4c3F)c12] |
| 20a | 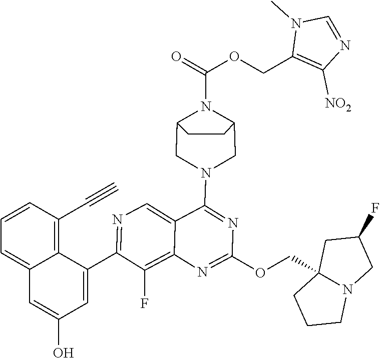 [SMILES: C#Cc1cccc2cc(O)cc(-c3ncc4c(N5CC6CCC(C5)N6C(=O)OCc5c([N+](=O)[O-])ncn5C)nc(OC[C@@]56CCCN5C[C@H](F)C6)nc4c3F)c12] |
| 21a | 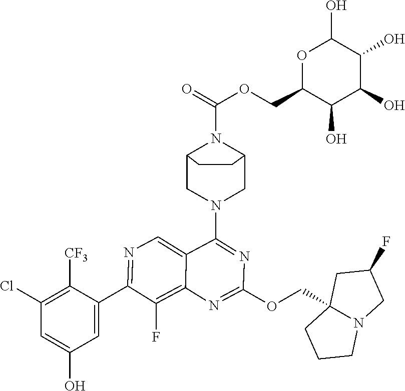 [SMILES: O=C(OC[C@H]1OC(O)[C@H](O)[C@@H](O)[C@H]1O)N1C2CCC1CN(c1nc(OC[C@@]34CCCN3C[C@H](F)C4)nc3c(F)c(-c4cc(O)cc(Cl)c4C(F)(F)F)ncc13)C2] |
| 22a | 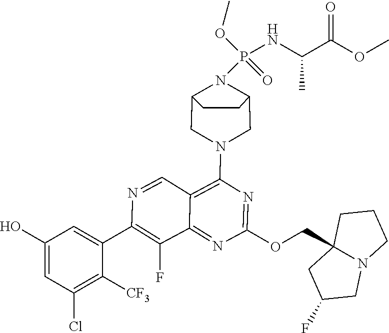 [SMILES: COC(=O)[C@H](C)NP(=O)(OC)N1C2CCC1CN(c1nc(OC[C@@]34CCCN3C[C@H](F)C4)nc3c(F)c(-c4cc(O)cc(Cl)c4C(F)(F)F)ncc13)C2] |
| 23a | 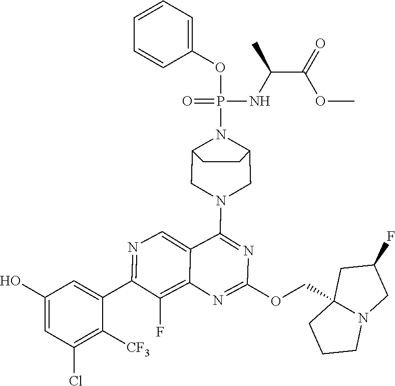 [SMILES: COC(=O)[C@H](C)NP(=O)(Oc1ccccc1)N1C2CCC1CN(c1nc(OC[C@@]34CCCN3C[C@H](F)C4)nc3c(F)c(-c4cc(O)cc(Cl)c4C(F)(F)F)ncc13)C2] |
| 24a | 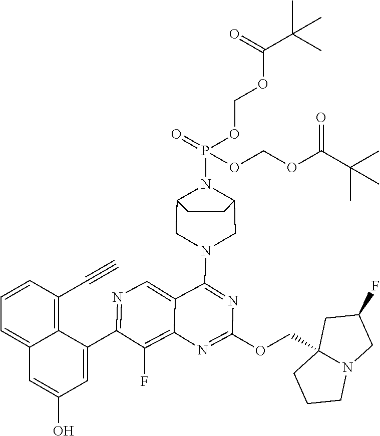 [SMILES: C#Cc1cccc2cc(O)cc(-c3ncc4c(N5CC6CCC(C5)N6P(=O)(OCOC(=O)C(C)(C)C)OCOC(=O)C(C)(C)C)nc(OC[C@@]56CCCN5C[C@H](F)C6)nc4c3F)c12] |
| 25a | 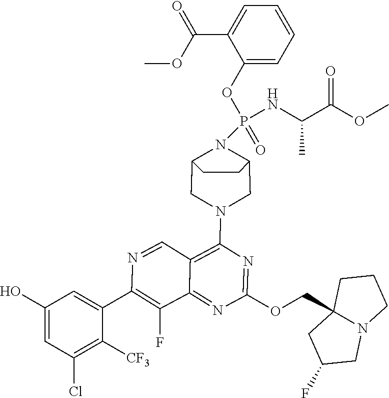 [SMILES: COC(=O)c1ccccc1OP(=O)(N[C@@H](C)C(=O)OC)N1C2CCC1CN(c1nc(OC[C@@]34CCCN3C[C@H](F)C4)nc3c(F)c(-c4cc(O)cc(Cl)c4C(F)(F)F)ncc13)C2] |
| 26a | 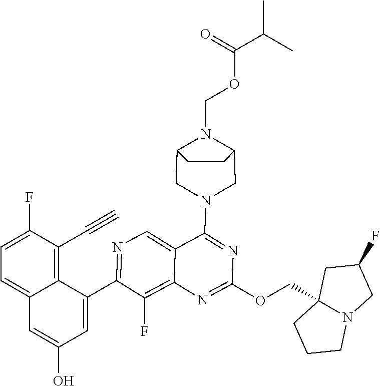 [SMILES: C#Cc1c(F)ccc2cc(O)cc(-c3ncc4c(N5CC6CCC(C5)N6COC(=O)C(C)C)nc(OC[C@@]56CCCN5C[C@H](F)C6)nc4c3F)c12] |
| 27a | 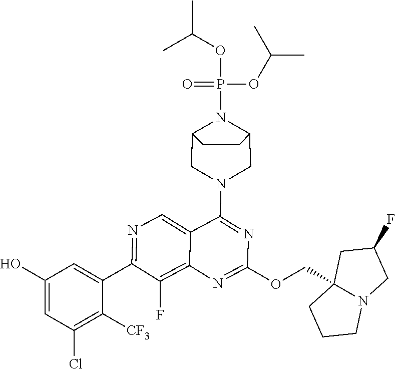 [SMILES: CC(C)OP(=O)(OC(C)C)N1C2CCC1CN(c1nc(OC[C@@]34CCCN3C[C@H](F)C4)nc3c(F)c(-c4cc(O)cc(Cl)c4C(F)(F)F)ncc13)C2] |
| 28a | 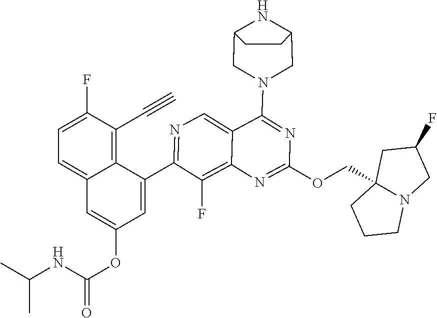 [SMILES: C#Cc1c(F)ccc2cc(OC(=O)NC(C)C)cc(-c3ncc4c(N5CC6CCC(C5)N6)nc(OC[C@@]56CCCN5C[C@H](F)C6)nc4c3F)c12] |
| 29a | 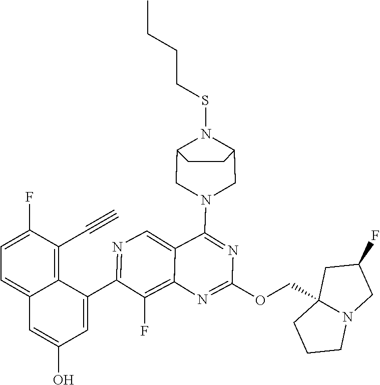 [SMILES: C#Cc1c(F)ccc2cc(O)cc(-c3ncc4c(N5CC6CCC(C5)N6SCCCC)nc(OC[C@@]56CCCN5C[C@H](F)C6)nc4c3F)c12] |
| 30a | 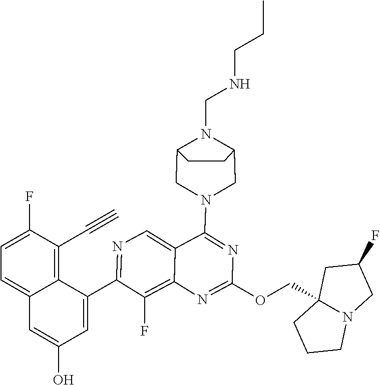 [SMILES: C#Cc1c(F)ccc2cc(O)cc(-c3ncc4c(N5CC6CCC(C5)N6CNCCC)nc(OC[C@@]56CCCN5C[C@H](F)C6)nc4c3F)c12] |
| 31a | 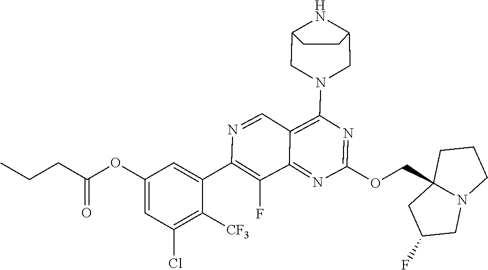 [SMILES: CCCC(=O)Oc1cc(Cl)c(C(F)(F)F)c(-c2ncc3c(N4CC5CCC(C4)N5)nc(OC[C@@]45CCCN4C[C@H](F)C5)nc3c2F)c1] |
| 32a | 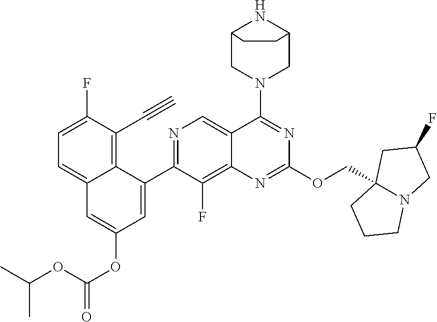 [SMILES: C#Cc1c(F)ccc2cc(OC(=O)OC(C)C)cc(-c3ncc4c(N5CC6CCC(C5)N6)nc(OC[C@@]56CCCN5C[C@H](F)C6)nc4c3F)c12] |
| 33a | 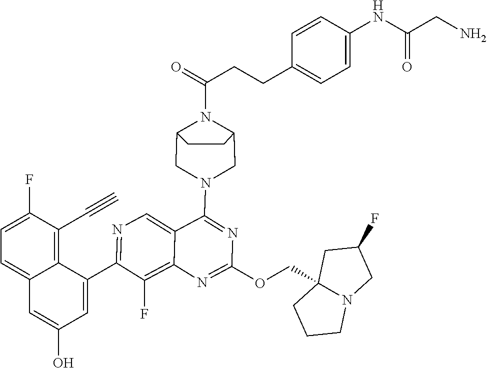 [SMILES: C#Cc1c(F)ccc2cc(O)cc(-c3ncc4c(N5CC6CCC(C5)N6C(=O)CCc5ccc(NC(=O)CN)cc5)nc(OC[C@@]56CCCN5C[C@H](F)C6)nc4c3F)c12] |
| 34a | 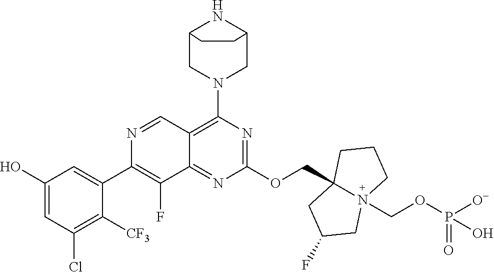 |
| 35a | 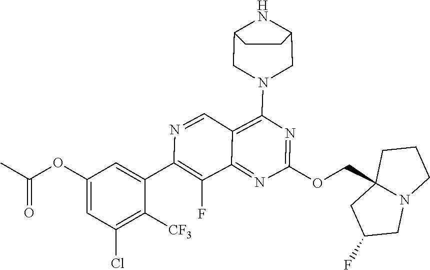 [SMILES: CC(=O)Oc1cc(Cl)c(C(F)(F)F)c(-c2ncc3c(N4CC5CCC(C4)N5)nc(OC[C@@]45CCCN4C[C@H](F)C5)nc3c2F)c1] |
| 36a | 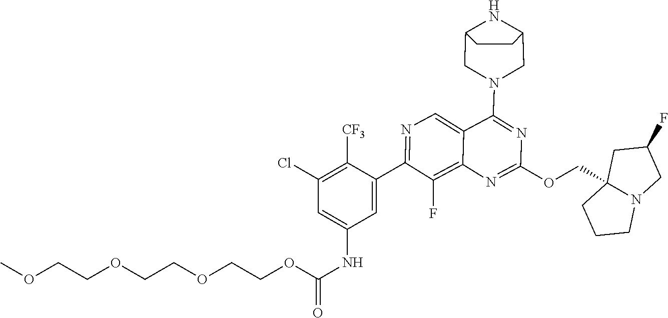 [SMILES: COCCOCCOCCOC(=O)Nc1cc(Cl)c(C(F)(F)F)c(-c2ncc3c(N4CC5CCC(C4)N5)nc(OC[C@@]45CCCN4C[C@H](F)C5)nc3c2F)c1] |
| 37a | 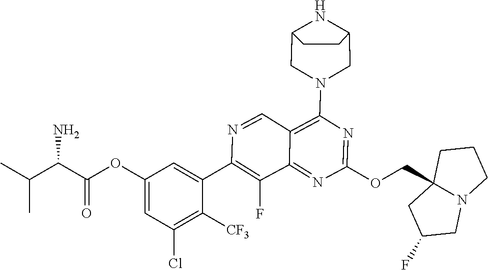 [SMILES: CC(C)[C@H](N)C(=O)Oc1cc(Cl)c(C(F)(F)F)c(-c2ncc3c(N4CC5CCC(C4)N5)nc(OC[C@@]45CCCN4C[C@H](F)C5)nc3c2F)c1] |
| 38a | 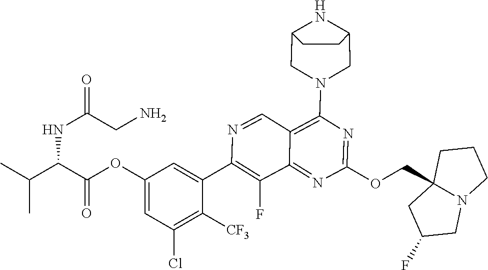 [SMILES: CC(C)[C@H](NC(=O)CN)C(=O)Oc1cc(Cl)c(C(F)(F)F)c(-c2ncc3c(N4CC5CCC(C4)N5)nc(OC[C@@]45CCCN4C[C@H](F)C5)nc3c2F)c1] |
| 39a | 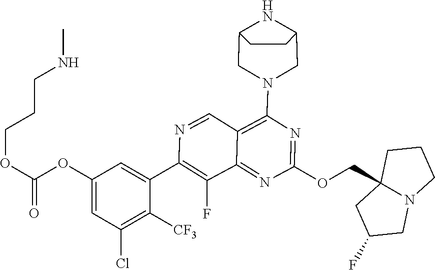 [SMILES: CNCCCOC(=O)Oc1cc(Cl)c(C(F)(F)F)c(-c2ncc3c(N4CC5CCC(C4)N5)nc(OC[C@@]45CCCN4C[C@H](F)C5)nc3c2F)c1] |
| 40a | 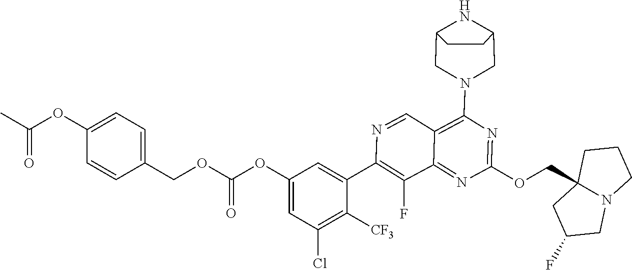 [SMILES: CC(=O)Oc1ccc(COC(=O)Oc2cc(Cl)c(C(F)(F)F)c(-c3ncc4c(N5CC6CCC(C5)N6)nc(OC[C@@]56CCCN5C[C@H](F)C6)nc4c3F)c2)cc1] |
| 41a | 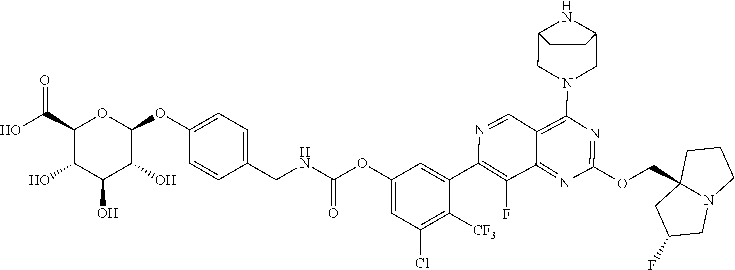 [SMILES: O=C(NCc1ccc(O[C@@H]2O[C@H](C(=O)O)[C@@H](O)[C@H](O)[C@H]2O)cc1)Oc1cc(Cl)c(C(F)(F)F)c(-c2ncc3c(N4CC5CCC(C4)N5)nc(OC[C@@]45CCCN4C[C@H](F)C5)nc3c2F)c1] |
| 42a | 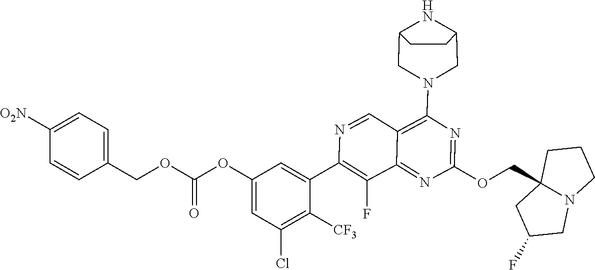 [SMILES: O=C(OCc1ccc([N+](=O)[O-])cc1)Oc1cc(Cl)c(C(F)(F)F)c(-c2ncc3c(N4CC5CCC(C4)N5)nc(OC[C@@]45CCCN4C[C@H](F)C5)nc3c2F)c1] |
| 43a | 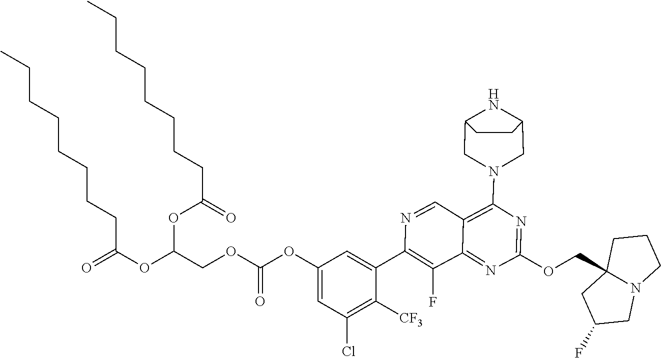 [SMILES: CCCCCCCCC(=O)OC(COC(=O)Oc1cc(Cl)c(C(F)(F)F)c(-c2ncc3c(N4CC5CCC(C4)N5)nc(OC[C@@]45CCCN4C[C@H](F)C5)nc3c2F)c1)OC(=O)CCCCCCCC] |
| 44a | 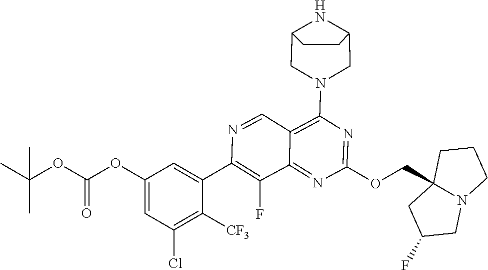 [SMILES: CC(C)(C)OC(=O)Oc1cc(Cl)c(C(F)(F)F)c(-c2ncc3c(N4CC5CCC(C4)N5)nc(OC[C@@]45CCCN4C[C@H](F)C5)nc3c2F)c1] |
| 45a | 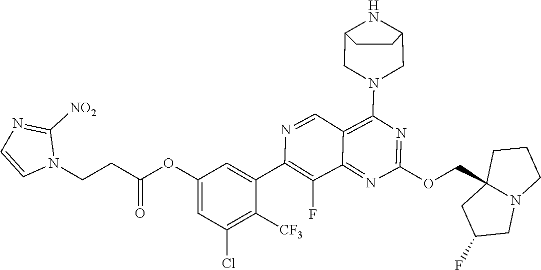 [SMILES: O=C(CCn1ccnc1[N+](=O)[O-])Oc1cc(Cl)c(C(F)(F)F)c(-c2ncc3c(N4CC5CCC(C4)N5)nc(OC[C@@]45CCCN4C[C@H](F)C5)nc3c2F)c1] |
| 46a | 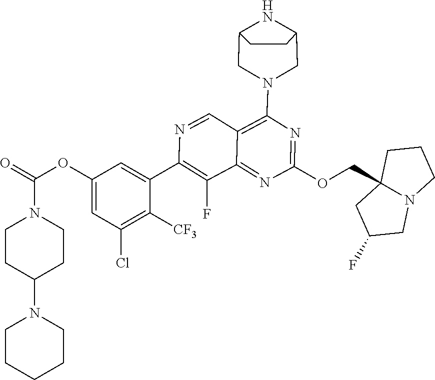 [SMILES: O=C(Oc1cc(Cl)c(C(F)(F)F)c(-c2ncc3c(N4CC5CCC(C4)N5)nc(OC[C@@]45CCCN4C[C@H](F)C5)nc3c2F)c1)N1CCC(N2CCCCC2)CC1] |
| 47a | 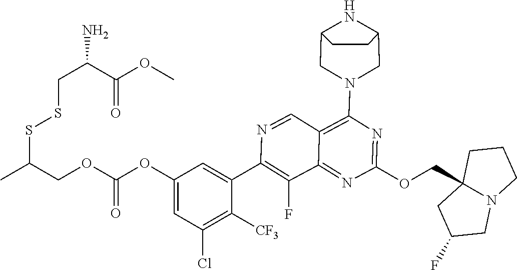 [SMILES: COC(=O)[C@@H](N)CSSC(C)COC(=O)Oc1cc(Cl)c(C(F)(F)F)c(-c2ncc3c(N4CC5CCC(C4)N5)nc(OC[C@@]45CCCN4C[C@H](F)C5)nc3c2F)c1] |
| 48a | 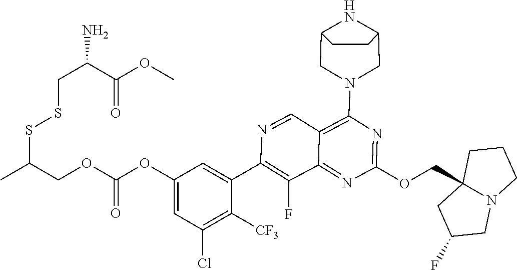 [SMILES: COC(=O)[C@@H](N)CSSC(C)COC(=O)Oc1cc(Cl)c(C(F)(F)F)c(-c2ncc3c(N4CC5CCC(C4)N5)nc(OC[C@@]45CCCN4C[C@H](F)C5)nc3c2F)c1] |
| 49a | 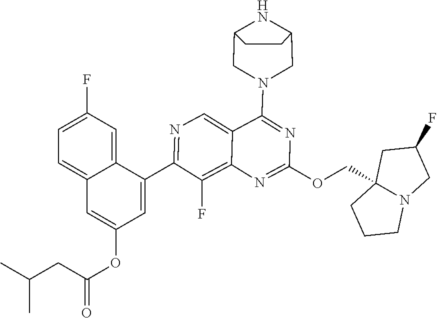 [SMILES: CC(C)CC(=O)Oc1cc(-c2ncc3c(N4CC5CCC(C4)N5)nc(OC[C@@]45CCCN4C[C@H](F)C5)nc3c2F)c2cc(F)ccc2c1] |

In some embodiments of compounds of the disclosure, one or more C, H, O, and/or N atoms in the compound are each independently selected from atoms of natural abundance and isotope-enriched atoms. Examples of isotopes of natural abundance include ¹²C, ¹H, ¹⁶O and ¹⁴N. Examples of isotope-enriched atoms include, without limitation, ¹³C and ¹⁴C for carbon; ²H (D) and ³H (T) for hydrogen; ¹⁷O and ¹⁸O for oxygen; and ¹⁵N for nitrogen. In some embodiments of compounds of the disclosure, all the elements or atoms in a compound are isotopes of natural abundance. In other embodiments, one or more elements or atoms in a compound are isotope-enriched.

Without wishing to be limited by theory, the compounds of the disclosure, as well as pharmaceutically acceptable salts, esters, hydrates, solvates or stereoisomers thereof, can act as KRAS^{G12D} inhibitors and can be used effectively to treat diseases associated with the KRAS^{G12D} mutation. In some embodiments, the compounds of the disclosure have anti-tumor/anti-cancer activity and can be used effectively for the inhibition, treatment or prevention of a hyperproliferative disorder, such as a KRAS^{G12D}-associated cancer or tumor.

In some embodiments, the compound disclosed herein may be administered to a subject in the form of a prodrug that is metabolized after administration into biologically active constituents, thereby effecting treatment or prevention of KRAS-G12D-associated diseases, disorders or conditions.

In another broad aspect, there are provided pharmaceutical compositions comprising a compound described herein, or a pharmaceutically acceptable salt, ester, hydrate, solvate or stereoisomer thereof. In some embodiments, the pharmaceutical compositions further comprise a pharmaceutically acceptable excipient, carrier or diluent.

In some embodiments, there are provided pharmaceutical compositions comprising a compound of Formula (A), or a pharmaceutically acceptable salt, ester, hydrate, solvate or stereoisomer thereof, and a pharmaceutically acceptable carrier.

In some embodiments, there are provided pharmaceutical compositions comprising a compound of Formula (B), or a pharmaceutically acceptable salt, ester, hydrate, solvate or stereoisomer thereof, and a pharmaceutically acceptable carrier.

In some embodiments, there are provided pharmaceutical compositions comprising a compound of Formula (I), or a pharmaceutically acceptable salt, ester, hydrate, solvate or stereoisomer thereof, and a pharmaceutically acceptable carrier.

In some embodiments, there are provided pharmaceutical compositions comprising a compound of Formula (II), or a pharmaceutically acceptable salt, ester, hydrate, solvate or stereoisomer thereof, and a pharmaceutically acceptable carrier.

In some embodiments, there are provided pharmaceutical compositions comprising a compound of Formula (IIA), or a pharmaceutically acceptable salt, ester, hydrate, solvate or stereoisomer thereof, and a pharmaceutically acceptable carrier.

In some embodiments, there are provided pharmaceutical compositions comprising a compound of Formula (III), or a pharmaceutically acceptable salt, ester, hydrate, solvate or stereoisomer thereof, and a pharmaceutically acceptable carrier.

In some embodiments, there are provided pharmaceutical compositions comprising a compound of Formula (IV), or a pharmaceutically acceptable salt, ester, hydrate, solvate or stereoisomer thereof, and a pharmaceutically acceptable carrier.

In some embodiments, there are provided pharmaceutical compositions comprising a compound of Formula (V), or a pharmaceutically acceptable salt, ester, hydrate, solvate or stereoisomer thereof, and a pharmaceutically acceptable carrier.

In some embodiments, there are provided pharmaceutical compositions comprising a compound of Formula (VI), or a pharmaceutically acceptable salt, ester, hydrate, solvate or stereoisomer thereof, and a pharmaceutically acceptable carrier.

In some embodiments, there are provided pharmaceutical compositions comprising a compound of Formula (VII), or a pharmaceutically acceptable salt, ester, hydrate, solvate or stereoisomer thereof, and a pharmaceutically acceptable carrier.

In some embodiments, there are provided pharmaceutical compositions comprising a compound which is a derivative of any one of the A1, A2, and A3 base compounds, or a pharmaceutically acceptable salt, ester, hydrate, solvate or stereoisomer thereof, and a pharmaceutically acceptable carrier.

In some embodiments, there are provided pharmaceutical compositions comprising a compound of Table 2 or 2a, or a pharmaceutically acceptable salt, ester, hydrate, solvate or stereoisomer thereof, and a pharmaceutically acceptable carrier.

In some such embodiments, the pharmaceutical composition comprises a pharmaceutically acceptable excipient comprising one or more adhesive, binder, filler, disintegrant, lubricant, glidant and/or dispersant. In some embodiments, the pharmaceutically acceptable carrier comprises one or more of a cream, an emulsion, a gel, a liposome, and a nanoparticle.

In some embodiments, the pharmaceutical composition is suitable for administration orally or by injection.

In one embodiment, the pharmaceutical composition is suitable for oral administration. In some such embodiments, the composition is in the form of a hard shell gelatin capsule, a soft shell gelatin capsule, a cachet, a pill, a tablet, a lozenge, a powder, a granule, a pellet, a pastille, or a dragee. In some embodiments, the composition is in the form of a solution, an aqueous liquid suspension, a non-aqueous liquid suspension, an oil-in-water liquid emulsion, a water-in-oil liquid emulsion, an elixir, or a syrup. In some embodiments, the composition is enteric coated. In some embodiments, the composition is formulated for controlled release.

In another embodiment, the pharmaceutical composition is suitable for administration by injection. For example, the pharmaceutical composition may be administered subcutaneously, intravenously, intramuscularly, or intraperitoneally. In one embodiment, the pharmaceutical composition is suitable for intravenous administration.

In another broad aspect, there are provided methods of inhibiting KRAS-G12D activity in a subject in need thereof, comprising administering to the subject an effective amount of a compound and/or a pharmaceutical composition described herein.

In certain embodiments, there are provided methods of treating or preventing a KRAS-G12D-associated disease, disorder or condition in a subject in need thereof, comprising administering an effective amount of a compound and/or a pharmaceutical composition described herein, such that the KRAS-G12D-associated disease, disorder or condition is treated or prevented in the subject.

In particular embodiments, the compounds described herein act to inhibit KRAS-G12D and are useful as therapeutic or prophylactic therapy when such inhibition is desired, e.g., for the prevention or treatment of KRAS-G12D-associated diseases, conditions and/or disorders. Unless otherwise indicated, when uses of the compounds of the present disclosure are described herein, it is to be understood that such compounds may be in the form of a composition (e.g., a pharmaceutical composition).

As used herein, the terms “KRAS-G12D inhibitor” and “KRAS^{G12D} inhibitor compound” are used interchangeably to refer to a compound of the disclosure capable of inhibiting the KRAS-G12D protein in a cellular assay, an in vivo model, and/or other assay means indicative of KRAS-G12D inhibition and potential therapeutic or prophylactic efficacy. The terms also refer to compounds that exhibit at least some therapeutic or prophylactic benefit in a human subject. Although the compounds of the present disclosure are believed to have effect by inhibiting KRAS-G12D activity in a cell, a precise understanding of the compounds' underlying mechanism of action is not required to practice the technology.

In some embodiments, there are provided methods for inhibiting, treating or preventing a KRAS-G12D-associated disease, disorder or condition in a subject in need thereof. The KRAS-G12D-associated disease, disorder or condition may be, for example and without limitation, a cancer or tumor or hyperplastic or hyperproliferative disease or disorder related to or associated with the KRAS-G12D mutation. In some embodiments, the KRAS-G12D-associated disease, disorder or condition is a hyperproliferative disorder. In some embodiments, the KRAS-G12D-associated disease, disorder or condition is a hyperplastic disorder. In some embodiments, the KRAS-G12D-associated disease, disorder or condition is a malignant cancer or tumor. In some embodiments, the KRAS-G12D-associated disease, disorder or condition is a cardiac, lung, gastrointestinal, genitourinary tract, biliary tract, large intestine, small intestine, liver, bone, nervous system, gynecological, hematologic, skin, or adrenal gland cancer or tumor. In some embodiments, the KRAS-G12D-associated disease, disorder or condition is a non-small-cell lung cancer (NSCLC), a small cell lung cancer, a pancreatic cancer, a colorectal cancer, a colon cancer, a bile duct cancer, a cervical cancer, a bladder cancer, a liver cancer or a breast cancer.

In some embodiments, there are provided methods for treating or preventing cancer in a subject (e.g., a human) comprising administering to the subject a therapeutically effective amount of at least one KRAS-G12D inhibitor compound or composition described herein. In some embodiments of such methods, the subject is administered at least one KRAS-G12D inhibitor compound or composition in an amount effective to reverse, slow or stop the progression of a KRAS-G12D-associated disease, disorder or condition.

The type of cancer or tumor that can be treated or prevented using the compounds and compositions described herein is not meant to be particularly limited. Examples of cancers and tumors that can be treated or prevented using the compounds and compositions described herein include, but are not limited to, cancers of the: (i) cardiac tissue or heart (including sarcoma, angiosarcoma, fibrosarcoma, rhabdomyosarcoma, liposarcoma, myxoma, rhabdomyoma, fibroma, lipoma, teratoma); (ii) lung (including bronchogenic carcinoma, squamous cell carcinoma, undifferentiated small cell carcinoma, undifferentiated large cell carcinoma, adenocarcinoma, alveolar (bronchiolar) carcinoma, bronchial adenoma, sarcoma, lymphoma, chondromatous hamartoma, mesothelioma); (iii) gastrointestinal system (including esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vasodilator tumors, Karposi's sarcoma, leiomyoma, hemangioma, lipoma, neurofibroma, fibroma), small intestine (adenocarcinoma, lymphoma, carcinoid tumor, Kaposi's sarcoma, leiomyoma, hemangioma, lipoma, neurofibroma, fibroma), large intestine (adenocarcinoma, tubular adenoma, villous adenoma, hematoma, leiomyoma), large bowel (adenocarcinomas, tubular adenoma, villous adenoma, hamartoma, leiomyoma)); (iv) genitourinary tract (including kidney (adenocarcinoma, WiIm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embroyonal carcinoma, teratocarcinoma, choriocarcinoma, sarcoma, stromal cell carcinoma, interstitial cell carcinoma, fibroma, fibroadenoma, adenoid tumors, lipoma); (v) liver (including hepatoma (hepatocellular carcinoma), cholangiocarcinoma, hepatoblastoma, angiosarcoma, hepatocellular adenoma, hemangioma); (vi) biliary tract (including gallbladder cancer, ampule cancer, bile duct carcinoma); (vii) bone (including osteogenic sarcoma (osteosarcoma), fibrosarcoma, malignant fibrous histocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors); (viii) nervous system (including skull (osteoma, angioma, hemangioma, granuloma, xanthoma, osteitis deformans), meninges (meningioma, meningiosarcoma, gliomatosis), brain (astrocytoma, medulloblastoma, myeloblastoma, glioma, ependymoma, epididymis tumor, germinoma (pinealoma), glioblastoma multiform, oligodendroglioma, schwannoma, retinoblastoma, congenitial neoplasm, congential tumors), spinal cord, neurofibroma, meningioma, glioma, sarcoma)); (ix) gynecological tissues (including uterus (endometrial carcinoma, serous bladder cancer, mucinous bladder cancer, carcinoma unclassified), granular sheath cell tumor, serous stromal cell tumor, dysplasia, malignant teratoma), cervix (cervical carcinoma, pre-tumor cervical dsplasia), ovaries (ovarian carcinoma [serous cystadenocarcinoma, mucinous cystadenocarcinoma], granulose-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma) vagina (clear cell carcinoma, squamous cell carcinoma, uveal or botryoid sarcoma (embryonal rhabdomyosarcoma), fallopian tubes carcinoma)); (x) hematologic system (including blood (myeloid leukemia (acute and chronic), (myelomatosis (acute and chronic), acute lymphocytic leukemia, chronic lymphocytic leukemia, disorder myeloproliferative, acute lymphoblastic leukemia, chronic lymphocytic leukemia, myeloproliferative diseases, multiple myeloma, myelodysplastic syndrome), Hodgkin's disease, non-Hodgkins's lymphoma, malignant lymphoma); (xi) skin (including malignant melanoma, basal cell carcinoma, squamous cell carcinoma, Kaposi's sarcoma, moles dysplastic nevus, lipoma, angioma, hemangioma, dermatofibroma, keloids, psoriasis, or adrenal neuroblastoma); and (xii) adrenal glands (including neuroblastoma).

In some embodiments of methods of the present disclosure, the cancer is non-small cell lung cancer (NSCLC), small cell lung cancer, pancreatic cancer, colorectal cancer, colon cancer, bile duct cancer, cervical cancer, bladder cancer, liver cancer or breast cancer.

In certain embodiments, there are provided methods for treating or preventing a hyperplastic or hyperproliferative disease or disorder (e.g., a cancer or a tumor) in a subject (e.g., a human) comprising administering to the subject a therapeutically effective amount of at least one KRAS-G12D inhibitor compound or composition provided herein. In some embodiments, the hyperplastic disorder is a cancer or a tumor, such as without limitation non-small cell lung cancer (NSCLC), pancreatic cancer, colorectal cancer, colon cancer, bile duct cancer, cervical cancer, bladder cancer, liver cancer or breast cancer.

Other diseases, disorders and conditions that can be treated or prevented, in whole or in part, by inhibition of KRAS-G12D activity are candidate indications for the KRAS-G12D inhibitor compounds and compositions provided herein and are encompassed by methods of the disclosure.

In some embodiments, the present disclosure provides a method for treating and/or preventing an immune-related disease, disorder, or condition, or symptoms thereof, in a subject comprising administering using at least one KRAS^{G12D} inhibitor compound or composition of the present disclosure to the subject.

In some embodiments, the present disclosure provides a method for treating and/or preventing an inflammatory disorder in a subject, comprising administering using at least one KRAS^{G12D} inhibitor compound or composition of the present disclosure to the subject.

In some embodiments, there is further provided the use of the KRAS-G12D inhibitor compounds and compositions described herein in combination with one or more additional agents. The one or more additional agents may have some KRAS-G12D-modulating activity and/or they may function through distinct mechanisms of action. In some embodiments, such agents comprise radiation (e.g., localized radiation therapy or total body radiation therapy) and/or other treatment modalities of anon-pharmacological nature. When combination therapy is utilized, the KRAS-G12D inhibitor(s) and one additional agent(s) may be in the form of a single composition or multiple compositions, and the treatment modalities can be administered concurrently, sequentially, or through some other regimen. By way of example, in some embodiments there is provided a treatment regimen wherein a radiation phase is followed by a chemotherapeutic phase. A combination therapy can have an additive or synergistic effect.

In some embodiments, there is provided the use of a KRAS-G12D inhibitor compound or composition described herein in combination with bone marrow transplantation, peripheral blood stem cell transplantation, or other types of transplantation therapy.

In particular embodiments, there is provided the use of the inhibitors of KRAS-G12D function described herein in combination with immune checkpoint inhibitors. The blockade of immune checkpoints, which results in the amplification of antigen-specific T cell responses, has been shown to be a promising approach in human cancer therapeutics. Non-limiting examples of immune checkpoints (ligands and receptors), some of which are selectively upregulated in various types of tumor cells, that are candidates for blockade include PD1 (programmed cell death protein 1); PDL1 (PD1 ligand); BTLA (B and T lymphocyte attenuator); CTLA4 (cytotoxic T-lymphocyte associated antigen 4); TIM3 (T-cell membrane protein 3); LAG3 (lymphocyte activation gene 3); A2aR (adenosine A2a receptor A2aR); and Killer Inhibitory Receptors. Non-limiting examples of immune checkpoint inhibitors include ipulimumab, nivolumab and lambrolizumab.

In other embodiments, there are provided methods for treating a cancer in a subject, comprising administering to the subject a therapeutically effective amount of at least one KRAS-G12D inhibitor compound or composition thereof and at least one chemotherapeutic agent, such agents including, but not limited to alkylating agents (e.g., nitrogen mustards such as chlorambucil, cyclophosphamide, isofamide, mechlorethamine, melphalan, and uracil mustard; aziridines such as thiotepa; methanesulphonate esters such as busulfan; nucleoside analogs (e.g., gemcitabine); nitroso ureas such as carmustine, lomustine, and streptozocin; topoisomerase 1 inhibitors (e.g., irinotecan); platinum complexes such as cisplatin and carboplatin; bioreductive alkylators such as mitomycin, procarbazine, dacarbazine and altretamine); DNA strand-breakage agents (e.g., bleomycin); topoisomerase II inhibitors (e.g., amsacrine, dactinomycin, daunorubicin, idarubicin, mitoxantrone, doxorubicin, etoposide, and teniposide); DNA minor groove binding agents (e.g., plicamydin); antimetabolites (e.g., folate antagonists such as methotrexate and trimetrexate; pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); tubulin interactive agents (e.g., vincristine, estramustine, vinblastine, docetaxol, epothilone derivatives, and paclitaxel); hormonal agents (e.g., estrogens; conjugated estrogens; ethinyl estradiol; diethylstilbesterol; chlortrianisen; idenestrol; progestins such as hydroxyprogesterone caproate, medroxyprogesterone, and megestrol; and androgens such as testosterone, testosterone propionate, fluoxymesterone, and methyltestosterone); adrenal corticosteroids (e.g., prednisone, dexamethasone, methylprednisolone, and prednisolone); leutinizing hormone releasing agents or gonadotropin-releasing hormone antagonists (e.g., leuprolide acetate and goserelin acetate); and antihormonal antigens (e.g., tamoxifen, antiandrogen agents such as flutamide; and antiadrenal agents such as mitotane and aminoglutethimide). There is also provided the use of the KRAS-G12D inhibitors in combination with other agents known in the art (e.g., arsenic trioxide) and other chemotherapeutic or anti-cancer agents that may be appropriate for treatment.

In some embodiments drawn to methods of treating cancer, the administration of a therapeutically effective amount of a KRAS-G12D inhibitor in combination with at least one chemotherapeutic agent results in a cancer survival rate greater than the cancer survival rate observed by administering either agent alone. In further embodiments drawn to methods of treating cancer, the administration of a therapeutically effective amount of a KRAS-G12D inhibitor in combination with at least one chemotherapeutic agent results in a reduction of tumor size or a slowing of tumor growth greater than reduction of the tumor size or slowing of tumor growth observed by administration of either agent alone.

In further embodiments, there are provided methods for treating or preventing cancer in a subject, comprising administering to the subject a therapeutically effective amount of at least one KRAS-G12D inhibitor compound or composition and at least one signal transduction inhibitor (STI). In a particular embodiment, the at least one STI is selected from the group consisting of bcr/abl kinase inhibitors, epidermal growth factor (EGF) receptor inhibitors, her-2/neu receptor inhibitors, and famesyl transferase inhibitors (FTIs).

In other embodiments, there are provided methods of augmenting the rejection of tumor cells in a subject comprising administering an KRAS-G12D inhibitor compound or composition in conjunction with at least one chemotherapeutic agent and/or radiation therapy, wherein the resulting rejection of tumor cells is greater than that obtained by administering either the KRAS-G12D inhibitor, the chemotherapeutic agent or the radiation therapy alone.

In further embodiments, there are provided methods for treating cancer in a subject, comprising administering to the subject a therapeutically effective amount of at least one KRAS-G12D inhibitor and at least one anti-cancer agent other than a KRAS-G12D inhibitor. It should be understood that, as used herein, a “KRAS-G12D inhibitor” refers to compounds of the disclosure, e.g., a compound of Formula (A), a compound of Formula (B), a compound of Formula (I), a compound of any one of Formulae (II), (IIA), (III), (IV), (V), (VI), (VII), a compound of Table 2 or 2a, or a pharmaceutically acceptable salt, ester, hydrate, or solvate thereof, or a stereoisomer thereof, and to pharmaceutical compositions thereof.

In some embodiments, there are provided methods of treating or preventing a KRAS-G12D-associated disease, disorder or condition in a subject in need thereof, comprising administering a therapeutically effective amount of at least one KRAS-G12D inhibitor or a pharmaceutical composition thereof to the subject, such that the KRAS-G12D-associated disease, disorder or condition is treated or prevented in the subject. In some embodiments, the compound is administered in an amount effective to reverse, slow or stop the progression of a KRAS-G12D-mediated cancer in the subject.

In some embodiments, the KRAS-G12D-associated disease, disorder or condition is a KRAS-G12D related cancer, tumor or hyperplastic or hyperproliferative disorder, such as, for example and without limitation, a cancer of the cardiac system, heart, lung, gastrointestinal system, genitourinary tract, biliary tract, small intestine, large intestine, liver, bone, nervous system, brain, gynecological system, hematologic tissues, skin, or adrenal glands, as described herein. In certain embodiments, the cancer, tumor or hyperplastic or hyperproliferative disorder is non-small cell lung cancer (NSCLC), small cell lung cancer, pancreatic cancer, colorectal cancer, colon cancer, bile duct cancer, cervical cancer, bladder cancer, liver cancer or breast cancer.

In some embodiments, methods provided herein further comprise administration of at least one additional therapeutic agent to the subject. The at least one additional therapeutic agent may be administered concomitantly or sequentially with the compound or composition described herein. In some embodiments, the at least one additional therapeutic agent is a chemotherapeutic agent or an anti-cancer agent. In an embodiment, the at least one additional therapeutic agent is an immune checkpoint inhibitor, such as, without limitation, ipulimumab, nivolumab or lambrolizumab.

In additional embodiments, methods provided herein further comprise administration of a tumor vaccine (e.g., a vaccine effective against melanoma); the tumor vaccine can comprise genetically modified tumor cells or a genetically modified cell line, including genetically modified tumor cells or a genetically modified cell line that has been transfected to express granulocyte-macrophage stimulating factor (GM-CSF). In particular embodiments, the vaccine includes one or more immunogenic peptides and/or dendritic cells.

In another broad aspect, there are provided kits comprising the compound or composition of the disclosure. Kits may include a compound described herein, or a pharmaceutically acceptable salt, ester, hydrate, solvate or stereoisomer thereof, for use to treat, prevent or inhibit a KRAS-G12D-associated disease, disorder or condition. Kits may further comprise a buffer or excipient, and/or instructions for use. In some embodiments, kits further comprise at least one additional therapeutic agent, such as without limitation a chemotherapeutic agent, an immune- and/or inflammation-modulating agent, an anti-hypercholesterolemia agent, an anti-infective agent, or an immune checkpoint inhibitor.

### BRIEF DESCRIPTION OF THE DRAWINGS

In order to enhance comprehension of the present invention and provide a detailed demonstration of its implementation, further elucidation will now be presented through exemplary illustrations accompanied by reference to the appended diagrams. These diagrams depict distinct aspects and features of the illustrative embodiments in accordance with the present invention, in which:

FIG. 1 shows concentration-time curves for compound A1 after oral administration at equimolar doses of compound 1, compound 61 and compound A1 in ICR mice.

FIG. 2 shows concentration-time curves for compound A1 after oral administration at equimolar doses of compound 4, compound 17, compound 69 in ICR mice.

FIG. 3 shows concentration-time curves for compound A1 after intravenous administration at equimolar doses of compound 32, compound 39, and compound 52 in ICR mice.

FIG. 4 shows concentration-time curves for for compound A1 after intravenous administration at equimolar doses of compound 55, compound 56 and compound A1 in ICR mice.

FIG. 5 shows comparative results of tumor growth inhibitory effect in mice after oral administration of compound 1, compound A1 and blank control (vehicle). Error bars indicate standard error of the mean (S.E.M.).

FIG. 6 shows comparative results of tumor growth inhibitory effect in mice after intraperitoneal administration of compound 52, intravenous administration of compound 52, and intraperitoneal administration of compound A1 and blank control (vehicle). Error bars indicate S.E.M.

### DETAILED DESCRIPTION

### Definitions

In order to provide a clear and consistent understanding of the terms used in the present specification, a number of definitions are provided below. Moreover, unless defined otherwise, all technical and scientific terms as used herein have the same meaning as commonly understood to one of ordinary skill in the art to which this invention pertains.

The use of the word “a” or “an” when used in conjunction with the term “comprising” in the claims and/or the specification may mean “one”, but it is also consistent with the meaning of “one or more”, “at least one”, and “one or more than one”. Similarly, the word “another” may mean at least a second or more.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The terms “about” and “approximately” are used to indicate that a value includes an inherent variation of error for the device or the method being employed to determine the value.

The term “derivative” as used herein, is understood as being a substance similar in structure to another compound but differing in some slight structural detail.

The term “KRAS-G12D” (also referred to as “KRAS^{G12D}”) refers to a mutant form of the mammalian KRAS protein, in which the glycine residue at position 12 is replaced by an aspartic acid residue.

The term “prodrug” or its equivalent refers to a reagent that is directly or indirectly converted into an active form in vitro or in vivo (see, for example, R. B. Silverman, 1992, “The Organic Chemistry of Drug Design and Drug Action,” Academic Press, Chap. 8; Bundgaard, Hans; Editor. Neth. (1985), “Design of Prodrugs” 360 pp. Elsevier, Amsterdam; Stella, V.; Borchardt, R.; Hageman, M.; Oliyai, R.; Maag, H.; Tilley, J. (Eds.) (2007), “Prodrugs: Challenges and Rewards, XVIII, 1470 p. Springer). A prodrug can be used to change the biological distribution of specific drugs (for example, to make the drug usually not enter the protease reaction site) or its pharmacokinetics. A variety of groups have been used to modify compounds to form prodrugs, such as esters, ethers, phosphate esters/salts, etc. When a prodrug is administered to a subject, the group is cleaved in the subject by an enzymatic or non-enzymatic process, e.g., by reduction, oxidation or hydrolysis, or in another way, to release the active compound. As used herein, “prodrug” may include pharmaceutically acceptable salts or esters, or pharmaceutically acceptable solvates or chelates, as well as crystalline forms of a compound.

The term “pharmaceutically acceptable”, as used in the present disclosure, means drugs, pharmaceutical products, inert ingredients etc., described by the term, which are suitable for use in contact with tissues of humans and lower animals without abnormal toxicity, incompatibility, instability, irritation, allergic reactions etc., proportional to a reasonable benefit/risk ratio.

A “pharmaceutically acceptable stereoisomer” of a compound refers to the isomer produced by the different spatial arrangement of atoms or groups in a molecule. Isomers caused by the same order of atoms or atomic groups in the molecule but with different spatial arrangement are called stereoisomers. Stereoisomers are mainly divided into two categories: stereoisomers caused by bond length, bond angle, intramolecular double bond, ring, and the like are called configuration stereoisomers. In general, isomers cannot or are difficult to convert into each other. Stereoisomers caused only by the rotation of a single bond are called conformational stereoisomers, sometimes also known as rotational isomers. When the rotation in the rotating isomer is blocked and cannot rotate, it becomes a “stereoisomer”, for example, in the biphenyl structure, when α- and α′-positions bear large and different substituents, the rotation of the single bond between the two phenyl rings stops due to the hindrance between the substituents, producing two stereoisomers.

The terms “substituted”, “with substituent” and “with substitution” mean that the parent compound or part thereof has at least one substituent group. Unless otherwise indicated, a “substituent” group can be at one or more substitutable positions of the parent group, and when there is more than one substituent present at different positions of a given structure, the substituents can be the same or different at each position. In certain embodiments, the terms “substituent” and “substituted group” include, but are not limited to, halogen (F, Cl, Br or I), hydroxyl, mercapto, amino, nitro, carbonyl, carboxyl, alkyl, alkoxy, alkylamino, aryl, aryloxy, arylamino, acyl, sulfinyl, sulfonyl, phosphonyl and other organic parts routinely used and accepted in organic chemistry.

Where multiple substituents are indicated as being attached to a structure, it is to be understood that the substituents can be the same or different. Thus for example “Rₘ optionally substituted with 1, 2 or 3 R_{q} groups” indicates that Rₘ is substituted with 1, 2, or 3 R_{q} groups where the R_{q} groups can be the same or different.

The terms “unsubstituted” and “without substitution” mean that a compound or part thereof has no substituent except the undetermined chemical saturation of hydrogen atom.

In some embodiments, alkyl, heteroazanyl, acyl, cycloalkyl, heterocycloalkyl, alkoxy, aryloxy, heteroalkoxy, heteroaryloxy, aryl, heteroaryl group, amino acid residues, oligopeptide (dipeptide, tripeptide, tetrapeptide) residues, phosphoryl, phosphonyl, aminophosphonyl, sulfonyl, thioacyl, benzyl, alkoxycarbonyl, aminocarbonyl, mercaptothiocarbonyl, alkylthio, thiocarbonyl, benzyloxycarbonyl, glucoside, and glucuronide, as mentioned in the present disclosure, may be optionally substituted (e.g., “substituted” or “unsubstituted” alkyl, “substituted” or “unsubstituted” heterocyclyl, “substituted” or “unsubstituted” aryl, or “substituted” or “unsubstituted” heteroaryl group).

Unless otherwise specified, a “substituted” group has one substituent at one or more substitutable positions, and the substituent is the same or different at each position when replacing more positions in any given structure.

As used herein, the term “hydrocarbyl” means a group only containing carbon and hydrogen atoms, which may be saturated or unsaturated. For example, alkyl, alkenyl, and alkynyl are all examples of “hydrocarbyl”. Non-limiting examples of hydrocarbyl include methyl, ethyl, propyl, n-butyl, isobutyl, vinyl, propynyl, etc.

Unless the number of carbons is otherwise specified, “lower” as in “lower aliphatic group”, “lower hydrocarbyl”, “lower alkyl”, “lower alkenyl”, and “lower alkynyl”, as used herein, means that the moiety has at least one (at least two for alkenyl and alkynyl) and ≤6 carbon atoms.

The term “cycloalkyl”, “alicyclic group”, “carbocycle”, “cyclic group” and equivalents mean a group containing saturated or partially unsaturated carbon rings in a monocyclic, Spiro (sharing one atom), or fused (sharing at least one bond) carbocyclic system, wherein the carbocyclic system has 3-15 carbon atoms. Examples of cycloalkyl include, but are not limited to, cyclopropyl, cyclobutyl, cyclopentyl, cyclopenten-1-yl, cyclopenten-2-yl, cyclopenten-3-yl, cyclohexyl, cyclohexen-1-yl, cyclohexen-2-yl, cyclohexen-3 cycloheptyl, bicyclo[4,3,0]nonyl, norbornyl, etc. The term “cycloalkyl” includes both unsubstituted and substituted cycloalkyl.

The terms “aryl” and “aromatic”, as used in the present disclosure, refers to aryl groups having “4n+2” (π) electrons and 6-14 ring atoms in a conjugated mono- or polycyclic system (fused or non-fused), wherein n is an integer from 1 to 3. The polycyclic system includes at least one aromatic ring. The aryl can be linked directly or via C₁-C₃ alkyl (also known as arylalkyl or alkylaryl). Examples of aryl include, but are not limited to, phenyl, benzyl, phenethyl, 1-phenylethyl, tolyl, naphthyl, biphenyl, terphenyl, indenyl, benzocyclooctenyl, benzocycloheptenyl, azulenyl, acenaphthyl, fluorenyl, phenanthryl, anthryl, etc. The term “aryl” includes unsubstituted and substituted aryl. When the aryl group is linked by hydrocarbyl, it is also known as aryl hydrocarbyl group.

The term “heterocycle” and equivalents, as used in the present disclosure, means a group comprising a saturated or partially unsaturated carbocyclic ring in a monocyclic, spiro (sharing one atom) or fused (sharing at least one bond) carbocyclic system, which has 3-15 carbon atoms, including 1-6 heteroatoms (e.g., N, O, S, P) or groups containing heteroatoms (e.g., NH, NRx (where Rx is alkyl, acyl, aryl, heteroaryl or cycloalkyl), PO₂, SO, SO₂, etc.). Heterocyclic hydrocarbyl groups may be linked to C or with heteroatoms (e.g., via nitrogen atoms). The term “heterocycle” or “heterocyclic” includes heterocycloalkyl and heteroaryl. Examples of heterocycles include, but are not limited to, acridinyl, azocinelyl, benzimidazolyl, benzofuranyl, benzothienyl, benzothienyl, benzoxazolyl, benzothiazolyl, benzotriazolyl, benzotetrazolyl, benzisoxazolyl, benzisothiazolyl, 4αH-carbazolyl, carbolinyl, chromanyl, chromenyl, misolinyl, decahydroquinolinyl, 2H, 6H-1, 5, 2-dithiazinyl, dihydrofuro [2, 3-b] tetrahydrofuran, furanyl, furazanyl, imidazolidinyl, imidazolinyl, 1H-indazolyl, dihydroindolyl, 3H-indolyl, isoquinolinyl, isothiazolyl, isoxazolyl, methylenedioxyphenyl, morpholinyl, naphthyridinyl, octahydroisoquinolinyl, oxadiazolyl, 1, 2, 3-oxadiazolyl, 1, 2, 4-oxadiazolyl, 1, 2, 5-oxadiazolyl, 1, 3, 4-oxadiazolyl, oxazolidinyl, oxazolyl, oxazolidinyl, pyrimidinyl, phenanthridinyl, phenanthroline, phenazinyl, phenothiazinyl, phenoxazinyl, phthalazinyl, piperazinyl, piperidinyl, piperidinyl, 4-piperidinyl, piperonyl, pteridinyl, purinyl, pyranyl, pyrazolinyl, pyrazolidinyl, pyrazolinyl, pyrazolinyl, pyridazinyl, pyridazinyl, pyridoxazole, pyridimidazole, pyridothiazole, pyridyl, pyridyl, pyrrolyl, pyrrolyl, quinazolinyl, quinolinyl, 4H-quinazinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthryl, thiazolyl, thienyl, thienothiazolyl, thienoxazolyl, thienoimidazolyl, thienyl, triazinyl, 1,2,3-triazolyl, 1,2,4-triazolyl, 1,2,5-triazolyl, 3,4-triazolyl, xanthenyl etc. The term “heterocycle” includes unsubstituted and substituted heterocyclyl. Heterocycles linked by hydrocarbyl are also known as heterocyclic hydrocarbyl.

The term “fused ring” means a polycyclic system containing fused rings. The fused ring system typically contains 2 or 3 rings and/or up to 18 ring atoms. As described above, cycloalkyl, aryl, and heterocyclic can form a fused ring system. Thus, the fused ring system may be aromatic, partially or non-aromatic, and may contain heteroatoms. According to the definition, the spiro ring system is not a fused polycyclic. However, the fused polycyclic system of the present disclosure may have a spiro ring linked thereto through a single ring atom of the system. Examples of fused ring systems include, but are not limited to, naphthyl (e.g., 2-naphthyl), indenyl, phenanthryl, anthryl, pyrenyl, benzimidazole, benzothiazole, etc.

The term “acyl”, as used in the present disclosure, refers to the fragment —C(═O)Rₐ obtained after the removal of a hydroxy from a carboxylic acid molecule. The term “acyl” refers to a compound or fragment in which at least one carbon or heteroatom is covalently bonded to the carbon atom of —C═O. Acyl includes substituted and unsubstituted groups.

The term “amine” or “amino”, as used in the present disclosure, refers to unsubstituted or substituted fragments of the general Formula —NRₐR_{b} in which Rₐ and R_{b} are independently substituted or unsubstituted hydrogen, hydrocarbyl, aryl, cyclic or heterocyclic, etc., or Rₐ and R_{b} together form a heterocyclic ring with the nitrogen atom to which they are linked. The term “amide” refers to the structure —C(═O)NR_{b}R_{c} in which the amino is directly linked to the acyl. The term “acylamino” means the covalent bonding of at least one carbon or heteroatom in a compound or fragment to a carbon atom on acylamino.

The term “alkanoyloxy” means that Rₐ on an acyl is an alkyl, and the oxygen atom of the alkyl is connected to a carbon atom in the acyl, while the other end is covalently bonded to at least one carbon or heteroatom in the compound or fragment.

The term “thioacyl” refers to a fragment of —C(═S)Rₐ, formed by substituting the oxygen atom on the acyl group with a sulfur atom.

The term “aliphatic acyl” means an acyl group to which an aliphatic group is linked to a carbon atom on the acyl, i.e., Rₐ is aliphatic. The term “aroyl” refers to an acyl to which the aryl is linked to a carbon atom on the acyl, i.e., Rₐ is aryl.

The term “phosphonyl” or “phosphoryl” means the fragment —P(═O)(OR_{d})Rₑ left after dehydroxylation of monomolecular phosphoric acid. The term “phosphonyl” means the covalent bonding of at least one carbon or heteroatom in a compound or fragment to a phosphorus atom on the phosphonyl. R_{d} is substituted or unsubstituted hydrogen, hydrocarbyl, aryl, cyclic or heterocyclic group, etc. The term “aminophosphonyl” means the linkage of amine to phosphonyl, i.e., Rₑ is amine.

The term “sulfonyl” refers to the fragment left after the dehydroxylation of monomolecular sulfonic acid, and the term “sulfonyl” refers to the covalent bonding of at least one carbon or heteroatom in a compound or fragment to a sulfur atom on the sulfonyl.

The term “carbonyl” refers to a C═OR_{f} fragment formed by carbon and oxygen atoms linked by a double bond, and the term “carbonyl” is a constituent of functional groups such as aldehydes, ketones, acids, etc. The term “carbonyl” refers to the covalent bonding of at least one carbon or heteroatom of a compound or fragment to a carbon atom on C═OR_{f}, and R_{f} is a substituted or unsubstituted hydrogen, hydrocarbyl, aryl, cyclic or heterocycloalkyl, etc. The term “alkoxycarbonyl” means that R_{f} is an alkoxy, wherein the oxygen atom of the alkoxy is linked to the carbon atom of the carbonyl. The term “aminocarbonyl” means that R_{f} is an amine, wherein the nitrogen atom of the amine is linked to the carbon atom of the carbonyl. The term “benzyloxycarbonyl” means the linkage of the oxygen atom of the benzyloxy to the carbon atom of the carbonyl.

The term “thiocarbonyl” refers to a fragment of —C(═S)R_{f} formed after the substitution of an oxygen atom on the carbonyl by a sulfur atom. The term “mercaptothiocarbonyl” means that R_{f} is a sulfhydryl, wherein the carbon atom of the thiocarbonyl is linked to the sulfur atom of the sulfhydryl.

The term “alkylthio” refers to an alkyl linked to a sulfhydryl thereon. A suitable alkylthio includes 1 to about 20 carbon atoms, preferably 1 to about 15 carbon atoms.

The term “alkoxy” or “lower alkoxy”, as used in the present disclosure, refers to a structure in which the alkyl is linked to an oxygen atom. A representative alkoxy includes a group having 1 to about 6 carbon atoms, such as methoxy, ethoxy, propoxy, t-butoxy, etc. Examples of alkoxy include, but are not limited to, methoxy, ethoxy, isopropoxy, propoxy, butoxy, pentoxy, fluoromethoxy, difluoromethoxy, trifluoromethoxy, chloromethoxy, dichloromethoxy, trichloromethoxy, etc. The term “alkoxy” includes unsubstituted or substituted alkoxy, as well as perhaloalkoxy, etc.

“Cholic acid substituents” in the present disclosure refer to bile acids synthesized by liver cells, also called primary bile acids, including cholic acid, ursodeoxycholic acid, chenodeoxycholic acid, glycocholic acid, taurocholic acid, glycochenodeoxycholic acid, taurochenodeoxycholic acid, especially substituted chenodeoxycholic acid and substituted ursodeoxycholic acid.

The term “base compound” or “base molecule”, as used in the present disclosure, refers to a particular compound or drug molecule with desirable biological activity. In addition to being a drug molecule by itself, it can also be further modified or derivatized to form new compounds, such as prodrug compounds or derivatized compounds.

The term “ester-forming group” or “ester”, as used in the present disclosure, refers to a structure in which the fragment contains an ester functional group —RCOOR′ (where R′ is generally another non-H group such as alkyl). Non-limiting examples for R include a lower alkyl or aryl, such as methylene, ethylene, isopropylene, isopropylidene, phenylene, etc. Non-limiting examples for R′ include a lower alkyl or aryl, such as methyl, ethyl, propyl, isopropyl, butyl, phenyl, etc. The term “ester alkyl” means that R′ is an alkyl, one end of which is directly connected with the oxygen on the ester, and the other end is covalently bonded with at least one carbon or heteroatom in a compound or fragment.

The term “amino acid” generally refers to an organic compound that contains both a carboxylic acid group and an amino group. The term “amino acid” includes both “natural” and “unnatural” amino acids. In addition, the term “amino acid” includes an O-alkylated amino acid or an N-alkylated amino acid, as well as an amino acid with a nitrogen-, sulfur-, or oxygen-containing side chain (e.g., Lys, Cys, or Ser), wherein the nitrogen, sulfur, or oxygen atom may or may not be acylated or alkylated. The amino acid may be a pure L-isomer or D-isomer, or a mixture of L-isomer and D-isomer, including (but not limited to) a racemic mixture.

The term “natural amino acid” and equivalent refers to L-amino acids normally found in naturally occurring proteins. Examples of natural amino acids include, but are not limited to, alanine (Ala), cysteine (Cys), aspartic acid (Asp), glutamic acid (Glu), phenylalanine (Phe), glycine (Gly), histidine (His), isoleucine (Ile), lysine (Lys), leucine (Leu), methionine (Met), asparagine (Asn), proline (Pro), glutamine (Gln), arginine (Arg), serine (Ser), threonine (Thr), valine (Val), tryptophan (Trp), tyrosine (Tyr), β-alanine (beta-Ala) and γ-aminobutyric acid (GABA).

The term “unnatural amino acid” refers to any derivative of a natural amino acid, including D-amino acid, as well as α-and β-amino acid derivatives. The terms “unnatural amino acid” and “not natural amino acid” are used interchangeably herein. It should be noted that certain amino acids (e.g., hydroxyproline), which may be classified as unnatural amino acids in the present disclosure, may also be present in certain biological tissues or specific proteins in nature. The amino acids with many different protecting groups and suitable for direct application in solid-phase peptide synthesis are available to purchase. In addition to twenty of the most common natural amino acids, the following exemplary unnatural amino acids and amino acid derivatives (common abbreviations in parentheses) may be used according to the present disclosure: 2-aminoadipic acid (Aad), 3-aminoadipic acid (β-Aad), 2-aminobutyric acid (2-Abu), α,β-dehydro-2-aminobutyric acid (8-AU), 1-aminocyclopropane-1-carboxylic acid (ACPC), aminoisobutyric acid (Aib), 3-aminoisobutyric acid (β-Aib), 2-amino-thiazoline-4-carboxylic acid, 5-aminopentanoic acid (5-Ava), 6-aminohexanoic acid (6-Ahx), 2-aminoheptanoic acid (Ahe), 8-aminooctanoic acid (8-Aoc), 11-aminoundecanoic acid (11-Aun), 12-aminododecanoic acid (12-Ado), 2-aminobenzoic acid (2-Abz), 3-aminobenzoic acid (3-Abz), 4-aminobenzoic acid (4-Abz), 4-amino-3-hydroxy-6-methylheptanoic acid (statine, Sta), aminooxyacetic acid (Aoa), 2-aminotetraline-2-carboxylic acid (ATC), 4-amino-5-cyclohexyl-3-hydroxyvaleric acid (ACHPA), P-aminophenylalanine (4-NH₂-Phe), 2-aminopimelic acid (Apm), biphenylalanine (Bip), P-bromophenylalanine (4-Br-Phe), O-chlorophenylalanine (2-Cl-Phe), M-chlorophenylalanine (3-Cl-Phe), P-chlorophenylalanine (4-Cl-Phe), M-chlorotyrosine (3-Cl-Tyr), P-benzoylphenylalanine (Bpa), t-butylglycine (TLG), cyclohexylalanine (Cha), cyclohexylglycine (Chg), desmosine (Des), 2,2-diaminopimelic acid (Dpm), 2,3-diaminopropionic acid (Dpr), 2,4-diaminobutyric acid (Dbu), 3,4-dichlorophenylalanine (3,4-Cl2-Phe), 3,4-difluorophenylalanine (3,4-F2-Phe), 3,5-diiodotyrosine (3,5-I2-Tyr), N-ethylglycine (EtGly), N-ethylasparagine (EtAsn), o-fluorophenylalanine (2-F-Phe), m-fluorophenylalanine (3-F-Phe), p-fluorophenylalanine (4-F-Phe), M-fluorotyrosine (3-F-Tyr), homoserine (Hse), homophenylalanine (Hfe), homotyrosine (Htyr), hydroxylysine (Hyl), isohydroxylysine (aHyl), 5-hydroxytryptophan (5-OH-Trp), 3- or 4-hydroxyproline (3- or 4-Hyp), p-iodophenylalanine-isotyrosine (3-I-Tyr), dihydroindole-2-carboxylic acid (Idc), isoiduramycin (Ide), isoleucine (α-Ile), isopipecolic Acid (Inp), N-methylisoleucine (MeLys), m-methyltyrosine (3-Me-Tyr), N-methylvaline (MeVal), 1-naphthylalanine (1-Nal), 2-naphthylalanine (2-Nal), p-nitrophenylalanine (4-NO2-Phe), 3-nitrotyrosine (3-NO2-Tyr), norleucine (Nle), norvaline (Nva), ornithine (Om), O-phosphotyrosine (H2PO3-Tyr), octahydroindole-2-carboxylic acid (Oic), penicillamine (Pen), pentafluorophenylalanine (F5-Phe), phenylglycine (Phg), pipecolic acid (Pip), propargylglycine (Pra), pyroglutamic acid (PGLU), sarcosine (Sar), tetrahydroisoquinoline-3-carboxylic acid (Tic), and thiazolidine-4-carboxylic acid (thioproline, Th).

The term “peptide” or “oligopeptide” refers to a compound formed by the intermolecular dehydration condensation of two or more amino acids linked together by an amide bond. In general, the number of amino acids constituting a peptide ranges from 2 (dipeptide) to 20 (eicosapeptide).

The term “residue” refers to the major part of the molecule after the removal of a group, such as amino acid residue (e.g., the structure H₂NCH₂CO—, i.e., glycyl, the part after the removal of hydroxyl from glycine) and peptide residue.

The term “solvate” refers to a physical association of a compound with one or more solvent molecules, whether organic or inorganic. This physical association includes hydrogen bonding. In certain instances, a solvate will be capable of isolation, for example when one or more solvent molecules are incorporated in the crystal lattice of a crystalline solid. “Solvate” encompasses both solution-phase and isolable solvates. Exemplary solvates include, without limitation, hydrates, ethanolates, methanolates, hemiethanolates, and the like.

The term “hydrate” refers to a compound that is bonded to one or more water (H₂O) molecule, e.g., by a hydrogen bond.

The term “salt-forming moiety”, as used in the present disclosure, refers to a moiety capable of forming a salt with an acidic group, such as a carboxyl, including but not limited to, sodium, potassium, tetraethylamine, tetrabutylamine, etc.

As used herein, a “pharmaceutically acceptable salt” of a compound means a salt of a compound that is pharmaceutically acceptable. Desirable are salts of a compound that retain or improve the biological effectiveness and desired biology activities or properties of the free acids and bases of the parent compound as defined herein, or that take advantage of an intrinsically basic, acidic or charged functionality on the molecule and that are not biologically or otherwise undesirable. Examples of pharmaceutically acceptable salts are also described, for example, in Berge et al., “Pharmaceutical Salts”, J. Pharm. Sci. 66, 1-19 (1977). Examples of pharmaceutically acceptable salts include but are not limited to.
(1) A salt formed by adding an acid to a basic or positively charged functional group. Inorganic acids include hydrochloric acid, hydrobromic acid, hydroiodic acid, sulfuric acid, sulfamic acid, nitric acid, phosphoric acid, carbonate, etc. Organic acids include acetic acid, propionic acid, lactic acid, oxalic acid, glycolic acid, pivalic acid, t-butyl acetic acid, P-hydroxybutyric acid, valeric acid, caproic acid, cyclopentane propionic acid, pyruvic acid, malonic acid, succinic acid, malic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1, 2-ethanedisulfonic acid, 2-hydroxyethanesulfonic acid, cyclohexylsulfamic acid, benzenesulfonic acid, sulfanilic acid, 4-chlorobenzenesulfonic acid, 2-naphthalenesulfonic acid, 4-toluenesulfonic acid, camphorsulfonic acid, 3-phenylpropionic acid, laurylsulfonic acid, laurylsulfuric acid, oleic acid, palmitic acid, stearic acid, lauric acid, fluric acid, pantothenic acid, lactobionic acid, alginic acid, galactonic acid, galacturonic acid, gluconic acid, glucoheptonic acid, glutamic acid, naphthoic acid, hydroxynaphthoic acid, salicylic acid, ascorbic acid, stearic acid, muconic acid, etc.
(2) When an acidic proton is present in the parent compound, or a metal ion replaces it, a base may be added to give a salt. The metal ions include alkaline metal ions (such as lithium, sodium, and potassium), alkaline earth metal ions (magnesium, calcium, barium), or other metal ions such as aluminum, zinc, iron, etc. Organic bases include, but are not limited to, N,N′-dibenzylethylenediamine, ethanolamine, diethanolamine, triethanolamine, tromethamine, N-methylglucamine, piperazine, chloroprocaine, procaine, choline, lysine, etc.

Pharmaceutically acceptable salts may be synthesized from a parent compound containing basic or acidic fragments by conventional chemical methods. Typically, such salts are prepared by reacting a compound (free acid or base) with an equal stoichiometric amount of a base or acid in water, an organic solvent, or a mixture of the two. Salts may be prepared in situ during the final isolation or purification of a compound or by separately reacting a compound in its free acid or base form alone with the desired corresponding base or acid and isolating the salt thus formed. The term “pharmaceutically acceptable salt” also includes zwitterionic compounds comprising a cationic group covalently bonded to an anionic group, called “inner salt” or “internal salt”. It should be understood that all acid, salt, base, and other ionic and non-ionic forms of compounds described herein are intended to be encompassed. For example, if a compound is shown as an acid herein, the salt forms of the compound are also encompassed. Likewise, if a compound is shown as a salt, the acid and/or basic forms are also encompassed.

### Compounds

As used herein, the term “compounds of the disclosure” and equivalent expressions refers to KRAS^{G12D} inhibitor compounds provided herein as being useful for at least one purpose of the disclosure, e.g., those encompassed by structural Formula (A), (B), (I), (II), (IIA), (III), (IV), (V), (VI), and (VII), and includes specific compounds mentioned herein such as those in Tables 2 and 2a as well as their pharmaceutically acceptable salts, esters, hydrates, solvates and stereoisomers.

As would be understood by a person of ordinary skill in the art, the recitation of “a compound” is intended to include salts, esters, solvates, hydrates, oxides, and inclusion complexes of that compound as well as any stereoisomeric form or polymorphic form, or a mixture of any such forms of that compound in any ratio. Thus, in accordance with some embodiments, a compound as described herein, including in the contexts of pharmaceutical compositions and methods of treatment, is provided as the salt form.

It should be understood that compounds described herein may contain one or more chiral centers and/or double bonds and therefore, may exist as stereoisomers, such as double-bond isomers (i.e., geometric isomers), enantiomers, or diastereomers. Chemical structures disclosed herein are intended to encompass all possible enantiomers and stereoisomers of the illustrated compounds including the stereoisomerically pure form (e.g., geometrically pure, enantiomerically pure, or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan, e.g., chiral chromatography (such as chiral HPLC), immunoassay techniques, or the use of covalently (such as Mosher's esters) and non-covalently (such as chiral salts) bound chiral reagents to respectively form a diastereomeric mixture which can be separated by conventional methods, such as chromatography, distillation, crystallization or sublimation, the chiral salt or ester is then exchanged or cleaved by conventional means, to recover the desired isomers. The compounds may also exist in several tautomeric forms including the enol form, the keto form, and mixtures thereof. The chemical structures depicted herein are also intended to encompass all possible tautomeric forms of the illustrated compounds.

Compounds may exist in unsolvated forms as well as solvated forms, including hydrated forms. In general, compounds may be hydrated or solvated. Certain compounds may exist in multiple crystalline or amorphous forms. In general, all physical forms are intended to be encompassed herein.

Compounds described herein include, but are not limited to, their optical isomers, racemates, and other mixtures thereof. In those situations, the single enantiomers or diastereomer, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral high-pressure liquid chromatography (HPLC) column. In addition, such compounds include Z- and E-forms (or cis- and trans-forms) of compounds with carbon-carbon double bonds. Where compounds described herein exist in various tautomeric forms, the term “compound” is intended to include all tautomeric forms of the compound. Such compounds also include crystal forms including polymorphs and clathrates. Similarly, the term “salt” is intended to include all tautomeric forms and crystal forms of the compound.

The configuration of any carbon-carbon double bond appearing herein is selected for convenience only and is not intended to designate a particular configuration; thus a carbon-carbon double bond depicted arbitrarily herein as E may be Z, E, or a mixture of the two in any proportion.

For compounds provided herein, it is intended that, in some embodiments, salts thereof are also encompassed, including pharmaceutically acceptable salts. Those skilled in the art will appreciate that many salt forms (e.g., TFA salt, tetrazolium salt, sodium salt, potassium salt, etc,) are possible; appropriate salts are selected based on considerations known in the art. The term “pharmaceutically acceptable salt” refers to salts prepared from pharmaceutically acceptable non-toxic acids or bases including inorganic acids and bases and organic acids and bases. For example, for compounds that contain a basic nitrogen, salts may be prepared from pharmaceutically acceptable non-toxic acids including inorganic and organic acids. Suitable pharmaceutically acceptable acid addition salts for the compounds of the present disclosure include without limitation acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. When the compounds contain an acidic side chain, suitable pharmaceutically acceptable base addition salts for the compounds of the present disclosure include without limitation metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine), and procaine.

For compounds provided herein, it is intended that, in some embodiments, compounds may contain unnatural proportions of atomic isotopes at one or more of the atoms that constitute such compounds. Unnatural proportions of an isotope may be defined as ranging from the amount found in nature to an amount consisting of 100% of the atom in question. For example, compounds may incorporate radioactive isotopes, such as for example tritium (³H), iodine-125 (¹²⁵I) or carbon-14 (¹⁴C), or non-radioactive isotopes, such as deuterium (²H) or carbon-13 (¹³C). Such isotopic variations can provide additional utilities to those described elsewhere within this application. For instance, isotopic variants of the compounds of the disclosure may find additional utility, including but not limited to, as diagnostic and/or imaging reagents, or as cytotoxic/radiotoxic therapeutic agents. Additionally, isotopic variants can have altered pharmacokinetic and pharmacodynamic characteristics which can contribute to enhanced safety, tolerability or efficacy during treatment. All isotopic variations of compounds provided herein, whether radioactive or not, are intended to be encompassed herein.

Isotopic enrichment is a process by which the relative abundance of the isotopes of a given element are altered, thus producing a form of the element that has been enriched (i.e., increased) in one particular isotope and reduced or depleted in its other isotopic forms. As used herein, an “isotope-enriched” compound or derivative refers to a compound in which one or more specific isotopic form has been increased, i.e., one or more of the elements has been enriched (i.e., increased) in one or more particular isotope. Generally, in an isotope-enriched compound or derivative, a specific isotopic form of an element at a specific position of the compound is increased. It should be understood however that isotopic forms of two or more elements in the compound may be increased. Further, an isotope-enriched compound may be a mixture of isotope-enriched forms that are enriched for more than one particular isotope, more than one element, or both. As used herein, an “isotope-enriched” compound or derivative possesses a level of an isotopic form that is higher than the natural abundance of that form. The level of isotope-enrichment will vary depending on the natural abundance of a specific isotopic form. In some embodiments, the level of isotope-enrichment for a compound, or for an element in a compound, may be from about 2 to about 100 molar percent (%), e.g., about 2%, about 5%, about 17%, about 30%, about 51%, about 83%, about 90%, about 95%, about 96%, about 97%, about 98%, greater than about 98%, about 99%, or 100%.

As used herein, an “element of natural abundance” and an “atom of natural abundance” refers to the element or atom respectively having the atomic mass most abundantly found in nature. For example, hydrogen of natural abundance is ¹H (protium); nitrogen of natural abundance is ¹⁴N; oxygen of natural abundance is ¹⁶O; carbon of natural abundance is ¹²C; and so on. A “non-isotope enriched” compound is a compound in which all the atoms or elements in the compound are isotopes of natural abundance, i.e., all the atoms or elements have the atomic mass most abundantly found in nature.

### Compositions

In certain embodiments, there are provided pharmaceutical compositions comprising a compound of the disclosure, e.g., a compound of Formula (A), Formula (B), Formula (I), Formula (II), Formula (IIA), Formula (III), Formula (IV), Formula (V), (Formula VI), or Formula (VII), or a pharmaceutically acceptable salt, ester, hydrate, solvate or stereoisomer thereof, and a pharmaceutically acceptable excipient, carrier or diluent. In an embodiment, there is provided a pharmaceutical composition comprising a compound of Formula (A), Formula (B), Formula (I), or Formula (II), (IIA), (III), (IV), (V), (VI), or (VII), or a compound in any one of Tables 2 and 2a, or a pharmaceutically acceptable salt thereof, and a pharmaceutically acceptable excipient, carrier, or diluent.

The preparation of pharmaceutical compositions can be carried out as known in the art (see, for example, Remington: The Science and Practice of Pharmacy, 20^{th} Edition, 2000). For example, a therapeutic compound and/or composition, together with one or more solid or liquid pharmaceutical carrier substances and/or additives (or auxiliary substances) and, if desired, in combination with other pharmaceutically active compounds having therapeutic or prophylactic action, are brought into a suitable administration form or dosage form which can then be used as a pharmaceutical in human or veterinary medicine. Pharmaceutical preparations can also contain additives, of which many are known in the art, for example fillers, disintegrants, binders, lubricants, wetting agents, stabilizers, emulsifiers, dispersants, preservatives, sweeteners, colorants, flavorings, aromatizers, thickeners, diluents, buffer substances, solvents, solubilizers, agents for achieving a depot effect, salts for altering the osmotic pressure, coating agents or antioxidants.

The term “pharmaceutical composition” means a composition comprising a compound as described herein and at least one component comprising pharmaceutically acceptable carriers, diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifungal agents, lubricating agents, dispersants and dispensing agents, depending on the nature of the mode of administration and dosage forms. It should be understood that, as used herein, a pharmaceutical composition comprises a compound disclosed herein (or a pharmaceutically acceptable salt, ester, hydrate, solvate, or stereoisomer thereof) and a pharmaceutically acceptable excipient, carrier, diluent, adjuvant, or vehicle. In certain embodiments, the amount of a compound in a composition is such that it is effective as an inhibitor of KRAS-G12D in a biological sample (e.g., in a cellular assay, in an in vivo model, etc.) or in a subject. In certain embodiments, the composition is formulated for administration to a subject in need of such composition. In some embodiments, the composition is an injectable formulation. In other embodiments, the composition is formulated for oral administration to a subject.

The term “pharmaceutically acceptable carrier” is used to mean any carrier, diluent, adjuvant, excipient, or vehicle, as described herein. Examples of suspending agents include ethoxylated isostearyl alcohols, polyoxyethylene sorbitol and sorbitan esters, microcrystalline cellulose, aluminum metahydroxide, bentonite, agar-agar and tragacanth, or mixtures of these substances. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monosterate and gelatin. Examples of suitable carriers, diluents, solvents, or vehicles include water, ethanol, polyols, suitable mixtures thereof, vegetable oils (such as olive oil), and injectable organic esters such as ethyl oleate. Examples of excipients include lactose, milk sugar, sodium citrate, calcium carbonate, and dicalcium phosphate. Examples of disintegrating agents include starch, alginic acids, and certain complex silicates. Examples of lubricants include magnesium stearate, sodium lauryl sulphate, talc, as well as high molecular weight polyethylene glycols.

A pharmaceutical composition provided herein can be administered orally, for example in the form of pills, tablets, lacquered tablets, sugar-coated tablets, granules, hard and soft gelatin capsules, aqueous, alcoholic or oily solutions, syrups, emulsions or suspensions, or rectally, for example in the form of suppositories. Administration can also be carried out parenterally, for example subcutaneously, intramuscularly or intravenously in the form of solutions for injection or infusion. Other suitable administration forms are, for example, percutaneous or topical administration, for example in the form of ointments, creams, tinctures, sprays or transdermal therapeutic systems, or the inhalative administration in the form of nasal sprays or aerosol mixtures, or, for example, microcapsules, implants or wafers.

In some embodiments, pharmaceutical compositions provided herein are suitable for oral administration. For example, a pharmaceutical composition may be in the form of a hard shell gelatin capsule, a soft shell gelatin capsule, a cachet, a pill, a tablet, a lozenge, a powder, a granule, a pellet, a pastille, or a dragee. Alternatively, a pharmaceutical composition may be in the form of a solution, an aqueous liquid suspension, a non-aqueous liquid suspension, an oil-in-water liquid emulsion, a water-in-oil liquid emulsion, an elixir, or a syrup. Pharmaceutical compositions may or may not be enteric coated. In some embodiments, pharmaceutical compositions are formulated for controlled release, such as delayed or extended release.

In further embodiments, compounds and compositions thereof may be formulated in multi-dose forms, i.e., in the form of multi-particulate dosage forms (e.g., hard gelatin capsules or conventional tablets prepared using a rotary tablet press) comprising one or more bead or minitab populations for oral administration. The conventional tablets rapidly disperse on entry into the stomach. The one or more coated bead or minitab populations may be compressed together with appropriate excipients into tablets (for example, a binder, a diluent/filler, and a disintegrant for conventional tablets.

Tablets, pills, beads, or minitabs of the compounds and compositions of the compounds may be coated or otherwise compounded to provide a dosage form affording the advantage of controlled release, including delayed or extended release, or to protect from the acid conditions of the stomach. For example, the tablet or pill can include an inner dosage and an outer dosage component, the latter being in the form of a coating over the former. The two components can be separated by a polymer layer that controls the release of the inner dosage.

In certain embodiments, the layer may comprise at least one enteric polymer. In further embodiments, the layer may comprise at least one enteric polymer in combination with at least one water-insoluble polymer. In still further embodiments, the layer may comprise at least one enteric polymer in combination with at least one water-soluble polymer. In yet further embodiments, the layer may comprise at least one enteric polymer in combination with a pore-former.

In certain embodiments, the layer may comprise at least one water-insoluble polymer. In still further embodiments, the layer may comprise at least one water-insoluble polymer in combination with at least one water-soluble polymer. In yet further embodiments, the layer may comprise at least one water-insoluble polymer in combination with a pore-former.

Representative examples of water-soluble polymers include polyvinylpyrrolidone (PVP), hydroxypropyl methylcellulose (HPMC), hydroxypropylcellulose (HPC), polyethylene glycol, and the like.

Representative examples of enteric polymers include esters of cellulose and its derivatives (cellulose acetate phthalate, hydroxypropyl methylcellulose phthalate, hydroxypropyl methylcellulose acetate succinate), polyvinyl acetate phthalate, pH-sensitive methacrylic acid-methylmethacrylate copolymers and shellac. These polymers may be used as a dry powder or an aqueous dispersion. Some commercially available materials that may be used are methacrylic acid copolymers sold under the trademark Eudragit (LI 00, S I 00, L30D) manufactured by Rohm Pharma, Cellacefate (cellulose acetate phthalate) from Eastman Chemical Co., Aquateric (cellulose acetate phthalate aqueous dispersion) from FMC Corp. and Aqoat (hydroxypropyl methylcellulose acetate succinate aqueous dispersion) from Shin Etsu K.K.

Representative examples of useful water-insoluble polymers include ethylcellulose, polyvinyl acetate (for example, Kollicoat SR #30D from BASF), cellulose acetate, cellulose acetate butyrate, neutral copolymers based on ethyl acrylate and methylmethacrylate, copolymers of acrylic and methacrylic acid esters with quaternary ammonium groups such as Eudragit NE, RS and RS30D, RL or RL30D and the like.

Any of the above polymers may be further plasticized with one or more pharmaceutically acceptable plasticizers. Representative examples of plasticizers include triacetin, tributyl citrate, triethyl citrate, acetyl tri-n-butyl citrate diethyl phthalate, castor oil, dibutyl sebacate, acetylated monoglycerides and the like or mixtures thereof. The plasticizer, when used, may comprise about 3 to 30 wt. % and more typically about 10 to 25 wt. % based on the polymer. The type of plasticizer and its content depends on the polymer or polymers and nature of the coating system (e.g., aqueous or solvent based, solution or dispersion based and the total solids).

Pharmaceutical compositions typically must be sterile and stable under the conditions of manufacture and storage. A composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of injectable compositions can be brought about by including in the composition an agent which delays absorption, for example, monostearate salts and gelatin. Moreover, a compound can be administered in a time release formulation, for example in a composition which includes a slow release polymer. The compound can be prepared with carriers that will protect against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG).

Pharmaceutical compositions can also include carriers to protect the composition against rapid degradation or elimination from the body, such as a controlled release formulation, including liposomes, hydrogels, and microencapsulated delivery systems. For example, a time delay material such as glyceryl monostearate or glyceryl stearate alone, or in combination with a wax, may be employed. Any drug delivery apparatus may be used to deliver compounds and compositions of the disclosure, including implants (e.g., implantable pumps) and catheter systems, slow injection pumps and devices, all of which are well known to the skilled artisan.

Pharmaceutical compositions may also be in the form of a sterile injectable aqueous or oleagenous (oily) suspension. This suspension may be formulated according to the known art using those suitable dispersing or wetting agents and suspending agents mentioned herein. The sterile injectable preparation may also be a sterile injectable solution or suspension in a non-toxic parenterally acceptable diluent or solvent, for example, as a solution in 1,3-butane diol. Acceptable diluents, solvents and dispersion media that may be employed include water, Ringer's solution, isotonic sodium chloride solution, Cremophor EL™ (BASF, Parsippany, NJ) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose any bland fixed oil may be employed, including synthetic mono- or diglycerides. Moreover, fatty acids such as oleic acid, can be used in the preparation of injectables. Prolonged absorption of particular injectable formulations can be achieved by including an agent that delays absorption (e.g., aluminum monostearate or gelatin).

Many methods for the preparation of such formulations are generally known to those skilled in the art. Sterile injectable solutions can be prepared by incorporating an active compound, such as a compound of Formula (A) provided herein, in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, common methods of preparation are vacuum drying and freeze-drying which yields a powder of the active ingredient plus any additional desired ingredient from a previously sterile-filtered solution thereof. Compounds may also be formulated with one or more additional compounds that enhance their solubility.

It is often advantageous to formulate compositions (such as parenteral compositions) in dosage unit form for ease of administration and uniformity of dosage. The term “unit dosage form” refers to a physically discrete unit suitable as unitary dosages for human subjects and other animals, each unit containing a predetermined quantity of active material calculated to produce the desired therapeutic effect, in association with a suitable pharmaceutical carrier. The specification for the dosage unit forms of the disclosure may vary and are dictated by and directly dependent on (a) the unique characteristics of the therapeutic compound and the particular therapeutic effect to be achieved, and (b) the limitations inherent in the art of compounding such a therapeutic compound for the prevention or treatment of a KRAS-G12D-associated disease, disorder or condition, such as a cancer or a tumor.

In some embodiments, the pharmaceutical composition is provided in a single-use container (e.g., a single-use vial, ampoule, syringe, or autoinjector), whereas a multi-use container (e.g., a multi-use vial) is provided in other embodiments.

Pharmaceutical compositions provided herein can be formulated to be compatible with the intended method or route of administration; exemplary routes of administration are set forth herein. Furthermore, the pharmaceutical compositions may be used in combination with other therapeutically active agents or compounds as described herein in order to treat or prevent the KRAS-G12D-associated diseases, disorders and conditions as contemplated herein.

Pharmaceutical compositions containing the active ingredient (e.g., a KRAS-G12D inhibitor compound) may be in a form suitable for oral use, for example, as tablets, capsules, troches, lozenges, aqueous or oily suspensions, dispersible powders or granules, emulsions, hard or soft capsules, or syrups, solutions, beads, microbeads or elixirs. Pharmaceutical compositions intended for oral use may be prepared according to any method known in the art for the manufacture of pharmaceutical compositions, and such compositions may contain one or more agents such as, for example, sweetening agents, flavoring agents, coloring agents and preserving agents in order to provide pharmaceutically acceptable preparations. Tablets, capsules and the like generally contain the active ingredient in admixture with non-toxic pharmaceutically acceptable carriers or excipients which are suitable for the manufacture of tablets. These carriers or excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin, gum arabic or acacia, and lubricating agents, for example magnesium stearate, stearic acid or talc.

Tablets, capsules and the like suitable for oral administration may be uncoated or coated using known techniques to delay disintegration and absorption in the gastrointestinal tract and thereby provide a sustained action. For example, a time-delay material such as glyceryl monostearate or glyceryl distearate may be employed. They may also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylenevinylacetate, methycellulose, carboxymethylcellulose, protamine sulfate, or lactide/glycolide copolymers, polylactide/glycolide copolymers, or ethylenevinylacetate copolymers in order to control delivery of an administered composition. For example, the oral agent can be entrapped in microcapsules prepared by coacervation techniques or by interfacial polymerization, by the use of hydroxymethylcellulose or gelatin-microcapsules or poly (methylmethacrolate) microcapsules, respectively, or in a colloid drug delivery system. Colloidal dispersion systems include macromolecule complexes, nano-capsules, microspheres, microbeads, and lipid-based systems, including oil-in-water emulsions, micelles, mixed micelles, and liposomes. Methods for the preparation of the above-mentioned formulations will be apparent to those skilled in the art.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil. Aqueous suspensions contain the active materials in admixture with excipients suitable for the manufacture thereof. Such excipients can be suspending agents, for example sodium carboxymethylcellulose, methykellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are known in the art.

Pharmaceutical compositions of the present disclosure may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

Pharmaceutical compositions typically comprise a therapeutically effective amount of a KRAS-G12D inhibitor compound provided herein and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bi sulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer, N-(2-Hydroxyethyl)piperazine-N′-(2-ethanesulfonic acid) (HEPES), 2-(N-MoqJholino)ethanesulfonic acid (MES), 2-(N-Morpholino)ethanesulfonic acid sodium salt (MES), 3-(N-Morpholino)propanesulfonic acid (MOPS), and Ntris[Hydroxymethyl]methyl-3-aminopropanesulfonic acid (TAPS). After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form.

In some embodiments, there are provided pharmaceutical compositions that comprise an effective amount of a compound and/or composition described herein, and a pharmaceutically acceptable excipient, carrier or diluent. In an embodiment, there are provided pharmaceutical compositions for the treatment or prevention of a KRAS-G12D-associated disease, disorder or condition, such as a cancer or a tumor, comprising a compound described herein, or a pharmaceutically acceptable salt, ester, hydrate, solvate or stereoisomer thereof, and a pharmaceutically acceptable carrier. In another embodiment, there is provided a pharmaceutical composition for the prevention or treatment of a KRAS-G12D-associated disease, disorder or condition, such as a cancer or a tumor, the composition comprising a compound described herein, or a pharmaceutically acceptable salt thereof, and a pharmaceutically acceptable carrier.

### Methods of Use of Compounds and Compositions

In certain embodiments, there are provided methods for inhibition, prevention or treatment of a KRAS-G12D-associated disease, disorder or condition in a subject by administering an effective amount of a compound or composition described herein. In a related aspect, there are provided methods for prevention or treatment of a KRAS-G12D-associated hyperplastic or hyperproliferative disorder, e.g., a cancer or a tumor, in a subject in need thereof by administering an effective amount of a compound or composition described herein.

In an embodiment, there is provided herein a method of treating a subject (e.g., a human) with cancer or a disorder mediated by KRAS-G12D comprising the step of administering to the subject a therapeutically effective amount of an KRAS-G12D inhibitor compound provided herein or a pharmaceutically acceptable composition thereof.

There is also provided a method of treating a subject (e.g., a human) with cancer or a hyperproliferative disorder mediated by KRAS-G12D comprising the step of administering to the subject a therapeutically effective amount of a compound provided herein, e.g., a compound provided herein or a pharmaceutically acceptable composition thereof. In certain embodiments, the amount of a compound in a composition is such that it is effective as an inhibitor of KRAS-G12D in a biological sample (e.g., in a cellular assay, in an in vivo model, etc.) or in a subject. In certain embodiments, the composition is formulated for administration to a subject in need of such composition. In some embodiments, the composition is an injectable formulation. In some embodiments, the composition is formulated for intravenous administration. In other embodiments, the composition is formulated for oral administration to a subject. In some embodiments, the composition is in the form of a hard shell gelatin capsule, a soft shell gelatin capsule, a cachet, a pill, a tablet, a lozenge, a powder, a granule, a pellet, a pastille, or a dragee. In some embodiments, the composition is in the form of a solution, an aqueous liquid suspension, a non-aqueous liquid suspension, an oil-in-water liquid emulsion, a water-in-oil liquid emulsion, an elixir, or a syrup. In some embodiments, the composition is enteric coated. In some embodiments, the composition is formulated for controlled release.

In further embodiments, there are provided methods for treating or preventing cancer in a subject, comprising administering to the subject a therapeutically effective amount of at least one compound of the disclosure and at least one additional signal transduction inhibitor (STI). In a particular embodiment, the at least one STI is selected from the group consisting of bcr/abl kinase inhibitors, epidermal growth factor (EGF) receptor inhibitors, her-2/neu receptor inhibitors, and famesyl transferase inhibitors (FTIs). There are also provided methods of augmenting the rejection of tumor cells in a subject comprising administering a compound of the disclosure in conjunction with at least one chemotherapeutic agent and/or radiation therapy, wherein the resulting rejection of tumor cells is greater than that obtained by administering either the compound, the chemotherapeutic agent or the radiation therapy alone. In further embodiments, there are provided methods for treating cancer in a subject, comprising administering to the subject a therapeutically effective amount of at least one compound of the disclosure and at least one immunomodulator.

In further embodiments, there are provided methods for treating, inhibiting or preventing a hyperproliferative or hyperplastic disease or disorder in a subject, comprising administering to the subject an effective amount of at least one compound or pharmaceutical composition of the disclosure.

The terms “patient” and “subject” are used interchangeably herein to refer to a human or a non-human animal (e.g., a mammal). Non-limiting examples of subjects include humans, monkeys, cows, rabbits, sheep, goats, pigs, dogs, cats, rats, mice, and transgenic species thereof. In some embodiments, a subject is in need of treatment by the methods provided herein, and is selected for treatment based on this need. A subject in need of treatment is art-recognized, and includes subjects that have been identified as having a disease or condition (e.g., cancer, tumor, hyperproliferative disorder), or having a symptom of such a disease or condition, or being at risk of such a disease or condition, and would be expected, based on diagnosis, e.g., medical diagnosis, to benefit from treatment (e.g., curing, healing, preventing, alleviating, relieving, altering, remedying, ameliorating, improving, or affecting the disease or disorder, the symptom of the disease or disorder, or the risk of the disease or disorder). In certain embodiments, a subject is a human. In some embodiments, a subject has a cancer or tumor carrying the KRAS-G12D mutation.

The term “in need of treatment” as used herein refers to a judgment made by a physician or other caregiver that a subject requires or will benefit from treatment. This judgment is made based on a variety of factors that are in the realm of the physician's or caregiver's expertise.

The terms “administration”, “administer” and the like, as they apply to, for example, a subject, cell, tissue, organ, or biological fluid, refer to contact of, for example, an inhibitor of KRAS-G12D, a pharmaceutical composition comprising same, or a diagnostic agent to the subject, cell, tissue, organ, or biological fluid. In the context of a cell, administration includes contact (e.g., in vitro or ex vivo) of a reagent to the cell, as well as contact of a reagent to a fluid, where the fluid is in contact with the cell.

The terms “treat”, “treating”, “treatment” and the like refer to a course of action (such as administering an inhibitor of KRAS-12 or a pharmaceutical composition comprising same) initiated after a disease, disorder or condition, or a symptom thereof, has been diagnosed, observed, and the like, so as to eliminate, alleviate, reduce, suppress, mitigate, improve, or ameliorate, either temporarily or permanently, at least one of the underlying causes of a disease, disorder, or condition afflicting a subject, or at least one of the symptoms associated with a disease, disorder, condition afflicting a subject. Thus, treatment includes inhibiting (e.g., arresting the development or further development of the disease, disorder or condition or clinical symptoms association therewith) an active disease. Specifically, the term “treatment”, as used in the present application, means that a therapeutic substance including a compound or composition according to the present disclosure is administered to a patient in need thereof. In certain embodiments, the term “treatment” also relates to the use of a compound or composition according to the present disclosure, optionally in combination with one or more anticancer agents, to alleviate one or more symptoms associated with KRAS-G12D, to slow down the development of one or more symptoms related to KRAS-G12D, to reduce the severity of one or more symptoms related to KRAS-G12D, to inhibit the clinical manifestations related to KRAS-G12D mutation, and/or to inhibit the expression of adverse symptoms associated with the KRAS-G12D mutation. In certain embodiments, “treating” any disease or condition means alleviating the disease or condition; treating may refer to physical (e.g., stabilization of distinguishable symptom) or physiological (e.g., stabilization of a physical parameter), inhibition of a disease or condition, or both. In certain embodiments, “treatment” refers to improving the quality of life or side effects of the disease in a subject in need.

The terms “prevent”, “preventing”, “prevention”, “prophylaxis” and the like refer to a course of action (such as administering a KRAS-G12D inhibitor or a pharmaceutical composition comprising same) initiated in a manner (e.g., prior to the onset of a disease, disorder, condition or symptom thereof) so as to prevent, suppress, inhibit or reduce, either temporarily or permanently, a subject's risk of developing a disease, disorder, condition or the like (as determined by, for example, the absence of clinical symptoms) or delaying the onset thereof: generally in the context of a subject predisposed to having a particular disease, disorder or condition. In certain instances, the terms also refer to slowing the progression of the disease, disorder or condition or inhibiting progression thereof to a harmful or otherwise undesired state. Specifically, the term “prevention”, as used in the present application, means that a therapeutic substance including a compound or composition according to the present disclosure is administered to a subject to prevent the occurrence of diseases related to the KRAS-G12D mutation.

The term “in need of prevention” as used herein refers to a judgment made by a physician or other caregiver that a subject requires or will benefit from preventative care. This judgment is made based on a variety of factors that are in the realm of a physician's or caregiver's expertise.

The terms “therapeutically effective amount” and “effective amount” are used interchangeably herein to refer to the administration of an agent to a subject, either alone or as part of a pharmaceutical composition and either in a single dose or as part of a series of doses, in an amount capable of having any detectable, positive effect on any symptom, aspect, or characteristic of a disease, disorder or condition when administered to the subject. The therapeutically effective amount can be ascertained by measuring relevant physiological effects, and it can be adjusted in connection with the dosing regimen and diagnostic analysis of the subject's condition, and the like. By way of example, measurement of the serum level of a KRAS-G12D inhibitor (or, e.g., a metabolite thereof) at a particular time post-administration may be indicative of whether a therapeutically effective amount has been used. In some embodiments, the terms “therapeutically effective amount” and “effective amount” refer to the amount or dose of a therapeutic agent, such as a compound, upon single or multiple dose administration to a subject, which provides the desired therapeutic, diagnostic, or prognostic effect in the subject. An effective amount can be readily determined by an attending physician or diagnostician using known techniques and by observing results obtained under analogous circumstances. In determining the effective amount or dose of compound administered, a number of factors are considered including, but not limited to: the size, age, and general health of the subject; the specific disease involved; the degree of or involvement or the severity of the disease or condition to be treated; the response of the individual subject; the particular compound administered; the mode of administration; the bioavailability characteristics of the preparation administered; the dose regimen selected; the use of concomitant medication(s); and other relevant considerations.

The term “substantially pure” is used herein to indicate that a component makes up greater than about 50% of the total content of the composition, and typically greater than about 60% of the total content. More typically, “substantially pure” refers to compositions in which at least 75′%, at least 85%), at least 90% or more of the total composition is the component of interest. In some cases, the component of interest will make up greater than about 90%), or greater than about 95%) of the total content of the composition.

As used herein, the terms “KRAS-G12D-associated disease, disorder or condition” and “disease, disorder or condition mediated by KRAS-G12D” are used interchangeably to refer to any disease, disorder or condition for which the KRAS-G12D mutation is known to play a role, and/or for which treatment with a KRAS-G12D inhibitor may be beneficial. In general, KRAS-G12D-associated or mediated diseases, disorders and conditions are those in which KRAS activity plays a biological, mechanistic, or pathological role. Non-limiting examples of KRAS-G12D-associated diseases, disorders and conditions include oncology-related disorders (cancers, tumors, etc.), including hyperproliferative disorders, hyperplastic diseases, and malignant tumors, such as without limitation lung cancer, non-small cell lung cancer (NSCLC), pancreatic cancer, colorectal cancer, colon cancer, cholangiocarcinoma, cervical cancer, bladder cancer, liver cancer or breast cancer. For example, a KRAS-G12D inhibitor (i.e., a compound or composition of the disclosure) may be used to prevent or treat a proliferative condition, cancer or tumor.

In some embodiments, a KRAS-G12D inhibitor is used to prevent or treat one or more of non-small cell lung cancer, pancreatic cancer, colorectal cancer, bile duct cancer, cervical cancer, bladder cancer, liver cancer and breast cancer.

In certain embodiments, a KRAS-G12D inhibitor is used to prevent or treat an immune-related and/or an inflammatory disease, disorder or condition in a subject.

KRAS-G12D inhibitor compounds and compositions provided herein may be administered to a subject in any appropriate manner known in the art. Suitable routes of administration include, without limitation: oral, parenteral (e.g., intramuscular, intravenous, subcutaneous (e.g., injection or implantation), intraperitoneal, intracisternal, intraarticular, intracerebral (intraparenchymal, intraventricular, and intracerebroventricular), extra-gastrointestinal, nasal, vaginal, sublingual, intraocular, rectal, topical (e.g., transdermal), buccal and inhalation. Depot injections, which are generally administered subcutaneously or intramuscularly, may also be utilized to release the KRAS-G12D inhibitors disclosed herein over a defined period of time. In certain embodiments, KRAS-G12D inhibitor compounds and compositions are administered orally to a subject in need thereof. In certain embodiments, KRAS-G12D inhibitor compounds and compositions are administered intravenously to a subject in need thereof.

KRAS-G12D inhibitor compounds and compositions provided herein may be administered to a subject in an amount that is dependent upon, for example, the goal of administration (e.g., the degree of resolution desired); the age, weight, sex, and health and physical condition of the subject to which the formulation is being administered; the route of administration; and the nature of the disease, disorder, condition or symptom thereof. The dosing regimen may also take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan. In general, dosing parameters dictate that the dosage amount be less than an amount that could be irreversibly toxic to the subject (the maximum tolerated dose (MID)) and not less than an amount required to produce a measurable effect on the subject. Such amounts are determined by, for example, the pharmacokinetic and pharmacodynamic parameters associated with ADME, taking into consideration the route of administration and other factors.

In some embodiments, an KRAS-G12D inhibitor may be administered (e.g., orally) at dosage levels of about 0.01 mg/kg to about 50 mg/kg, or about 1 mg/kg to about 25 mg/kg, of subject body weight per day, one or more times a day, to obtain the desired therapeutic effect. For administration of an oral agent, the compositions can be provided in the form of tablets, capsules and the like containing from 1.0 to 1000 milligrams of the active ingredient, particularly 1, 3, 5, 10, 15, 20, 25, 50, 75, 100, 150, 200, 250, 300, 400, 500, 600, 750, 800, 900, or 1000 milligrams of the active ingredient.

In some embodiments, the dosage of the desired KRAS-G12D inhibitor is contained in a “unit dosage form”. The phrase “unit dosage form” refers to physically discrete units, each unit containing a predetermined amount of the KRAS-G12D inhibitor, either alone or in combination with one or more additional agents, sufficient to produce the desired effect. It will be appreciated that the parameters of a unit dosage form will depend on the particular agent(s) and the effect to be achieved.

### Kits

There are also provided herein kits comprising a KRAS-G12D inhibitor compound or composition of the disclosure. Kits are generally in the form of a physical structure housing various components and may be used, for example, in practicing the methods provided herein. For example, a kit may include one or more KRAS-G12D inhibitor disclosed herein (provided in, e.g., a sterile container), which may be in the form of a pharmaceutical composition suitable for administration to a subject. The KRAS-G12D inhibitor can be provided in a form that is ready for use (e.g., a tablet or capsule) or in a form requiring, for example, reconstitution or dilution (e.g., a powder) prior to administration. When the KRAS-G12D inhibitors are in a form that needs to be reconstituted or diluted by a user, the kit may also include diluents (e.g., sterile water), buffers, pharmaceutically acceptable excipients, and the like, packaged with or separately from the KRAS-G12D inhibitors. When combination therapy is contemplated, the kit may contain several therapeutic agents separately or they may already be combined in the kit. Each component of the kit may be enclosed within an individual container, and all of the various containers may be within a single package. A kit of the present disclosure may be designed for conditions necessary to properly maintain the components housed therein (e.g., refrigeration or freezing).

A kit may also contain a label or packaging insert including identifying information for the components therein and instructions for their use (e.g., dosing parameters, clinical pharmacology of the active ingredient(s), including mechanism of action, pharmacokinetics and pharmacodynamics, adverse effects, contraindications, etc.). Labels or inserts can include manufacturer information such as lot numbers and expiration dates. The label or packaging insert may be, e.g., integrated into the physical structure housing the components, contained separately within the physical structure, or affixed to a component of the kit (e.g., an ampule, tube or vial).

For a better understanding of the present invention and to demonstrate its implementation more clearly, the features of the embodiments according to the present disclosure are described in detail through examples and accompanying drawings.

### EXAMPLES

The present invention will be more readily understood by referring to the following examples, which are provided to illustrate the invention and are not to be construed as limiting the scope thereof in any manner.

Unless defined otherwise or the context clearly dictates otherwise, all technical and scientific terms used herein have the same meaning as commonly understood by one of ordinary skill in the art to which this invention belongs. It should be understood that any methods and materials similar or equivalent to those described herein can be used in the practice or testing of the invention. Unless otherwise stated, the materials and instruments used in this invention are commercially available.

### Compound Synthesis

Synthesis of Compound 1

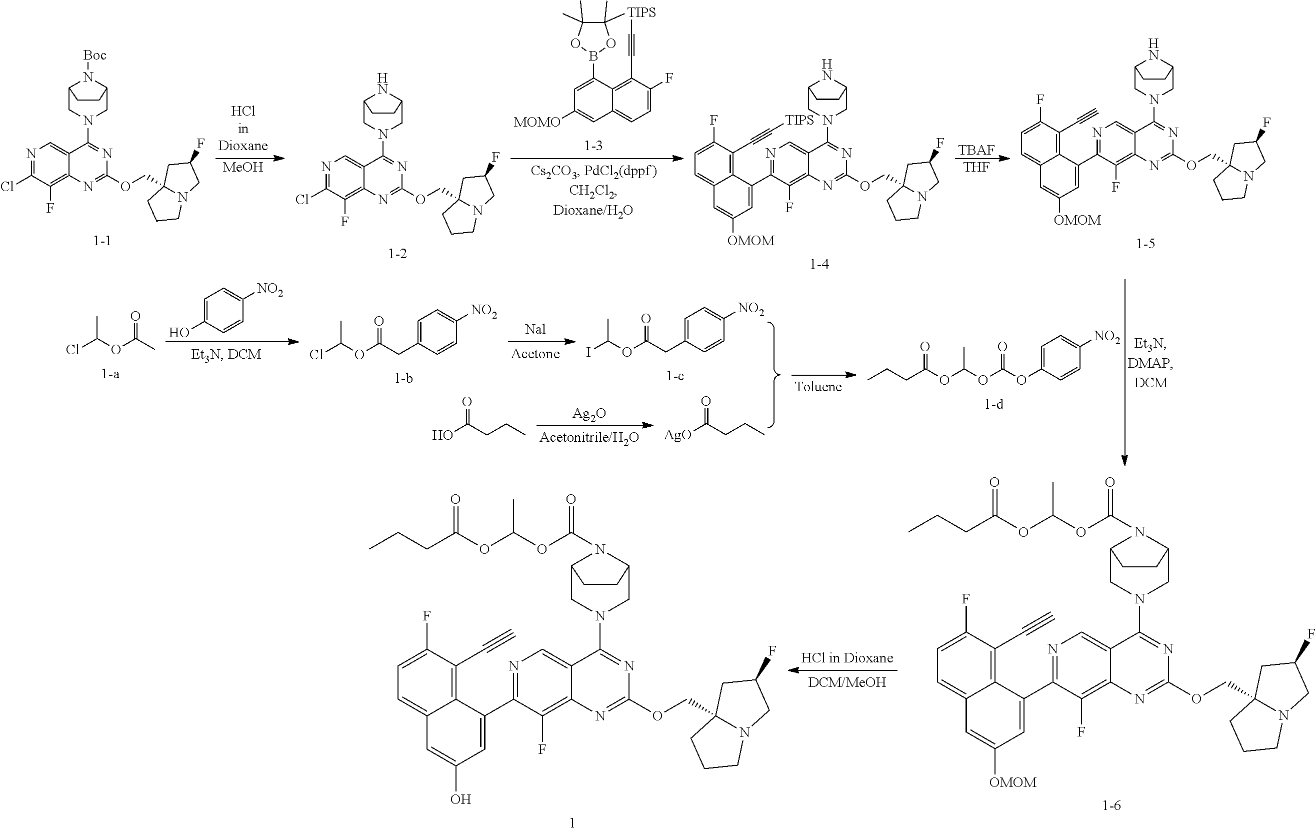

4M HCl in Dioxane (50 mL) was added to a solution of compound 1-1 (7 g, 12.7 mmol, 1.0 eq) in MeOH (30 mL). The mixture was stirred at room temperature for 2 h, then concentrated in vacuo. The pH of residue was adjusted to 8 with NaHCO₃ aqueous. The mixture was diluted with MeOH and concentrated in vacuo. The residue was dissolved with DCM and filtered. The filtrate was concentrated in vacuo to afford crude compound 1-2 (6.0 g, 100% yield).

Compound 1-2 was added to a solution of compound 1-3 (4.09 g, 7.98 mmol, 1.2 eq) in dioxane (50 mL), followed by addition of another solution of Cs₂CO₃ (6.5 g, 19.96 mmol, 3 eq) in water (20 mL), Pd(dppf)Cl₂·CH₂C₁₂ (810 mg, 1 mmol, 0.15 eq). The mixture was stirred at 100° C. for 2 h under nitrogen atmosphere, then cooled to room temperature. The mixture was diluted with water and DCM. The organic phase was washed with water and brine, dried over Na₂SO₄, filtered and concentrated. The residue was purified by flash column chromatography (MeOH/DCM with 0.10% TEA=0%˜5%) to afford compound 1-4 (4 g, 75.06% yield).

1M TBAF in THF (25 mL, 25 mmol, 5 eq) was added to a solution of compound 1-4 (4 g, 5 mmol, 1 eq) in THF (40 mL). The reaction mixture was stirred at room temperature for 1 h and concentrated in vacuo. The residue was purified by flash column chromatography (MeOH/DCM with 0.10% TEA=0%˜6%) to afford compound 1-5 (1.9 g, 59.02% yield).

Et₃N (16.99 g, 167.87 mmol, 1.2 eq) was added to a solution of p-nitrophenol (21.41 g, 153.88 mmol, 1.1 eq) in DCM (96.23 mL). Compound 1-a (20 g, 139.89 mmol, 1 eq) was added to the mixture at 0° C. The mixture was warmed to room temperature and stirred at this temperature for 1 hour (h), then diluted with water. The organic phase was washed with brine, dried over Na₂SO₄, filtered and concentrated. The residue was purified by flash column chromatography (PE/EA=0%˜6%) to afford compound 1-b (17.5 g, 50.93% yield).

NaI (24.41 g, 162.86 mmol, 4 eq) was added to a solution of compound 1-b (10 g, 40.71 mmol, 1 eq) in acetone (100 mL). The mixture was stirred at 50° C. for 30 h under nitrogen atmosphere, then cooled to room temperature and filtered. The filtrate was concentrated and the residue was purified by flash column chromatography (PE/EA=0%˜3%) to afford compound 1-c (8.3 g, 60.48% yield).

Ag₂O (31.56 g, 136 mmol, 0.6 eq) was added to a solution of n-butyric acid (20 g, 227 mmol, 1 eq) in acetonitrile (200 mL) and water (100 mL). The mixture was stirred at room temperature for 16 h and filtered. The filtrated was concentrated in vacuo to afford silver butyrate (11 g, 24.85% yield).

Silver butyrate (2.53 g, 12.96 mmol, 1.15 eq) was added to a solution of compound 1-c (3.8 g, 11.27 mmol, 1 eq) in toluene (38 mL). The mixture was heated to 50° C. and stirred at this temperature overnight, then cooled to room temperature, filtered. The filtrate was concentrated and the residue was purified by flash column chromatography (PE/EA=0%˜3%) to afford compound 1-d (1.59 g, 47.51% yield).

Compound 1-d (1.58 g, 5.3 mmol, 1.8 eq) was added to a solution of compound 1-5 (1.9 g, 2.95 mmol, 1 eq) in DCM (19 mL), followed by addition of Et₃N (745.56 mg, 7.37 mmol, 2.5 eq) and DMAP (72.01 mg, 0.589 mmol, 0.2 eq). The mixture was stirred at 40° C. for 1 h, then concentrated. The residue was purified by flash column chromatography (MeOH/DCM=0%˜3%) to afford compound 1-6 (1.56 g, 65.93% yield).

MeOH (0.5 mL) was added to a solution of compound 1-6 (1.56 g, 1.94 mmol, 1.0 eq) in DCM (100 mL), followed by addition of another solution of 4M HCl in dioxane (6 mL). The reaction mixture was stirred at room temperature for 15 min and concentrated in vacuo. The residue was diluted with DCM and the pH was basified with Et₃N, then concentrated again. The residue was diluted with DCM, washed with water and brine, dried over Na₂SO₄, filtered and concentrated. The residue was purified by flash column chromatography (MeOH/DCM=0%˜5%) to afford compound 1 (1.2 g, 80.07% yield). ¹H NMR (500 MHz, CD₃OD) δ ppm 0.96-1.05 (m, 3H), 1.57 (s, 3H), 1.69 (s, 2H), 1.84-2.13 (m, 7H), 2.18-2.48 (m, 5H), 3.06-3.18 (m, 1H), 3.38-3.50 (m, 3H), 3.70-3.88 (m, 2H), 4.19-4.34 (m, 2H), 4.29-4.45 (m, 2H), 4.46-4.56 (m, 2H), 5.33 (s, 0.5H), 5.44 (s, 0.5H), 6.84-6.95 (m, 1H), 7.24 (s, 1H), 7.32-7.42 (m, 2H), 7.87-7.94 (m, 1H), 9.06 (s, 1H). m/z (ESI⁺): 759.3.

Synthesis of Compound 2

Compound 2 was synthesized according to the procedure of compound 1 using isovaleric acid as start material. ¹H NMR (500 MHz, CD₃OD) δ ppm 9.14 (s, 1H), 7.92 (dd, J=9.0, 6.0 Hz, 1H), 7.41 (d, J=2.5 Hz, 1H), 7.38 (t, J=9.0 Hz, 1H), 7.25 (d, J=2.5 Hz, 1H), 6.87 (q, J=5.5 Hz, 1H), 5.61 (d, J=52 Hz, 1H), 4.82-4.62 (m, 4H), 4.54 (s, 2H), 4.12-3.72 (m, 5H), 3.56-3.46 (m, 1H), 3.38 (d, J=9.2 Hz, 1H), 2.82-2.55 (m, 3H), 2.52-2.44 (m, 1H), 2.43-2.34 (m, 2H), 2.27-2.16 (m, 1H), 2.14-1.96 (m, 2H), 1.87 (d, J=7.9 Hz, 2H), 1.58 (d, J=4.3 Hz, 3H), 1.21 (s, 6H). m/z (ESI⁺): 759.6.

Synthesis of Compound 3

Compound 3 was synthesized according to the procedure of compound 1 using acetic acid as start material. ¹H NMR (500 MHz, CD₃OD) δ ppm 9.14 (s, 1H), 7.91 (dd, J=9.0, 6.0 Hz, 1H), 7.41 (d, J=2.5 Hz, 1H), 7.37 (t, J=9.0 Hz, 1H), 7.25 (d, J=2.5 Hz, 1H), 6.87 (q, J=5.0 Hz, 1H), 5.61 (d, J=51.0 Hz, 1H), 4.84-4.64 (m, 4H), 4.53 (s, 2H), 4.11-3.71 (m, 5H), 3.57-3.47 (m, 1H), 3.38 (d, J=9.1 Hz, 1H), 2.81-2.57 (m, 2H), 2.52-2.43 (m, 1H), 2.43-2.34 (m, 2H), 2.27-1.98 (m, 6H), 1.86 (d, J=8.0 Hz, 2H), 1.57 (s, 3H). m/z (ESI⁺): 731.57.

Synthesis of Compound 4

Isovaleric acid (6.99 mg, 0.068 mmol, 1 eq) was added to a solution of compound 3 (50 mg, 0.068 mmol, 1 eq) in DCM (5 mL), followed by addition of DCC (14.12 mg, 0.068 mmol, 1 eq) and DMAP (8.36 mg, 0.068 mmol, 1 eq). The mixture was stirred at room temperature for 1 h, then concentrated in vacuo. The residue was purified by flash column chromatography (MeOH/DCM=0%˜3%) to afford compound 4 (36.1 mg, 64.3% yield). ¹H NMR (500 MHz, CD₃OD) δ ppm 9.08 (s, 1H), 8.13 (dd, J=9.0, 6.0 Hz, 1H), 7.91 (d, J=2.5 Hz, 1H), 7.51 (t, J=9.0 Hz, 1H), 7.47 (s, 1H), 6.86 (d, J=5.5 Hz, 1H), 5.38 (d, J=53.5 Hz, 1H), 4.82-4.62 (m, 2H), 4.57-4.46 (m, 2H), 4.46-4.30 (m, 2H), 3.94-3.70 (m, 2H), 3.57-3.38 (m, 3H), 3.16-3.09 (m, 1H), 2.57 (d, J=7.0 Hz, 2H), 2.49-2.18 (m, 4H), 2.13-1.93 (m, 8H), 1.93-1.80 (m, 2H), 1.56 (s, 3H), 1.11 (d, J=6.5 Hz, 6H). m/z (ESI⁺): 815.5.

Synthesis of Compound 5

Compound 5 was synthesized according to the procedure of compound 1 using pivalic acid as start material. ¹H NMR (500 MHz, CD₃OD) δ ppm 9.05 (s, 1H), 7.89 (dd, J=9.0, 5.5 Hz, 1H), 7.38 (d, J=2.0 Hz, 2H), 7.35 (t, J=9.0 Hz, 1H), 7.24 (s, 1H), 6.85 (q, J=5.0 Hz, 1H), 5.37 (d, J=53.5 Hz, 1H), 4.80-4.63 (m, 2H), 4.58-4.44 (m, 2H), 4.42-4.28 (m, 2H), 3.93-3.64 (m, 2H), 3.50-3.36 (m, 2H), 3.32-3.26 (m, 1H), 3.16-3.05 (m, 1H), 2.47-2.15 (m, 3H), 2.13-1.82 (m, 7H), 1.57 (s, 3H), 1.24 (d, J=6.5 Hz, 9H). m/z, (ESI⁺): 773.3.

Synthesis of Compound 6

Compound 6 was synthesized according to the procedure of compound 1 using 2-propylpentanoic acid as start material. ¹H NMR (500 MHz, DMSO-d6) δ ppm 10.19 (s, 1H), 9.04 (s, 1H), 8.00 (m, 1H), 7.62-7.36 (m, 2H), 7.19 (s, 1H), 6.81 (q, J=5.0 Hz, 1H), 5.30 (d, J=53.5 Hz, 1H), 4.49 (m, 4H), 4.17-3.95 (m, 3H), 3.81-3.40 (m, 2H), 3.07 (m, 3H), 2.85 (m, 1H), 2.08 (m, 3H), 1.92-1.76 (m, 6H), 1.50 (m, 5H), 1.41 (m, 2H), 1.26 (m, 5H), 1.07 (m, 1H), 0.90 (m, 6H). (ESI⁺): 815.3.

Synthesis of Compound 7

Compound 7 was synthesized according to the procedure of compound 4 using pivalic acid as start material. ¹H NMR (500 MHz, CD₃OD) δ ppm 0.93-1.01 (m, 3H), 1.40 (s, 9H), 1.49-1.58 (m, 3H), 1.60-1.72 (m, 2H), 1.80-2.07 (m, 7H), 2.10-2.40 (m, 5H), 2.99-3.08 (m, 1H), 3.14-3.28 (m, 3H), 3.43-3.52 (m, 1H), 3.73-3.85 (m, 1H), 4.21-4.36 (m, 2H), 4.42-4.52 (m, 2H), 4.59-4.75 (m, 2H), 5.26 (s, 0.5H), 5.37 (s, 0.5H), 6.81-6.90 (m, 1H), 7.41 (s, 1H), 7.46-7.53 (m, 1H), 7.87 (s, 1H), 8.06-8.13 (m, 1H), 9.04 (s, 1H). m/z (ESI⁺): 843.4.

Synthesis of Compound 8

Compound 8 was synthesized according to the procedure of compound 1 using 3-cyclopentylpropionic acid as start material. ¹H NMR (500 MHz, CD₃OD) δ ppm 9.06 (s, 1H), 7.89 (dd, J=9.0, 5.5 Hz, 1H), 7.38 (d, J=2.5 Hz, 1H), 7.36 (t, J=9.0 Hz, 1H), 7.24 (s, 1H), 6.88 (d, J=5.5 Hz, 1H), 5.37 (d, J=54.0 Hz, 1H), 4.80-4.60 (m, 2H), 4.51 (s, 2H), 4.44-4.27 (m, 2H), 3.92-3.68 (m, 2H), 3.50-3.37 (m, 2H), 3.17-3.04 (m, 1H), 2.48-2.24 (m, 4H), 2.24-2.16 (m, 1H), 2.13-2.00 (m, 4H), 2.00-1.76 (m, 6H), 1.74-1.48 (m, 9H), 1.20-1.08 (m, 2H). m/z (ESI⁺): 813.3.

Synthesis of Compound 9

Compound 9 was synthesized according to the procedure of compound 1 using cyclopentylacetic acid as start material. ¹H NMR (500 MHz, CD₃OD) δ ppm 9.06 (s, 1H), 7.89 (dd, J=9.0, 5.5 Hz, 1H), 7.42-7.31 (m, 2H), 7.23 (s, 1H), 6.88 (d, J=5.0 Hz, 1H), 5.39 (d, J=53.5 Hz, 1H), 4.8-4.63 (m, 2H), 4.56-4.46 (m, 2H), 4.45-4.31 (m, 2H), 3.90-3.70 (m, 2H), 3.51-3.37 (m, 3H), 3.19-3.08 (m, 1H), 2.52-2.18 (m, 6H), 2.15-1.94 (m, 5H), 1.93-1.81 (m, 4H), 1.76-1.49 (m, 7H), 1.29-1.18 (m, 2H). m/z (ESI⁺): 799.3.

Synthesis of Compound 10

Compound 10 was synthesized according to the procedure of compound 1 using isovaleric acid as start material. ¹H NMR (500 MHz, CD₃OD) δ ppm 0.94-1.03 (m, 6H), 1.48-1.58 (m, 3H), 1.81-2.06 (m, 7H), 2.06-2.39 (m, 6H), 2.99-3.08 (m, 1H), 3.20-3.29 (m, 2H), 3.34-3.40 (m, 1H), 3.64-3.84 (m, 2H), 4.21-4.36 (m, 2H), 4.43-4.53 (m, 2H), 4.60-4.76 (m, 3H), 5.27 (s, 0.5H), 5.38 (s, 0.5H), 6.82-6.91 (m, 1H), 7.21 (d, J=2.0 Hz, 1H), 7.29-7.39 (m, 2H), 7.83-7.90 (m, 1H), 9.02 (s, 1H). m/z (ESI⁺): 773.4.

Synthesis of Compound 11

Compound 11 was synthesized according to the procedure of compound 1 using hexanoic acid as start material. ¹H NMR (500 MHz, CD₃OD) δ ppm 0.84-0.97 (m, 3H), 1.31-1.41 (m, 4H), 1.45-1.58 (m, 3H), 1.58-1.69 (m, 2H), 1.80-2.11 (m, 7H), 2.13-2.43 (m, 5H), 3.02-3.13 (m, 1H), 3.23-3.29 (m, 1H), 3.34-3.43 (m, 2H), 3.63-3.85 (m, 2H), 4.25-4.40 (m, 2H), 4.43-4.52 (m, 2H), 4.60-4.75 (m, 2H), 5.29 (s, 0.5H), 5.39 (s, 0.5H), 6.82-6.89 (m, 1H), 7.20 (s, 1H), 7.28-7.39 (m, 2H), 7.83-7.91 (m, 1H), 9.02 (s, 1H). m/z (ESI⁺): 787.3.

Synthesis of Compound 12

Compound 12 was synthesized according to the procedure of compound 1 using 1-adamantaneacetic acid as start material. ¹H NMR (500 MHz, CD₃OD) δ ppm 1.53 (s, 3H), 1.66-1.73 (m, 12H), 1.87-2.35 (m, 15H), 3.02 (s, 1H), 3.17-3.36 (m, 4H), 3.70-3.78 (m, 2H), 4.22-4.32 (m, 2H), 4.48 (s, 2H), 4.67 (s, 2H), 5.25-5.36 (m, 1H), 6.86 (s, 1H), 7.20 (s, 1H), 7.30-7.35 (m, 2H), 7.84-7.87 (m, 1H), 9.00 (s, 1H). m/z (ESI⁺): 866.4.

Synthesis of Compound 13

Compound 13 was synthesized according to the procedure of compound 1 using 2-methylbutyric acid as start material. ¹H NMR (500 MHz, CD₃OD) δ ppm 0.93 (s, 3H), 1.15 (s, 3H), 1.29 (s, 1H), 1.54-1.67 (m, 6H), 1.86-2.40 (m, 12H), 3.07-3.39 (m, 2H), 3.72-3.78 (m, 2H), 4.26-4.37 (m, 2H), 4.47 (s, 2H), 4.67 (s, 2H), 5.28-5.39 (m, 1H), 6.86 (s, 1H), 7.20 (s, 1H), 7.31-7.35 (m, 2H), 7.85-7.88 (m, 1H), 9.02 (s, 1H). m/z (ESI⁺): 773.3.

Synthesis of Compound 14

Compound 14 was synthesized according to the procedure of compound 1 using cyclohexanecarboxylic acid as start material. ¹H NMR (500 MHz, CD₃OD) δ ppm 9.06 (s, 1H), 7.90 (dd, J=9.0, 5.5 Hz, 1H), 7.42-7.33 (m, 2H), 7.24 (s, 1H), 6.86 (q, J=5.0 Hz, 1H), 5.37 (d, J=53.5 Hz, 1H), 4.82-4.60 (m, 2H), 4.57-4.45 (m, 2H), 4.43-4.29 (m, 2H), 3.90-3.67 (m, 2H), 3.46-3.37 (m, 2H), 3.17-3.06 (m, 1H), 2.50-2.15 (m, 4H), 2.15-1.84 (m, 9H), 1.83-1.74 (m, 2H), 1.74-1.62 (m, 1H), 1.61-1.53 (m, 3H), 1.53-1.43 (m, 2H), 1.42-1.24 (m, 5H). m/z (ESI⁺): 799.3.

Synthesis of Compound 15

Compound 15 was synthesized according to the procedure of compound 1 using octanoic acid as start material. ¹H NMR (500 MHz, CD₃OD) δ ppm 0.81-0.95 (m, 3H), 1.27-1.36 (m, 8H), 1.49-1.68 (m, 5H), 1.80-2.55 (m, 12H), 3.12-3.21 (m, 1H), 3.34-3.50 (m, 3H), 3.68-3.87 (m, 2H), 4.32-4.53 (m, 4H), 4.60-4.78 (m, 2H), 5.34 (s, 0.5H), 5.45 (s, 0.5H), 6.80-6.90 (m, 1H), 7.20 (s, 1H), 7.29-7.39 (m, 2H), 7.83-7.91 (m, 1H), 9.04 (s, 1H). m/z (ESI⁺): 815.3.

Synthesis of Compound 16

Compound 16 was synthesized according to the procedure of compound 1 using 2,2-dimethylbutyric acid as start material. ¹H NMR (500 MHz, CD₃OD) δ ppm 9.05 (s, 1H), 7.94-7.85 (m, 1H), 7.44-7.30 (m, 2H), 7.23 (s, 1H), 6.87-6.88 (m, 1H), 5.31-5.41 (m, 1H), 4.71 (s, 2H), 4.50 (m, 2H), 4.41-4.26 (m, 2H), 3.81 (m, 2H), 3.41 (s, 1H), 3.30 (m, 2H), 3.09 (s, 1H), 2.19-2.39 (m, 3H), 2.13-1.84 (m, 7H), 1.57-1.62 (m, 5H), 1.31-1.33 (m, 1H), 1.21 (s, 6H), 0.91 (s, 3H). (ESI⁺): 787.4.

Synthesis of Compound 17

Compound 17 was synthesized according to the procedure of compound 1 using 3,3-dimethylbutyric acid as start material. ¹H NMR (500 MHz, CD₃OD) δ ppm 1.06 (s, 9H), 1.54 (s, 3H), 1.86-2.34 (m, 13H), 3.03-3.04 (m, 1H), 3.22-3.38 (m, 3H), 3.75-3.78 (m, 2H), 4.23-4.33 (m, 2H), 4.48 (s, 2H), 4.70 (s, 2H), 5.26-5.37 (m, 1H), 6.86 (s, 1H), 7.20 (s, 1H), 7.31-7.35 (m, 2H), 7.85-7.88 (m, 1H), 9.01 (s, 1H). (ESI⁺): 755.4.

Synthesis of Compound 18

Compound 18 was synthesized according to the procedure of compound 1 using 2-ethylhexanoic acid as start material. ¹H NMR (500 MHz, CD₃OD) δ ppm 0.87-1.02 (m, 6H), 1.28-1.31 (m, 4H), 1.47-1.68 (m, 7H), 1.82-2.42 (m, 11H), 2.99-3.09 (m, 1H), 3.23-3.28 (m, 1H), 3.35-3.47 (m, 3H), 3.68-3.86 (m, 1H), 4.22-4.37 (m, 2H), 4.42-4.53 (m, 2H), 4.62-4.76 (m, 2H), 5.27 (s, 0.5H), 5.38 (s, 0.5H), 6.82-6.94 (m, 1H), 7.21 (s, 1H), 7.28-7.39 (m, 2H), 7.82-7.91 (m, 1H), 9.02 (s, 1H). m/z (ESI⁺): 815.4.

Synthesis of Compound 19

Compound 19 was synthesized according to the procedure of compound 1 using propionic acid as start material. ¹H NMR (500 MHz, CD₃OD) δ ppm 1.07-1.18 (m, 3H), 1.49-1.59 (m, 3H), 1.80-2.46 (m, 12H), 2.98-3.09 (m, 1H), 3.21-3.27 (m, 2H), 3.36-3.47 (m, 3H), 3.74-3.87 (m, 1H), 4.19-4.37 (m, 2H), 4.42-4.54 (m, 2H), 4.59-4.74 (m, 1H), 5.27 (s, 0.5H), 5.37 (s, 0.5H), 6.81-6.90 (m, 1H), 7.21 (s, 1H), 7.29-7.40 (m, 2H), 7.82-7.93 (m, 1H), 9.02 (s, 1H). m/z (ESI⁺): 745.3.

Synthesis of Compound 20

Compound 20 was synthesized according to the procedure of compound 1 using 2-butylhexanoic acid as start material. ¹H NMR (500 MHz, CD₃OD) δ ppm 0.85 (s, 6H), 1.25 (s, 8H), 1.48-1.55 (m, 7H), 1.88-1.90 (m, 7H), 2.12 (s, 1H), 2.20-2.42 (m, 3H), 3.03 (s, 1H), 3.34 (s, 3H), 3.65-3.74 (m, 2H), 4.26-4.62 (m, 7H), 5.24-5.35 (m, 1H), 6.83 (s, 1H), 7.15 (s, 1H), 7.27-7.29 (m, 2H), 7.80 (s, 1H), 8.97 (s, 1H). m/z (ESI⁺): 843.4.

Synthesis of Compound 21

Compound 21 was synthesized according to the procedure of compound 1 using pentanoic acid as start material. ¹H NMR (500 MHz, CD₃OD) δ ppm 0.95-0.98 (t, J=7.5 Hz, 3H), 1.32-1.42 (m, 3H), 1.57-1.64 (m, 5H), 1.86-1.87 (m, 2H), 2.06-2.19 (m, 3H), 2.39-2.48 (m, 6H), 2.59-2.74 (m, 2H), 3.39-3.51 (m, 2H), 3.82-4.09 (m, 5H), 4.33 (s, 2H), 4.73-4.78 (m, 4H), 5.56-5.66 (m, 1H), 6.88-6.89 (m, 1H), 7.25 (s, 1H), 7.36-7.41 (m, 2H), 7.90-7.93 (m, 1H), 9.14 (s, 1H). m/z (ESI⁺): 773.3.

Synthesis of Compound 22

Compound 22 was synthesized according to the procedure of compound 1 using nicotinic acid as start material. ¹H NMR (500 MHz, CD₃OD) δ ppm 1.71 (s, 3H), 1.87-2.34 (m, 11H), 3.04-3.30 (m, 3H), 3.81 (s, 2H), 4.27-4.32 (m, 2H), 4.50-4.65 (m, 5H), 5.27-5.38 (m, 1H), 7.14 (s, 1H), 7.20 (s, 1H), 7.33-7.35 (m, 2H), 7.60 (s, 1H), 7.86 (s, 1H), 8.44 (s, 1H), 8.78 (s, 1H), 9.02 (s, 1H), 9.16 (s, 1H). m/z (ESI⁺): 794.3.

Synthesis of Compound 23

Compound 23 was synthesized according to the procedure of compound 1 using 1-naphthoic acid as start material. ¹H NMR (500 MHz, CD₃OD) δ ppm 1.73 (s, 3H), 1.82-2.22 (m, 11H), 3.00 (s, 1H), 3.18-3.22 (m, 3H), 3.78-3.80 (m, 2H), 4.22-4.26 (m, 2H), 4.49-4.62 (m, 4H), 5.22-5.33 (m, 1H), 7.20 (m, 2H), 7.27-7.29 (m, 2H), 7.53-7.55 (m, 3H), 7.82-7.93 (m, 2H), 8.10-8.23 (m, 2H), 8.85-9.00 (m, 2H). m/z (ESI⁺): 843.3.

Synthesis of Compound 24

Compound 24 was synthesized according to the procedure of compound 1 using benzoic acid as start material. ¹H NMR (500 MHz, CD₃OD) δ ppm 9.04 (d, J=15.5 Hz, 1H), 8.08 (s, 2H), 7.89 (dd, J=9.0, 5.5 Hz, 1H), 7.67 (s, 1H), 7.53 (t, J=7.5 Hz, 2H), 7.41-7.32 (m, 2H), 7.23 (s, 1H), 7.14 (d, J=5.5 Hz, 1H), 5.36 (d, J=53.5 Hz, 1H), 4.84-4.61 (m, 2H), 4.60-4.47 (m, 2H), 4.42-4.25 (m, 2H), 3.93-3.71 (m, 2H), 3.40 (d, J=8.5 Hz, 1H), 3.31-3.25 (m, 1H), 3.15-3.03 (m, 1H), 2.48-2.14 (m, 4H), 2.13-2.01 (m, 4H), 2.00-1.79 (m, 3H), 1.79-1.65 (m, 3H). m/z (ESI⁺): 793.3.

Synthesis of Compound 25

Compound 25 was synthesized according to the procedure of compound 1 using 2-butyloctanoic acid as start material. ¹H NMR (500 MHz, CD₃OD) δ ppm 0.94 (s, 6H), 1.33 (s, 12H), 1.57 (t, J=14.0 Hz, 7H), 1.90 (s, 3H), 2.03 (s, 3H), 2.13-2.34 (m, 3H), 2.36-2.38 (m, 2H), 3.06 (s, 1H), 3.24-3.27 (m, 3H), 3.39 (m, 1H), 3.74-3.82 (m, 2H), 4.24-4.37 (m, 2H), 4.50 (s, 2H), 4.70 (s, 2H), 5.34 (d, J=53.5 Hz, 1H), 6.91 (s, 1H), 7.23 (s, 1H), 7.34-7.35 (m, 2H), 7.86-7.93 (m, 1H), 9.04 (s, 1H). m/z (ESI⁺): 871.4.

Synthesis of Compound 26

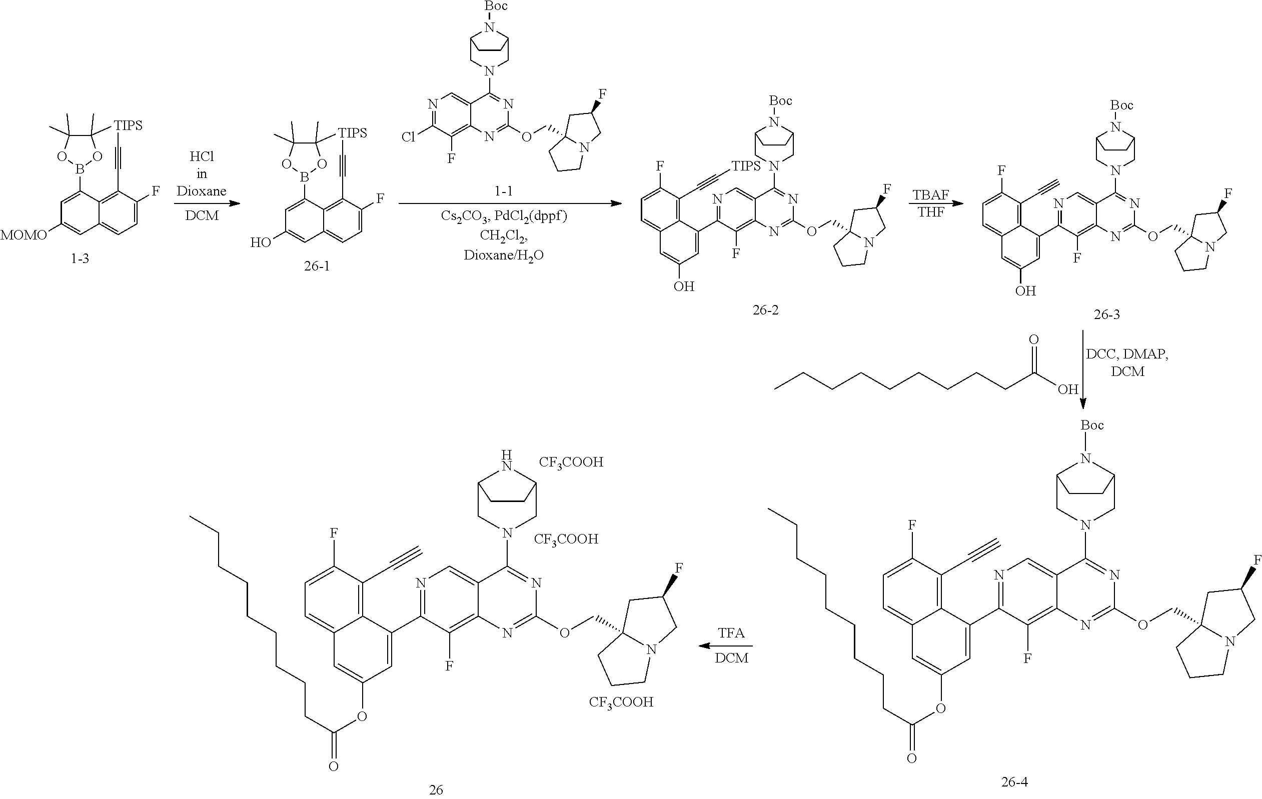

4M HCl in Dioxane (3 mL) was added to a solution of compound 1-3 (1.2 g, 2.34 mmol, 1 eq) in DCM (10 mL). The reaction mixture was stirred at room temperature for 1 h, then concentrated in vacuo. The residue was purified by flash column chromatography (EA/PE=0%˜5%) to afford compound 26-1 (1.02 g, 93.27% yield).

Compound 1-1 (1 g, 1.81 mmol, 1 eq) was added to a solution of compound 26-1 (1.02 g, 2.18 mmol, 1.2 eq) in dioxane (25 mL), followed by addition of another solution of Cs₂CO₃ (1.77 g, 5.44 mmol, 1 eq) in water (10 mL), Pd(dppf)Cl₂·CH₂C₁₂ (294.21 mg, 0.36 mmol, 0.2 eq). The mixture was stirred at 100° C. for 2 h under nitrogen atmosphere, then cooled to room temperature. The mixture was diluted with water and EA. The organic phase was washed with water and brine, dried over Na₂SO₄, filtered and concentrated. The residue was purified by flash column chromatography (MeOH/DCM=0%˜4%) to afford compound 26-2 (759 mg, 48.8% yield).

1M TBAF in THF (4.43 mL) was added to a solution of compound 26-2 (759 mg, 0.88 mmol, 1 eq) in THF (7.6 mL). The reaction mixture was stirred at room temperature for 1.5 h and concentrated in vacuo. The residue was purified by flash column chromatography (MeOH/DCM=0%˜10%) to afford compound 26-3 (639 mg, 100% yield).

Decanoic acid (7.67 mg, 0.044 mmol, 1 eq) was added to a solution of compound 26-3 (31.2 mg, 0.044 mmol, 1 eq) in DCM (2.99 mL), followed by addition of DMAP (0.54 mg, 0.004 mmol, 0.1 eq) and DCC (9.19 mg, 0.044 mmol, 1 eq). The mixture was stirred at room temperature for 1.5 h, then concentrated in vacuo. The residue was purified by flash column chromatography (MeOH/DCM=0%˜4%) to afford compound 26-4 (29 mg, 76.18% yield).

Trifluoroacetic acid (1 mL) was added to as solution of compound 26-4 (29 mg, 0.033 mmol, 1 eq) in DCM (1 mL). The mixture was stirred at room temperature for 5 min and concentrated in vacuo. The residue was purified by preparative HPLC to afford compound 26 (12.2 mg, 32.32% yield). ¹H NMR 500 MHz, CD₃OD) δ ppm 9.16 (s, 1H), 8.14 (dd, J=9.0, 5.5 Hz, 1H), 7.92 (s, 1H), 7.52 (t, J=9.0 Hz, 1H), 7.48 (s, 1H), 5.61 (d, J=51.5 Hz, 1H), 4.75 (s, 2H), 4.31 (d, J=10.0 Hz, 2H), 4.15-3.82 (m, 5H), 3.49 (dd, J=18.4, 9.0 Hz, 2H), 2.83-2.56 (m, 4H), 2.53-2.43 (m, 1H), 2.43-2.32 (m, 2H), 2.17 (dd, J=33.0, 9.0 Hz, 5H), 1.84-1.75 (m, 2H), 1.57-1.27 (m, 14H), 0.91 (t, J=6.5 Hz, 3H). m/z (ESI⁺): 755.7.

Synthesis of Compound 27

Compound 27 was synthesized according to the procedure of compound 26 using isovaleric acid as start material. ¹H NMR (500 MHz, CD₃OD) δ ppm 1.08 (d, J=5.0 Hz, 7H), 2.03-2.47 (m, 10H), 2.55-2.74 (m, 2H), 2.57-2.75 (m, 2H), 3.44-3.50 (m, 2H), 3.85-4.05 (m, 5H), 4.28 (d, J=10.0 Hz, 2H), 4.72-4.74 (m, 2H), 5.53-5.63 (m, 1H), 7.44 (s, 1H), 7.50 (d, J=10.0 Hz, 1H), 7.90 (s, 1H), 8.12 (q, J=10.0 Hz, 1H), 9.13 (s, 1H). m/z (ESI⁺): 785.5.

Synthesis of Compound 28

Compound 28 was synthesized according to the procedure of compound 26 using pivalic acid as start material. ¹H NMR (500 MHz, CD₃OD) δ 9.16 (s, 1H), 8.15 (dd, J=9.0, 5.5 Hz, 1H), 7.92 (d, J=2.0 Hz, 1H), 7.53 (t, J=9.0 Hz, 1H), 7.44 (s, 1H), 5.61 (d, J=51.5 Hz, 1H), 4.81-4.71 (m, 2H), 4.32 (s, 2H), 4.12-3.87 (m, 5H), 3.59-3.44 (m, 2H), 2.87-2.61 (m, 2H), 2.55-2.44 (m, 1H), 2.43-2.34 (m, 2H), 2.28-2.10 (m, 5H), 1.43 (s, 9H). m/z (ESI⁺): 685.6.

Synthesis of Compound 29

Compound 29 was synthesized according to the procedure of compound 26 using dodecanoic acid as start material. ¹H NMR (500 MHz, CD₃OD) δ ppm 0.53-0.94 (m, 3H), 1.28-1.48 (m, 16H), 1.71-1.82 (m, 2H), 2.01-2.24 (m, 5H), 2.29-2.50 (m, 3H), 2.56-2.77 (m, 4H), 3.42-3.52 (m, 2H), 3.81-4.07 (m, 5H), 4.23-4.34 (m, 2H), 4.66-4.77 (m, 2H), 4.86 (s, 1H), 5.53 (s, 0.5H), 5.63 (s, 0.5H), 7.46 (s, 1H), 7.50 (d, J=9.0 Hz, 1H), 7.91 (d, J=2.0 Hz, 1H), 8.08-8.17 (m, 1H), 9.13 (s, 1H). m/z (ESI⁺): 783.4.

Synthesis of Compound 30

Compound 30 was synthesized according to the procedure of compound 26 using 3-cyclopentylpropionic acid as start material. ¹H NMR (500 MHz, CD₃OD) δ ppm 9.16 (s, 1H), 8.14 (dd, J=9.0, 5.5 Hz, 1H), 7.93 (d, J=2.2 Hz, 1H), 7.53 (t, J=9.0 Hz, 1H), 7.49 (s, 1H), 5.61 (d, J=51.5 Hz, 1H), 4.90 (d, J=13.0 Hz, 1H), 4.75 (s, 2H), 4.38-4.27 (m, 2H), 4.12-3.87 (m, 5H), 3.55-3.45 (m, 2H), 2.82-2.58 (m, 4H), 2.55-2.32 (m, 3H), 2.31-2.07 (m, 5H), 1.99-1.78 (m, 5H), 1.77-1.54 (m, 4H), 1.28-1.17 (m, 2H). m/z (ESI⁺): 725.4.

Synthesis of Compound 31

Compound 31 was synthesized according to the procedure of compound 26 using palmitic acid as start material. ¹H NMR (500 MHz, CD₃OD) δ ppm 0.87-0.91 (m, 3H), 1.28-1.50 (m, 24H), 1.72-1.82 (m, 2H), 1.96-2.20 (m, 6H), 2.20-2.52 (m, 3H), 2.53-2.63 (m, 1H), 2.63-2.72 (m, 2H), 3.43-3.51 (m, 3H), 3.74-4.01 (m, 4H), 4.15-4.26 (m, 2H), 4.58-4.71 (m, 2H), 4.79-4.85 (m, 2H), 5.49 (s, 0.5H), 5.60 (s, 0.5H), 7.42-7.56 (m, 2H), 7.91 (s, 1H), 8.07-8.20 (m, 1H), 9.12 (s, 1H). m/z (ESI⁺): 839.6.

Synthesis of Compound 32

Compound 32 was synthesized according to the procedure of compound 26 using 2-hexyldecanoic acid as start material. ¹H NMR (500 MHz, CD₃OD) δ ppm 0.87 (t, J=5.0 Hz, 6H), 1.28-1.46 (m, 22H), 1.64-1.66 (m, 2H), 75-1.81 (m, 2H), 2.10-2.16 (m, 5H), 2.35-2.44 (m, 3H), 2.57-2.75 (m, 3H), 3.45=3.50 (m, 2H), 3.86-4.02 (m, 5H), 4.27-4.29 (m, 2H), 4.72 (s, 2H), 5.53-5.63 (m, 1H), 7.38 (s, 1H), 7.51 (t, J=10.0 Hz, 1H), 7.87-7.88 (m, 1H), 8.12-8.15 (m, 1H), 9.14 (s, 1H). m/z (ESI⁺): 839.5.

Synthesis of Compound 33

Compound 33 was synthesized according to the procedure of compound 26 using isobutyric acid as start material. ¹H NMR (500 MHz, CD₃OD) δ ppm 1.36 (s, 3H), 1.38 (s, 3H), 2.13-2.21 (m, 5H), 2.35-2.50 (m, 3H), 2.60-2.78 (m, 2H), 2.91-2.97 (m, 1H), 3.47-3.53 (m, 2H), 3.90-4.06 (m, 2H) 4.31 (s, 2H), 4.74-4.75 (d, J=5.0 Hz, 2H), 4.88-4.91 (m, 2H), 5.56-5.67 (m, 1H), 7.47 (s, 1H), 7.51-7.55 (m, 1H), 7.93 (s, 1H), 8.14-8.16 (m, 1H), 9.17 (s, 1H). m/z (ESI⁺): 671.3.

Synthesis of Compound 34

Compound 34 was synthesized according to the procedure of compound 26 using ursodeoxycholic acid as start material. ¹H NMR (500 MHz, CD₃OD) δ ppm 0.73 (s, 3H), 0.97 (s, 3H), 1.04 (d, J=5.5 Hz, 3H), 1.14-1.39 (m, 9H), 1.40-1.67 (m, 12H), 1.77-2.26 (m, 12H), 2.29-2.49 (m, 3H), 2.54-2.79 (m, 4H), 3.41-3.55 (m, 4H), 3.83-4.08 (m, 5H), 4.23-4.33 (m, 2H), 4.64-4.76 (m, 2H), 5.53 (s, 0.5H), 5.64 (s, 0.5H), 7.42-7.55 (m, 2H), 7.90 (s, 1H), 8.07-8.15 (m, 1H), 9.13 (s, 1H). m/z (ESI⁺): 975.5.

Synthesis of Compound 35

Compound 35 was synthesized according to the procedure of compound 26 using arachidonic acid as start material. ¹H NMR (500 MHz, CD₃OD) δ ppm 0.89-0.92 (t, J=7.5 Hz, 3H), 1.31-1.37 (m, 10H), 1.85-1.91 (m, 2H), 2.04-2.08 (m, 2H), 2.13-2.50 (m, 10H), 2.75-2.91 (m, 9H), 3.48-3.52 (m, 2H), 3.89-4.08 (m, 6H), 4.31-4.33 (m, 2H), 4.75 (s, 4H), 5.29-5.48 (m, 8H), 5.57-5.67 (m, 1H) 7.50-7.55 (m, 2H) 7.94 (s, 1H), 8.12-8.15 (m, 1H), 9.16 (s, 1H). m/z (ESI⁺): 887.5.

Synthesis of Compound 36

Compound 36 was synthesized according to the procedure of compound 26 using 2-methylbutyric acid as start material. ¹H NMR (500 MHz, CD₃OD) δ1.06 (t, J=7.5 Hz, 3H), 1.32 (d, J=7.0 Hz, 3H), 1.63-1.75 (m, 1H), 1.81-1.88 (m, 1H), 2.10-2.18 (m, 5H), 2.31-2.39 (m, 2H), 2.45 (s, 1H), 2.56-2.79 (m, 3H), 3.45-3.51 (m, 2H), 3.85-4.08 (m, 5H), 4.29 (d, J=10.0 Hz, 2H), 4.68-4.76 (m, 2H), 4.86 (s, 2H), 5.53 (s, 1H), 5.64 (s, 1H), 7.43 (s, 1H), 7.51 (t, J=9.0 Hz, 1H), 7.91 (d, J=2.0 Hz, 1H), 8.13 (dd, J=9.0, 5.5 Hz, 1H), 9.14 (s, 1H). m/z (ESI⁺): 685.3.

Synthesis of Compound 37

Compound 37 was synthesized according to the procedure of compound 26 using myristic acid as start material. ¹H NMR (500 MHz, CD₃OD) δ 9.16 (s, 1H), 8.13-8.14 (m, 1H), 7.93 (s, 1H), 7.49-7.55 (m, 2H), 5.56-5.61 (m, 1H), 4.91 (m, 2H), 4.75 (s, 2H), 4.31 (s, 2H), 4.09-3.86 (m, 4H), 3.48-3.50 (d, 2H), 2.60-2.75 (m, 4H), 2.37-2.47 (m, 3H), 2.07-2.19 (m, 5H), 1.78-1.79 (m, 2H), 1.31-1.48 (m, 21H), 0.90-0.93 (m, 3H). m/z (ESI⁺): 811.5.

Synthesis of Compound 38

Compound 38 was synthesized according to the procedure of compound 26 using 2-propylpentanoic acid as start material. ¹H NMR (500 MHz, CD₃OD) δ ppm 0.99 (t, J=5.0 Hz, 6H), 1.45-1.79 (m, 8H), 2.11-2.76 (m, 11H), 3.48-3.49 (m, 2H), 3.86-4.05 (m, 5H), 4.26-4.29 (m, 2H), 4.72 (s, 2H), 4.84-4.93 (m, 2H), 5.54-5.64 (m, 1H), 7.38 (s, 1H), 7.51 (t, J=10.0 Hz, 1H), 7.88-7.89 (m, 1H), 8.13-8.16 (m, 1H), 9.13 (s, 1H). m/z (ESI⁺): 727.3.

Synthesis of Compound 39

Compound 39 was synthesized according to the procedure of compound 26 using nonanoic acid as start material. ¹H NMR (500 MHz, CD₃OD) δ ppm 0.91-0.93 (m, 3H), 1.35-1.47 (m, 12H), 1.61-1.64 (m, 2H), 1.76-1.81 (m, 2H), 2.13-2.23 (m, 4H), 2.29-2.49 (m, 4H), 2.68-2.7 (m, 2H), 3.49-3.50 (m, 2H), 3.89-4.07 (m, 4H), 4.30-4.32 (m, 2H), 4.75 (s, 2H), 5.56-5.67 (m, 1H), 7.49 (s, 1H), 7.51-7.55 (t, J=18.0 Hz, 1H), 7.91-7.94 (d, J=2.0 Hz, 1H), 8.13-8.16 (m, 1H), 9.16 (s, 1H). m/z (ESI⁺): 741.3.

Synthesis of Compound 40

Compound 40 was synthesized according to the procedure of compound 26 using oleic acid acid as start material. ¹H NMR (500 MHz, CD₃OD) δ ppm 9.16 (s, 1H), 8.15-8.16 (m, 1H), 7.94 (s, 1H), 7.49-7.55 (m, 2H), 5.56-5.66 (m, 1H), 5.37 (s, 2H), 4.89-4.95 (m, 1H), 4.72-4.78 m, 2H), 4.32 (s, 2H), 3.82-4.06 (m, 4H), 3.48-3.52 (m, 2H), 2.81-2.55 (m, 4H), 2.31-2.47 (m, 3H), 2.01-2.19 (m, 8H), 1.78-1.81 (m, 2H), 1.63 (s, 1H), 1.31-1.48 (m, 22H), 0.91-0.93 (m, 3H). m/z (ESI⁺): 865.4.

Synthesis of Compound 41

Compound 41 was synthesized according to the procedure of compound 26 using 2-butyloctanoic acid as start material. ¹H NMR (500 MHz, CD₃OD) δ ppm 0.88-0.96 (m, 6H), 1.29-1.42 (m, 13H), 1.65-1.78 (m, 4H), 2.09-2.15 (m, 5H), 2.35-2.76 (m, 5H), 3.46-3.48 (m, 2H), 3.86-4.02 (m, 5H), 4.26-4.29 (m, 2H), 4.72 (s, 2H), 4.85-4.94 (m, 2H), 5.53-5.64 (m, 1H), 7.38 (s, 1H), 7.51 (t, J=10.0 Hz, 1H), 7.88 (s, 1H), 8.13-8.15 (m, 1H), 9.14 (s, 1H). m/z (ESI⁺): 783.4.

Synthesis of Compound 42

Compound 42 was synthesized according to the procedure of compound 26 using 2-butylhexanoic acid as start material. ¹H NMR (500 MHz, CD₃OD) δ ppm 0.95 (t, J=5.0 Hz, 6H), 1.39-1.42 (s, 8H), 1.65-1.66 (m, 2H), 1.79 (t, J=5.0 Hz, 2H), 2.10-2.15 (m, 5H), 2.35-2.45 (m, 3H), 2.57-2.75 (m, 3H), 3.46-3.50 (m, 2H), 3.90-4.04 (m, 5H), 4.27-4.29 (m, 2H), 4.69-4.75 (m, 2H), 4.85-4.93 (m, 2H), 5.53-5.64 (m, 1H), 7.38 (s, 1H), 7.51 (t, J=10.0 Hz, 1H), 7.88-7.89 (m, 1H), 8.13-8.16 (m, 1H), 9.14 (s, 1H). m/z (ESI⁺): 755.4.

Synthesis of Compound 43

Compound 43 was synthesized according to the procedure of compound 26 using octanoic acid as start material. ¹H NMR (500 MHz, CD₃OD) δ ppm 0.91 (t, J=6.5 Hz, 3H), 1.32-1.48 (m, 8H), 1.71-1.82 (m, 2H), 2.08-2.22 (m, 5H), 2.28-2.50 (m, 3H), 2.55-2.78 (m, 4H), 3.46 (d, J=8.0 Hz, 2H), 3.86-4.02 (m, 5H), 4.29 (s, 2H), 4.72 (s, 2H), 4.87 (d, J=12.5 Hz, 2H), 5.58 (d, J=51.7 Hz, 1H), 7.49 (dd, J=20.0, 11.1 Hz, 2H), 7.91 (d, J=2.0 Hz, 1H), 8.12 (dd, J=9.0, 5.5 Hz, 1H), 9.13 (s, 1H). m/z (ESI⁺): 727.4.

Synthesis of Compound 44

Compound 44 was synthesized according to the procedure of compound 26 using 1-adamantaneacetic acid as start material. ¹H NMR (500 MHz, CD₃OD) δ ppm 1.66-1.84 (m, 15H), 2.09-2.15 (m, 5H), 2.34-2.36 (m, 2H), 2.39 (s, 2H), 2.44-2.45 (m, 1H), 2.59-2.72 (m, 2H), 3.45-3.49 (m, 2H), 3.85-4.04 (m, 5H), 4.26-4.29 (m, 2H), 4.72 (s, 2H), 4.84-4.93 (m, 2H), 5.53-5.63 (m, 1H), 7.43 (s, 1H), 7.50 (t, J=10.0 Hz, 1H), 7.88-7.89 (m, 1H), 8.12-8.14 (m, 1H), 9.13 (s, 1H). m/z (ESI⁺): 777.3.

Synthesis of Compound 45

Compound 45 was synthesized according to the procedure of compound 26 using nicotinic acid as start material. ¹H NMR (500 MHz, CD₃OD) δ ppm 2.10-2.22 (m, 5H), 2.33-2.35 (m, 2H), 2.45-2.47 (m, 1H), 2.56-2.77 (m, 2H), 3.46-3.51 (m, 2H), 3.85-4.03 (m, 5H), 4.27-4.29 (m, 2H), 4.72-4.75 (m, 2H), 4.87-4.88 (m, 2H), 5.54-5.64 (m, 1H), 7.52-7.55 (m, 1H), 7.69 (s, 1H), 7.73-7.76 (m, 1H), 8.12-8.18 (m, 2H), 8.69-8.70 (m, 1H), 8.89 (s, 1H), 9.15 (s, 1H), 9.38 (s, 1H). m/z (ESI⁺): 706.2.

Synthesis of Compound 46

Compound 46 was synthesized according to the procedure of compound 26 using 3,3-dimethylbutyric acid as start material. ¹H NMR (500 MHz, CD₃OD) δ ppm 9.16 (s, 1H), 8.15-8.18 (m, 1H), 7.94 (s, 1H), 7.54 (t, J=8.5 Hz, 1H), 7.46 (s, 1H), 5.56-5.66 (m, 1H), 4.75 (m, 2H), 4.31 (d, J=11.0 Hz, 2H), 4.10-3.88 (m, 6H), 3.49-3.50 (m, 2H), 2.63-2.78 (m, 2H), 2.58 (s, 2H), 2.48 (m, 1H), 2.35-2.41 (m, 2H), 2.28-2.08 (m, 6H), 1.19 (s, 9H). m/z (ESI⁺): 699.3.

Synthesis of Compound 47

Compound 47 was synthesized according to the procedure of compound 26 using 1-naphthoic acid as start material. ¹H NMR (500 MHz, CD₃OD) δ ppm 2.10-2.15 (m, 5H), 2.35-2.45 (m, 3H), 2.57-2.75 (m, 2H), 3.49-3.51 (m, 2H), 3.86-4.02 (m, 5H), 4.27-4.30 (m, 2H), 4.69-4.73 (m, 2H), 4.89-4.93 (m, 2H), 5.54-5.64 (m, 1H), 7.53-7.56 (m, 1H), 7.60-7.70 (m, 4H), 8.02 (d, J=5.0 Hz, 1H), 8.14-8.24 (m, 3H), 8.58 (d, J=5.0 Hz, 1H), 8.99 (d, J=5.0 Hz, 1H), 9.16 (s, 1H). m/z (ESI⁺): 755.3.

Synthesis of Compound 48

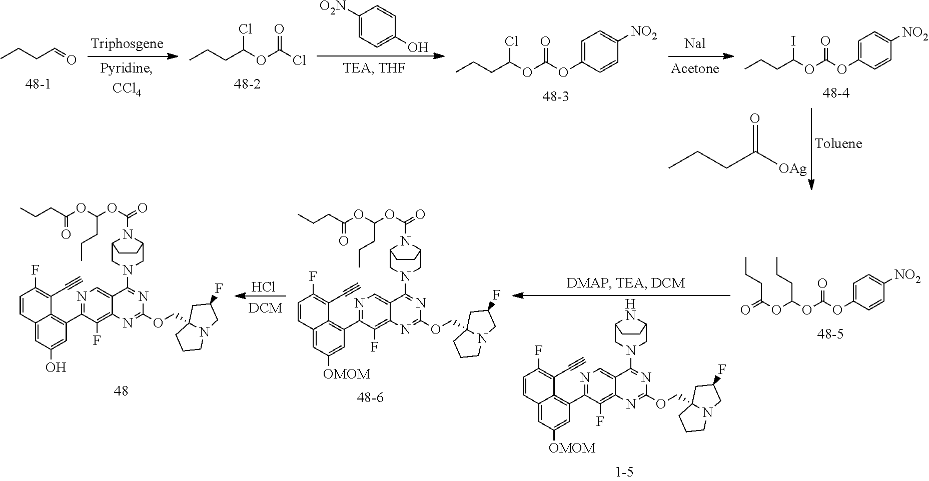

Pyridine (109.7 mg, 1.39 mmol, 0.1 eq) was added to a solution of compound 48-1 (1 g, 13.87 mmol, 1 eq) in CCl₄ (10 mL) and the mixture was cooled to −20˜−10° C. under nitrogen atmosphere. Triphosgene (2.36, 6.93 mmol, 0.5 eq) was added to this mixture slowly. The mixture was warmed to room temperature, then heated to 40° C. and stirred at this temperature for 1 h. The mixture was cooled to room temperature and filtered. The filtrate was concentrated in vacuo to afford compound 48-2 (1.5 g, 63.24% yield).

Et₃N (1.07 g, 10.52 mmol, 1.3 eq) was added to a solution of p-nitrophenol (1.34 g, 9.65 mmol, 1.1 eq) in THF (20 mL), followed by addition of compound 48-2 (1.5 g, 8.77 mmol, 1 eq). The mixture was stirred at room temperature for 1.5 h, then diluted with DCM and water. The organic phase was washed with water and brine, dried over Na₂SO₄, filtered and concentrated. The residue was purified by flash column chromatography (DCM) to afford compound 48-3 (2.2 g, 91.66% yield).

NaI (3.01 g, 20.1 mmol, 2.5 eq) was added to a solution of compound 48-3 (2.2 g, 8.04 mmol, 1 eq) in acetone (20 mL). The mixture was stirred at 50° C. for 15 h under nitrogen atmosphere, then cooled to room temperature and filtered. The filtrated was concentrated and the residue was purified by flash column chromatography (DCM) to afford compound 48-4 (1.6 g, 60% yield).

Silver butyrate (240.3 mg, 1.23 mmol, 1.5 eq) was added to a solution of compound 48-4 (0.5 g, 0.82 mmol, 1 eq) in toluene (10 mL). The mixture was heated to 50° C. and stirred at this temperature for 15 h, then cooled to room temperature, filtered. The filtrate was concentrated and the residue was purified by flash column chromatography (DCM) to afford compound 48-5 (120 mg, 44.89% yield).

Compound 48-5 (60.55 mg, 0.18 mmol, 3 eq) was added to a solution of compound 1-5 (40 mg, 0.062 mmol, 1 eq) in DCM (3 mL), followed by addition of Et₃N (18.84 mg, 0.18 mmol, 3 eq) and DMAP (1.52 mg, 0.012 mmol, 0.2 eq). The mixture was stirred at 40° C. for 1.5 h, then concentrated. The residue was purified by flash column chromatography (MeOH/DCM=0%˜4%) to afford compound 48-6 (40 mg, 77.59% yield).

4M HCl in dioxane (0.5 mL) was added to a solution of compound 48-6 (40 mg, 0.048 mmol, 1.0 eq) in DCM (5 mL). The reaction mixture was stirred at room temperature for 15 min and concentrated in vacuo. The residue was diluted with DCM and the pH was basified with Et₃N, then concentrated again. The residue was diluted with DCM, washed with water and brine, dried over Na₂SO₄, filtered and concentrated. The residue was purified by flash column chromatography (MeOH/DCM=0%˜6%) to afford compound 1 (16.8 mg, 43.5% yield). ¹H NMR (500 MHz, CD₃OD) δ ppm 9.12 (s, 1H), 7.96-7.88 (m, 1H), 7.40 (d, J=2.5 Hz, 1H), 7.37 (t, J=9.0 Hz, 1H), 7.24 (s, 1H), 6.83 (s, 1H), 5.58 (d, J=50.5 Hz, 1H), 4.75-4.59 (m, 2H), 4.57-4.47 (m, 2H), 4.02-3.76 (m, 4H), 3.50-3.38 (m, 4H), 2.77-2.51 (m, 2H), 2.49-2.28 (m, 5H), 2.25-1.98 (m, 3H), 1.94-1.80 (m, 4H), 1.76-1.60 (m, 2H), 1.60-1.45 (m, 2H), 1.42-1.26 (m, 4H), 1.11-0.96 (m, 6H). m/z (ESI⁺): 787.3.

Synthesis of Compound 49

Compound 49 was synthesized according to the procedure of compound 48 using isobutyraldehyde as start material. ¹H NMR (500 MHz, CD₃OD) δ ppm 9.04 (s, 1H), 7.89 (dd, J=9.0, 5.5 Hz, 1H), 7.45-7.31 (m, 2H), 7.23 (s, 1H), 6.65 (d, J=4.3 Hz, 1H), 5.34 (d, J=54.0 Hz, 1H), 4.79-4.62 (m, 2H), 4.59-4.46 (m, 2H), 4.38-4.22 (m, 2H), 3.92-3.60 (m, 2H), 3.40 (d, J=8.0 Hz, 1H), 3.32-3.18 (m, 3H), 3.09-2.98 (m, 1H), 2.48-2.09 (m, 4H), 2.09-1.99 (m, 4H), 1.98-1.83 (m, 3H), 1.77-1.60 (m, 2H), 1.16-0.96 (m, 9H). m/z (ESI⁺): 787.4.

Synthesis of Compound 50

Compound 50 was synthesized according to the procedure of compound 48 using cyclohexanecarboxaldehyde as start material. ¹H NMR (500 MHz, CD₃OD) δ ppm 0.97 (t, J=5.0 Hz, 3H), 1.21-1.69 (m, 6H), 1.66-1.72 (m, 3H), 1.80-2.01 (m, 12H), 2.13-2.37 (m, 5H), 3.02-3.03 (m, 1H), 3.22-3.38 (m, 3H), 3.78 (s, 2H), 4.23-4.33 (m, 2H), 4.47 (s, 2H), 4.69 (s, 2H), 5.26-5.37 (m, 1H), 6.61-6.61 (m, 1H), 7.21 (s, 1H), 7.31-7.35 (s, 2H), 7.85-7.88 (m, 1H), 9.02 (s, 1H). m/z (ESI⁺): 827.4.

Synthesis of Compound 51

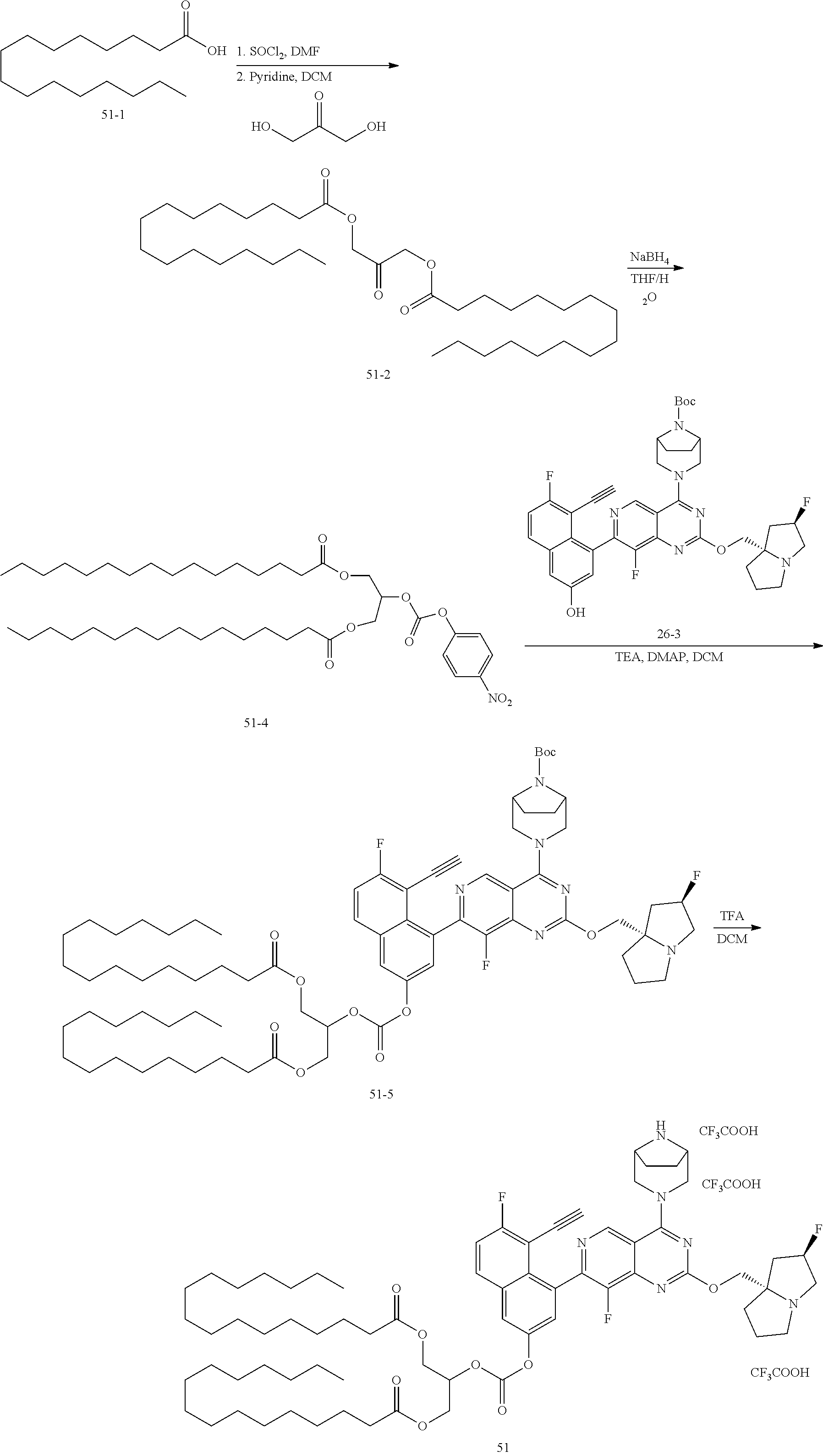

DMF (13.3 mg, 0.18 mmol, 0.0008 eq) was added to a solution of compound 51-1 (5.8 g, 22.6 mmol, 1 eq) in SOCl₂ (50 mL). The mixture was heated to 85° C. and stirred at this temperature for 3 h, then cooled to room temperature. The mixture was concentrated in vacuo to afford palmitoyl chloride. Pyridine (1.87 g, 23.7 mmol, 2.05 eq) was added to a solution of dihydroxyacetone (1.04 g, 11.56 mmol, 1 eq) in DCM (30 mL), followed by addition of palmitoyl chloride. The reaction mixture was stirred at room temperature overnight, then diluted with water and DCM. The organic phase was washed with water and brine, dried over Na₂SO₄, filtered and concentrated. The residue was purified by flash column chromatography (EA/PE=0%˜25%) to afford compound 51-2 (2.32 g, 35.41% yield).

Water (15 mL) was added to a solution of compound 51-2 (2.22 g, 3.92 mmol, 1 eq) in THF (22 mL). NaBH₄ (222 mg, 5.87 mmol, 1.5 eq) was added to this mixture at 0° C. and the mixture was stirred at this temperature for 2 min. The pH was adjusted with 0.5 N HCl aqueous to 7 while keeping the temperature at 0′° C. The mixture was extracted with DCM and the organic phase was dried over Na₂SO₄, filtered and concentrated. The residue was purified by flash column chromatography (EA/PE=0%˜10%) to afford compound 51-3 (1.02 g, 45.78% yield).

Et₃N (80 mg, 0.79 mmol, 1.5 eq) was added to a solution of compound 51-3 (300 mg, 0.53 mmol, 1 eq) in DCM (3 mL), followed by addition of DMAP (6.44 mg, 0.05 mmol, 0.1 eq) and 4-nitrophenyl chloroformate (127 mg, 0.63 mmol, 1.2 eq). The mixture was stirred at temperature for 5 h, then concentrated in vacuo. The residue was purified by flash column chromatography (EA/PE=%10%) to afford compound 51-4 (123 mg, 31.78% yield).

Et₃N (8.66 mg, 0.085 mmol, 1.5 eq) was added to a solution of compound 26-3 (40 mg, 0.057 mmol, 1 eq) in DCM (4 mL), followed by addition of DMAP (0.7 mg, 0.005 mmol, 0.1 eq) and compound 51-4 (50.28 mg, 0.068 mmol, 1.2 eq). The mixture was stirred at room temperature for 3 h, then concentrated in vacuo. The residue was purified by flash column chromatography (MeOH/DCM=0%˜4%) to afford compound 51-5 (73 mg, 100% yield).

TFA (3 mL) was added to a solution of compound 51-5 (73 mg, 0.056 mmol, 1 eq) in DCM (3 mL). The mixture was stirred at room temperature for 10 min, then concentrated in vacuo. The residue was purified by thin layer chromatography (MeOH/DCM=0%˜4%) to afford compound 51 (15.6 mg, 16.48% yield). ¹H NMR (500 MHz, CD₃OD) δ ppm 0.87-0.91 (m, 6H), 1.20-1.31 (m, 48H), 1.58-1.66 (m, 4H), 1.96-2.24 (m, 5H), 2.24-2.51 (m, 7H), 2.51-2.74 (m, 2H), 3.82-4.03 (m, 5H), 4.16-4.46 (m, 4H), 4.49-4.59 (m, 2H), 4.63-4.75 (m, 2H), 5.21 (s, 1H), 5.51 (s, 0.5H), 5.62 (s, 0.5H), 7.48-7.55 (m, 1H), 7.58 (s, 1H), 8.02 (s, 1H), 8.11-8.17 (m, 1H), 9.14 (s, 1H). m/z (ESI⁺): 1196.0.

Synthesis of Compound 52

Compound 52 was synthesized according to the procedure of compound 51 using nonanoic acid as start material. ¹H NMR (500 MHz, CD₃OD) δ ppm 9.18 (s, 1H), 8.17 (dd, J=9.0, 5.5 Hz, 1H), 8.06 (d, J=2.0 Hz, 1H), 7.61 (s, 1H), 7.56 (t, J=9.0 Hz, 1H), 5.62 (d, J=51.5 Hz, 1H), 5.29-5.21 (m, 1H), 4.75 (s, 2H), 4.57 (d, J=10.0 Hz, 2H), 4.40-4.28 (m, 4H), 4.14-3.81 (m, 5H), 3.61-3.45 (m, 2H), 2.84-2.57 (m, 2H), 2.55-2.44 (m, 1H), 2.40 (t, J=6.6 Hz, 6H), 2.32-2.12 (m, 5H), 1.74-1.61 (m, 4H), 1.44-1.18 (m, 20H), 0.87 (t, J=6.0 Hz, 6H). m/z (ESI⁺): 999.5.

Synthesis of Compound 53

Compound 53 was synthesized according to the procedure of compound 51 using lauric acid as start material. ¹H NMR (500 MHz, CD₃OD) δ ppm 0.86 (t, J=5.0 Hz, 6H), 1.23-1.36 (m, 32H), 1.59-1.62 (m, 5H), 2.08-2.15 (m, 6H), 2.35-2.43 (m, 8H), 2.54-2.75 (m, 1H), 3.45-3.48 (m, 2H), 3.81-4.03 (m, 5H), 4.27-4.30 (m, 4H), 4.52-4.55 (m, 2H), 4.69 (s, 2H), 5.21 (s, 1H), 5.52-5.62 (m, 1H), 7.51 (t, J=5.0 Hz, 1H), 7.58 (s, 1H), 8.01 (s, 1H), 8.11-8.14 (m, 1H), 9.13 (s, 1H). m/z (ESI⁺): 1083.7.

Synthesis of Compound 54

Compound 54 was synthesized according to the procedure of compound 51 using oleic acid as start material. ¹H NMR (500 MHz, CD₃OD) δ ppm 0.89 (t, J=5.0 Hz, 6H), 1.21-1.27 (m, 40H), 1.60-1.62 (m, 5H), 1.97-2.16 (m, 13H), 2.36-2.73 (m, 10H), 3.46-3.47 (m, 2H), 3.86-4.05 (m, 5H), 4.23-4.44 (m, 4H), 4.53-55 (m, 2H), 4.72 (s, 2H), 5.20-5.41 (m, 5H), 5.53-5.64 (m, 1H), 7.52 (t, J=5.0 Hz, 1H), 7.58 (s, 1H), 8.02 (s, 1H), 8.11-8.14 (m, 1H), 9.14 (s, 1H). m/z (ESI⁺): 1248.5.

Synthesis of Compound 55

Compound 55 was synthesized according to the procedure of compound 51 using butyric acid as start material. ¹H NMR (500 MHz, CD₃OD) δ ppm 0.91-0.95 (m, 6H), 1.61-1.66 (m, 4H), 2.10-2.18 (m, 5H), 2.31-2.73 (m, 10H), 3.46-3.50 (m, 2H), 3.85-4.06 (m, 5H), 4.24-4.30 (m, 4H), 4.38-4.42 (m, 1H), 4.51-4.56 (m, 2H), 4.72 (s, 2H), 5.21-5.64 (m, 2H), 7.52 (t, J=5.0 Hz, 1H), 7.57 (s, 1H), 8.01-8.02 (m, 1H), 8.12-8.15 (m, 1H), 9.14 (s, 1H). m/z (ESI⁺): 859.4.

Synthesis of Compound 56

Compound 56 was synthesized according to the procedure of compound 51 using decanoic acid as start material. ¹H NMR (500 MHz, CD₃OD) δ ppm 0.84 (t, J=5.0 Hz, 6H), 1.24-1.31 (m, 24H), 1.59-1.62 (m, 4H), 2.10-2.76 (m, 14H), 3.47-3.48 (m, 2H), 3.86-4.06 (m, 5H), 4.28-4.30 (m, 4H), 4.53-55 (m, 2H), 4.72 (s, 2H), 4.86-4.90 (m, 2H), 5.21 (s, 1H), 5.54-5.64 (m, 1H), 7.52 (t, J=5.0 Hz, 1H), 7.58 (s, 1H), 8.02 (s, 1H), 8.12-8.15 (m, 1H), 9.14 (s, 1H). m/z (ESI⁺): 1027.5.

Synthesis of Compound 57

Compound 57 was synthesized according to the procedure of compound 51 using undecanoic acid as start material. ¹H NMR (500 MHz, CD₃OD) δ ppm 0.85 (t, J=5.0 Hz, 6H), 1.21-1.29 (m, 28H), 1.59-1.62 (m, 4H), 2.10-2.18 (m, 6H), 2.35-2.47 (m, 8H), 2.57-2.76 (m, 2H), 3.46-3.48 (m, 2H), 3.86-4.06 (m, 5H), 4.28-4.29 (m, 4H), 4.52-4.55 (m, 2H), 4.72 (s, 2H), 5.21 (s, 1H), 5.53-5.64 (m, 1H), 7.52 (t, J=10.0 Hz, 1H), 7.58 (s, 1H), 8.02 (s, 1H), 8.12-8.15 (m, 1H), 9.14 (s, 1H). m/z (ESI⁺): 1055.5.

Synthesis of Compound 58

Compound 58 was synthesized according to the procedure of compound 51 using myristic acid as start material. ¹H NMR (500 MHz, CD₃OD) δ ppm 0.88 (t, J=7.0 Hz, 6H), 1.21-1.29 (m, 40H), 1.55-1.66 (m, 4H), 2.09-2.16 (m, 5H), 2.28-2.48 (m, 7H), 2.61-2.66 (m, 2H), 3.47 (d, J=9.0 Hz, 2H), 3.84-3.99 (m, 5H), 4.21-4.34 (m, 4H), 4.50-4.57 (m, 2H), 4.71 (s, 4H), 5.21 (s, 1H), 5.53 (s, 1H), 5.63 (s, 1H), 7.52 (t, J=9.0 Hz, 1H), 7.58 (s, 1H), 8.02 (d, J=2.0 Hz, 1H), 8.13 (dd, J=9.0, 6.0 Hz, 1H), 9.14 (s, 1H). m/z (ESI⁺): 1139.7.

Synthesis of Compound 59

Compound 59 was synthesized according to the procedure of compound 51 using palmitic acid as start material. ¹H NMR (500 MHz, MeOD) δ ppm 0.89 (t, J=7.0 Hz, 6H), 1.21-1.30 (m, 44H), 1.60-1.61 (m, 4H), 2.00-2.21 (m, 5H), 2.35-2.38 (m7H), 2.49-2.72 (m, 2H), 3.46 (d, J=9.0 Hz, 2H), 3.88-3.97 (m, 3H), 4.25-4.29 (m, 2H), 4.53-4.68 (m, 10H), 5.21 (s, 1H), 5.56 (d, J=51.5 Hz, 1H), 7.52 (t, J=9.0 Hz, 1H), 7.58 (s, 1H), 8.02 (s, 1H), 8.15 (d, J=5.5 Hz, 1H), 9.14 (s, 1H). m/z (ESI⁺): 1167.7.

Synthesis of Compound 60

Compound 60 was synthesized according to the procedure of compound 51 using tridecanoic acid as start material. ¹H NMR (500 MHz, CD₃OD) δ ppm 0.87 (t, J=5.0 Hz, 6H), 1.21-1.30 (m, 36H), 1.58-1.62 (m, 4H), 2.10-2.16 (m, 5H), 2.35-2.47 (m, 7H), 2.57-2.76 (m, 2H), 3.47-3.49 (m, 2H), 3.86-4.06 (m, 5H), 4.27-4.29 (m, 4H), 4.52-4.55 (m, 2H), 4.72 (s, 2H), 4.85-4.92 (m, 2H), 5.21 (s, 1H), 5.54-5.64 (m, 1H), 7.52 (t, J=10.0 Hz, 1H), 7.58 (s, 1H), 8.02 (s, 1H), 8.12-8.15 (m, 1H), 9.14 (s, 1H). m/z (ESI⁺): 1111.7.

Synthesis of Compound 61

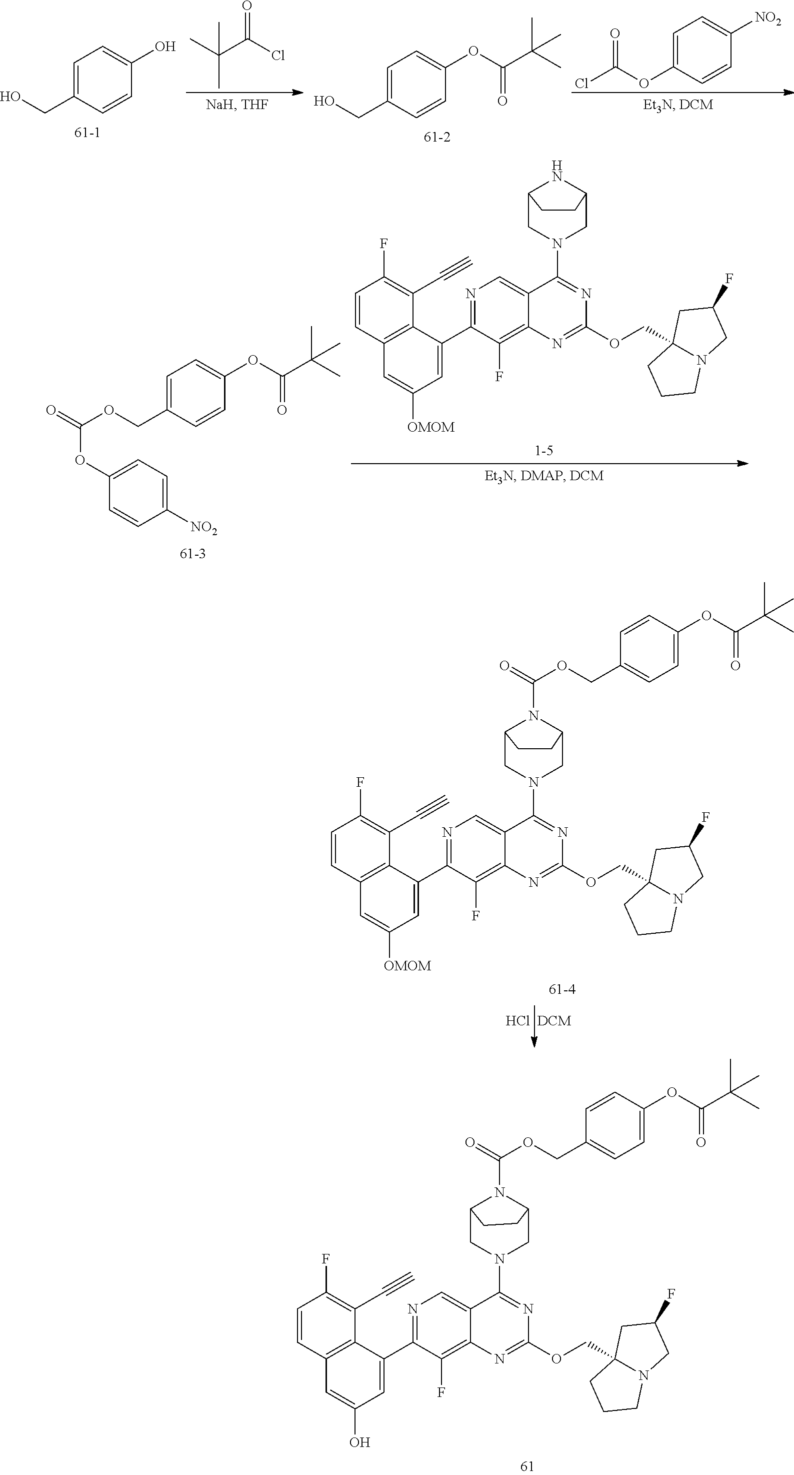

60% NaH (116 mg, 2.9 mmol, 1.2 eq) was added to anhydrous THF (10 mL) and the mixture was cooled to 0° C. under nitrogen atmosphere. Compound 61-1 (300 mg, 2.42 mmol, 1 eq) was added to this mixture at 0° C. slowly. The mixture was warmed to room temperature after the bubbles disappear and stirred at this temperature for 30 min, then cooled to 0° C. again. Pivaloyl chloride (320 mg, 2.66 mmol, 1.1 eq) was added to this mixture at 0° C. The reaction mixture was stirred at 0° C. for 30 min, then warmed to room temperature and stirred at this temperature for 2 h. The reaction was quenched with NaHCO₃ aqueous, then diluted with water and EA. The organic phase was washed with water and brine, dried over Na₂SO₄, filtered and concentrated. The residue was purified by flash column chromatography (EA/Hexane=0%˜40%) to afford compound 61-2 (381 mg, 75.7% yield).

Et₃N (58.31 mg, 0.57 mmol, 1.2 eq) was added to a solution of compound 61-2 (100 mg, 0.48 mmol, 1 eq) in DCM (2 mL), followed by addition of 4-nitrophenyl chloroformate (106.5 mg, 0.53 mmol, 1.1 eq). The mixture was stirred at room temperature for 6 h and concentrated in vacuo. The residue was purified by flash column chromatography (EA/Hexane=0%˜10%) to afford compound 61-3 as a colorless oil (124 mg, 69.17% yield).

Et₃N (9.42 mg, 0.093 mmol, 1.5 eq) was added to a solution of compound 1-5 (40 mg, 0.062 mmol, 1 eq) in DCM (4 mL), followed by addition of DMAP (0.758 mg, 0.006 mmol, 0.1 eq) and compound 61-3 (27.8 mg, 0.074 mmol, 1.2 eq). The mixture was heated to 40° C. and stirred at this temperature for 3 h, then cooled to room temperature. The mixture was concentrated and the residue was purified by flash column chromatography (MeOH/DCM=0%˜4%) to afford compound 61-4 (79 mg, 100% yield).

4M HCl in dioxane (1 mL) was added to a solution of compound 61-4 (79 mg, 0.089 mmol, 1 eq) in DCM (10 mL). The mixture was stirred at room temperature for 2 min, then concentrated in vacuo. The residue was purified by preparative HPLC to afford compound 61 (20.7 mg, 27.24% yield). ¹H NMR (500 MHz, CD₃OD) δ ppm 1.35 (s, 9H), 1.79-1.94 (m, 3H), 1.95-2.05 (m, 4H), 2.10-2.37 (m, 3H), 2.97-3.05 (m, 1H), 3.14-3.29 (m, 3H), 3.34-3.38 (m, 1H), 3.76 (s, 2H), 4.19-4.34 (m, 2H), 4.50 (s, 2H), 4.66 (s, 2H), 5.19-5.28 (m, 2.5H), 5.36 (s, 0.5H), 7.08 (d, J=8.5 Hz, 2H), 7.20 (s, 1H), 7.28-7.38 (m, 2H), 7.48 (d, J=8.5 Hz, 2H), 7.82-7.91 (m, 1H), 9.01 (s, 1H). m/z (ESI⁺): 835.4.

Synthesis of Compound 62

Compound 62 was synthesized according to the procedure of compound 61 using butyryl chloride as start material. ¹H NMR (500 MHz, CD₃OD) δ ppm 1.03 (t, J=5.0 Hz, 3H), 1.73-1.79 (m, 2H), 1.83-1.92 (m, 4H), 1.96-2.00 (m, 5H), 2.12-2.36 (m, 3H), 2.56 (t, J=10.0 Hz, 2H), 2.99-3.02 (m, 1H), 3.17-3.24 (m, 3H), 3.75 (s, 2H), 4.22-4.32 (m, 2H), 4.50-4.65 (m, 4H), 5.22 (s, 2H), 5.25-5.36 (m, 1H), 7.11 (d, J=10 Hz, 2H), 7.20 (s, 1H), 7.30-7.35 (m, 2H), 7.47 (d, J=5.0 Hz, 2H), 7.84-7.87 (m, 1H), 9.00 (s, 1H). m/z (ESI⁺): 821.6.

Synthesis of Compound 63

Compound 63 was synthesized according to the procedure of compound 61 using acetyl chloride as start material. ¹H NMR (500 MHz, CD₃OD) δ ppm 1.85-2.01 (m, 8H), 2.15-2.23 (m, 2H), 2.27 (s, 3H), 3.02-3.03 (m, 5H), 3.76 (s, 1H), 4.26-4.32 (m, 1H), 4.51-4.59 (m, 7H), 5.22 (s, 2H), 5.26-5.37 (m, 2H), 7.12-7.13 (d, J=5.0 Hz, 2H), 7.20 (s, 1H), 7.31-7.35 (m, 2H), 7.47-7.48 (m, 2H), 7.86 (s, 1H), 9.01 (s, 1H). m/z (ESI⁺): 793.59.

Synthesis of Compound 64

Compound 64 was synthesized according to the procedure of compound 61 using decanoyl chloride as start material. ¹H NMR (500 MHz, CD₃OD) δ ppm 0.90 (t, J=10.0 Hz, 3H), 1.31-1.42 (m, 14H), 1.69-2.00 (m, 9H), 2.12-2.33 (m, 3H), 2.57 (t, J=5.0 Hz, 2H), 3.01-3.03 (m, 1H), 3.20-3.24 (m, 2H), 3.33 (t, J=10.0 Hz, 1H), 3.75 (s, 2H), 4.22-4.32 (m, 2H), 4.50-4.65 (m, 4H), 5.22 (s, 2H), 5.25-5.36 (m, 1H), 7.10 (d, J=5.0 Hz, 2H), 7.20 (s, 1H), 7.32 (d, J=10.0 Hz, 1H), 7.35 (s, 1H), 7.47 (d, J=5.0 Hz, 2H), 7.85 (q, J=5 Hz, 1H), 9.00 (s, 1H). m/z (ESI⁺): 905.7.

Synthesis of Compound 65

Compound 65 was synthesized according to the procedure of compound 61 using isovaleryl chloride as start material. ¹H NMR (500 MHz, CD₃OD) δ ppm 1.05 (d, J=5.0 Hz, 7H), 1.82 (d, J=10.0 Hz, 2H), 2.02 (s, 2H), 2.16-2.21 (m, 2H), 2.32-2.35 (m, 2H), 2.45 (m, J=5.0 Hz, 2H), 2.55-2.74 (m, 2H), 3.44-3.50 (m, 1H), 3.75-4.05 (m, 5H), 4.52 (s, 2H), 4.62-4.71 (m, 4H), 5.23 (s, 2H), 5.52-5.62 (m, 1H), 7.10 (d, J=5.0 Hz, 2H), 7.21 (s, 1H), 7.34 (d, J=10.0 Hz, 1H), 7.37 (s, 1H), 7.48 (d, J=10.0 Hz, 2H), 7.88 (q, J=5.0 Hz, 1H), 9.09 (s, 1H). m/z (ESI⁺): 835.7.

Synthesis of Compound 66

Compound 66 was synthesized according to the procedure of compound 61 using 2-propylpentanoyl chloride as start material. ¹H NMR (500 MHz, CD₃OD) δ ppm 0.97 (t, J=10.0 Hz, 6H), 1.41-1.46 (m, 4H), 1.53-1.58 (m, 2H), 1.68-1.75 (m, 2H), 1.82-1.89 (m, 3H), 1.99-2.35 (m, 7H), 2.63 (t, J=5.0 Hz, 1H), 3.17-3.36 (m, 4H), 3.75 (s, 2H), 4.22-4.31 (m, 2H), 4.49-4.64 (m, 4H), 5.22 (s, 2H), 5.25-5.36 (m, 1H), 7.07 (d, J=10.0 Hz, 2H), 7.20 (s, 1H), 7.30-7.34 (m, 2H), 7.48 (d, J=10.0 Hz, 2H), 7.83-7.86 (m, 1H), 9.00 (s, 1H). m/z (ESI⁺): 877.5.

Synthesis of Compound 67

Compound 67 was synthesized according to the procedure of compound 61 using valeryl chloride as start material. ¹H NMR (500 MHz, CD₃OD) δ ppm 0.97 (t, J=5.0 Hz, 3H), 1.42-1.48 (m, 2H), 1.67-1.70 (m, 2H), 1.83-1.99 (m, 7H), 2.11-2.22 (m, 3H), 2.58 (t, J=5.0 Hz, 2H), 2.99-3.02 (m, 1H), 3.19-3.36 (m, 4H), 3.75 (s, 2H), 4.21-4.28 (m, 2H), 4.49-4.63 (m, 4H), 5.22 (s, 2H), 5.25-5.35 (m, 1H), 7.10 (d, J=10.0 Hz, 2H), 7.20 (s, 1H), 7.30-7.35 (m, 2H), 7.47 (d, J=10.0 Hz, 2H), 7.84-7.86 (m, 1H), 9.00 (s, 1H). m/z (ESI⁺): 835.4.

Synthesis of Compound 68

Compound 68 was synthesized according to the procedure of compound 61 using cyclopentaneacetyl chloride as start material. ¹H NMR (500 MHz, CD₃OD) δ ppm 1.25-1.29 (m, 2H), 1.60-1.70 (m, 4H), 1.82-2.03 (m, 9H), 2.11-2.35 (m, 4H), 2.57 (d, J=5.0 Hz, 2H), 2.99-3.02 (m, 1H), 3.20-3.36 (m, 4H), 3.74 (s, 2H), 4.21-4.31 (m, 2H), 4.49-4.64 (m, 4H), 5.22 (s, 2H), 5.24-5.35 (m, 1H), 7.10 (d, J=10.0 Hz, 2H), 7.20 (s, 1H), 7.30-7.34 (m, 2H), 7.47 (d, J=10.0 Hz, 2H), 7.84-7.87 (m, 1H), 8.99 (s, 1H). m/z (ESI⁺): 861.5.

Synthesis of Compound 69

Compound 69 was synthesized according to the procedure of compound 61 using 3-cyclopentylpropionyl chloride as start material. ¹H NMR (500 MHz, CD₃OD) δ ppm 1.17-1.18 (m, 2H), 1.58-1.76 (m, 6H), 1.84-2.00 (m, 10H), 2.14-2.36 (m, 3H), 2.59 (t, J=5.0 Hz, 2H), 3.01-3.02 (m, 1H), 3.17-3.36 (m, 4H), 3.75 (s, 2H), 4.22-4.32 (m, 2H), 4.50-4.66 (m, 4H), 5.22 (s, 2H), 5.25-5.36 (m, 1H), 7.11 (d, J=10.0 Hz, 2H), 7.20 (s, 1H), 7.31-7.35 (m, 2H), 7.48 (d, J=10.0 Hz, 2H), 7.85-7.88 (m, 1H), 9.00 (s, 1H). m/z (ESI⁺): 875.5.

Synthesis of Compound 70

Compound 70 was synthesized according to the procedure of compound 61 using 2-hexyldecanoyl chloride as start material. ¹H NMR (500 MHz, CD₃OD) δ ppm 0.89 (t, J=5.0 Hz, 6H), 1.32-1.39 (m, 20H), 1.58-1.60 (m, 2H), 1.70-1.73 (m, 2H), 1.82-1.89 (m, 3H), 1.99-2.32 (m, 7H), 2.58-2.60 (m, 1H), 3.01-3.02 (m, 1H), 3.19-3.35 (m, 4H), 3.75 (s, 2H), 4.22-4.31 (m, 2H), 4.49-4.64 (m, 4H), 5.23-5.36 (m, 3H), 7.06 (d, J=5.0 Hz, 2H), 7.20 (s, 1H), 7.30-7.34 (m, 2H), 7.49 (d, J=5.0 Hz, 2H), 7.84-7.87 (m, 1H), 9.00 (s, 1H). m/z (ESI⁺): 989.5.

Synthesis of Compound 71

Compound 71 was synthesized according to the procedure of compound 61 using dodecanoyl chloride as start material. ¹H NMR (500 MHz, CD₃OD) δ ppm 0.87-0.93 (m, 3H), 1.28-1.44 (m, 16H), 1.67-1.76 (m, 2H), 1.81-2.05 (m, 7H), 2.11-2.37 (m, 3H), 2.54-2.60 (m, 2H), 2.98-3.06 (m, 1H), 3.13-3.29 (m, 3H), 3.35 (d, J=8.5 Hz, 1H), 3.75 (s, 2H), 4.19-4.34 (m, 2H), 4.44-4.54 (m, 2H), 4.58-4.73 (m, 2H), 5.22 (s, 2H), 5.26 (s, 0.5H), 5.37 (s, 1H), 7.10 (d, J=8.5 Hz, 2H), 7.20 (s, 1H), 7.28-7.37 (m, 2H), 7.48 (d, J=8.5 Hz, 2H), 7.83-7.89 (m, 1H), 9.00 (s, 1H). m/z (ESI⁺): 933.5.

Synthesis of Compound 72

Compound 72 was synthesized according to the procedure of compound 61 using palmitoyl chloride as start material. ¹H NMR (500 MHz, CD₃OD) δ ppm 0.85-0.93 (m, 3H), 1.28-1.44 (m, 24H), 1.66-1.77 (m, 2H), 1.81-2.06 (m, 7H), 2.11-2.40 (m, 3H), 2.58 (t, J=7.5 Hz, 2H), 3.00-3.09 (m, 1H), 3.13-3.29 (m, 3H), 3.36 (d, J=8.5 Hz, 1H), 3.70-3.82 (m, 2H), 4.20-4.37 (m, 2H), 4.45-4.55 (m, 2H), 4.58-4.73 (m, 2H), 5.18-5.29 (m, 2.5H), 5.38 (s, 0.5H), 7.11 (d, J=8.5 Hz, 2H), 7.20 (s, 1H), 7.29-7.38 (m, 2H), 7.48 (d, J=8.5 Hz, 2H), 7.84-7.90 (m, 1H), 9.01 (s, 1H). m/z (ESI⁺): 989.5.

Synthesis of Compound 73

Compound 73 was synthesized according to the procedure of compound 61 using myristoyl chloride as start material. ¹H NMR (500 MHz, DMSO-d6) δ ppm 10.18 (s, 1H), 9.05 (s, 1H), 7.99-7.81 (m, 1H), 7.48-7.50 (m, 3H), 7.42 (s, 1H), 7.19 (s, 1H), 7.14-7.16 (m, 2H), 5.27-5.38 (m, 1H), 5.19 (s, 1H), 4.60-4.61 (m, 1H), 4.45 (s, 3H), 4.20-3.89 (m, 2H), 3.78-3.63 (m, 2H), 3.14 (m, 3H), 2.89 (s, 1H), 2.57-2.60 (m, 2H), 2.12 (m, 3H), 1.80-1.89 (m, 7H), 1.63-1.64 (m, 2H), 1.32 (s, 2H), 1.29 (s, 20H), 0.85-0.87 (m, 3H). m/z (ESI⁺): 961.5.

Synthesis of Compound 74

Et₃N (10.41 mg, 0.102 mmol, 1.5 eq) was added to a solution of compound 69 (60 mg, 0.068 mmol, 1 eq) in DCM (6 mL), followed by addition of DMAP (0.84 mg, 0.007 mmol, 0.1 eq) and pivaloyl chloride (9.92 mg, 0.082 mmol, 1.2 eq). The mixture was stirred at room temperature for 1.5 h, then diluted with DCM and water. The organic phase was washed with water and brine, dried over Na₂SO₄, filtered and concentrated. The residue was purified by flash column chromatography (MeOH/DCM=0%˜4%) to afford compound 74 (44 mg, 65.5% yield). ¹H NMR (500 MHz, CD₃OD) δ ppm 9.05 (s, 1H), 8.11 (dd, J=9.0, 5.5 Hz, 1H), 7.89 (d, J=2.5 Hz, 1H), 7.55-7.47 (m, 3H), 7.45 (s, 1H), 7.13 (d, J=8.5 Hz, 2H), 5.37 (d, J=53.5 Hz, 1H), 5.25 (s, 2H), 4.84-4.58 (m, 2H), 4.58-4.45 (m, 2H), 4.43-4.26 (m, 2H), 3.86-3.70 (m, 2H), 3.50 (d, J=8.0 Hz, 1H), 3.17-3.03 (m, 1H), 2.62 (t, J=7.6 Hz, 2H), 2.43-2.15 (m, 3H), 2.15-1.81 (m, 10H), 1.81-1.54 (m, 6H), 1.46-1.40 (m, 9H), 1.19 (d, J=11.0 Hz, 2H). m/z (ESI⁺): 959.3.

Synthesis of Compound 75

Compound 75 was synthesized according to the procedure of compound 74 using compound 61 as start material. ¹H NMR (500 MHz, CD₃OD) δ ppm 9.07 (s, 1H), 8.12 (dd, J=9.1, 5.7 Hz, 1H), 7.89 (d, J=2.2 Hz, 1H), 7.55-7.48 (m, 3H), 7.45 (s, 1H), 7.11 (d, J=8.4 Hz, 2H), 5.42 (d, J=52.9 Hz, 1H), 5.25 (s, 2H), 4.78-4.61 (m, 2H), 4.58-4.36 (m, 4H), 3.88-3.69 (m, 2H), 3.64-3.41 (m, 4H), 3.26-3.14 (m, 1H), 2.56-2.22 (m, 3H), 2.20-2.09 (m, 2H), 2.09-1.95 (m, 3H), 1.93-1.78 (m, 2H), 1.42 (s, 9H), 1.38 (s, 9H). m/z (ESI⁺): 919.48.

Synthesis of Compound 76

Compound 76 was synthesized according to the procedure of compounds 74 and 51 using compound 1 as start material. ¹H NMR (500 MHz, CD₃OD) δ ppm 0.80-0.88 (m, 6H), 0.93-1.01 (m, 3H), 1.20-1.36 (m, 20H), 1.50-1.70 (m, 9H), 1.82-2.08 (m, 7H), 2.09-2.40 (m, 9H), 2.99-3.07 (m, 1H), 3.15-3.27 (m, 3H), 3.51 (d, J=7.0 Hz, 1H), 3.68-3.84 (m, 2H), 4.18-4.35 (m, 4H), 4.43-4.56 (m, 4H), 4.59-4.76 (m, 2H), 5.16-5.28 (m, 1.5H), 5.37 (s, 0.5H), 6.81-6.90 (m, 1H), 7.47-7.58 (m, 2H), 8.00 (s, 1H), 8.09-8.15 (m, 1H), 9.05 (s, 1H). m/z (ESI⁺): 1157.6.

Synthesis of Compound 77

Compound 77 was synthesized according to the procedure of compound 76 using compound 3 as start material. ¹H NMR (500 MHz, CD₃OD) δ ppm 9.07 (s, 1H), 8.12 (dd, J=9.1, 5.4 Hz, 1H), 8.01 (s, 1H), 7.59 (d, J=2.3 Hz, 1H), 7.52 (t, J=8.8 Hz, 1H), 6.92-6.81 (m, 1H), 5.34 (d, J=54.4 Hz, 1H), 5.27-5.22 (m, 1H), 4.79-4.59 (m, 2H), 4.58-4.42 (m, 4H), 4.39-4.23 (m, 4H), 3.94-3.65 (m, 2H), 3.52 (d, J=5.9 Hz, 1H), 3.33-3.17 (m, 3H), 3.11-2.99 (m, 1H), 2.42-2.33 (m, 4H), 2.32-2.14 (m, 3H), 2.14-1.98 (m, 7H), 1.97-1.81 (m, 3H), 1.69-1.52 (m, 7H), 1.34-1.19 (m, 20H), 0.86 (t, J=5.2 Hz, 6H). m/z (ESI⁺): 1129.4.

The synthesis methods of other compounds was similar to the syntheses shown in the previous text, and their identification was carried out using nuclear magnetic resonance (NMR) and mass spectrometry. The obtained NMR and mass spectrometry data for synthesized compounds are shown in Table 3.

TABLE 3
Mass spectromety and NMR data for exemplary synthesized compounds of the disclosure.

| Compound | [M + 1]⁺ | NMR |
|---|---|---|
| 78 | 702.5 | ¹H NMR (500 MHz, CDCl₃) δ ppm 9.12 (s, 1H), 7.92 (dd, J = 9.1, 5.7 Hz, |
| | | 1H), 7.41 (d, J = 2.4 Hz, 1H), 7.38 (t, J = 8.9 Hz, 1H), 7.25 (d, J = 2.2 Hz, |
| | | 1H), 5.61 (d, J = 51.8 Hz, 1H), 4.79-4.66 (m, 2H), 4.66-4.41 (m, 4H), |
| | | 4.16-3.75 (m, 5H), 3.55-3.47 (m, 1H), 3.42 (t, J = 4.9 Hz, 2H), 3.39-3.35 (m, |
| | | 2H), 2.83 (s, 3H), 2.81-2.56 (m, 2H), 2.53-2.43 (m, 1H), 2.43-2.32 (m, 2H), |
| | | 2.31-2.14 (m, 1H), 2.07 (s, 2H), 1.88 (d, J = 9.1 Hz, 2H) |
| 79 | 780.5 | ¹H NMR (500 MHz, CD₃OD) δ ppm 1.88-2.29 (m, 14H), 2.99 (s, 1H), |
| | | 3.14-3.29 (m, 4H), 3.62 (s, 2H), 4.22(s, 1H), 4.45-4.52 (m, 2H), 5.28(s, |
| | | 2H), 7.07-7.17 (m, 3H), 7.53 (d, J = 10 Hz, 2H), 7.60 (s, 1H), 8.23 (d, J = 10 |
| | | Hz, 2H), 8.85 (d, J = 15 Hz, 1H). |
| 80 | 972.6 | ¹H NMR (500 MHz, CD₃OD) δ ppm 1.83 (d, J = 5 Hz, 3H), 1.99-2.04 (m, |
| | | 3H), 2.35 (s, 3H), 2.59-2.64 (m, 1H), 3.45-3.56 (m, 4H), 3.61-3.65 (m, 1H), |
| | | 3.75-3.95 (s, 3H), 4.03-4.05 (m, 2H), 4.53 (s, 2H), 4.62-4.66 (m, 2H), 4.72 |
| | | (d, J = 10 Hz, 7H), 5.18 (s, 1H), 5.20 (s, 1H), 5.23 (s, 2H), 7.21 (s, 1H), |
| | | 7.32-7.37 (m, 2H), 7.44 (d, J = 10 Hz, 1H), 7.69 (d, J = 10 Hz, 1H), 7.87-7.90 |
| | | (m, 1H), 7.93(s, 2H), 9.10 (s, 1H). |
| 81 | 784.6 | ¹H NMR (500 MHz, CD₃OD) δ ppm 1.84-1.89 (m, 3H), 1.96-2.00 (m, 5H), |
| | | 2.11-2.32 (m, 2H), 2.09-3.22 (m, 1H), 3.18-3.27 (m, 3H), 3.80 (s, 2H), 4.07 |
| | | (s, 3H), 4.22-4.30 (m, 2H), 4.50 (s, 2H), 4.59 (s, 4H), 5.35 (s, 2H), 7.20 (s, |
| | | 1H), 7.30-7.35 (m, 2H), 7.84 (d, J = 5 Hz, 1H), 8.00 (s, 1H), 9.01(s, 1H). |
| 82 | 716.5 | ¹H NMR (500 MHz, CD₃OD) δ ppm 9.13 (s, 1H), 7.92 (dd, J = 9.1, 5.7 Hz, |
| | | 1H), 7.41 (d, J = 2.5 Hz, 1H), 7.37 (t, J = 8.9 Hz, 1H), 7.25 (d, J = 2.3 Hz, |
| | | 1H), 5.61 (d, J = 51.7 Hz, 1H), 4.83-4.62 (m, 4H), 4.55 (s, 2H), 4.32 (t, J = |
| | | 6.0 Hz, 2H), 4.13-3.70 (m, 5H), 3.55-3.45 (m, 1H), 3.37 (s, 2H), 3.22-3.15 |
| | | (m, 2H), 2.78 (s, 3H), 2.76-2.56 (m, 2H), 2.54-2.43 (m, 1H), 2.43-2.34 (m, |
| | | 2H), 2.29-2.17 (m, 1H), 2.17-2.10 (m, 2H), 2.09-1.98 (m, 2H), 1.87 (d, J = |
| | | 8.7 Hz, 2H) |
| 83 | 823.6 | ¹H NMR (500 MHz, CD₃OD) δ ppm 1.83 (d, J = 8.5 Hz, 2H), 1.95-2.11 (m, |
| | | 5H), 2.11-2.23 (m, 1H), 2.30-2.40 (m, 2H), 2.40-2.51 (m, 1H), 2.54-2.77 |
| | | (m, 2H), 2.99 (t, J = 6.5 Hz, 2H), 3.06 (t, J = 6.0 Hz, 2H), 3.41-3.54 (m, 1H), |
| | | 3.75-4.07 (m, 5H), 4.34 (t, J = 6.5 Hz, 2H), 4.41-4.56 (m, 4H), 4.60-4.77 |
| | | (m, 4H), 5.44-5.66 (m, 1H), 7.22 (s, 1H), 7.27-7.39 (m, 2H), 7.88 (dd, J = |
| | | 9.0, 5.5 Hz, 1H), 9.12 (s, 1H). |
| 84 | 880.6 | ¹H NMR (500 MHz, CD₃OD) δ ppm 1.02-1.16 (m, 6H), 1.78-1.90 (m, 2H), |
| | | 1.97-2.09 (m, 2H), 2.11-2.22 (m, 1H), 2.25-2.39 (m, 3H), 2.40-2.49 (m, |
| | | 1H), 2.53-2.77 (m, 2H), 3.02-3.12 (m, 4H), 3.40-3.52 (m, 1H), 3.73-4.08 |
| | | (m, 6H), 4.39-4.57 (m, 5H), 4.57-4.78 (m, 4H), 5.47-5.66 (m, 1H), 7.21 (s, |
| | | 1H), 7.27-7.41 (m, 2H), 7.89 (dd, J = 9.0, 5.5 Hz, 1H), 9.11 (s, 1H). |
| 85 | 765.6 | ¹H NMR (500 MHz, CD₃OD) δ ppm 1.38 (d, J = 6.0 Hz, 12H), 1.80 (d, J = |
| | | 8.0 Hz, 2H), 1.94-2.05 (m, 2H), 2.10-2.25 (m, 1H), 2.30-2.40 (m, 2H), |
| | | 2.40-2.49 (m, 1H), 2.54-2.78 (m, 2H), 3.43-3.51 (m, 1H), 3.72-4.07 (m, 5H), |
| | | 4.08-4.18 (m, 2H), 4.58-4.77 (m, 6H), 5.48-5.67 (m, 1H), 7.22 (s, 1H), |
| | | 7.29-7.42 (m, 2H), 7.85-7.91 (m, 1H), 9.11 (s, 1H). ³¹P NMR (203 MHz, |
| | | CD₃OD) δ ppm 3.36. |
| 86 | 807.7 | ¹H NMR (500 MHz, CD₃OD) δ ppm 1.79-1.93 (m, 3H), 1.94-2.08 (m, |
| | | 4H), 2.09-2.38 (m, 3H), 2.93-3.04 (m, 1H), 3.12-3.25 (m, 3H), 3.33-3.36 |
| | | (m, 1H), 3.41-3.53 (m, 1H), 3.68-3.92 (m, 3H), 4.05 (s, 0.1H), 4.18-4.36 |
| | | (m, 2H), 4.41-4.51 (m, 1H), 4.53-4.72 (m, 7.5H), 5.09-5.38 (m, 1.5H), 7.20 |
| | | (s, 1H), 7.27-7.36 (m, 2H), 7.82-7.88 (m, 1H), 9.01 (s, 1H). |
| 87 | 900.73 | ¹H NMR (500 MHz, CD₃OD) δ ppm 1.31 (d, J = 7.5 Hz, 2H), 1.38(d, J = |
| | | 7.0 Hz, 1H), 1.82 (d, J = 7.0 Hz, 2H), 1.96-2.07 (m, 2H), 2.10-2.22 (m, 1H), |
| | | 2.30-2.39 (m, 2H), 2.41-2.49 (m, 1H), 2.54-2.77 (m, 2H), 3.34-3.38 (m, |
| | | 1H), 3.43-3.51 (m, 1H), 3.68 (s, 1H), 3.71 (s, 2H), 3.83-4.09 (m, 9H), 4.36-4.51 |
| | | (m, 2H), 4.62-4.77 (m, 5H), 5.49-5.65 (m, 1H), 7.22 (s, 1H), 7.27-7.40 |
| | | (m, 3H), 7.51-7.65 (m, 2H), 7.85-7.93 (m, 2H), 9.13 (s, 1H). ³¹P NMR (203 |
| | | MHz, CD₃OD) δ ppm 6.19, 6.69. |
| 88 | 842.66 | ¹H NMR (500 MHz, CD₃OD) δ ppm 1.37 (d, J = 7.5 Hz, 2H), 1.42 (d, J = |
| | | 7.0 Hz, 1H), 1.74-1.84 (m, 2H), 1.91-2.05 (m, 2H), 2.10-2.21 (m, 1H), 2.29-2.39 |
| | | (m, 2H), 2.40-2.48 (m, 1H), 2.54-2.76 (m, 2H), 3.32-3.37 (m, 1H), |
| | | 3.43-3.51 (m, 1H), 3.72 (s, 1H), 3.73 (s, 2H), 3.75-4.08 (m, 6H), 4.26-4.41 |
| | | (m, 2H), 4.61-4.75 (m, 4H), 5.46-5.66 (m, 1H), 7.16-7.24 (m, 2H), 7.25-7.42 |
| | | (m, 6H), 7.85-7.91 (m, 1H), 9.10 (s, 1H). ³¹P NMR (203 MHz, |
| | | CD₃OD) δ ppm 6.56, 7.06. |
| 89 | 768.5 | ¹H NMR (500 MHz, CD₃OD) δ ppm 1.84-1.93 (m, 3H), 2.02-2.46 (m, 5H), |
| | | 2.56-2.82 (m, 2H), 3.11-3.15 (m, 2H), 3.34-3.36 (m, 1H), 3.44-3.48 (m, |
| | | 1H), 3.67-3.98 (m, 5H), 4.49-4.52 (m, 1H), 4.68-4.76 (m, 7H), 5.52-5.63 |
| | | (m, 1H), 7.14 (s, 1H), 7.22 (s, 1H), 7.34 (t, J = 10 Hz, 1H), 7.37 (s, 1H), 7.57 |
| | | (s, 1H), 7.88 (q, J = 10 Hz, 1H), 9.09(s, 1H). |
| 90 | 787.6 | ¹H NMR (500 MHz, CD₃OD) δ ppm 0.93 (d, J = 5 Hz, 3H), 0.97 (d, J = 5 Hz |
| | | 3H), 1.84-2.00 (m, 9H), 2.11-2.36 (m, 3H), 2.99-3.02 (m, 1H), 3.10 (d, |
| | | J = 10 Hz, 1H), 3.16-3.28 (m, 3H), 3.35 (d, J = 10 Hz, 1H), 3.52-3.57 (m, |
| | | 2H), 3.79 (s, 2H), 4.22-4.32 (m, 4H), 4.48-4.60 (m, 5H), 5.25-5.36 (m, 1H), |
| | | 7.21 (s, 1H), 7.32 (t, J = 10 Hz,1H), 7.35 (s, 1H), 7.84-7.87 (m, 1H), 9.02 (s, |
| | | 1H). |
| 91 | 700.5 | ¹H NMR (500 MHz, CD₃OD) δ ppm 9.15 (s, 1H), 8.17 (dd, J = 9.1, 5.7 Hz, |
| | | 1H), 8.04 (s, 1H), 7.57-7.53 (m, 2H), 5.60 (d, J = 51.7 Hz, 1H), 4.77-4.66 |
| | | (m, 2H), 4.38-4.22 (m, 3H), 4.12-3.83 (m, 5H), 3.54-3.43 (m, 2H), 2.81-2.50 |
| | | (m, 3H), 2.49-2.41 (m, 1H), 2.40-2.29 (m, 2H), 2.28-2.08 (m, 5H), |
| | | 1.28-1.22 (m, 6H). |
| 92 | 780.5 | ¹H NMR (500 MHz, CD₃OD) δ ppm 1.45 (d, J = 7.0 Hz, 3H), 1.74-1.82 (m, |
| | | 2H), 1.94-2.03 (m, 2H), 2.11-2.23 (m, 1H), 2.27-2.39 (m, 2H), 2.41-2.48 |
| | | (m, 1H), 2.53-2.75 (m, 2H), 3.32-3.36 (m, 1H), 3.43-3.52 (m, 1H), 3.70-3.78 |
| | | (m, 6H), 3.78-3.86 (m, 2H), 3.87-4.07 (m, 4H), 4.11-4.24 (m, 2H), |
| | | 4.61-4.75 (m, 4H), 5.52 (s, 0.5H), 5.63 (s, 0.5H), 7.22 (s, 1H), 7.34 (t, J = |
| | | 9.0 Hz, 1H), 7.37 (d, J = 2.0 Hz, 1H), 7.88 (dd, J = 5.5, 9.0 Hz, 1H), 9.10 (s, |
| | | 1H). ³¹P NMR (203 MHz, CD₃OD) δ ppm 12.37,12.65. |
| 93 | 825.5 | ¹H NMR (500 MHz, CD₃OD) δ ppm 1.88-2.23 (m, 5H), 2.26 (s, 3H), |
| | | 2.30-2.51 (m, 3H), 2.53-2.79 (m, 2H), 3.35-3.38 (m, 1H), 3.43-3.57 (m, 1H), |
| | | 3.81-4.14 (m, 5H), 4.61-4.81 (m, 6H), 5.07 (s, 1H), 5.52 (s, 0.5H), 5.63 (s, |
| | | 1.5H), 7.00-7.11 (m, 2H), 7.15-7.25 (m, 1H), 7.29-7.41 (m, 2H), 7.44-7.52 |
| | | (m, 2H), 7.84-7.94 (m, 1H), 9.11 (s, 1H). |
| 94 | 791.7 | ¹H NMR (500 MHz, CD₃OD) δ ppm 2.06-2.23 (m, 5H), 2.31-2.48 (m, 3H), |
| | | 2.56-2.76 (m, 2H), 3.33 (s, 3H), 3.42-3.46 (m, 2H), 3.47-3.54 (m, 3H), |
| | | 3.59-3.66 (m, 4H), 3.67-3.70 (m, 2H), 3.77-3.82 (m, 2H), 3.86-4.05 (m, |
| | | 5H), 4.24-4.32 (m, 2H), 4.40-4.46 (m, 2H), 4.65-4.78 (m, 2H), 4.89-4.93 |
| | | (m, 2H), 5.53 (s, 0.5H), 5.63 (s, 0.5H), 7.49-7.55 (m, 1H), 7.57 (s, 1H), 8.03 |
| | | (d, J = 2.0 Hz, 1H), 8.11-8.19 (m, 1H), 9.15 (s, 1H). |
| 95 | 821.7 | ¹H NMR (500 MHz, CD₃OD) δ ppm 1.04 (d, J = 5 Hz, 3H), 1.72-1.80 (m, |
| | | 2H), 2.10-2.18 (m, 5H), 2.34-2.44 (m, 3H), 2.57 (d, J = 5 Hz, 2H), 2.60-2.75 |
| | | (m, 2H), 3.44-3.50 (m, 2H), 3.86-4.04 (m, 5H), 4.27-4.29 (m, 2H), 4.69-4.75 |
| | | (m, 2H), 4.87 (s, 2H), 5.32 (s, 2H), 5.53-5.63 (m, 1H), 7.13 (d, J = 5 Hz, |
| | | 2H), 7.50-7.55 (m, 4H), 8.01 (s, 1H), 8.12 (q, J = 5 Hz, 1H), 9.13 (s, 1H). |
| 96 | 842.7 | ¹H NMR (500 MHz, CD₃OD) δ ppm 1.32 (d, J = 7.0 Hz, 3H), 2.05-2.24 (m, |
| | | 5H), 2.30-2.50 (m, 3H), 2.55-2.77 (m, 2H), 3.41-3.52 (m, 2H), 3.54-3.62 |
| | | (m, 3H), 3.69 (d, J = 5.5 Hz, 0.2H), 3.84-4.14 (m, 6.4H), 4.29 (s, 2H), |
| | | 4.66-4.76 (m, 2H), 4.89-4.92 (m, 1H), 5.53 (s, 0.5H), 5.64 (s, 0.5H), 7.18-7.31 |
| | | (m, 3H), 7.35-7.42 (m, 2H), 7.48-7.54 (m, 1H), 7.56-7.63 (m, 1H), 7.99 (s, |
| | | 0.6H), 8.05 (s, 0.4H), 8.07-8.14 (m, 1H), 9.14 (s, 1H). ³¹P NMR (203 MHz, |
| | | CD₃OD) δ ppm −0.99, −0.94, −0.83, −0.78. |
| 97 | 905.7 | ¹H NMR (500 MHz, CD₃OD) δ ppm 0.90 (t, J = 5 Hz, 3H), 1.29-1.32 (m, |
| | | 14H), 1.70-1.76 (m, 2H), 2.10-2.18 (m, 5H), 2.34-2.45 (m, 3H), 2.57-2.75 |
| | | (m, 4H), 3.44-3.51 (m, 2H), 3.86-4.03 (m, 5H), 4.27-4.29 (m, 2H), 4.69-4.75 |
| | | (m, 2H), 4.87 (s, 2H), 5.32 (s, 2H), 5.53-5.63 (m, 1H), 7.13 (d, J = 5 Hz, |
| | | 2H), 7.50-7.56 (m, 4H), 8.02 (s, 1H), 8.13 (q, J = 5 Hz, 1H), 9.13 (s, 1H). |
| 98 | 823.5 | ¹H NMR (500 MHz, CD₃OD) δ ppm 2.02 (s, 3H), 2.07-2.24 (m, 5H), |
| | | 2.28-2.50 (m, 3H), 2.51-2.78 (m, 2H), 2.99 (t, J = 6.0 Hz, 2H), 3.09 (t, J = 6.0 Hz, |
| | | 2H), 3.41-3.52 (m, 2H), 3.83-4.05 (m, 5H), 4.24-4.36 (m, 4H), 4.50-4.58 |
| | | (m, 2H), 4.66-4.76 (m, 2H), 5.53 (s, 0.5H), 5.63 (s, 0.5H), 7.52 (t, J = 9.0 |
| | | Hz, 1H), 7.57 (s, 1H), 8.03 (s, 1H), 8.09-8.18 (m, 1H), 9.14 (s, 1H). |
| 99 | 793.7 | ¹H NMR (500 MHz, CD₃OD) δ ppm 2.10-2.16 (m, 5H), 2.28 (s, 3H), |
| | | 2.35-2.45 (m, 3H), 2.60-2.72 (m, 2H), 3.44-3.48 (m, 2H), 3.86-4.03 (m, 5H), |
| | | 4.27-4.29 (m, 2H), 4.69-4.75 (m, 4H), 5.32 (s, 2H), 5.53-5.63 (m, 1H), 7.14 |
| | | (d, J = 5 Hz, 2H), 7.49-7.55(m, 4H), 8.02 (s, 1H), 8.13 (q, J = 5 Hz, 1H), 9.13 |
| | | (s, 1H). |
| 100 | 731.6 | ¹H NMR (500 MHz, CD₃OD) δ ppm 9.17 (s, 1H), 8.18 (dd, J = 9.1, 5.7 Hz, |
| | | 1H), 8.06 (d, J = 2.4 Hz, 1H), 7.59 (s, 1H), 7.56 (t, J = 8.9 Hz, 1H), 6.85 |
| | | (q, J = 5.4 Hz, 1H), 5.62 (d, J = 51.4 Hz, 1H), 4.79-4.70 (m, 2H), 4.32 (d, |
| | | J = 10.2 Hz, 2H), 4.11-3.87 (m, 5H), 3.57-3.44 (m, 2H), 2.82-2.54 (m, 2H), |
| | | 2.52-2.33 (m, 3H), 2.28-2.09 (m, 8H), 1.62 (d, J = 5.4 Hz, 3H). |
| 101 | 787.6 | ¹H NMR (500 MHz, CD₃OD) δ ppm 1.06 (t, J = 5 Hz, 7H), 2.10-2.20 (m, |
| | | 7H), 2.34-2.47 (m, 3H), 2.57-2.77 (m, 2H), 3.46-3.55 (m, 3H), 3.65 (d, J = 5 |
| | | Hz, 1H), 3.75-3.78 (m, 1H), 3.85-4.06 (m, 5H), 4.29 (s, 1H), 4.34-4.42 (m, |
| | | 2H), 4.72 (s, 2H), 5.53-5.64 (m, 1H), 7.52 (t, J = 10 Hz, 1H), 7.57 (s, 1H), |
| | | 8.01 (s, 1H), 8.12 (q, J = 5 Hz, 1H), 9.13 (s, 1H). |
| 102 | 835.5 | ¹H NMR (500 MHz, CD₃OD) δ ppm 1.06 (d, J = 10 Hz, 7H), 2.10-2.21 (m, |
| | | 7H), 2.34-2.47 (m, 5H), 2.57-2.75 (m, 2H), 3.44-3.50 (m, 2H), 3.85-4.03 |
| | | (m, 5H), 3.44-3.51 (m, 2H), 3.86-4.03 (m, 5H), 4.27-4.29 (m, 2H), 4.72 (s, |
| | | 2H), 5.32 (s, 2H), 5.53-5.63 (m, 1H), 7.13 (d, J = 5 Hz, 2H), 7.50-7.55 (m, |
| | | 4H), 8.02 (s, 1H), 8.13 (q, J = 5 Hz, 1H), 9.13 (s, 1H). |
| 103 | 795.6 | ¹H NMR (500 MHz, CD₃OD) δ ppm 9.16 (s, 1H), 8.13 (dd, J = 9.0, 5.8 Hz, |
| | | 1H), 7.95 (d, J = 2.1 Hz, 1H), 7.61-7.49 (m, 2H), 5.62 (d, J = 51.9 Hz, 1H), |
| | | 4.92-4.84 (m, 2H), 4.75 (s, 2H), 4.60 (d, J = 12.1 Hz, 1H), 4.41 (d, J = 12.6 |
| | | Hz, 1H), 4.31 (s, 2H), 4.14-3.85 (m, 5H), 3.63-3.43 (m, 5H), 3.20 (s, 1H), |
| | | 3.16-2.96 (m, 3H), 2.83-2.56 (m, 2H), 2.52-2.32 (m, 3H), 2.30-2.10 (m, |
| | | 7H), 2.08-1.97 (m, 2H), 1.95-1.73 (m, 5H), 1.65-1.48 (m, 1H). |
| 104 | 807.5. | ¹H NMR (500 MHz, CD₃OD) δ ppm 1.74-1.90 (m, 2H), 1.96-2.08 (m, 2H), |
| | | 2.10-2.22 (m, 1H), 2.28-2.51 (m, 3H), 2.54-2.80 (m, 2H), 3.07-3.20 (m, |
| | | 1H), 3.35-3.59 (m, 7H), 3.65-4.10 (m, 6H), 4.51 (s, 2H), 4.60-4.82 (m, 3H), |
| | | 5.10 (s, 1H), 5.30-5.68 (m, 1H), 7.21 (s, 1H), 7.29-7.42 (m, 2H), 7.82-7.93 |
| | | (m, 1H), 9.06-9.17 (m, 1H). |
| 105 | 843.6 | ¹H NMR (500 MHz, CD₃OD) δ ppm 1.37 (t, J = 7.0 Hz, 3H), 1.78-2.22(m, |
| | | 5H), 2.30-2.48 (m, 3H), 2.53-2.77 (m, 2H), 3.33-3.38 (m, 1H), 3.43-3.51 |
| | | (m, 1H), 3.80-4.07 (m, 8H), 4.21-4.29 (m, 2H), 4.30-4.39 (m, 2H), 4.60-4.78 |
| | | (m, 4H), 5.53 (s, 0.5H), 5.63 (s, 0.5H), 7.22 (d, J = 2.0 Hz, 1H), |
| | | 7.29-7.41 (m, 3H), 7.50-7.56 (m, 1H), 7.58-7.65 (m, 1H), 7.85-7.93 (m, 2H), |
| | | 9.12 (s, 1H). ³¹P NMR (203 MHz, CD₃OD) δ ppm −0.66. |
| 106 | 1112.4 | ¹H NMR (500 MHz, CD₃OD) δ ppm 1.83-1.94 (m, 3H), 1.99-2.06 (m, |
| | | 11H), 2.11-2.35 (m, 4H), 3.01-3.02 (m, 1H), 3.20-3.28 (m, 3H), 3.35-3.36 |
| | | (m, 2H), 3.71 (s, 3H), 3.74-3.76 (m, 2H), 4.21-4.31 (m, 2H), 4.50-4.64 (m, |
| | | 5H), 5.21-5.24 (m, 5H), 5.43 (t, J = 10 Hz, 1H), 5.51 (d, J = 10 Hz, 1H), 7.20 |
| | | (s, 1H), 7.30-7.34 (m, 2H), 7.47 (d, J = 5 Hz, 1H), 7.69 (d, J = 5 Hz, 1H), |
| | | 7.84-7.87( m,1H), 7.90 (s, 1H), 9.01 (s, 1H). |
| 107 | 715.4 | ¹H NMR (500 MHz, CD₃OD) δ ppm 9.04 (s, 1H), 7.88 (dd, J = 8.3, 5.8 Hz, |
| | | 1H), 7.44-7.30 (m, 2H), 7.23 (s, 1H), 5.33 (d, J = 53.4 Hz, 1H), 4.77-4.61 |
| | | (m, 2H), 4.57-4.44 (m, 2H), 4.38-4.23 (m, 2H), 4.19 (t, J = 6.4 Hz, 2H), |
| | | 3.90-3.70 (m, 2H), 3.39 (d, J = 8.1 Hz, 1H), 3.32-3.16 (m, 3H), 3.12-2.96 |
| | | (m, 1H), 2.43-2.11 (m, 3H), 2.10-1.81 (m, 7H), 1.79-1.67 (m, 2H), |
| | | 1.51-1.37 (m, 4H), 0.98 (t, J = 6.5 Hz, 3H). |
| 108 | 843.3 | ¹H NMR (500 MHz, CD₃OD) δ ppm 1.39 (t, J = 7.0 Hz, 3H), 2.05-2.26 (m, |
| | | 5H), 2.30-2.50 (m, 3H), 2.55-2.78 (m, 2H), 3.43-3.53 (m, 2H), 3.74-3.82 |
| | | (m, 3H), 3.84-4.07 (m, 5H), 4.22-4.34 (m, 2H), 4.42-4.51 (m, 2H), 4.65-4.77 |
| | | (m, 2H), 5.53 (s, 0.5H), 5.64 (s, 0.5H), 7.36 (t, J = 7.5 Hz, 1H), |
| | | 7.40-7.45 (m, 1H), 7.50-7.56 (m, 1H), 7.57-7.65 (m, 2H), 7.91 (d, J = 8.0 Hz, |
| | | 1H), 8.04 (s, 1H), 8.10-8.17 (m, 1H), 9.14 (s, 1H). ³¹P NMR (203 MHz, |
| | | CD₃OD) δ ppm −12.75. |
| 109 | 799.4 | ¹H NMR (500 MHz, CD₃OD) δ ppm 1.37 (t, J = 7.0 Hz, 3H), 1.67-2.08 (m, |
| | | 4H), 2.11-2.48 (m, 4H), 2.54-2.76 (m, 2H), 3.34-3.38 (m, 1H), 3.42-3.51 |
| | | (m, 1H), 3.56-3.65 (m, 1H), 3.68-3.86 (m, 1H), 3.86-3.99 (m, 2H), 4.00-4.30 |
| | | (m, 5H), 4.56-4.75 (m, 4H), 5.12 (d, J = 9.0 Hz, 1.7H), 5.26 (d, J = 9.0 |
| | | Hz, 0.2H), 5.52 (s, 0.5H), 5.62 (s, 0.5H), 7.22 (s, 1H), 7.31-7.50 (m, 7H), |
| | | 7.83-7.93 (m, 1H), 9.04 (s, 1H). ³¹P NMR (203 MHz, CD₃OD) δ ppm 5.01. |
| 110 | 809.4 | ¹H NMR (500 MHz, CD₃OD) δ ppm 1.27 (t, J = 7.0 Hz, 3H), 1.37 (t, J = 7.0 |
| | | Hz, 3H), 1.73-1.86 (m, 2H), 1.96-2.07 (m, 2H), 2.11-2.21 (m, 1H), |
| | | 2.28-2.48 (m, 3H), 2.55-2.79 (m, 4H), 3.34-3.38 (m, 1H), 3.42-3.52 (m, 1H), |
| | | 3.74-4.08 (m, 5H), 4.10-4.22 (m, 6H), 4.26-4.40 (m, 2H), 4.60-4.77 (m, |
| | | 4H), 5.53 (s, 0.5H), 5.63 (s, 0.5H), 7.21 (d, J = 2.0 Hz, 1H), 7.30-7.41 (m, |
| | | 2H), 7.84-7.93 (m, 1H), 9.11 (s, 1H). ³¹P NMR (203 MHz, CD₃OD) δ ppm |
| | | 4.72. |
| 111 | 791.5 | ¹H NMR (500 MHz, CD₃OD) δ ppm 1.84-1.91 (m, 3H), 1.96-2.01 (m, 4H), |
| | | 2.12-2.36 (m, 3H), 2.99-3.02 (m, 1H), 3.16-3.37 (m, 4H), 3.51-3.53 (m, |
| | | 2H), 3.62-3.67 (m, 7H), 3.75-3.80 (m, 4H), 4.23-4.33 (m, 4H), 4.49-4.61 |
| | | (m, 6H), 5.25-5.36 (m, 1H), 7.21 (s, 1H), 7.31-7.35 (m,2H), 7.85-7.88 (m, |
| | | 1H), 9.03 (s, 1H). |
| 112 | 672.3 | ¹H NMR (500 MHz, CD₃OD) δ ppm 2.12-2.20 (m, 6H), 2.38-2.47 (m, |
| | | 3H), 2.66-2.76 (m, 2H), 3.05 (s, 3H),3.20 (s, 3H), 3.44-3.51 (m, 2H), |
| | | 3.92-4.04 (m, 5H), 4.33 (s, 2H), 4.71-4.75(m, 3H), 5.56-5.65(m, 1H), 7.52(s, |
| | | 2H), 7.93 (s, 1H), 8.12-8.15 (m, 1H), 9.17(s, 1H). |
| 113 | 813.4 | ¹H NMR (500 MHz, CD₃OD) δ ppm 1.34 (t, J = 7.0 Hz, 3H), 1.69-1.78 (m, |
| | | 2H), 1.81-1.97 (m, 3H), 2.11-2.11 (m, 3H), 2.14-2.43 (m, 2H), 3.03 (t, J = |
| | | 6.0 Hz, 2H), 3.33-3.38 (m, 2H), 3.43-3.68 (m, 3H), 3.87-4.12 (m, 5H), |
| | | 4.23-4.65 (m, 6H), 5.31 (s, 0.5H), 5.42 (s, 0.5H), 7.23-7.39 (m, 8H), 7.85-7.90 |
| | | (m, 1H), 8.95 (s, 1H). ³¹P NMR (203 MHz, CD₃OD) δ ppm 4.85. |
| 114 | 827.4 | ¹H NMR (500 MHz, CD₃OD) δ ppm 1.41 (t, J = 7.0 Hz, 3H), 1.79-1.89 (m, |
| | | 2H), 1.96-2.12 (m, 4H), 2.14-2.25 (m, 1H), 2.30-2.50 (m, 3H), 2.56-2.75 |
| | | (m, 2H), 2.76-2.82 (m, 2H), 3.38 (s, 1H), 3.45-3.54 (m, 1H), 3.68-4.24 (m, |
| | | 11H), 4.60-4.81 (m, 4H), 5.55 (s, 0.5H), 5.66 (s, 0.5H), 6.97-7.52 (m, 8H), |
| | | 7.86-7.97 (m, 1H), 9.13 (s, 1H). ³¹P NMR (203 MHz, CD₃OD) δ ppm 5.09. |
| 115 | 700.41 | ¹H NMR (500 MHz, CD₃OD) δ ppm 1.01-1.12 (m, 6H), 1.92-2.37 (m, |
| | | 12H), 2.99-3.04 (m, 1H), 3.24-3.38 (m, 3H), 3.66-3.90 (m, 3H), 4.28-4.33 |
| | | (m, 2H), 4.59-4.81 (m, 4H), 5.27-5.37 (m, 1H), 7.21 (s, 1H), 7.31-7.35 (m, |
| | | 2H), 7.85-7.37 (m, 1H), 9.01 (s, 1H). |
| 116 | 927.3 | ¹H NMR (500 MHz, CD₃OD) δ ppm 0.89 (t, J = 7.0 Hz, 3H), 1.21-1.43 (m, |
| | | 12H), 1.63-1.72 (m, 2H), 1.80-1.90 (m, 2H), 2.00-2.22 (m, 2H), 2.28-2.68 |
| | | (m, 4H), 3.35-3.38 (m, 1H), 3.41-3.58 (m, 2H), 3.78-4.08 (m, 7H), 4.13-4.23 |
| | | (m, 2H), 4.29-4.41 (m, 2H), 4.58-4.79 (m, 4H), 5.53 (s, 0.4H), 5.63 (s, |
| | | 0.6H), 7.21 (d, J = 2.5 Hz, 1H), 7.29-7.41 (m, 3H), 7.49-7.56 (m, 1H), |
| | | 7.58-7.66 (m, 1H), 7.82-7.96 (m, 2H), 9.12 (s, 1H). ³¹P NMR (203 MHz, |
| | | CD₃OD) δ ppm −0.55. |
| 117 | 757.33 | ¹H NMR (500 MHz, CD₃OD) δ ppm 1.89 (d, J = 8.0 Hz, 2H), 1.93-2.13 (m, |
| | | 6H), 2.20-2.46 (m, 5H), 3.11-3.17 (m, 1H), 3.37-3.51 (m, 3H), 3.76-3.88 |
| | | (m, 2H), 4.28-4.78 (m, 6H), 5.06 (s, 2H), 5.33 (s, 0.5H), 5.44 (s, 0.5H), 7.24 |
| | | (s, 1H), 7.34-7.43 (m, 2H), 7.86-7.94 (m, 1H), 9.07 (s, 1H). |
| 118 | 714.5 | ¹H NMR (500 MHz, CD₃OD) δ ppm 9.46-9.45 (m, 1H), 8.43-8.40 (m, 1H), |
| | | 8.24 (s, 1H), 7.94-7.76 (m, 2H), 5.96-5.85 (m, 1H), 5.24-5.17 (m, 3H), |
| | | 5.03-5.01 (m, 2H), 4.59 (d, J = 13.0 Hz, 2H), 4.41-4.14 (m, 6H), 3.93-3.73 |
| | | (m, 5H), 3.63 (s, 2H), 3.11-2.86 (m, 2H), 2.82-2.61 (m, 4H), 2.53-2.40 (m, |
| | | 6H). |
| 119 | 700.3 | ¹H NMR (500 MHz, DMSO-d6) δ 9.13 (s, 1H), 8.24-8.21 (m, 1H), 7.99 (s, |
| | | 2H), 7.65 t, J = 9.0 Hz, 1H), 7.47 (s, 1H), 5.37-5.26 (m, 1H), 4.64 (d, J = |
| | | 13.0 Hz, 1H), 4.48 (d, J = 13.0 Hz, 1H), 4.22-4.05 (m, 6H), 3.87-3.75 (m, |
| | | 2H), 3.19-3.07 (m, 5H), 2.16-2.04 (m, 3H), 1.94 (s, 5H), 1.82 (m, 2H), |
| | | 1.54-1.45 (m, 2H), 1.40-1.30 (m, 2H), 0.91 (t, J = 7.0 Hz, 3H). |
| 120 | 927.6 | ¹H NMR (500 MHz, CD₃OD) δ ppm 0.84 (t, J = 7.0 Hz, 3H), 1.09-1.35 (m, |
| | | 12H), 1.64-1.73 (m, 2H), 2.03-2.24 (m, 5H), 2.30-2.48 (m, 3H), 2.54-2.78 |
| | | (m, 2H), 3.43-3.53 (m, 2H), 3.76-3.82 (m, 3H), 3.83-4.07 (m, 5H), 4.25-4.31 |
| | | (m, 2H), 4.35-4.45 (m, 2H), 4.68-4.77 (m, 2H), 5.53 (s, 0.5H), 5.64 (s, |
| | | 0.5H), 7.36 (t, J = 7.5 Hz, 1H), 7.41-7.47 (m, 1H), 7.53 (t, J = 9.0 Hz, 1H), |
| | | 7.57-7.64 (m, 2H), 7.92 (d, J = 7.5 Hz, 1H), 8.04 (s, 1H), 8.10-8.15 (m, 1H), |
| | | 9.14 (s, 1H). ³¹P NMR (203 MHz, CD₃OD) δ ppm −12.66. |
| 121 | 997.6 | ¹H NMR (500 MHz, CD₃OD) δ ppm 9.05 (s, 1H), 7.89 (dd, J = 8.3, 5.9 Hz, |
| | | 1H), 7.42-7.31 (m, 2H), 7.23 (s, 1H), 5.43-5.21 (m, 2H), 4.72-4.59 (m, 2H), |
| | | 4.57-4.39 (m, 4H), 4.38-4.23 (m, 4H), 3.88-3.75 (m, 2H), 3.38 (d, J = 6.4 |
| | | Hz, 1H), 3.30-3.23 (m, 2H), 3.12-3.02 (m, 1H), 2.48-2.12 (m, 7H), 2.11-1.98 |
| | | (m, 4H), 1.98-1.83 (m, 3H), 1.71-1.54 (m, 4H), 1.44-1.16 (m, 20H), |
| | | 1.01-0.81 (m, 6H). |
| 122 | 1083.6 | ¹H NMR (500 MHz, CD₃OD) δ ppm 0.86-0.90 (m, 6H), 1.17 (d, J = 6.5 Hz, |
| | | 2H), 1.26 (s, 24H) 1.57-1.60 (m, 3H), 2.13-2.25 (m, 5H), 2.36-2.43 (m, |
| | | 6H), 2.50-2.52 (m, 2H), 2.56-2.67 (m, 4H), 2.76-2.81(m, 2H) 3.48-3.50 (m, |
| | | 2H), 3.89-4.09 (m, 4H), 4.18-4.21 (m, 2H), 4.31-4.33 (m, 2H), 4.38-4.41 |
| | | (m, 2H), 4.75 (s, 2H), 5.32-5.33 (m, 1H), 5.56-5.67 (m, 1H), 7.51-7.55 (m, |
| | | 2H), 7.95 (s, 1H), 8.14-8.17 (m, 1H), 9.17 (s, 1H). |
| 123 | 997.5 | ¹H NMR (500 MHz, CD₃OD) δ ppm 0.87 (s, 6H), 1.22-1.31 (m 20H), 1.50 |
| | | (s, 3H), 1.62 (s, 4H), 2.02-2.84 (m, 14H), 3.50 (s, 2H), 4.01 (m, 5H), 4.32 |
| | | (s, 2H), 4.46 (s, 4H), 4.75 (s, 4H), 5.62 (d, J = 52.0 Hz, 1H), 7.50-7.54 (m, |
| | | 2H), 7.96 (s, 1H), 8.15 (s, 1H), 9.17 (s, 1H). |
| 124 | 645.2 | ¹H NMR (500 MHz, CD₃OD) δ ppm 1.73-2.05 (m, 7H), 2.08-2.39 (m, 3H), |
| | | 2.97-3.06 (m, 1H), 3.14-3.29 (m, 3H), 3.52 (s, 3H), 3.62-3.80 (m, 4H), |
| | | 4.15-4.36 (m, 2H), 4.56-4.70 (m, 2H), 5.26 (s, 0.5H), 5.37 (s, 2.5H), |
| | | 7.34-7.47 (m, 2H), 7.68 (d, J = 2.5 Hz, 1H), 7.94-8.04 (m, 1H), 9.02 (s, 1H). |
| 125 | 889.2 | ¹H NMR (500 MHz, CD₃OD) δ ppm 9.08 (d, J = 3.8 Hz, 1H), 7.90 (dd, |
| | | J = 8.6, 5.9 Hz, 1H), 7.44-7.27 (m, 7H), 7.24 (s, 1H), 5.35 (d, J = 54.0 Hz, |
| | | 1H), 5.17-5.09 (m, 2H), 4.91-4.58 (m, 5H), 4.41-4.23 (m, 3H), 4.13 (t, J = |
| | | 17.0 Hz, 1H), 4.02-3.91 (m, 1H), 3.91-3.77 (m, 1H), 3.77-3.60 (m, 2H), |
| | | 3.31-3.16 (m, 3H), 3.12-2.98 (m, 1H), 2.43-1.83 (m, 14H), 1.43-1.26 (m, |
| | | 1H). |
| 126 | 749.3 | ¹H NMR (500 MHz, CD₃OD) δ ppm 2.06-2.22(m, 5H), 2.28 (s, 3H), |
| | | 2.31-2.79 (m, 5H), 3.37 (d, J = 8.0 Hz, 1H), 3.43-3.53 (m, 1H), 3.83-4.07 (m, |
| | | 5H), 4.23-4.33 (m, 2H), 4.66-4.76 (m, 2H), 4.82-4.87 (m, 1H), 5.29 (s, 2H), |
| | | 5.53 (s, 0.5H), 5.63 (s, 0.5H), 7.14 (d, J = 8.5 Hz, 2H), 7.34-7.44 (m, 2H), |
| | | 7.55 (d, J = 8.5 Hz, 2H), 7.66 (d, J = 2.5 Hz, 1H), 7.99-8.06 (m, 1H), 9.12 |
| | | (s, 1H). |
| 127 | 1069.6 | ¹H NMR (500 MHz, CD₃OD) δ ppm 9.17 (s, 1H), 8.15 (dd, J = 8.9, 5.7 Hz, |
| | | 1H), 7.96 (s, 1H), 7.54 (d, J = 13.6 Hz, 2H), 5.62 (d, J = 51.7 Hz, 1H), |
| | | 5.39-5.28 (m, 1H), 4.75 (s, 2H), 4.41 (d, J = 11.5 Hz, 2H), 4.37-4.28 (m, 2H), |
| | | 4.21 (dd, J = 12.0, 6.1 Hz, 2H), 4.13-3.88 (m, 5H), 3.58-3.45 (m, 2H), 2.79 |
| | | (t, J = 6.9 Hz, 2H), 2.77-2.59 (m, 2H), 2.55 (t, J = 7.0 Hz, 2H), 2.52-2.44 |
| | | (m, 1H), 2.43-2.30 (m, 6H), 2.29-2.01 (m, 7H), 1.70-1.51 (m, 4H), |
| | | 1.35-1.22 (m, 20H), 0.89 (t, J = 6.8 Hz, 6H). |
| 128 | 1097.6 | ¹H NMR (500 MHz, CD₃OD D) δ ppm 0.85 (t, J = 6.9 Hz, 6H), 1.24-1.32 |
| | | (m, 18H), 1.53 (s, 4H), 1.71 (d, J = 13.7 Hz, 4H), 1.85 (d, J = 9.4 Hz, 4H), |
| | | 2.02-2.19 (m, 6H), 2.27 (t, J = 7.3 Hz, 4H), 2.40 (m, 3H), 2.55-2.66 (m, |
| | | 3H), 2.79 (s, 2H), 3.44-3.53 (m, 4H), 3.87-3.96 (m, 5H), 4.17 (dd, J = 11.8, |
| | | 6.3 Hz, 2H), 4.25-4.40 (m, 4H), 4.67-4.77 (m, 2H), 5.29-5.38 (m, 1H), 5.58 |
| | | (d, J = 51.6 Hz, 1H), 7.51 (t, J = 11.7 Hz, 2H), 7.92 (s, 1H), 8.14 (dd, J = |
| | | 8.8, 5.8 Hz, 1H), 9.15 (s, 1H). |
| 129 | 1083.6 | ¹H NMR (500 MHz, CD₃OD) δ ppm 0.91-0.93 (t, J = 5.0 Hz, 6H), |
| | | 1.11-1.15 (m, 3H), 1.33 (s, 20H), 1.63-1.65 (m, 5H), 2.39-2.82 (m, 15H), |
| | | 3.93-4.07 (m, 4H), 4.21-4.41 (m, 4H), 4.71-4.89 (m, 3H), 5.31 (s, 1H), |
| | | 5.57-5.67 (m, 1H), 7.37 (s, 1H), 7.41-7.44 (t, J = 9.0 Hz, 1H), 7.49 (s, 1H), |
| | | 7.95-7.98 (m, 1H), 9.29 (s, 1H). |
| 130 | 783.4 | ¹H NMR (500 MHz, CD₃OD) δ ppm 0.97-0.99 (t, J = 5.0 Hz, 3H), 1.32 |
| | | (s, 2H), 1.43-1.47 (m, 2H), 1.68-1.70 (m, 2H), 1.86 (s, 1H), 1.94-1.95 (m, |
| | | 2H), 2.02 (s, 3H), 2.06 (m, 4H), 2.11 (s, 3H), 2.18-2.42 (m, 3H), 3.09 (s, |
| | | 1H), 3.30-3.41 (m, 2H), 3.87-3.90 (m, 2H), 4.16-4.23 (m, 3H), 4.30-4.40 |
| | | (m, 2H), 4.60-4.66 (m, 1H), 4.88 (s, 2H), 5.32-5.42 (m, 1H), 7.23-7.25 (d, |
| | | J = 5.0 Hz, 1H), 7.34-7.39 (m, 2H), 7.89-7.91 (m, 1H), 9.05 (s, 1H). |
| 131 | 1069.5 | ¹H NMR (500 MHz, CD₃OD) δ ppm 9.08 (s, 1H), 7.90 (dd, J = 9.1, 5.7 Hz, |
| | | 1H), 7.39 (d, J = 2.4 Hz, 1H), 7.36 (t, J = 9.0 Hz, 1H), 7.24 (s, 1H), 5.39 |
| | | (d, J = 53.7 Hz, 1H), 5.32-5.26 (m, 1H), 4.89-4.64 (m, 4H), 4.64-4.56 (m, |
| | | 1H), 4.51-4.31 (m, 4H), 4.26-4.18 (m, 2H), 4.01-3.71 (m, 2H), 3.52-3.36 |
| | | (m, 3H), 3.19-3.07 (m, 1H), 2.60 (t, J = 7.1 Hz, 2H), 2.51 (t, J = 6.7 Hz, |
| | | 2H), 2.48-2.19 (m, 7H), 2.18-1.84 (m, 8H), 1.69-1.58 (m, 4H), 1.37-1.30 |
| | | (m, 20H), 0.92 (t, J = 6.1 Hz, 6H). |
| 132 | 1097.6 | ¹H NMR (500 MHz, CD₃OD) δ ppm 0.92 (d, J = 6.0 Hz, 6H), 1.23 (s, 6H), |
| | | 1.33 (s, 20H), 1.63 (s, 4H), 1.99-2.13 (m, 4H), 2.31-2.34 (m, 6H), 2.50-2.86 |
| | | (m, 6H), 3.49 (s, 1H), 3.61-3.79 (m, 4H), 3.84-4.08 (m, 4H), 4.23 (s, 2H), |
| | | 4.39 (d, J = 9.5 Hz, 2H), 4.72 (s, 2H), 4.90 (s, 2H), 5.31 (s, 1H), 5.64 (d, |
| | | J = 51.7 Hz, 1H), 7.45 (d, J = 8.1 Hz, 2H), 7.52 (s, 1H), 7.99 (s, 1H), 9.36 (s, |
| | | 1H). |
| 133 | 783.2 | ¹H NMR (500 MHz, CD₃OD) δ ppm 1.18-1.23 (m, 1H), 1.33 (m, 2H), 1.48 |
| | | (s, 9H), 1.90-1.96 (m, 2H), 2.03-2.07 (m, 2H), 2.12-2.20 (m, 2H), 2.38-2.47 |
| | | (m, 3H), 2.63-2.75 (m, 2H), 3.48-3.52 (m, 2H), 3.62-3.66 (m, 1H), |
| | | 3.86-4.17 (m, 4H), 4.68-4.95 (m, 4H), 6.86-6.89 (m, 1H), 7.45 (s, 1H), |
| | | 7.52-7.59 (m, 2H) 7.92-7.97 (m, 1H), 8.14-8.17 (m, 1H), 9.16 (s, 1H). |
| 134 | 757.3 | ¹H NMR (500 MHz, CD₃OD) δ ppm 1.10-1.13 (m, 3H), 1.28-1.31 (m, 3H), |
| | | 1.32-1.35 (m, 1H), 1.94-2.02 (m, 2H), 2.15 (s, 1H), 2.27-2.50 (m, 8H), |
| | | 2.59-2.80 (m, 3H), 3.48-3.53 (m, 1H), 3.69-3.77 (m, 2H), 3.93-4.21 (m, |
| | | 6H), 4.70-4.95 (m, 3H), 5.58-5.69 (m, 1H), 7.41 (s, 1H), 7.93 (s, 1H), |
| | | 7.42-7.46 (t, J = 10.0 HZ, 1H), 7.51(s, 1H), 7.97-8.00 (m, 1H), 9.32 (s, 1H). |
| 135 | 757.3 | ¹H NMR (500 MHz, CD₃OD) δ ppm 1.15-1.16 (d, J = 6.5 Hz, 3H), |
| | | 1.26-1.29 (t, J = 7.0 Hz, 3H), 2.13-2.18 (m, 5H), 2.37-2.42 (m, 3H), 2.48-2.67 (m, |
| | | 6H), 2.76-2.80 (m, 2H), 3.48-3.50 (m, 2H), 3.90-4.04 (m, 5H), 4.14-4.18 |
| | | (m, 2H), 4.33 (s, 2H), 4.73-4.78 (m, 2H), 5.56-5.66 (m, 1H), 7.51-7.55 (m, |
| | | 2H), 7.95-7.96 (d, J = 2.5 Hz, 1H), 8.14-8.17 (m, 1H), 9.16 (s, 1H). |
| 136 | 771.4 | ¹H NMR (500 MHz, CD₃OD) δ ppm 1.15-1.16 (d, J = 6.0 Hz, 3H), |
| | | 1.25-1.27 (m, 6H), 2.13-2.18 (m, 5H), 2.34-2.52 (m, 5H), 2.56-2.67 (m, 4H), |
| | | 2.76-2.80 (m, 2H), 3.48-3.50 (m, 2H), 3.90-4.04 (m, 5H), 3.82-4.33 (s, 2H), |
| | | 4.73-4.78 (m, 2H), 4.90-5.04 (m, 4H), 5.56-5.66 (m, 1H), 7.51-7.55 (m, |
| | | 2H), 7.95-7.96 (d, J = 2.5 Hz, 1H), 8.14-8.17 (m, 1H), 9.17 (s, 1H). |
| 137 | 787.3 | ¹H NMR (500 MHz, CD₃OD) δ 1.55 (s, 3H), 1.85 (s, 2H), 2.01 (s, 3H), |
| | | 2.20 (m, 5H), 2.32-2.54 (m, 2H), 3.20 (s, 2H), 3.35-3.66 (m, 4H), 3.70-4.01 |
| | | (m, 6H), 4.35-4.55 (m, 4H), 4.69 (m, 2H), 5.42 (m, 1H), 6.86 (m, 1H), 7.21 |
| | | (s, 1H), 7.34 (m, 2H), 7.87 (dd, J = 9.0, 5.5 Hz, 1H), 9.05 (s, 1H). |
| 138 | 815.2. | ¹H NMR (500 MHz, CD₃OD) δ 1.36-1.40 (m, 3H), 1.57 (s, 3H), 1.71 (d, J = |
| | | 10 Hz, 2H), 1.87-1.97 (m, 3H), 2.01-2.10 (m, 5H), 2.15-2.23 (m, 1H), |
| | | 2.24-2.41 (m, 4H), 3.01-3.04 (m, 1H), 3.21-3.30 (m, 3H), 3.41-3.52 (m, |
| | | 3H), 3.71-3.83 (m, 2H), 3.94 (s, 2H), 4.25-4.37 (m, 2H), 4.46-4.54 (m, 2H), |
| | | 4.62-4.75(m, 2H), 5.32-5.40 (m, 1H), 6.87-6.93 (m, 1H), 7.24 (s, 1H), |
| | | 7.35-7.39 (m, 2H), 7.87-7.93 (m, 1H), 9.05 (s, 1H). |
| 139 | 801.4 | ¹H NMR (500 MHz, CD₃OD) δ ppm 1.28-1.30 (m, 2H), 1.54 (s, 3H), |
| | | 1.72-1.74 (m, 2H), 1.86-1.93 (m, 5H), 2.02-2.03 (m, 4H), 2.15-2.39 (m, 3H), |
| | | 2.64 (s, 1H), 3.04-3.07 (m, 1H), 3.26-3.28 (m, 1H), 3.36-3.38 (m, 1H), 3.46 |
| | | (d, J = 10.5 Hz, 2H), 3.71-3.78 (m, 2H), 3.90-3.92 (m, 2H), 4.26-4.36 (m, |
| | | 2H), 4.47-4.49 (m, 2H), 4.64-4.74 (m, 2H), 5.28 (s, 0.5H), 5.39 (s, 0.5H), |
| | | 6.86 (q, J = 5.5 Hz, 1H), 7.20 (s, 1H), 7.31-7.35 (m, 2H), 6.86 (dd, J = 6.0 Hz, |
| | | 1H), 9.02 (s, 1H) |
| 140 | 805.3 | ¹H NMR (500 MHz, CD₃OD) δ 1.59 (s, 3H), 1.81-2.13 (m, 7H), 2.15-2.47 |
| | | (m, 3H), 3.05-3.18 (m, 1H), 3.35-3.42 (m, 5H), 3.59 (s, 2H), 3.68-3.90 (m, |
| | | 4H), 4.23 (d, J = 17.6 Hz, 2H), 4.29-4.43 (m, 2H), 4.50 (s, 2H), 4.60-4.83 |
| | | (m, 2H), 5.32 (s, 0.5H), 5.43 (s, 0.5H), 6.95 (d, J = 5.3 Hz, 1H), 7.23 (s, |
| | | 1H), 7.32-7.41 (m, 2H), 7.89 (dd, J = 9.0, 5.7 Hz, 1H), 8 9.06 (s, 1H). |
| 141 | 773.4 | ¹H NMR (500 MHz, CD₃OD) δ ppm 0.99-1.05 (m, 6H), 1.33 (s, 1H), 1.69 |
| | | (s, 2H), 1.86-1.90 (m, 4H), 2.06-2.23 (m, 3H), 2.38-2.50 (m, 5H), 2.59-2.78 |
| | | (m, 2H), 3.39-3.41 (m, 1H), 3.48-3.53 (m, 1H), 3.77-4.09 (m, 5H), |
| | | 4.52-4.55 (m, 2H), 4.68-4.78 (m, 4H), 5.56-5.66 (m,1H), 6.76-6.77 (m, 1H), |
| | | 7.25-7.26 (m, 1H), 7.36-7.41 (m,2H), 7.90-7.93 (m, 1H), 9.15 (s, 1H). |
| 143 | 799.4 | ¹H NMR (500 MHz, CD₃OD) δ ppm 0.99-1.01 (m, 3H), 1.33 (s, 1H), 1.69 |
| | | (s, 2H), 1.86-1.95 (m, 3H), 2.06-2.09 (m, 7H), 2.20-2.24 (m, 1H), 2.37-2.50 |
| | | (m, 5H), 2.60-2.84 (m, 3H), 3.38-3.40 (m, 1H), 3.48-3.53 (m, 1H), |
| | | 3.83-4.09 (m, 5H), 4.52-4.53 (m, 2H), 4.67-4.78 (m, 4H), 5.56-5.67 (m,1H), |
| | | 6.81-6.82 (m, 1H), 7.25 (s, 1H), 7.36-7.41 (m, 2H), 7.90-7.93 (m, 1H), 9.14 |
| | | (s, 1H) |
| 147 | 843.3 | ¹H NMR (500 MHz, CD₃OD) δ 1.32 (s, 2H), 1.77 (s, 3H), 1.88 (s, 2H), |
| | | 1.96 (s, 1H), 2.07 (s, 4H), 2.22-2.31 (m, 4H), 3.17 (s, 1H), 3.42 (d, J = 3.5 |
| | | Hz, 2H), 3.85 (s, 2H), 4.35-4.40 (m, 2H), 4.54 (s, 2H), 4.68-4.71 (m, 2H), |
| | | 5.33-5.43 (m, 1H), 7.15-7.25 (m, 2H), 7.35-7.38 (m, 2H), 7.58-7.67 (m, |
| | | 2H), 7.85-7.89 (m, 1H), 7.99-8.05 (m, 4H), 8.68 (s, 1H), 8.86-9.22(m, 1H). |
| 148 | 1063.5 | ¹H NMR (500 MHz, CD₃OD) 8 0.69-0.78 (m, 3H), 0.94-1.02 (m, 8H), |
| | | 1.25-1.33 (m, 5H), 1.47 (s, 6H), 1.58-1.62 (m, 7H), 1.78-1.88 (m, 8H), 2.07-2.20 |
| | | (m, 5H), 2.30-2.43 (m, 6H), 2.63-2.74 (m, 2H), 3.45-3.52 (m, 3H), |
| | | 3.82-4.06 (m, 5H), 4.53 (s, 2H), 4.92-4.65 (m, 4H), 5.56-5.67 (m, 1H), 6.89 (s, |
| | | 1H), 7.23-7.27 (m, 1H), 7.36-7.50 (m, 2H), 7.87-7.95 (m, 1H), 9.15 (s, 1H). |
| 149 | 785.3 | ¹H NMR (500 MHz, CD₃OD) δ 1.49 (s, 3H), 1.53-1.70 (m, 4H), 1.71-1.92 |
| | | (m, 7H), 1.93-2.04 (m, 4H), 2.10-2.40 (m, 3H), 2.68-2.83 (m, 1H), |
| | | 2.98-3.08 (m, 1H), 3.31-3.42 (m, 2H), 3.60-3.83 (m, 2H), 4.20-4.35 (m, 2H), |
| | | 4.43 (s, 2H), 4.54-4.73 (m, 2H), 5.24 (s, 0.5H), 5.35 (s, 0.5H), 6.79 (d, J = |
| | | 5.5 Hz, 1H), 7.15 (s, 1H), 7.21-7.37 (m, 2H), 7.81 (dd, J = 9.0, 5.5 Hz, 1H), |
| | | 8.97 (s, 1H). |
| 150 | 745.3 | ¹H NMR (500 MHz, CD₃OD) δ ppm 0.96 (t, J = 7.5 Hz, 3H), 1.64-1.68 (m, |
| | | 3H), 1.84-1.86 (m, 2H), 1.93-2.07 (s, 6H), 2.19-2.33 (m, 2H), 2.38 (t, |
| | | J = 7.0 Hz, 2H), 3.10-3.11 (m, 1H), 3.35-3.36 (m, 3H), 3.74-3.80 (m, 2H), |
| | | 4.30-4.40 (m, 2H), 4.50 (s, 2H), 4.63-4.68 (m, 3H), 5.31 (s, 0.5H), 5.41 (s, |
| | | 0.5H), 5.83 (s, 2H), 7.20 (s, 1H), 7.31-7.35 (m, 2H), 7.85-7.88 (m, 1H), |
| | | 9.03 (s, 1H). |
| 151 | 785.3 | ¹H NMR (500 MHz, CD₃OD) δ ppm 1.25-1.42 (m, 3H), 1.44-1.56 (m, 2H), |
| | | 1.64-1.73 (m, 1H), 1.75-1.81 (m, 2H), 1.85-1.99 (m, 5H), 1.99-2.11 (m, |
| | | 4H), 2.14-2.48 (m, 4H), 3.02-3.16 (m, 1H), 3.22-3.33 (m, 3H), 3.69-3.94 |
| | | (m, 2H), 4.26-4.41 (m, 2H), 4.48-4.60 (m, 2H), 4.62-4.78 (m, 2H), 5.30 (s, |
| | | 0.5H), 5.41 (s, 0.5H), 5.86 (s, 2H), 7.23 (s, 1H), 7.35 (t, J = 9.0 Hz, 1H), |
| | | 7.38 (d, J = 2.5 Hz, 1H), 7.89 (dd, J = 9.0, 5.5 Hz, 1H), 9.05 (s, 1H). |
| 152 | 759.2 | ¹H NMR (500 MHz, CD₃OD) δ ppm 0.98 (d, J = 6.5 Hz, 6H), 1.85-2.34 (m, |
| | | 13H), 3.00-3.02 (m, 1H), 3.20-3.27 (m, 3H), 3.35 (d, J = 8.0 Hz, 1H), |
| | | 3.72-3.78 (m, 2H), 4.22-4.31 (m, 2H), 4.50-4.67 (m, 4H), 5.25 (s, 0.5H), 5.35 (s, |
| | | 0.5H), 5.83 (s, 2H), 7.20 (s, 1H), 7.30-7.34 (m, 2H), 7.84-7.87 (m, 1H), |
| | | 9.01 (s, 1H). m/z, |
| 153 | 717.2 | ¹H NMR (500 MHz, CD₃OD) δ ppm 1.83 (d, J = 8.0 Hz, 2H), 1.97-2.07 |
| | | (m, 2H), 2.11 (s, 3H), 2.13-2.23 (m, 1H), 2.30-2.39 (m, 2H), 2.40-2.50 (m, |
| | | ¹H), 2.54-2.78 (m, 2H), 3.42-3.53 (m, 1H), 3.71-4.08 (m, 5H), 4.47-4.57 |
| | | (m, 2H), 4.61-4.77 (m, 4H), 5.52 (s, 0.5H), 5.62 (s, 0.5H), 5.82 (s, 2H), 7.21 |
| | | (s, 1H), 7.34 (t, J = 9.0 Hz, 1H), 7.37 (s, 1H), 7.88 (dd, J = 9.0, 6.0 Hz, 1H), |
| | | 9.10 (s, 1H). m/z, |
| 154 | 788.5 | ¹H NMR (500 MHz, CD₃OD) δ ppm 9.13 (s, 1H), 7.92 (dd, J = 9.0, 6.0 Hz, |
| | | 1H), 7.41 (s, 1H), 7.38 (t, J = 9.0 Hz, 1H), 7.25 (s, 1H), 6.98-7.10 (m, 1H), |
| | | 5.66 (s, 1H), 5.56 (s, 1H), 4.67-4.82 (m, 3H), 4.49-4.61 (m, 2H), 3.78-4.11 |
| | | (m, 5H), 3.47-3.56 (m, 1H), 2.56-2.83 (m, 2H), 2.28-2.54 (m, 4H), |
| | | 2.14-2.26 (m, 1H), 1.99-2.14 (m, 2H), 1.81-1.98 (m, 2H), 1.67 (s, 3H), 1.14 (d, |
| | | J = 5.0 Hz, 6H). |
| 156 | 816.5 | ¹H NMR (500 MHz, CD₃OD) δ ppm 9.05 (s, 1H), 7.90 (dd, J = 8.9, 5.5 Hz, |
| | | 1H), 7.39 (s, 1H), 7.36 (t, J = 9.1 Hz, 1H), 7.24 (s, 1H), 6.96 (s, 1H), 5.40 |
| | | (s, 0.5H), 5.30 (s, 0.5H), 4.78-4.66 (m, 2H), 4.63 (s, 1H), 4.57-4.48 (m, |
| | | 2H), 4.41-4.25 (m, 2H), 3.89-3.67 (m, 2H), 3.39 (d, J = 7.6 Hz, 1H), |
| | | 3.33-3.18 (m, 3H), 3.12-3.01 (m, 1H), 2.87-2.72 (m, 1H), 2.45-2.14 (m, 9H), |
| | | 2.12-1.98 (m, 5H), 1.98-1.85 (m, 3H), 1.60 (s, 3H), 1.08-0.86 (m, 6H). |
| 158 | 830.6 | ¹H NMR (500 MHz, CD₃OD) δ ppm 9.01 (s, 1H), 7.86 (dd, J = 8.8, 5.9 Hz, |
| | | 1H), 7.35 (d, J = 2.0 Hz, 1H), 7.32 (t, J = 9.2 Hz, 1H), 7.20 (s, 1H), 6.93 (s, |
| | | 1H), 5.36 (s, 0.5H), 5.25 (s, 0.5H), 4.75-4.58 (m, 2H), 4.55-4.43 (m, 2H), |
| | | 4.36-4.20 (m, 2H), 3.84-3.67 (m, 2H), 3.36 (d, J = 7.6 Hz, 1H), 3.29-3.16 |
| | | (m, 3H), 3.08-2.98 (m, 1H), 2.93-2.81 (m, 1H), 2.41-2.20 (m, 8H), 2.19-2.10 |
| | | (m, 1H), 2.08-1.95 (m, 4H), 1.93-1.82 (m, 4H), 1.72-1.61 (m, 1H), |
| | | 1.57 (s, 3H), 1.25-1.10 (m, 1H), 0.91 (t, J = 7.2 Hz, 6H). |
| 159 | 774.5 | ¹H NMR (500 MHz, CD₃OD) δ ppm 1.56 (s, 3H), 1.82-1.94 (m, 3H), |
| | | 1.95-2.05 (m, 4H), 2.10-2.30 (m, 3H), 2.35 (s, 6H), 3.02 (dd, J = 14.8, 9.5 Hz, 1H), |
| | | 3.16-3.24 (m, 4H), 3.36 (d, J = 8.0 Hz, 1H), 3.70-3.85 (m, 2H), 4.28 (dt, |
| | | J = 16.6, 10.2 Hz, 2H), 4.48 (s, 2H), 4.59-4.73 (m, 3H), 5.25 (s, 0.5H), 5.36 |
| | | (s, 0.5H), 6.90 (d, J = 4.5 Hz, 1H), 7.21 (s, 1H), 7.29-7.35 (m, 2H), 7.86 (dd, |
| | | J = 8.7, 5.7 Hz, 1H), 9.02 (s, 1H). |
| 160 | 830.6 | ¹H NMR (500 MHz, CD₃OD) δ ppm 9.01 (s, 1H), 7.85 (dd, J = 8.7, 6.0 Hz, |
| | | 1H), 7.32 (dd, J = 16.6, 7.4 Hz, 2H), 7.20 (s, 1H), 6.91 (s, 1H), 5.35 (s, |
| | | 0.5H), 5.25 (s, 0.5H), 4.67 (s, 2H), 4.47 (s, 2H), 4.27 (dt, J = 16.5, 10.4 Hz, |
| | | 2H), 3.76 (t, J = 10.4 Hz, 2H), 3.35 (d, J = 7.2 Hz, 1H), 3.29-3.14 (m, 4H), |
| | | 3.06-2.91 (m, 1H), 2.35 (s, 6H), 2.20 (ddd, J = 45.0, 22.1, 7.0 Hz, 3H), |
| | | 2.07-1.80 (m, 7H), 1.74-1.43 (m, 6H), 0.94 (d, J = 6.4 Hz, 6H). |
| 161 | 788.6 | ¹H NMR (500 MHz, CD₃OD) δ ppm 9.03 (s, 1H), 7.86 (dd, J = 8.9, 5.8 Hz, |
| | | 1H), 7.38-7.30 (m, 2H), 7.21 (s, 1H), 6.89 (s, 1H), 5.39 (s, 0.5H), 5.28 (s, |
| | | 0.5H), 4.64 (d, J = 48.6 Hz, 3H), 4.49 (s, 2H), 4.32 (dt, J = 17.3, 10.2 Hz, |
| | | 2H), 3.79 (s, 2H), 3.36 (d, J = 7.6 Hz, 1H), 3.28-3.14 (m, 2H), 3.06 (d, J = |
| | | 4.9 Hz, 1H), 2.43-2.12 (m, 9H), 2.09-1.81 (m, 7H), 1.57 (s, 3H), 1.30 (d, |
| | | J = 6.3 Hz, 3H). |
| 162 | 816.6 | ¹H NMR (500 MHz, CD₃OD) δ ppm 9.10 (s, 1H), 7.88 (dd, J = 8.9, 5.8 Hz, |
| | | 1H), 7.37 (s, 1H), 7.34 (t, J = 8.9 Hz, 1H), 7.22 (s, 1H), 7.03 (s, 1H), 5.63 |
| | | (s, 0.5H), 5.52 (s, 0.5H), 4.83-4.63 (m, 4H), 4.53 (s, 2H), 4.18-3.64 (m, |
| | | 6H), 3.54-3.44 (m, 1H), 2.83-2.53 (m, 5H), 2.47-2.39 (m, 1H), 2.38-2.29 |
| | | (m, 2H), 2.23-2.13 (m, 1H), 2.11-1.94 (m, 3H), 1.93-1.81 (m, 2H), |
| | | 1.75-1.54 (m, 4H), 1.52-1.31 (m, 1H), 1.18-0.94 (m, 6H). |
| 163 | 802.6 | ¹H NMR (500 MHz, CD₃OD) δ ppm 9.09 (d, J = 13.6 Hz, 1H), 7.89 (dd, J = |
| | | 8.8, 5.9 Hz, 1H), 7.38 (d, J = 2.0 Hz, 1H), 7.35 (t, J = 8.9 Hz, 1H), 7.22 |
| | | (s, 1H), 7.09-6.98 (m, 1H), 5.63 (s, 0.5H), 5.53 (s, 0.5H), 4.81-4.64 (m, |
| | | 4H), 4.61-4.46 (m, 2H), 4.09-3.70 (m, 6H), 3.53-3.45 (m, 1H), 2.82-2.75 |
| | | (m, 3H), 2.74-2.54 (m, 2H), 2.50-2.41 (m, 1H), 2.40-2.29 (m, 3H), 2.26-2.14 |
| | | (m, 1H), 2.10-1.95 (m, 2H), 1.94-1.80 (m, 2H), 1.66 (s, 3H), 1.25-1.05 |
| | | (m, 6H). |
| 164 | 816.6 | ¹H NMR (500 MHz, CD₃OD) δ ppm 9.02 (s, 1H), 7.86 (dd, J = 9.0, 5.8 Hz, |
| | | 1H), 7.35 (d, J = 1.7 Hz, 1H), 7.32 (t, J = 9.0 Hz, 1H), 7.21 (s, 1H), 6.90 |
| | | (q, J = 5.1 Hz, 1H), 5.37 (s, 0.5H), 5.26 (s, 0.5H), 4.76-4.58 (m, 2H), |
| | | 4.54-4.43 (m, 2H), 4.36-4.20 (m, 2H), 3.85-3.63 (m, 6H), 3.36 (d, J = 7.8 Hz, |
| | | 1H), 3.29-3.16 (m, 4H), 3.07-2.99 (m, 1H), 2.61 (s, 4H), 2.38-2.10 (m, 3H), |
| | | 2.09-1.96 (m, 4H), 1.94-1.81 (m, 3H), 1.56 (s, 3H). |
| 165 | 864.6 | ¹H NMR (500 MHz, CD₃OD) δ ppm 1.51 (d, J = 5.0 Hz, 1H), 1.81-2.07 (m, |
| | | 7H), 2.20-2.41 (m, 10H), 3.00-3.10 (m, 3H), 3.34-3.71 (m, 7H), 4.27-4.47 |
| | | (m, 5H), 4.59-4.68 (m, 2H), 5.30 (s, 0.5H), 5.41 (s, 0.5H), 6.73-6.83 (m, |
| | | 1H), 7.21-7.35 (m, 8H), 7.85-7.88 (m, 1H), 9.01 (s, 1H). |
| 166 | 755.3 | ¹H NMR (500 MHz, CD₃OD) δ 2.14-2.20 (m, 6H), 2.40-2.48 (m, 3H), |
| | | 2.61-2.75 (m, 2H), 3.52 (d, J = 5 Hz, 2H), 3.88-4.11 (m, 6H), 4.31-4.33 (m, |
| | | 2H), 4.74-4.79 (m, 2H), 5.57-5.67 (m, 1H), 7.58 (t, J = 10 Hz, 1H), 7.66-7.69 |
| | | (m, 1H), 7.72-7.75(m, 2H), 8.04 (d, J = 10 Hz, 1H), 8.06-8.14 (m, 2H), |
| | | 8.17 (s, 1H), 8.22-8.26 (m, 2H), 8.92 (s, 1H), 9.19 (s, 1H). |
| 167 | 727.4 | ¹H NMR (500 MHz, CD₃OD) δ ppm 0.91-0.98 (m, 3H), 1.02-1.10 (m, 3H), |
| | | 1.36-1.47 (m, 4H), 1.64-1.84 (m, 4H), 2.06-2.26 (m, 5H), 2.29-2.52 (m, |
| | | 3H), 2.52-2.81 (m, 3H), 3.40-3.53 (m, 2H), 3.81-4.07 (m, 5H), 4.21-4.36 |
| | | (m, 2H), 4.69-4.75 (m, 2H), 4.82-4.90 (m, 2H), 5.53 (s, 0.5H), 5.64 (s, |
| | | 0.5H), 7.39 (s, 1H), 7.47-7.56 (m, 1H), 7.85-7.93 (m, 1H), 8.09-8.18 (m, |
| | | 1H), 9.13 (s, 1H). |
| 168 | 711.3 | ¹H NMR (500 MHz, CD₃OD) δ 1.30-1.53 (m, 3H), 1.57-1.92 (m, 5H), |
| | | 2.04-2.29 (m, 7H), 2.31-2.53 (m, 3H), 2.55-2.85 (m, 3H), 3.50 (d, J = 8.5 Hz, |
| | | 2H), 3.88-4.12 (m, 5H), 4.24-4.37 (m, 2H), 4.75 (s, 2H), 4.85-4.94 (m, 2H), |
| | | 5.56 (s, 0.5H), 5.67 (s, 0.5H), 7.46 (s, 1H), 7.53 (t, J = 9.0 Hz, 1H), 7.92 (d, |
| | | J = 2.0 Hz, 1H), 8.15 (dd, J = 9.0, 6.0 Hz, 1H), 9.16 (s, 1H). |
| 169 | 713.3 | ¹H NMR (500 MHz, CD₃OD) δ ppm 1.86-1.96 (m, 2H), 2.03-2.07(m, 2H), |
| | | 2.12-2.16 (m, 2H), 2.16-2.28 (m, 3H), 2.35-2.43 (m, 2H), 2.48-2.51 (m, |
| | | 1H), 2.58-2.68 (m, 1H), 2.70-2.80 (m, 1H), 2.98-3.02 (m, 2H), 3.49 (d, J = |
| | | 5 Hz, 2H), 3.59 (t, J = 10 Hz, 2H), 4.03-4.05 (m, 3H), 4.03-4.05 (m, |
| | | 4H), 4.25-4.35(m, 2H), 4.76 (s, 2H), 4.89 (s, 1H), 5.57-5.67(m, 1H), 7.48-7.51 |
| | | (m, 1H), 7.54 (t, J = 10 Hz, 1H), 7.95-7.98 (m, 1H), 8.16 (q, J = 10 |
| | | Hz, 1H), 9.17 (s, 1H). |
| 170 | 727.3 | ¹H NMR (500 MHz, CD₃OD) δ ppm 1.41-1.49 (m, 2H), 1.77-1.80 (m, 2H), |
| | | 2.10-2.19 (m, 6H), 2.36-2.44 (m, 3H), 2.62-2.63 (m, 3H), 3.44-3.49 (m, |
| | | 4H), 3.90-4.01 (m, 8H), 4.27-4.30 (m, 2H), 4.69-4.72 (m, 2H), 5.53-5.63(m,1H), |
| | | 7.46(s, 1H), 7.49-7.52 (m,1H), 7.92 (s, 1H), 8.11-8.14 (m,1H), |
| | | 9.13(s, 1H). |
| 172 | 859.3 | ¹H NMR (500 MHz, CD₃OD) δ ppm 1.14-1.16 (m, 12H), 2.09-2.15 (m, |
| | | 5H), 2.34-2.46 (m, 3H), 2.57-2.76 (m, 4H), 3.48-3.49 (m, 2H), 3.85-4.02 |
| | | (m, 5H), 4.27-4.31 (m, 4H), 4.49-4.52 (m, 2H), 4.71-4.72 (m, 2H), 4.87-4.94 |
| | | (m, 2H), 5.23 (s, 1H), 5.53 (s, 0.5H), 5.63 (s, 0.5H), 7.50-7.56 (m,2H), |
| | | 8.01 (s, 1H), 8.12-8.15 (m, 1H), 9.14 (s, 1H). |
| 173 | 888.3 | ¹H NMR (500 MHz, CD₃OD) δ 1.21-1.23 (m, J = 5 Hz, 18H), 1.28-1.36 (m, |
| | | 1H), 2.13-2.21 (m, 6H), 2.32-2.40 (m, 2H), 2.44-2.50 (m, 1H), 2.60-2.79 |
| | | (m, 2H), 3.49-3.53 (m, 2H), 3.87-4.10 (m, 5H), 4.28-4.36 (m, 4H), 4.49-4.52 |
| | | (m 2H), 4.75 (s, 2H), 5.27-5.30 (m, 1H), 5.56-5.67 (m,1H), 7.56-7.58 |
| | | (s, 2H), 8.03 (s, 1H), 8.17 (dd, J = 5 Hz, 1H), 9.17 (s, 1H) |
| 174 | 971.7 | ¹H NMR (500 MHz, CD₃OD) δ ppm 0.78-0.87 (m, 6H), 1.19-1.36 (m, |
| | | 16H), 1.55-1.64 (m, 4H), 2.05-2.26 (m, 5H), 2.29-2.40 (m, 6H), 2.41-2.49 |
| | | (m, 1H), 2.55-2.78 (m, 2H), 3.14-3.25 (m, 1H), 3.42-3.53 (m, 2H), 3.84-4.08 |
| | | (m, 5H), 4.24-4.34 (m, 4H), 4.49-4.58 (m, 2H), 4.69-4.77 (m, 2H), |
| | | 5.18-5.25 (m, 1H), 5.53 (s, 0.5H), 5.64 (s, 0.5H), 7.50-7.56 (m, 1H), 7.58 |
| | | (s, 1H), 8.01-8.06 (m, 1H), 8.14 (dd, J = 9.0, 6.0 Hz, 1H), 9.14 (s, 1H). |
| 175 | 815.3 | ¹H NMR (500 MHz, CD₃OD) δ 1.35 (s, 9H), 1.49 (s, 3H), 1.75-1.84 (m, |
| | | 2H), 1.87-2.11 (m, 8H), 2.16-2.44 (m, 3H), 3.07-3.15 (m, 1H), (m, 4H), |
| | | 3.64-3.80 (m, 2H), 4.24-4.50 (m, 4H), 4.55-4.72 (m, 3H), 5.29 (s, 0.5H), |
| | | 5.39 (s, 0.5H), 6.78 (q, J = 5.0 Hz, 1H), 7.37 (s, 1H), 7.44 (t, J = 8.9 Hz, |
| | | 1H), 7.82 (d, J = 2.0 Hz, 1H), 8.05 (dd, J = 9.0, 6.0 Hz, 1H), 9.01 (s, 1H). |
| 176 | 875.3 | ¹H NMR (500 MHz, CD₃OD) δ ppm 0.89-1.00 (m, 3H), 1.44-1.56 (m, 6H), |
| | | 1.57-1.70 (m, 2H), 1.76-2.06 (m, 7H), 2.07-2.42 (m, 5H), 2.95-3.05 (m, |
| | | 1H), 3.14-3.27 (m, 3H), 3.33-3.40 (m, 1H), 3.62-3.85 (m, 4H), 4.15-4.35 |
| | | (m, 2H), 4.39-4.54 (m, 2H), 4.57-4.80 (m, 2H), 5.56 (s, 0.5H), 5.66 (s, |
| | | 0.5H), 6.80-6.90 (m, 1H), 7.47-7.54 (m, 2H), 7.90-7.94 (m, 1H), 8.05-8.15 |
| | | (m, 1H), 9.15 (s, 1H). |
| 177 | 857.68 | ¹H NMR (500 MHz, CD₃OD) δ ppm 1.15-1.27 (m, 9H), 1.40 (s, 9H), |
| | | 1.49-1.59 (m, 3H), 1.77-2.14 (m, 7H), 2.17-2.49 (m, 3H), 3.05-3.19 (m, 1H), |
| | | 3.37-3.55 (m, 4H), 3.67-3.86 (m, 2H), 4.30-4.54 (m, 4H), 4.58-4.78 (m, |
| | | 2H), 5.33 (s, 0.5H), 5.43 (s, 0.5H), 6.77-6.85 (m, 1H), 7.41 (s, 1H), 7.49 (t, |
| | | J = 9.0 Hz, 1H), 7.84-7.89 (m, 1H), 8.06-8.14 (m, 1H), 9.06 (s, 1H). |
| 178 | 941.5 | ¹H NMR (500 MHz, CD₃OD) δ ppm 0.85-1.05 (m, 12H), 1.31-1.67 (m, |
| | | 16H), 1.69-2.06 (m, 9H), 2.09-2.46 (m, 4H), 2.65-2.75 (m, 1H), 2.97-3.07 |
| | | (m, 1H), 3.19-3.28 (m, 2H), 3.62-3.83 (m, 2H), 4.19-4.36 (m, 2H), 4.37-4.52 |
| | | (m, 2H), 4.57-4.75 (m, 2H), 5.25 (s, 0.5H), 5.36 (s, 0.5H), 6.83-6.93 |
| | | (m, 1H), 7.35-7.53 (m, 2H), 7.84 (s, 1H), 8.05-8.15 (m, 1H), 9.02 (s, 1H). |
| 179 | 841.3 | ¹H NMR (500 MHz, CD₃OD) δ ppm 1.40 (s, 9H), 1.82-1.84 (m, 2H), |
| | | 2.03-2.16 (m, 3H), 2.21 (s, 3H), 2.35-2.73 (m, 5H), 3.44-3.46 (m, 2H), 3.77-3.92 |
| | | (m, 5H), 4.51 (s, 2H), 4.63-4.72 (m, 4H), 5.03 (s, 2H), 5.52 (s, 0.5H), 5.63 |
| | | (s, 0.5H), 7.41 (s, 1H), 7.49 (d, J = 8.5 Hz, 1H), 7.88-7.89 (m, 1H), 8.10-8.13 |
| | | (m, 1H), 9.12 (s, 1H). |
| 180 | 1157.8 | ¹H NMR (500 MHz, CD₃OD) δ ppm 0.84 (t, J = 7.5 Hz, 6H), 1.22-1.25 (m, |
| | | 24H), 1.53 (s, 3H), 1.56-1.59 (m, 4H), 1.83-1.84 (m, 2H), 2.02-2.09 (m, |
| | | 7H), 2.21-2.32 (m, 3H), 2.35 (t, J = 7.0 Hz, 4H), 2.41-2.58 (m, 2H), |
| | | 3.65-3.82 (m, 5H), 4.26-4.29 (m, 2H), 4.49-4.54 (m, 7H), 4.64-4.73 (m, 2H), |
| | | 5.21-5.22 (m, 1H), 5.42 (s, 0.5H), 5.52 (s, 0.5H), 6.82-6.84 (m, 1H), 7.51 |
| | | (t, J = 9.0 Hz, 1H), 7.56 (s, 1H), 8.00-8.01 (m, 1H), 8.11-8.13 (m, 1H), 9.10 |
| | | (s, 1H). |
| 181 | 1101.8. | ¹H NMR (500 MHz, CD₃OD) δ ppm 0.78-0.89 (m, 6H), 1.17-1.31 (m, |
| | | 16H), 1.49-1.66 (m, 7H), 1.78-2.14 (m, 10H), 2.16-2.44 (m, 7H), 3.05-3.15 |
| | | (m, 1H), 3.35-3.54 (m, 3H), 3.67-3.87 (m, 2H), 4.19-4.43 (m, 4H), 4.44-4.56 |
| | | (m, 4H), 4.58-4.76 (m, 3H), 5.18-5.26 (m, 1H), 5.31 (s, 0.5H), 5.42 (s, |
| | | 0.5H), 6.79-6.89 (m, 1H), 7.48-7.54 (m, 1H), 7.56 (s, 1H), 7.97-8.03 (m, |
| | | 1H), 8.09-8.16 (m, 1H), 9.06 (s, 1H). |
| 182 | 1213.8 | ¹H NMR (500 MHz, CD₃OD) δ 0.87 (t, J = 5 Hz, 6H), 0.95 (t, J = 10 Hz, |
| | | 6H), 1.21-1.40 (m, 26H), 1.46-1.55 (m, 2H), 1.56-1.67 (m, 9H), 1.85-1.90 |
| | | (m, 2H), 1.96-2.07 (m, 4H), 2.20-2.28 (m, 1H), 2.33-2.47 (m, 5H), 3.18 (s, |
| | | 1H), 3.38-3.57 (m, 3H), 3.70-3.75 (m, 2H), 4.26-4.33(m, 2H), 4.35-4.41 |
| | | (m, 1H), 4.45-4.54 (m, 5H), 4.65-4.80 (m, 2H), 5.20-5.23 (m, 1H), 5.35-5.50 |
| | | (m, 1H), 6.87-6.90 (m, 1H), 7.58 (t, J = 10 Hz, 1H), 7.59 (s, 1H), |
| | | 8.01-8.05 (m, 1H), 8.15 (dd, J = 5 Hz, 1H), 9.08 (s, 1H). |
| 183 | 1297.9 | ¹H NMR (500 MHz, CD₃OD) δ ppm 0.86 (t, J = 7.0 Hz, 6H), 0.91 (t, |
| | | J = 6.5 Hz, 6H), 1.20-1.28 (m, 36H), 1.45-1.58 (m, 12H), 1.85-2.07 (m, 7H), |
| | | 2.20-2.40 (m, 8H), 3.11-3.12 (m, 1H), 3.36-3.49 (m, 3H), 3.70-3.77 (m, |
| | | 2H), 4.25-4.67 (m, 10H), 5.21 (s, 1H), 5.31 (s, 0.5H), 5.42 (s, 0.5H), 6.88 |
| | | (s, 1H), 7.49 (t, J = 8.5 Hz, 1H), 7.55 (s, 1H), 7.98 (s, 1H), 8.08-8.11 (m, 1H), |
| | | 9.05 (s, 1H). |
| 184 | 1213.7 | ¹H NMR (500 MHz, CD₃OD) δ ppm 0.89 (t, J = 7.0 Hz, 6H), 1.20-1.37 (m, |
| | | 32H), 1.50-1.70 (m, 7H), 1.79-1.98 (m, 3H), 1.99-2.32 (m, 9H), 2.33-2.45 |
| | | (m, 5H), 3.01-3.11 (m, 1H), 3.17-3.31 (m, 3H), 3.53 (d, J = 7.0 Hz, 1H), |
| | | 3.69-3.91 (m, 2H), 4.24-4.40 (m, 4H), 4.46-4.62 (m, 4H), 4.62-4.82 (m, |
| | | 2H), 5.22-5.27 (m, 1H), 5.29 (s, 0.5H), 5.39 (s, 0.5H), 6.86 (q, J = 5.0 Hz, |
| | | 1H), 7.53 (t, J = 9.0 Hz, 1H), 7.58 (s, 1H), 8.02 (s, 1H), 8.13 (dd, J = 9.0, |
| | | 6.0 Hz, 1H), 9.08 (s, 1H). |
| 185 | 1172.7 | ¹H NMR (500 MHz, CD₃OD) δ ppm 0.83 (t, J = 6.5 Hz, 6H), 0.97 (d, |
| | | J = 6.0 Hz, 6H), 1.21-1.28 (m, 20H), 1.53-1.58 (m, 7H), 1.83-1.89 (m, 3H), |
| | | 2.00-2.07 (m, 5H), 2.12-2.26 (m, 5H), 2.32-2.34 (m, 5H), 3.00-3.03 (m, |
| | | 1H), 3.18-3.25 (m, 2H), 3.47-3.48 (m, 1H), 3.70-3.76 (m, 2H), 4.23-4.33 |
| | | (m, 4H), 4.45-4.51 (m, 4H), 4.65 (s, 2H), 5.21 (s, 1H), 5.25 (s, 0.5H), 5.36 |
| | | (s, 0.5H), 6.85-6.86 (m, 1H), 7.48 (t, J = 8.5 Hz, 1H), 7.56 (s,1H), 7.97 (s, |
| | | 1H), 8.07-8.10 (m, 1H), 9.03 (s, 1H). |
| 186 | 815.4 | ¹H NMR (500 MHz, CD₃OD) δ ppm 0.93-1.03 (m, 6H), 1.49-1.61 (m, 3H), |
| | | 1.79-2.13 (m, 8H), 2.16-2.44 (m, 8H), 3.05-3.14 (m, 1H), 3.33-3.39 (m, |
| | | 2H), 3.40-3.50 (m, 1H), 3.65-3.84 (m, 2H), 4.22-4.42 (m, 2H), 4.42-4.53 |
| | | (m, 2H), 4.56-4.76 (m, 2H), 5.30 (s, 0.5H), 5.40 (s, 0.5H), 6.79-6.90 (m, |
| | | 1H), 7.42-7.53 (m, 2H), 7.84-7.92 (m, 1H), 8.08 (dd, J = 5.5, 9.0 Hz, 1H), |
| | | 9.04 (s, 1H). |
| 187 | 743.3 | ¹H NMR (500 MHz, CD₃OD) δ ppm 0.99-1.02 (m, 6H), 1.45-1.51 (m, 3H), |
| | | 1.66-1.73 (m, 4H), 2.07-2.21 (m, 6H), 2.35-2.42 (m, 2H), 2.60-2.78 (m, |
| | | 2H), 3.49-3.54 (m, 2H), 3.90-4.07 (m, 5H), 4.31-4.33 (m, 2H), 4.72-4.78 |
| | | (m, 2H), 4.89-4.95 (m, 2H), 5.56-5.67 (m, 1H), 7.53-7.57 (m, 2H), |
| | | 8.03-8.04 (m, 1H), 8.17-8.20 (m, 1H), 9.17 (s, 1H). |
| 188 | 779.4 | ¹H NMR (500 MHz, CD₃OD) δ ppm 1.68-1.70 (m, 2H), 1.85-1.88 (m, 6H), |
| | | 1.98-2.02 (m, 2H), 2.07-2.15 (m, 6H), 2.19 (s, 4H), 2.35-2.42 (m, 2H), |
| | | 2.60-2.78 (m, 2H), 3.48-3.51 (m, 2H), 3.91-4.01 (m, 5H), 4.31-4.33 (m, |
| | | 2H), 4.72-4.78 (m, 2H), 4.90-4.95 (m, 2H), 5.56-5.67 (m,1H), 7.53-7.56 (t, |
| | | J = 17.5 Hz, 1H), 7.59 (s,1H), 8.06-8.07 (m, 1H), 8.16-8.19 (m, 1H), 9.17(s, |
| | | 1H). |
| 189 | 701.3 | ¹H NMR (500 MHz, CD₃OD) δ 1.58 (s, 9H), 1.80-1.94 (m, 5H), 2.03-2.04 |
| | | (m, 2H), 2.14-2.42 (m, 3H), 3.02-3.09 (m, 1H), 3.24-3.33 (m, 3H), 3.52 (d, |
| | | J = 8.5 Hz, 1H), 3.71-3.77 (m, 4H), 4.24-4.27 (m, 1H), 4.31-4.37 (m, 1H), |
| | | 4.65 (t, J = 12.0 Hz, 2H), 5.34 (d, J = 54.0 Hz, 1H), 7.49-7.56 (m, 2H), 7.96 |
| | | (d, J = 2.0 Hz, 1H), 8.13 (dd, J = 9.0, 5.5 Hz, 1H), 9.06 (s, 1H). |
| 190 | 629.3 | ¹H NMR (500 MHz, CD₃OD) δ 1.23 (t, J = 7.0 Hz, 3H), 1.32 (s, 1H), 1.72 |
| | | (d, J = 9.0 Hz, 2H), 1.88-2.03 (m, 4H), 2.14-2.38 (m, 3H), 2.59 (q, J = 7 |
| | | Hz, 2H), 3.03-3.04 (m, 1H), 3.22-3.30 (m, 3H), 3.39 (d, J = 9.5 Hz, 1H), |
| | | 3.54 (s, 2H), 3.76-3.85 (m, 2H), 4.23-4.29 (m, 1H), 4.29-4.36 (m, 1H), |
| | | 4.60-4.68 (m, 2H), 5.34 (d, J = 53.5 Hz, 1H), 7.23 (s, 1H), 7.32-7.39 (m, |
| | | 2H), 7.88 (dd, J = 8.5, 6 Hz, 1H), 9.05 (s, 1H). |
| 191 | 775.3 | ¹H NMR (500 MHz, MeOD) δ 1.19 (t, J = 5.7 Hz, 6H), 2.14 (m, 5H), |
| | | 2.31-2.39 (m, 2H), 2.45 (s, 1H), 2.55-2.78 (m, 2H), 3.48 (m, 2H), 3.56 (m, 4H), |
| | | 3.68 (d, J = 5.1 Hz, 4H), 3.86-4.07 (m, 5H), 4.29 (s, 2H), 4.67-4.77 (m, |
| | | 2H), 4.88 (m, 2H), 5.05-5.11 (m, 1H), 5.59 (m, 1H), 7.52 (t, J = 9.0 Hz, |
| | | 1H), 7.57 (s, 1H), 8.02 (d, J = 2.0 Hz, 1H), 8.14 (dd, J = 9.0, 5.5 Hz, 1H), |
| | | 9.14 (s, 1H). |
| 192 | 713.3 | ¹H NMR (500 MHz, CD₃OD) δ 1.32-1.34 (m, 3H), 1.90-1.93 (m, 2H), 2.09 |
| | | (s, 3H), 2.09-2.32 (m, 3H), 2.41-2.52 (m, 3H), 3.31 (s, 1H), 3.41 (s, 1H), |
| | | 3.63-3.733 (m, 3H), 3.77-3.94 (m, 3H), 4.08-4.14 (m, 1H), 4.45-4.57 (m, |
| | | 4H), 4.65-4.77 (m, 2H), 5.40-5.51 (m, 1H), 7.24 (s, 1H), 7.30-7.38 (m, 2H), |
| | | 7.91 (dd, J = 5 Hz, 1H), 9.12-9.13 (m, 1H). |
| 193 | 879.4 | ¹H NMR (500 MHz, CD₃OD) δ ppm 2.10-2.18 (m, 5H), 2.34-2.72 (m, 5H), |
| | | 3.32 (s, 3H), 3.45-3.51 (m, 4H), 3.58-3.69 (m, 16H), 3.79-3.81 (m, 2H), |
| | | 3.85-4.03 (m, 5H), 4.26-4.31 (m, 2H), 4.42-4.44 (m, 2H), 4.72 (s, 2H), 5.53 |
| | | (s, 0.5H), 5.63 (s, 0.5H), 7.51 (t, J = 8.5 Hz, 1H), 7.57 (s, 1H), 8.02-8.03 (m, |
| | | 1H), 8.12-8.15 (m, 1H), 9.14 (s, 1H). |
| 194 | 975.5 | ¹H NMR (500 MHz, CD₃OD) δ ppm 1.97-2.19 (m, 17H), 2.31-2.49 (m, |
| | | 3H), 2.55-2.77 (m, 2H), 3.42-3.53 (m, 2H), 3.84-4.07 (m, 5H), 4.14 (d, J = |
| | | 9.0 Hz, 1H), 4.25-4.33 (m, 1H), 4.35-4.41 (m, 1H), 4.47-4.55 (m, 1H), 4.66-4.76 |
| | | (m, 2H), 5.05-5.11 (m, 1H), 5.11-5.19 (m, 1H), 5.39 (t, J = 9.5 Hz, 1H), |
| | | 5.53 (s, 0.5H), 5.63 (s, 0.5H), 5.86 (d, J = 8.5 Hz, 1H), 7.48-7.56 (m, 1H), |
| | | 7.56-7.63 (m, 1H), 8.03 (s, 1H), 8.10-8.17 (m, 1H), 9.15 (d, J = 2.5 Hz, 1H). |
| 195 | 615.3 | ¹H NMR (500 MHz, CD₃OD) δ ppm 1.95-2.05 (m, 5H), 2.11-2.22 (m, 2H), |
| | | 2.29-2.33 (m, 1H), 2.39-2.57 (m, 2H), 3.24-3.30 (m, 1H), 3.40-3.41 (d, J = |
| | | 6.5 Hz, 1H), 3.52-3.69 (m, 3H), 3.83-3.90 (m, 2H), 3.97-3.99 (m, 2H), 4.02 |
| | | (s, 1H), 4.46-4.56 (m, 2H), 4.74-4.79 (m, 2H), 5.41-5.52 (m,1H), 7.31-7.32 |
| | | (m, 1H), 7.40-7.44 (t, J = 18 Hz, 1H), 7.57-7.57 (d, J = 2 Hz, 1H), 8.03-8.06 |
| | | (m, 1H), 9.10 (s, 1H). |
| 196 | 945.4 | ¹H NMR (500 MHz, CD₃OD) δ ppm 1.99-2.04 (m, 9H), 2.08-2.23 (m, |
| | | 10H), 2.32-2.38 (m, 2H), 2.42-2.46 (m, 1H), 2.56-2.74 (m, 2H), 3.45-3.50 |
| | | (m, 2H), 3.86-4.03 (m, 5H), 4.27-4.29 (m, 2H), 4.68-4.74 (m, 2H), 4.78-4.80 |
| | | (m, 1H), 5.13-5.16 (m, 1H), 5.35-5.39 (m, 1H), 5.52-5.54 (m, 1H), |
| | | 5.56 (s, 0.5H), 5.63 (s, 0.5H), 6.00 (d, J = 7.5 Hz, 1H), 7.46 (s, 1H), 7.52 (t, |
| | | J = 9.0 Hz, 1H), 7.93 (s, 1H), 8.12-8.15 (m, 1H), 9.13 (s, 1H). |
| 197 | 850.3 | ¹H NMR (500 MHz, CD₃OD) δ ppm 1.00 (t, J = 7.4 Hz, 3H), 1.32 (s, 2H), |
| | | 1.42-1.50 (m, 3H), 1.65-1.74 (m, 3H), 1.86 (d, J = 7.5 Hz, 2H), 1.90-1.96 |
| | | (m, 1H), 2.00-2.11 (m, 4H), 2.16-2.43 (m, 3H), 3.10 (s, 1H), 3.35-3.39 (m, |
| | | 1H), 3.78 (s, 2H), 4.16 (t, J = 6.5 Hz, 2H), 4.28-4.35 (m, 1H), 4.35-4.41 (m, |
| | | 1H), 4.52 (s, 2H), 4.69 (s, 2H), 5.19 (s, 2H), 5.35-5.45(m, 1H), 7.24 (s, 1H), |
| | | 7.38-7.42 (m, 4H), 7.45-7.49(m, 2H), 7.89 (dd, J = 5 Hz, 1H), 9.04 (s, 1H). |
| 198 | 1084.0 | ¹H NMR (500 MHz, CD₃OD) δ ppm 9.02 (s, 1H), 7.85 (dd, J = 8.9, 5.8 Hz, |
| | | 1H), 7.34 (d, J = 2.1 Hz, 1H), 7.31 (t, J = 8.9 Hz, 1H), 7.20 (s, 1H), 5.36 (s, |
| | | 0.5H), 5.26 (s, 0.5H), 5.24-5.19 (m, 1H), 4.78-4.53 (m, 3H), 4.52-4.35 (m, |
| | | 4H), 4.35-4.21 (m, 4H), 3.87-3.67 (m, 2H), 3.33 (d, J = 6.3 Hz, 1H), |
| | | 3.28-3.21 (m, 2H), 3.07-3.00 (m, 1H), 2.43-2.09 (m, 7H), 2.08-1.95 (m, 4H), |
| | | 1.95-1.78 (m, 3H), 1.66-1.54 (m, 4H), 1.34-1.16 (m, 32H), 0.86 (s, 6H). |
| 199 | 817.7 | ¹H NMR (500 MHz, CD₃OD) δ ppm 0.86 (t, J = 7.0 Hz, 3H), 1.21-1.32 (m, |
| | | 16H), 1.57-1.63 (m, 2H), 2.10-2.22 (m, 5H), 2.35 (t, J = 7.5 Hz, 4H), |
| | | 2.42-2.46 (m, 1H), 2.56-2.75 (m, 2H), 3.44-3.50 (m, 2H), 3.85-4.05 (m, |
| | | 5H),4.27-4.30 (m, 2H), 4.40-4.41 (m, 2H), 4.48-4.49 (m, 2H), 4.72 (s, 2H), |
| | | 4.85-4.90 (m, 2H), 5.53 (s, 0.5H), 5.63 (s, 0.5H), 7.51 (t, J = 8.5 Hz, 1H), |
| | | 7.57 (s, 1H), 8.01 (s, 1H), 8.13 (dd, J = 8.8, 5.7 Hz, 1H), 9.14 (s, 1H). |
| 200 | 927.8 | ¹H NMR (500 MHz, CD₃OD) δ ppm 0.89 (t, J = 7.0 Hz, 3H), 1.21-1.28 (m, |
| | | 24H), 1.60 (dd, J = 14.7, 7.5 Hz, 2H), 2.10-2.75 (m, 13H), 3.45-3.50 (m, |
| | | 2H), 3.85-4.05 (m, 5H),4.27-4.30 (m, 2H), 4.40-4.41 (m, 2H), 4.48-4.49 |
| | | (m, 2H), 4.72 (s, 2H), 4.85 (s, 1H), 5.53 (s, 0.5H), 5.64 (s, 0.5H), 7.51 (t, J = |
| | | 8.9 Hz, 1H), 7.57 (s, 1H), 8.01 (d, J = 2.1 Hz, 1H), 8.13 (dd, J = 9.1, 5.9 |
| | | Hz, 1H), 9.14 (s, 1H). |

### Biological Assays

### Example 1. Pharmacokinetic (PK) Study

Mouse PK Study

A total 126 ICR mice (male, 30-34 g) were randomly divided into 42 groups with 3 animals/group (oral or intravenous administration). Blood samples were collected at 0.167, 0.5, 1, 2, 4, 6, 8, and 24 hours post-dose for oral group and 0.083, 0.25, 0.5, 1, 2, 4, 6, and 8 hours post-dose for intravenous group. The test compounds were dissolved into vehicle solutions (5% DMSO+5% Solutol+90% 20% SBE-β-CD solvents).

Animals were administered with compound A1 or exemplary test compounds by intravenous bolus administration at 3 mg/kg (equimolar dose to compound A1) and oral administration at 10 mg/kg (equimolar dose to compound A1) after fasted 12 hours, respectively. Post-dose blood samples were collected at preset time point (50 μL/animal/time point), and 20 μL blood was accurately separated into 200 μL eppendorf (EP) tube containing acetonitrile and internal standard. The supernatant was prepared by centrifuging the blood samples at approximately 4° C., 12000 rpm for 5 minutes. All samples were then quickly frozen over dry ice and kept at −60° C. or lower until analysis. The supernatant was analyzed by LC-MS/MS to detect the concentration of test compounds and metabolites.

Table 4 shows AUC_{0-∞} (area under mean blood concentration-time curve) for compound A1 after oral administration of either A1 directly or various test compounds, as indicated, in ICR mice.

Table 5 shows AUC_{0-∞} (area under mean blood concentration-time curve) for compound A1 after intravenous administration of either A1 directly or various test compounds, as indicated, in ICR mice.

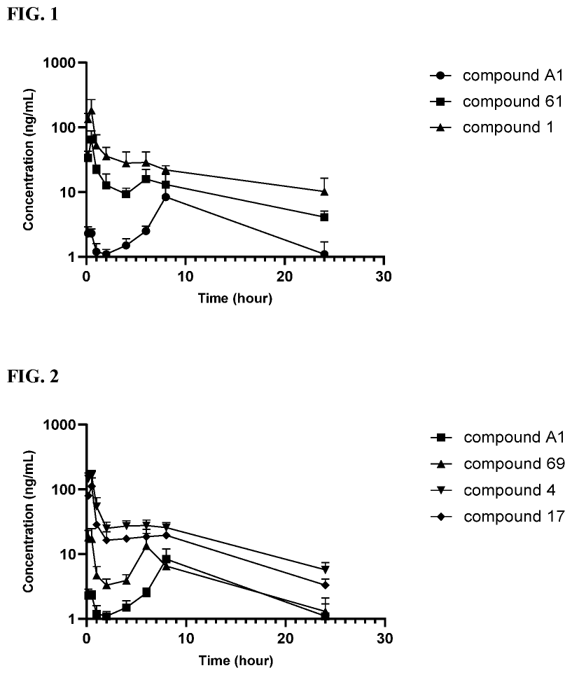

FIGS. 1-4 show the concentration-time (c-t) curves for compound A1 after administration of A1 and various test compounds, as indicated, in ICR mice.

TABLE 4
AUC_{0-∞} of compound A1 in ICR mice after oral administration
of A1 and exemplary compounds of the disclosure at an equimolar dose.

| | AUC_{0-∞} (PO) |
|---|---|
| Compound | (h*ng/mL) |
| A1 | 113 |
| 1 | 784 |
| 4 | 637 |
| 6 | 758 |
| 7 | 442 |
| 8 | 403 |
| 9 | 607 |
| 10 | 964 |
| 11 | 289 |
| 12 | 148 |
| 17 | 396 |
| 52 | 108 |
| 61 | 339 |
| 69 | 140 |
| 71 | 84 |
| 77 | 344 |
| 156 | 628 |
| 158 | 599 |
| 159 | 171 |
| 160 | 702 |
| 161 | 193 |
| 162 | 649 |
| 163 | 568 |
| 164 | 837 |
| 165 | 592 |

As shown in Table 4, the AUC_{0-∞} of A1 after oral administration of test compounds was significantly higher than that of A1 (the control compound) after direct oral administration of A1. In particular, compound 10 greatly improved the oral bioavailability of A1 since the AUC of A1 was increased by more than 8.5 times compared to direct oral administration of A1.

TABLE 5
AUC_{0-∞} of compound A1 in ICR mice after
intravenous administration of A1 and exemplary
compounds of the disclosure at an equimolar dose.

| | AUC_{0-∞} (IV) |
|---|---|
| Compound | h*ng/mL |
| A1 | 2614 |
| 1 | 1912 |
| 4 | 1818 |
| 7 | 2421 |
| 17 | 733 |
| 32 | 3057 |
| 33 | 3668 |
| 36 | 9239 |
| 39 | 3959 |
| 52 | 4784 |
| 53 | 4203 |
| 55 | 3342 |
| 56 | 5344 |
| 61 | 2417 |
| 69 | 2610 |
| 71 | 2655 |
| 77 | 2386 |

As shown in Table 5, the AUC_{0-∞} of A1 after intravenous administration of test compounds was higher than that of the control compound A1 after direct administration of A1. In particular, the drug exposure of A1 was increased by more than 50% after administration of compounds 36, 39, 52, 53 and 56 compared to direct intravenous administration of A1, and compound 36 gave a nearly 4-fold increase.

FIG. 1 shows the concentration-time curve for compound A1 after oral administration of compound 1, compound 61 and compound A1.

FIG. 2 shows the concentration-time curve for compound A1 after oral administration of compound 4, compound 17, compound 69 and compound A1.

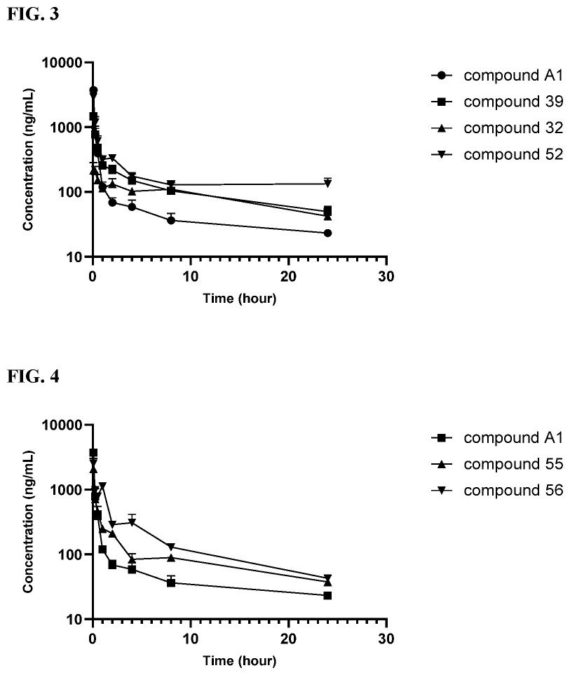

FIG. 3 shows the concentration-time curve for compound A1 after intravenous administration of compound 32, compound 39, compound 25 and compound A1.

FIG. 4 shows the concentration-time curve for compound A1 after intravenous administration of compound 55, compound 56, and compound A1.

### Example 2. Efficacy Study in Mouse AsPC-1 CDX Model

I. Efficacy Study in Mice Receiving Compounds Orally

Balb/C nude mice, aged 6-8 weeks, were inoculated subcutaneously with 5×10⁶ AsPC-1 pancreatic cancer cells, in 0.1 ml PBS. Eleven days post-inoculation, tumor bearing animals were randomized into Vehicle (four mice), Compound A1 (four mice) and Compound 1 (five mice), followed by oral dosing twice daily at 100 mg/kg for Compound A1 and 126 mg/kg for Compound 1 (Table 6). Tumor volume was measured every three days using a caliper, and tumor size was calculated using the formula TV=length×width²×0.5. Tumor growth inhibition (TGI) was calculated using the given formula TGI=[1−(Vtx_t−Vtx_0)/(Vct_t−Vct_0)]×100%, where Vtx_t means tumor volume in treatment group measured at time point t, Vtx_0 means tumor volume of treatment group measured at time point 0, Vct_t means tumor volume of vehicle group measured at time point t, and Vct_0 means tumor volume of vehicle group measured at time point 0.

Table 6 shows that oral dosing of Compound 1 twice a day at 126 mg/kg (molar equivalent to 100 mg/kg of Compound A1) for 16 days significantly inhibited tumor growth (TGI=53.5%, P=0.0146), whereas, Compound A1, administered orally twice a day at a dosage of 100 mg/kg for 12 days, resulted in only 9.07% tumor growth inhibition. Compared to Compound A1, Compound 1 significantly suppressed tumor growth in mice (P=0.003). Neither Compound 1 nor A1 adversely affected mouse body weight when given orally.

TABLE 6
Dosage regimen and inhibitory effect
in mouse AsPC-1 CDX model

| | | | Treatment | |
|---|---|---|---|---|
| Compound and | | Animal | duration | |
| dosing route | Dosage (mg/kg) | number | (BID) | TGI (%) |
| Vehicle | 0 | 4 | 16 | NA |
| Compound A1, oral | 100 | 4 | 12 | 9.07 |
| Compound 1, oral | 126 | 5 | 16 | 53.95 |

P: mean ± SEM; P < 0.05 means significant difference.

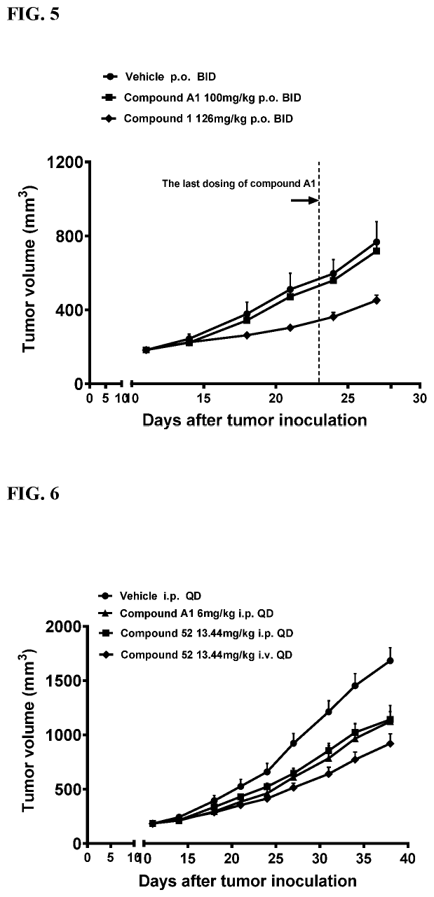

FIG. 5 shows the comparative results of tumor growth inhibitory effect in mice after oral administration of Compound 1, Compound A1 and blank control (vehicle).

II. Tumor Growth Inhibition (TGI) in Mice Receiving Compounds Parenterally

Balb/C nude mice bearing tumors derived from AsPC-1 cells as described previously, were grouped into Vehicle intraperitoneal (IP) (five mice), Compound A1 IP (five mice), Compound A1 intravenous (IV) (three mice), Compound 52 IP (five mice) and Compound 52 IV injections (six mice). All compounds were administered once daily at equal molarity (Compound A1, 6 mg/kg) for 27 days. Tumor volume was measured and calculated as described previously.

As shown in Table 7, intraperitoneal administration of Compound A1 at 6 mg/kg once daily significantly suppressed tumor growth (TGI=37.27%, P=0.018). Compound 52 administered at 13.44 mg/kg (molar equivalent to 6 mg/kg of Compound A1) once a day either through IP or IV resulted in 36.12% TGI (P=0.0047) and 47.22% TGI (P=0.027), respectively. However, the intravenous injection of Compound A1 at 6 mg/kg caused the death of all mice within 24 hours. Mice in other groups maintained a stable body weight during the treatment period.

TABLE 7
Dosage regimen and inhibitory effect in mouse AsPC-1 CDX model

| Compound | | Dosage | Animal | Treatment | | P value(Treatment |
|---|---|---|---|---|---|---|
| dosing/route/frequency | | (mg/kg) | number | duration | TGI (%) | vs vehicle) |
| Vehicle | IP, qd | 0 | 5 | 27 | NA | NA |
| Compound A1 | IP, qd | 6 | 5 | 27 | 37.27 | * |
| Compound A1 | IV, qd | 6 | 3 | 1 | NA^{D} | NA^{D} |
| Compound 52 | IP, qd | 13.44 | 5 | 27 | 36.12 | *** |
| Compound 52 | IV, qd | 13.44 | 6 | 27 | 47.22 | * |

D: animal death observed;
* p < 0.05;
*** p < 0.001;
qd: once daily.

FIG. 6 shows that intraperitoneal administration of Compound 52 and Compound A1, as well as intravenous injection of Compound 52 resulted in significant tumor growth inhibition compared to vehicle control. These results show that compounds of the disclosure are efficacious in suppressing the activity of the KRAS^{G12D} mutant in an animal model and support the potential benefit of compounds of the disclosure for treating diseases associated with the KRAS^{G12D} mutation.

### Example 3. Efficacy Study in Mouse GP2D CDX Model

I. Inhibitory Effect on Tumor Growth in Mice after Intraperitoneal Injection of Compounds

After Balb/C nude mice (aged 6-8 weeks) were inoculated subcutaneously with 5×10⁶ GP2D colonrectal cancer cells, in 0.1 ml PBS, they were grouped 11 days post-inoculation and treated with intraperitoneal injections of the given compounds for 21 days. Note that Compound 52 at 40.3 mg/kg and Compound 53 at 43 mg/kg are molar equivalent to 18 mg/kg of Compound A1. Tumor volume and TGI were measured and calculated as described before (Table 8).

II. Inhibitory Effect on Tumor Growth in Mice after Intravenous Injection of Compounds

Balb/C nude mice, aged 6-8 weeks, were inoculated subcutaneously with 5×10⁶ GP2D colonrectal cancer cells, in 0.1 ml PBS. Eleven days post the inoculation, tumor bearing animals were randomly grouped based on given compounds and vehicle. Compound A1, as positive control, was IP administered at 18 mg/kg qd for 21 days. Compound 52 at 40.3 mg/kg (molar equivalent to 18 mg/kg of Compound A1) was IV administered q3d, and subsequently switched to 27 mg/kg for 21 days. Compound 53 at 43 mg/kg (molar equivalent to 18 mg/kg of Compound A1) was either IV or IP dosed q3d for 21 days (Table 8).

TABLE 8
Dosage regimen and inhibitory effect in mouse AsPC-1 CDX model

| | | | | | | P value |
|---|---|---|---|---|---|---|
| Compound | | Dosage | Animal | Treatment | | (Treatment |
| dosing/route/frequency | | (mg/kg) | number | duration | TGI (%) | vs vehicle) |
| Vehicle | IP, qd | 0 | 6 | 21 | NA | NA |
| CompoundA1 | IP, qd | 18 | 6 | 21 | 85.0 | * |
| Compound 52 | IP, qd | 40.3 | 6 | 21 | 87.1 | *** |
| Compound 52 | IV, q3d | 40.3 | 6 | 3 | NA^{D} | NA^{D} |
| (18 mg/kg) | | | | | | |
| Compound 52 | IV, q3d | 27 | 6 | 21 | 80.0 | *** |
| (12 mg/kg) | | | | | | |
| Compound 53 | IP, q3d | 43 | 6 | 21 | 90.0 | *** |
| Compound 53 | IV, q3d | 43 | 6 | 21 | 75.6 | *** |

D: animal death observed;
* p < 0.05;
** p < 0.01;
*** p < 0.001;
qd: once daily;
q3d: once every 3 days.

In vivo efficacy data in Table 8 shows that Compounds 52 and 53, when parenterally administered with the depicted dosages in mice, resulted in significant growth inhibition of tumors harboring the KRAS^{G12D} mutation. It is noted that mice intravenously dosed with Compound 52 at 40.3 mg/kg (molar equivalent to 18 mg/kg of Compound A1) induced death of 50% of recipient animals. However, when the IV dosage of Compound 52 was reduced to 27 mg/kg (molar equivalent to 12 mg/kg of Compound A1) and was administered once every 3 days, it resulted in obvious tumor suppression (TGI=80.0%), while the body weight of mice remained normal. These results demonstrate strong inhibitory activity for compounds of the disclosure and suggest potential beneficial effect of the compounds for diseases associated with the KRAS^{G12D} mutation.

The contents of all documents and references cited herein are hereby incorporated by reference in their entirety.

Although this invention is described in detail with reference to embodiments thereof, these embodiments are offered to illustrate but not to limit the invention. It is possible to make other embodiments that employ the principles of the invention and that fall within its spirit and scope as defined by the claims appended hereto.

What is claimed is:

1. A compound having the structure of Formula (A) or a pharmaceutically acceptable salt, ester, hydrate, solvate or stereoisomer thereof: 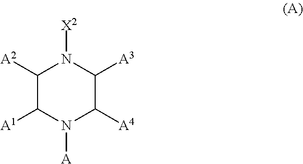
wherein:
X² is hydrogen, substituted or unsubstituted alkyl, substituted or unsubstituted heteroalkyl, substituted or unsubstituted acyl, one or more amino acid residues, substituted or unsubstituted oligopeptide, phosphoryl, phosphonyl, aminophosphonyl, sulfonyl, thioacyl, substituted or unsubstituted benzyl, substituted or unsubstituted alkoxycarbonyl, substituted or unsubstituted aminocarbonyl, substituted or unsubstituted mercaptothiocarbonyl, substituted or unsubstituted alkylthio, substituted or unsubstituted ester alkyl, substituted or unsubstituted benzyloxycarbonyl, glucoside, glucuronide, or cholic acid substituent;
A is an organic group containing a cyclic structure which is a monocyclic ring, a bicyclic ring, a fused ring, a bridged ring, a spiro ring, a heterocyclic ring, an aromatic ring, an aromatic heterocyclic ring, an aliphatic ring, or any combination thereof, wherein the cyclic structure contains two or more substituent groups; and
A¹, A², A³ and A⁴ are independently hydrogen or a C₁-C₆ hydrocarbyl group, or
one or two of the A¹, A², A³ and A⁴ groups is connected to the piperazine ring and forms a bridged ring, a fused ring, or a spiro ring and the remaining one or two of the A¹, A², A³ and A⁴ groups are independently hydrogen or a C₁-C₆ hydrocarbyl group.

2. The compound of claim 1, wherein the compound has the structure of Formula (B) or a pharmaceutically acceptable salt, ester, hydrate, solvate or stereoisomer thereof: 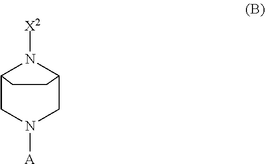
wherein:
X² is hydrogen, substituted or unsubstituted alkyl, substituted or unsubstituted heteroalkyl, substituted or unsubstituted acyl, one or more amino acid residues, substituted or unsubstituted oligopeptide, phosphoryl, phosphonyl, aminophosphonyl, sulfonyl, thioacyl, substituted or unsubstituted benzyl, substituted or unsubstituted alkoxycarbonyl, substituted or unsubstituted aminocarbonyl, substituted or unsubstituted mercaptothiocarbonyl, substituted or unsubstituted alkylthio, substituted or unsubstituted ester alkyl, substituted or unsubstituted benzyloxycarbonyl, glucoside, glucuronide, or cholic acid substituent; and
A is an organic group containing a cyclic structure which is a monocyclic ring, a bicyclic ring, a fused ring, a bridged ring, a spiro ring, a heterocyclic ring, an aromatic ring, a heteroaromatic ring, an aliphatic ring, or any combination thereof, wherein the cyclic structure contains two or more substituent groups;
optionally wherein the substituted or unsubstituted acyl is saturated or unsaturated aliphatic acyl or aroyl: the substituted or unsubstituted oligopeptide is a dipeptide, a tripeptide, or a tetrapeptide; and/or the substituted or unsubstituted alkylthio is thiocarbonyl.

3-8. (canceled)

9. The compound of claim 1, wherein the compound has the structure of Formula (I) or a pharmaceutically acceptable salt, ester, hydrate, solvate or stereoisomer thereof: 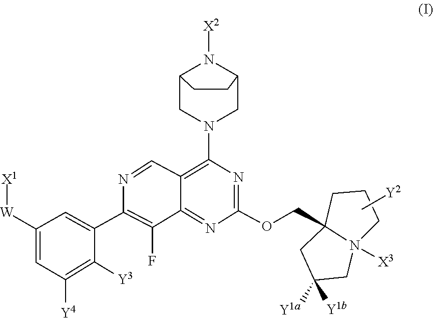
wherein:
W is oxygen (O), sulfur (S) or nitrogen (NH);
X¹ and X² are independently hydrogen, substituted or unsubstituted alkyl, substituted or unsubstituted heteroalkyl, substituted or unsubstituted acyl, one or more amino acid residue, substituted or unsubstituted oligopeptide, phosphoryl, phosphonyl, aminophosphonyl, sulfonyl, thioacyl, substituted or unsubstituted benzyl, substituted or unsubstituted alkoxycarbonyl, substituted or unsubstituted aminocarbonyl, substituted or unsubstituted mercaptothiocarbonyl, substituted or unsubstituted alkylthio, substituted or unsubstituted ester alkyl, substituted or unsubstituted benzyloxycarbonyl, glucoside, glucuronide, or cholic acid substituent;
X³ is 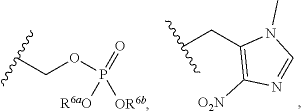
or lone pair electrons, wherein when X³ is lone pair electrons, X¹ and X² cannot both be hydrogen, and when X³ is 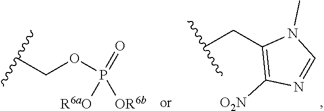
the N atom links to X³ and forms a quaternary ammonium ion with a positive charge which further forms an inner salt with an intramolecular negative ion or a salt with additional acid molecule(s), where R^{6a} and R^{6b} are independently selected from hydrogen, C₁-C₂₀ hydrocarbyl, cyclohydrocarbyl, and 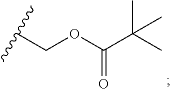 [SMILES: *COC(=O)C(C)(C)C]
Y^{1a} and Y^{1b} are independently hydrogen, halogen, hydroxy, amino, amine, hydroxymethyl, alkoxy, or acyloxy;
Y² is hydrogen, halogen, hydroxy, amino, amine, hydroxymethyl, alkoxy, acyloxy, or C₁-C₆ hydrocarbyl;
Y³ and Y⁴ are independently hydrogen, halogen, or halomethyl, or Y³ and Y⁴ are connected to the benzene ring structure and form a substituted or unsubstituted benzo fused ring.

10. (canceled)

11. The compound of claim 9, wherein X¹ and X² are independently hydrogen, C₁-C₂₀ saturated or unsaturated alkoxycarbonyl, C₁-C₂₀ saturated or unsaturated alkyl acyl, 6- to 15-membered (hetero) arylcarbonyl, 4- to 15-membered (hetero) cycloalkylcarbonyl, C₁-C₂₀ alkylthio, 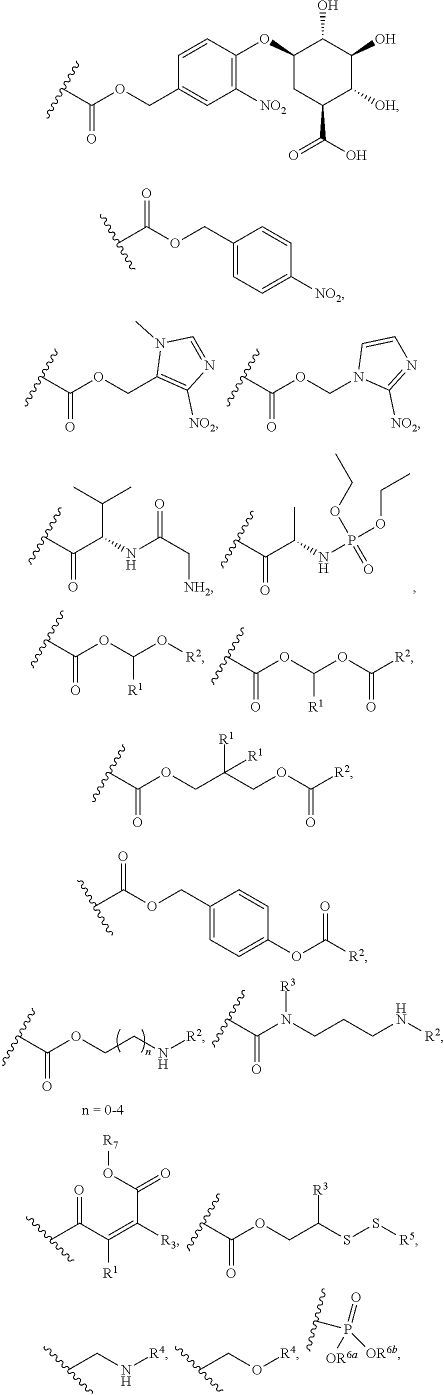 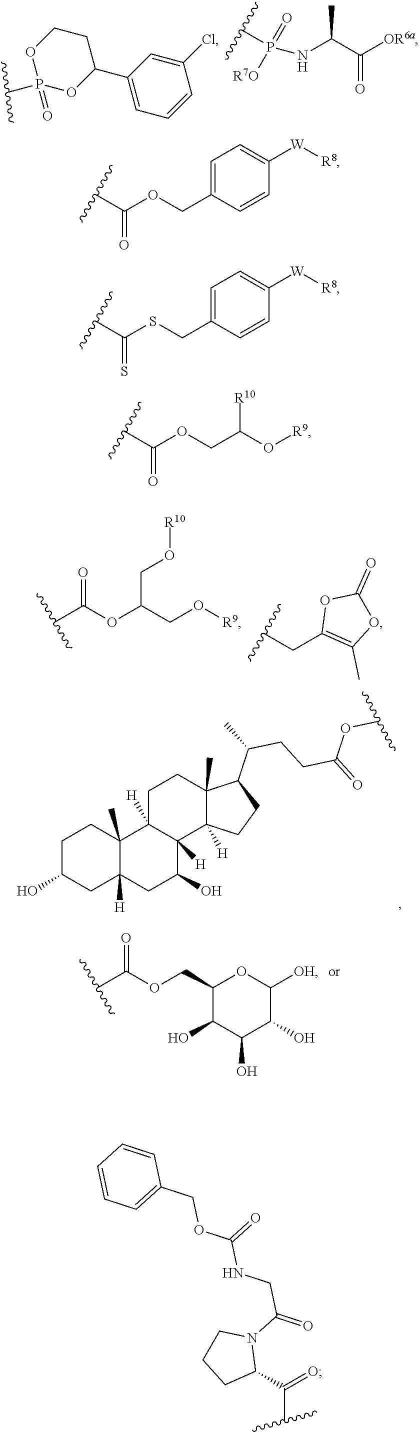
wherein,
R¹ is hydrogen, methyl, ethyl, propyl, isopropyl, C₃-C₆ cycloalkyl, or aryl;
R² is hydrogen, C₁-C₂₀ saturated or unsaturated alkyl, C₁-C₂₀ saturated or unsaturated alkyl acyl, heteroazanyl, aryl hydrocarbyl, heterocyclic aromatic hydrocarbyl, C₃-C₈ carbocyclic or heterocyclic hydrocarbyl, fused ring, naphthalene ring, bridged ring hydrocarbyl, one or more amino acid residues, 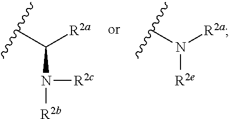
wherein,
R^{2a}, R^{2b}, R^{2c}, R^{2d} and R^{2e} are independently hydrogen, C₁-C₆ substituted or unsubstituted alkyl or C₁-C₆ substituted or unsubstituted hydrocarbyl;
R³ is hydrogen, methyl, ethyl or propyl;
R⁴ is hydrogen, C₂-C₂₀ alkyl, isopropyl, isobutyl, aryl hydrocarbyl, carbocyclic hydrocarbyl, heterocyclic hydrocarbyl, or C₂-C₂₀ alkanoyloxy;
R⁵ is ethyl with a 2-position substituted, wherein the substituents at the 2-position are amino, alkoxycarbonyl, alkanoyloxy, or acyloxy derived from amino acid;
R^{6a} and R^{6b} are independently hydrogen, C₁-C₂₀ hydrocarbyl, C₁-C₂₀ cyclic hydrocarbyl, aryl, or 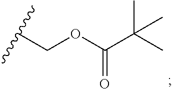 [SMILES: *COC(=O)C(C)(C)C]
R⁷ is C₁-C₆ alkyl or substituted or unsubstituted aryl;
R⁸ is C₂-C₂₀ substituted or unsubstituted saturated or unsaturated alkanoyl or saturated or unsaturated alkoxycarbonyl;
R⁹ is lower alkyl, substituted or unsubstituted benzyl, substituted or unsubstituted imidazol-5-methyl, oligoglycolyl (—[CH₂CH₂O]ₙCH₃, where n is an integer from 0 to 4), or C₂-C₂₀ saturated or unsaturated alkanoyl; and
R¹⁰ is hydrogen, C₁-C₆ alkoxy, C₂-C₂₀ saturated or unsaturated alkanoyl, C₂-C₂₀ substituted or unsubstituted saturated or unsaturated alkanoyl, or saturated or unsaturated alkoxycarbonyl.

12. The compound of claim 11, wherein:
W is oxygen (O);
X¹ is hydrogen (H) or 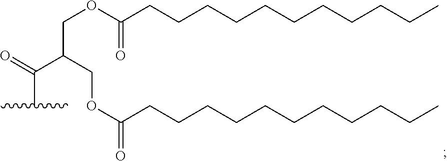 [SMILES: *C(=O)C(COC(=O)CCCCCCCCCCC)COC(=O)CCCCCCCCCCC]
and/or
X² is 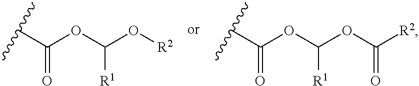
wherein R¹ is methyl, and R² is C₁-C₂₀ saturated or unsaturated alkyl or C₁-C₂₀ saturated or unsaturated alkyl acyl, optionally C₁-C₆ saturated alkyl or C₁-C₆ saturated alkyl acyl, optionally C₃ alkyl or C₃ acyl.

13.-18. (canceled)

19. The compound of a claim 11, wherein X¹ is 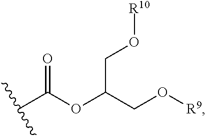
wherein R⁹ is lower alkyl, substituted or unsubstituted benzyl, substituted or unsubstituted imidazol-5-methyl, oligoglycolyl (—[CH₂CH₂O]ₙCH₃, where n is an integer from 0 to 4), or C₂-C₂₀ saturated or unsaturated alkanoyl; and R¹⁰ is hydrogen, C₁-C₆ alkoxy, C₂-C₂₀ saturated or unsaturated alkanoyl, C₂-C₂₀ substituted or unsubstituted saturated or unsaturated alkanoyl, or saturated or unsaturated alkoxycarbonyl.

20.-22. (canceled)

23. The compound of claim 9, wherein:
the substituted or unsubstituted acyl is saturated or unsaturated aliphatic acyl or aroyl;
the substituted or unsubstituted oligopeptide is a dipeptide, a tripeptide, or a tetrapeptide;
the substituted or unsubstituted alkylthio is thiocarbonyl;
the salt formed with the additional acid molecule(s) is hydrohalic acid salt;
the halogen is F, Cl, or Br;
the halomethyl is monohalomethyl, dihalomethyl, or trihalomethyl; and/or
the benzo fused ring is a naphthalene ring structure.

24. (canceled)

25. The compound of claim 1, wherein the compound has the structure of Formula (II), Formula (IIA), or Formula (III), or a pharmaceutically acceptable salt, ester, hydrate, solvate or stereoisomer thereof: 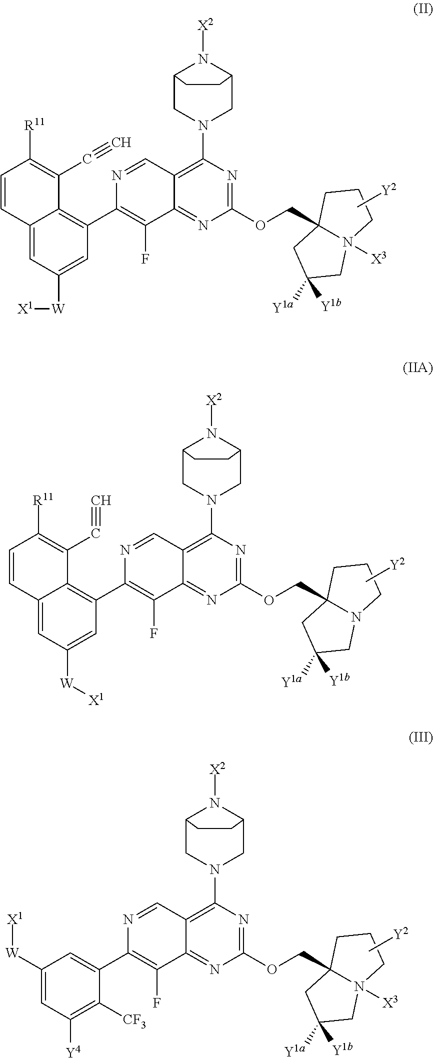
wherein:
in Formula (II), R¹¹ is hydrogen, halogen, hydroxy, substituted hydroxy, or lower alkyl;
in Formula (IIA), R¹¹ is hydrogen, halogen, hydroxy, substituted hydroxy, or lower alkyl; and
in Formula (III), Y⁴ is hydrogen, halogen, hydroxyl, substituted hydroxyl, or lower alkyl.

26. The compound of claim 25, wherein W is oxygen; X¹ is hydrogen (H); R¹¹ is hydrogen (H) or fluorine (F): Y² is fluorine (F); and/or Y^{1a} and Y^{1b} are both hydrogen.

27.-36. (canceled)

37. The compound of claim 25, wherein X³ is 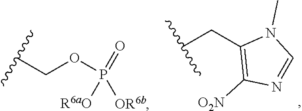
or lone pair electrons.

38.-39. (canceled)

40. The compound of at claim 25, wherein in Formula (III), W is oxygen and Y⁴ is chlorine, or W is NH and Y⁴ is hydrogen or chlorine; optionally, wherein Y^{1b} and Y² are both hydrogen.

41.-42. (canceled)

43. The compound of claim 1, wherein the compound has the structure of any one of Formulae (IV) to (VII), or a pharmaceutically acceptable salt, ester, hydrate, solvate or stereoisomer thereof:

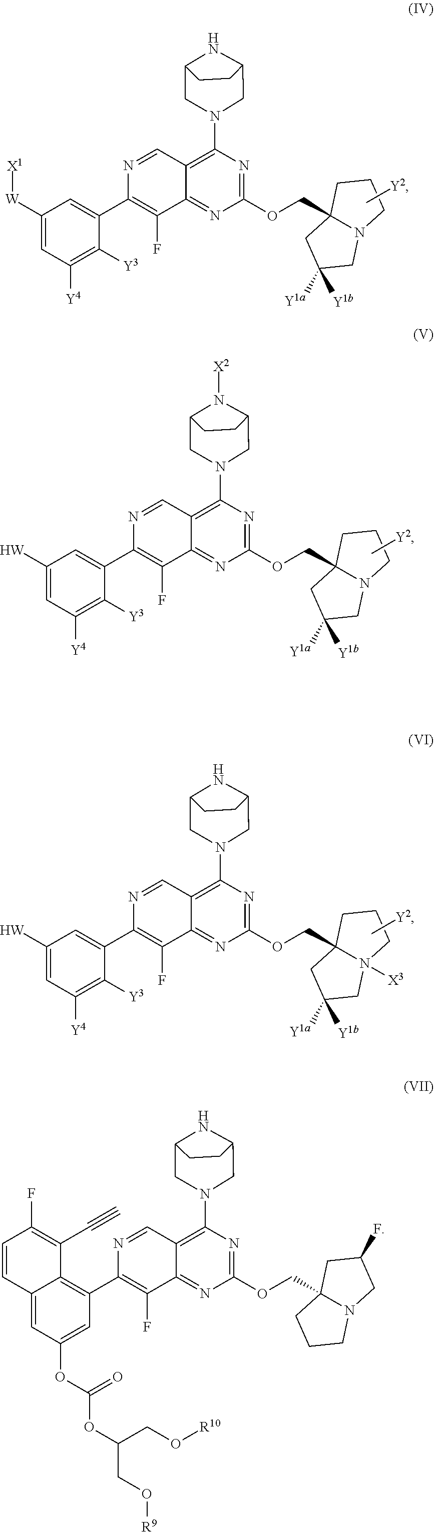

44. The compound of claim 43, wherein R⁹ is lower alkyl, substituted or unsubstituted benzyl, substituted or unsubstituted imidazol-5-methyl, oligoglycolyl (—[CH₂CH₂O]ₙCH₃, where n is an integer from 0 to 4), or C₂-C₂₀ saturated or unsaturated alkanoyl; and R¹⁰ is hydrogen, C₁-C₆ alkoxy, C₂-C₂₀ saturated or unsaturated alkanoyl, C₂-C₂₀ substituted or unsubstituted saturated or unsaturated alkanoyl, or saturated or unsaturated alkoxycarbonyl.

45.-47. (canceled)

48. A compound which is: 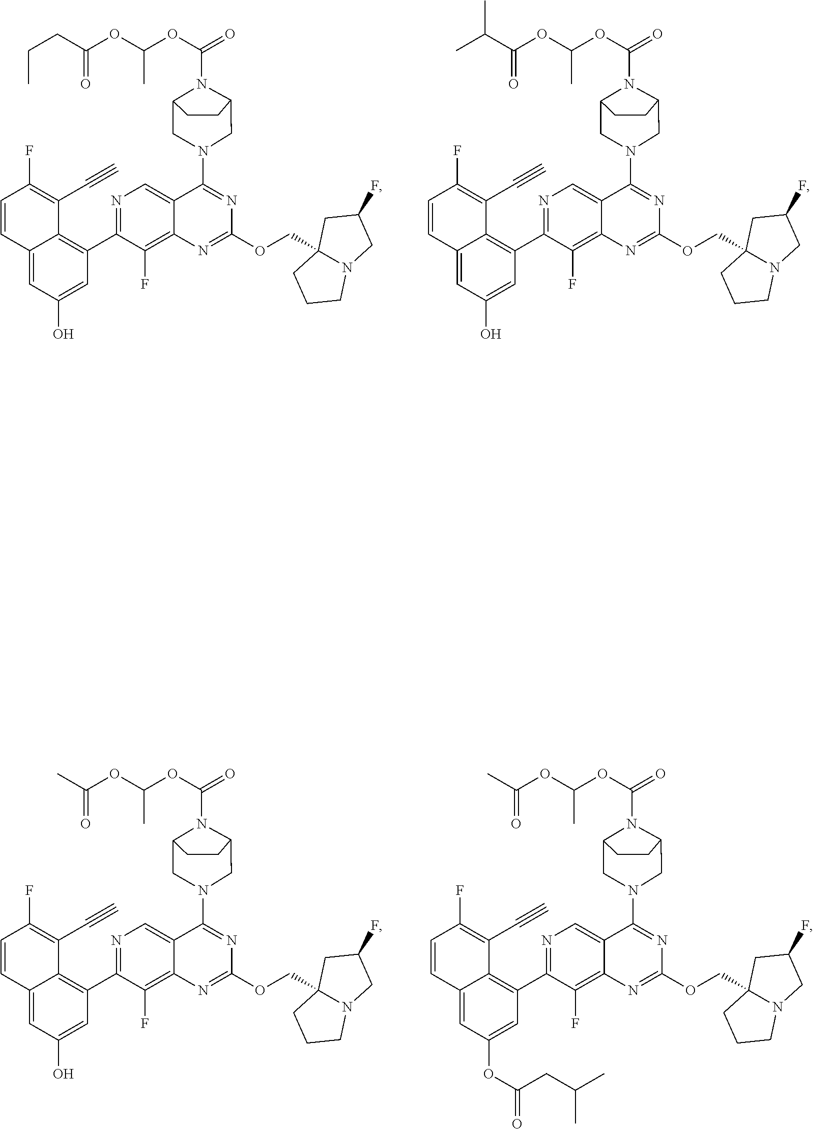 [SMILES: C#Cc1c(F)ccc2cc(O)cc(-c3ncc4c(N5CC6CCC(C5)N6C(=O)OC(C)OC(=O)CCC)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)OC(C)OC(=O)C(C)C)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)OC(C)OC(C)=O)nc(OC[C@@]56CCCN5C[C@H](F)C6)nc4c3F)c12 ; C#Cc1c(F)ccc2cc(OC(=O)CC(C)C)cc(-c3ncc4c(N5CC6CCC(C5)N6C(=O)OC(C)OC(C)=O)nc(OC[C@@]56CCCN5C[C@H](F)C6)nc4c3F)c12] 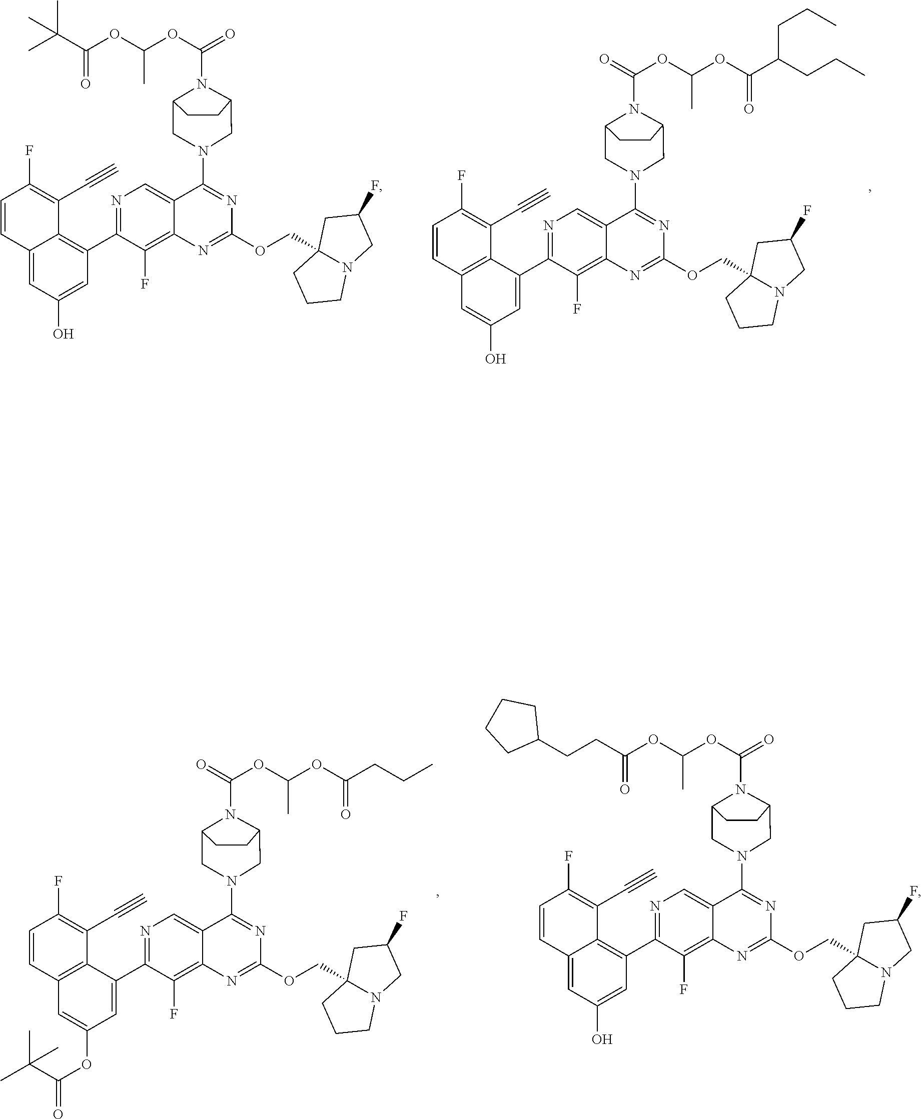 [SMILES: C#Cc1c(F)ccc2cc(O)cc(-c3ncc4c(N5CC6CCC(C5)N6C(=O)OC(C)OC(=O)C(C)(C)C)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)OC(C)OC(=O)C(CCC)CCC)nc(OC[C@@]56CCCN5C[C@H](F)C6)nc4c3F)c12 ; C#Cc1c(F)ccc2cc(OC(=O)C(C)(C)C)cc(-c3ncc4c(N5CC6CCC(C5)N6C(=O)OC(C)OC(=O)CCC)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)OC(C)OC(=O)CCC5CCCC5)nc(OC[C@@]56CCCN5C[C@H](F)C6)nc4c3F)c12] 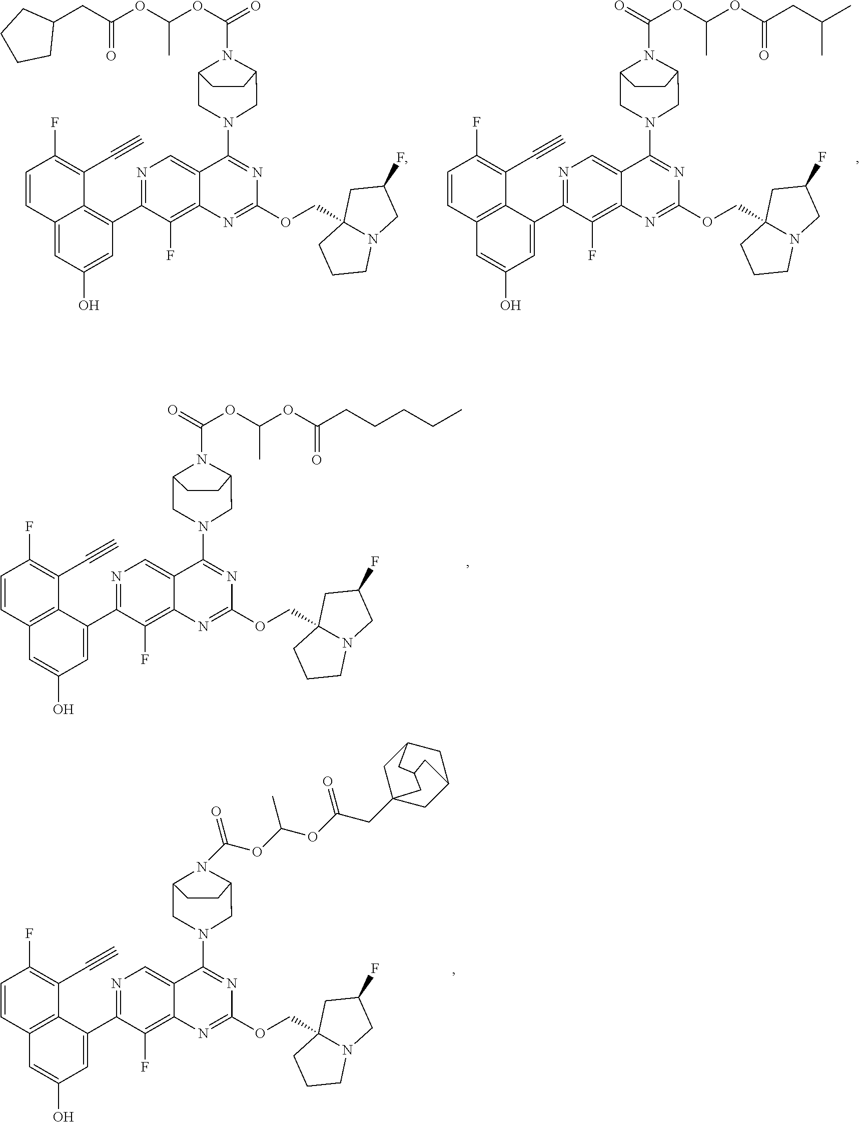 [SMILES: C#Cc1c(F)ccc2cc(O)cc(-c3ncc4c(N5CC6CCC(C5)N6C(=O)OC(C)OC(=O)CC5CCCC5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)OC(C)OC(=O)CC(C)C)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)OC(C)OC(=O)CCCCC)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)OC(C)OC(=O)CC56CC7CC(CC(C7)C5)C6)nc(OC[C@@]56CCCN5C[C@H](F)C6)nc4c3F)c12] 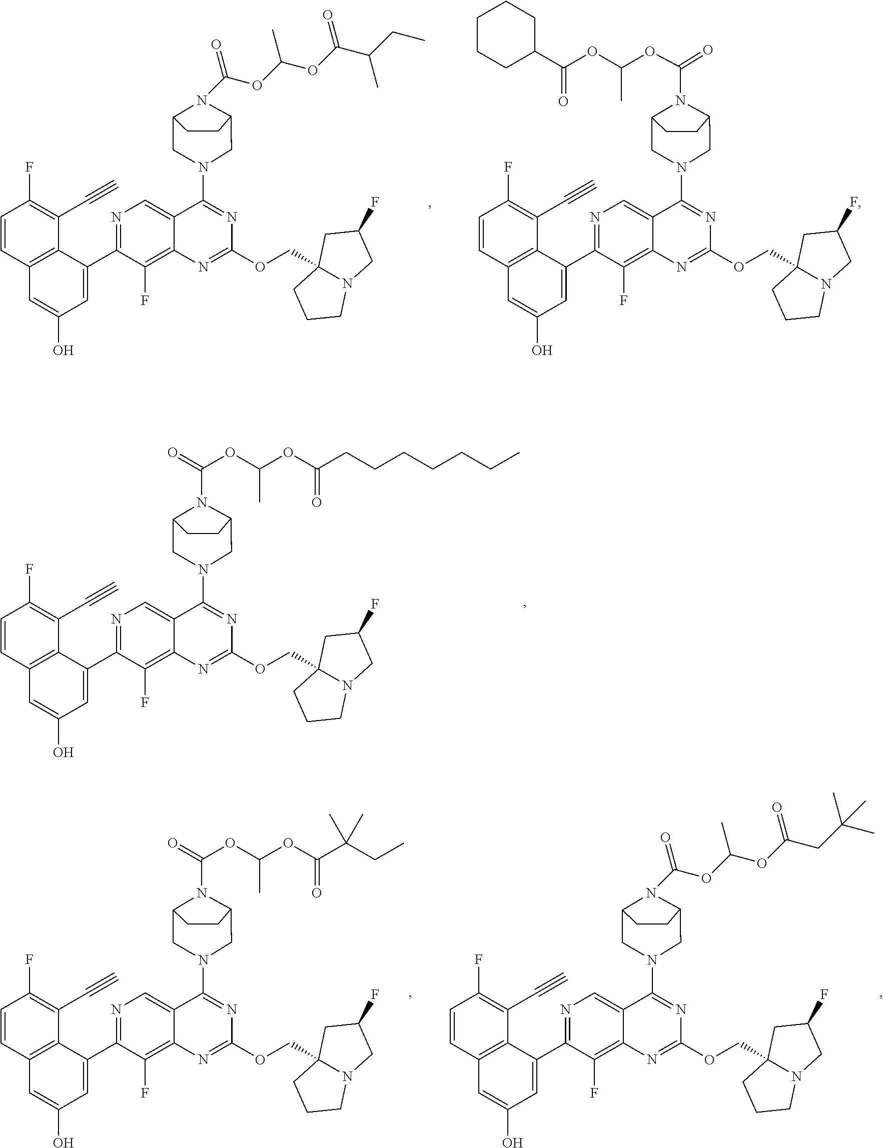 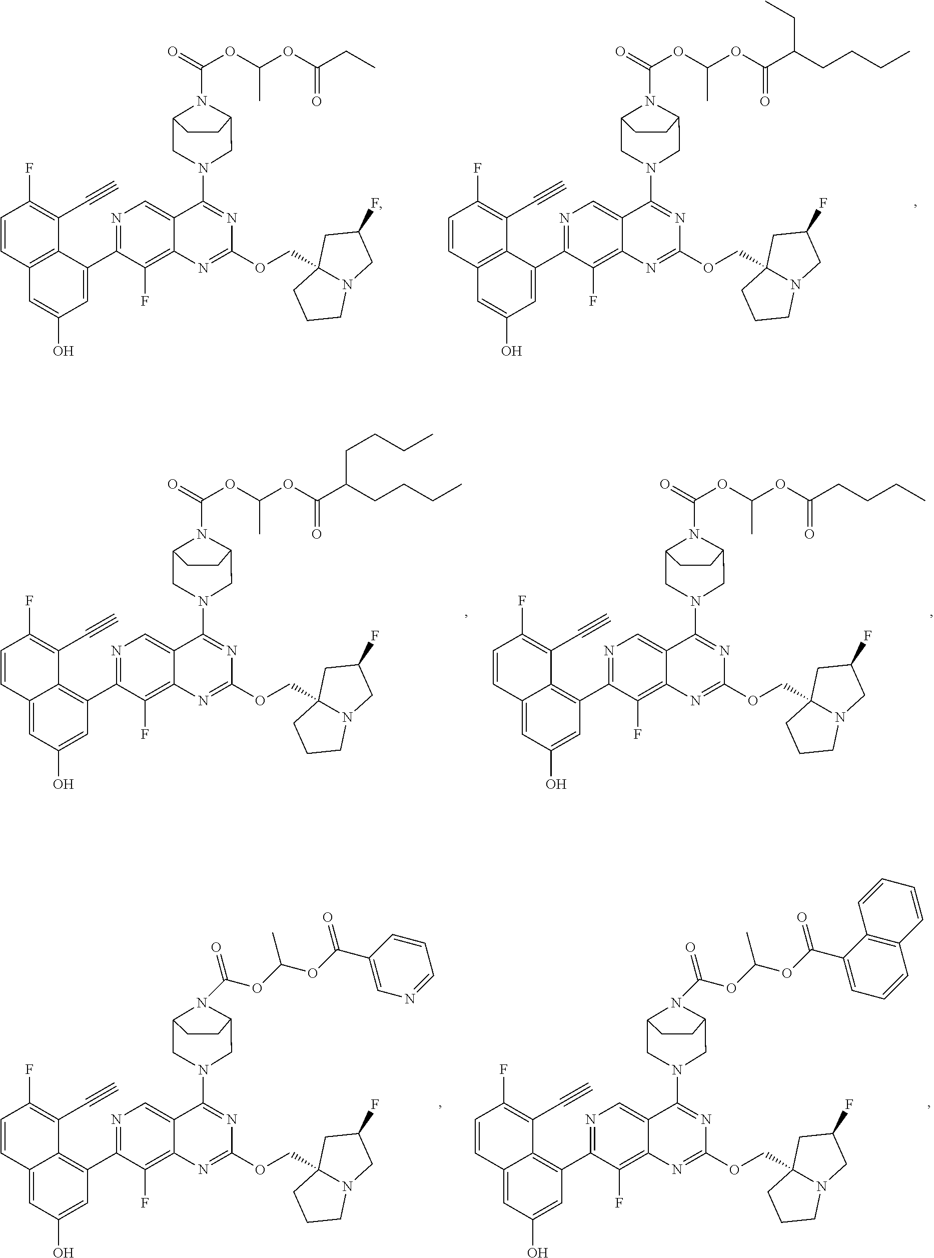 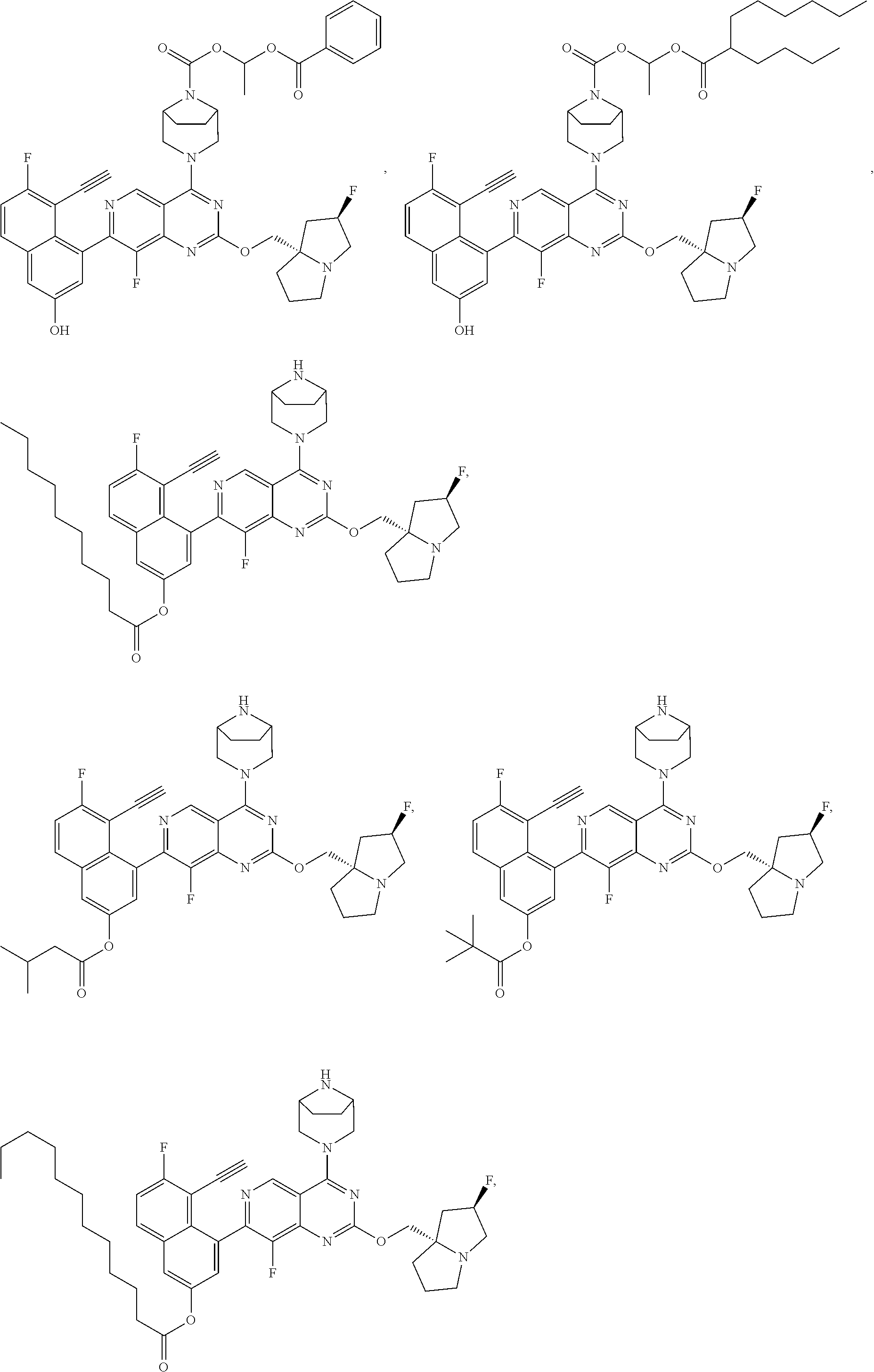 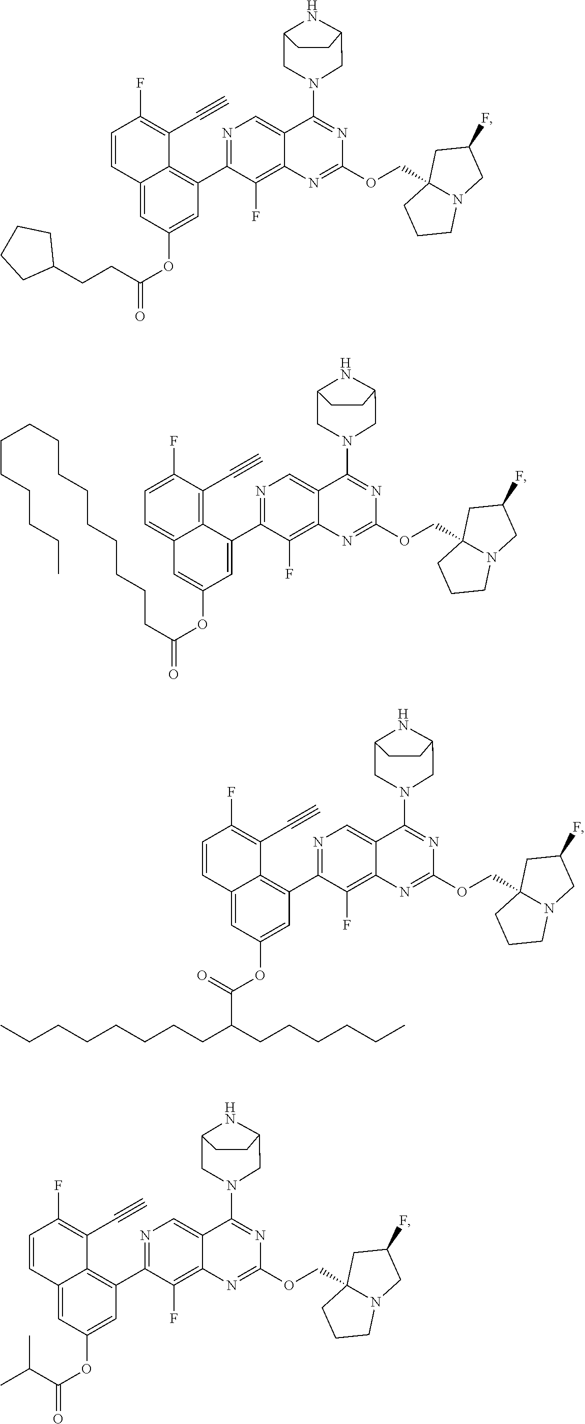 [SMILES: C#Cc1c(F)ccc2cc(OC(=O)CCC3CCCC3)cc(-c3ncc4c(N5CC6CCC(C5)N6)nc(OC[C@@]56CCCN5C[C@H](F)C6)nc4c3F)c12 ; C#Cc1c(F)ccc2cc(OC(=O)CCCCCCCCCCCCCCC)cc(-c3ncc4c(N5CC6CCC(C5)N6)nc(OC[C@@]56CCCN5C[C@H](F)C6)nc4c3F)c12 ; C#Cc1c(F)ccc2cc(OC(=O)C(CCCCCC)CCCCCCCC)cc(-c3ncc4c(N5CC6CCC(C5)N6)nc(OC[C@@]56CCCN5C[C@H](F)C6)nc4c3F)c12 ; C#Cc1c(F)ccc2cc(OC(=O)C(C)C)cc(-c3ncc4c(N5CC6CCC(C5)N6)nc(OC[C@@]56CCCN5C[C@H](F)C6)nc4c3F)c12] 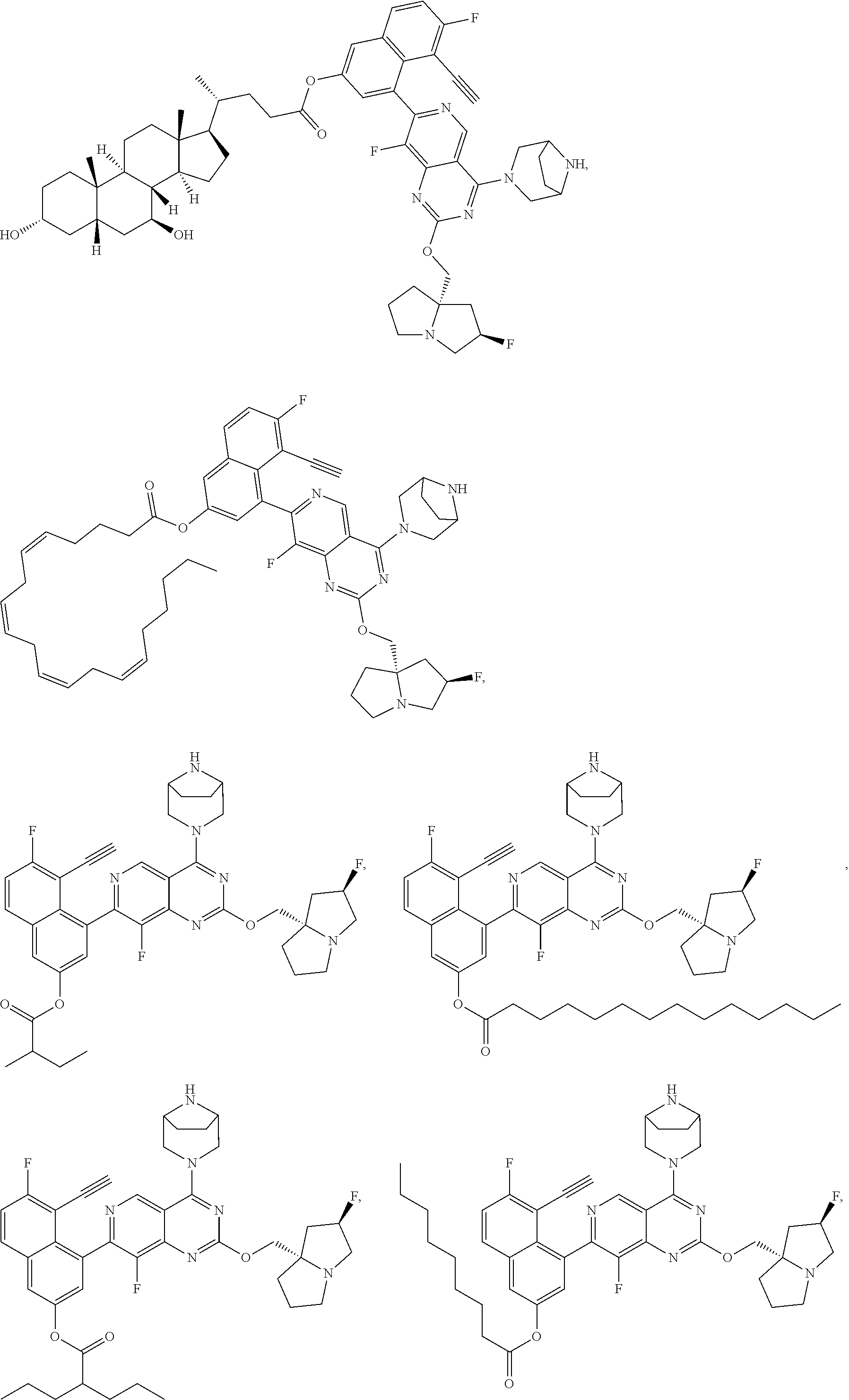 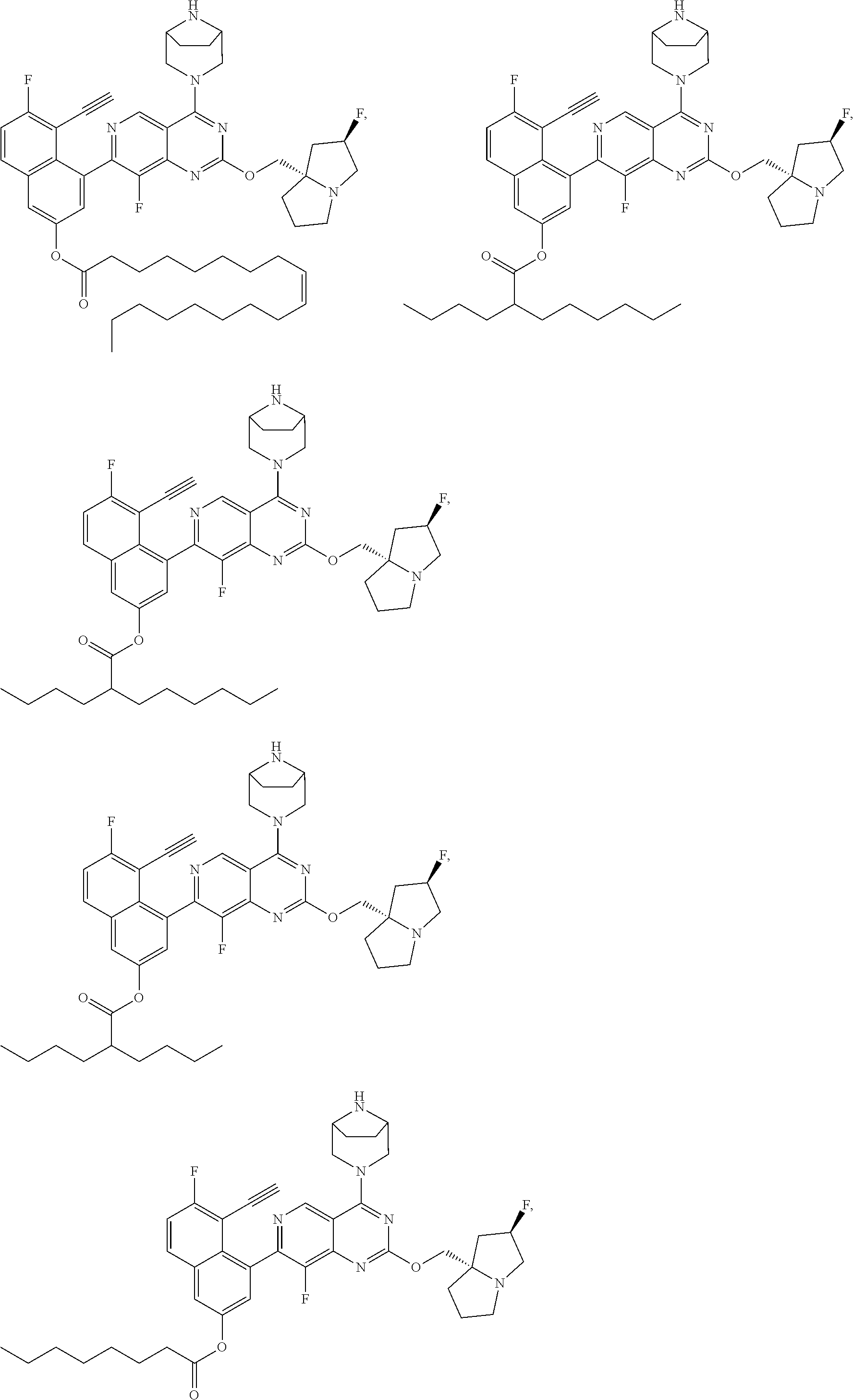 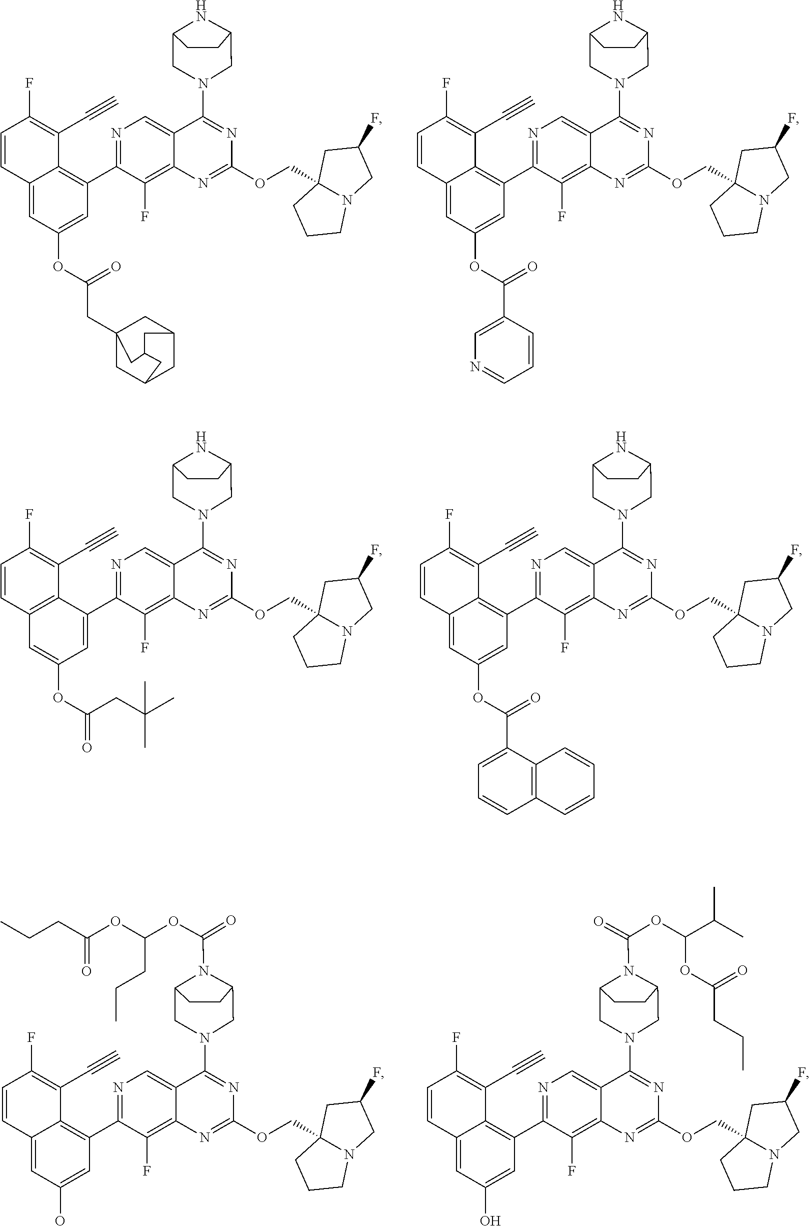 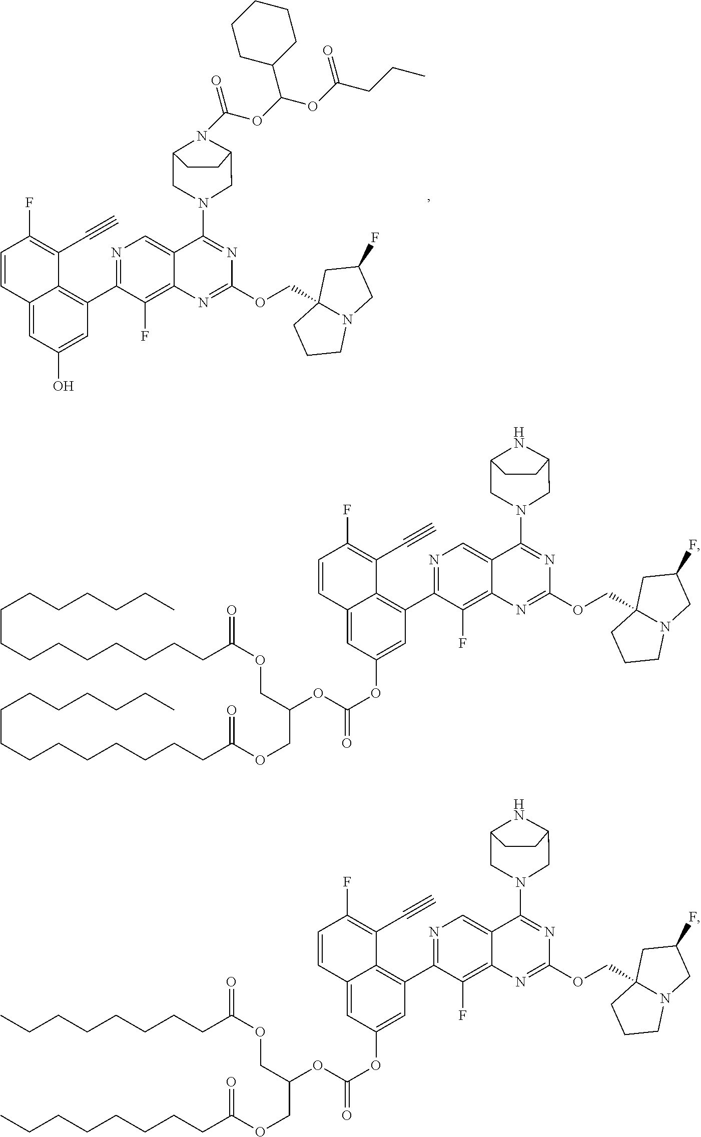 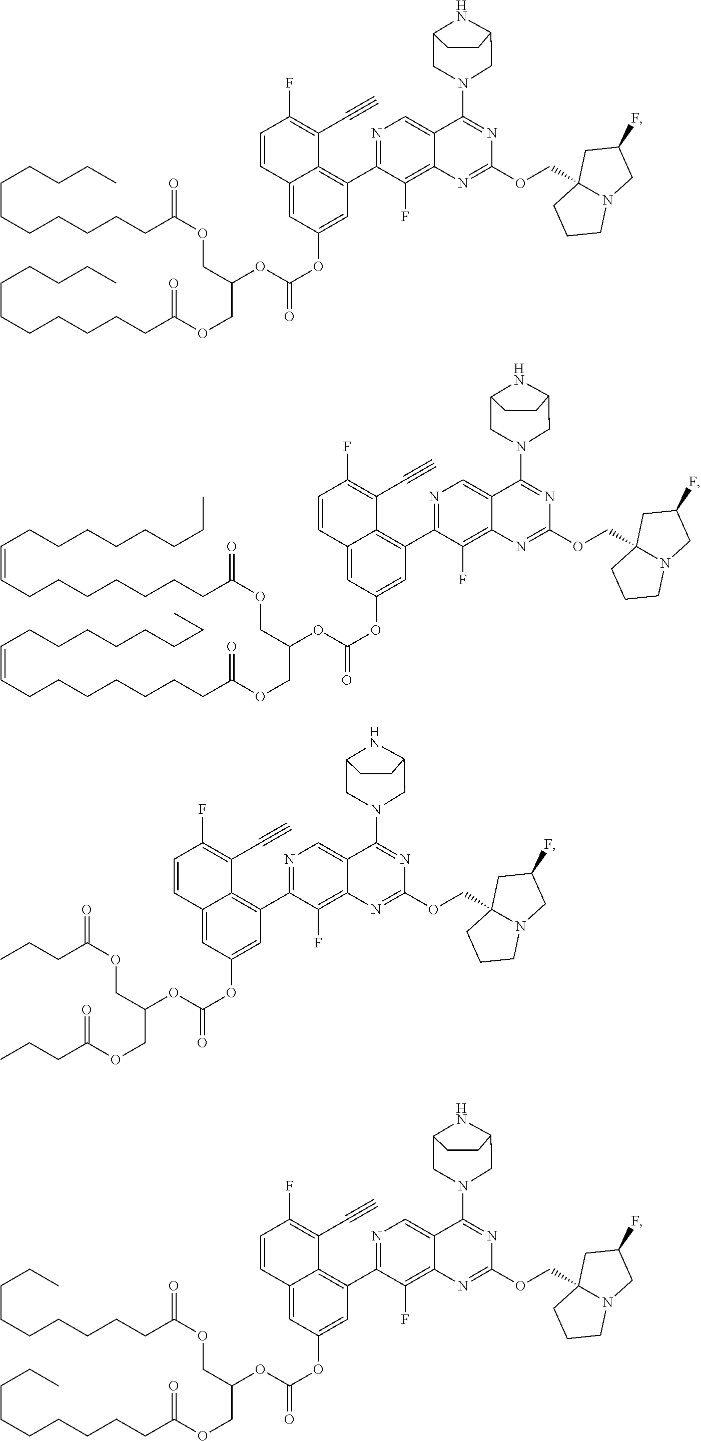 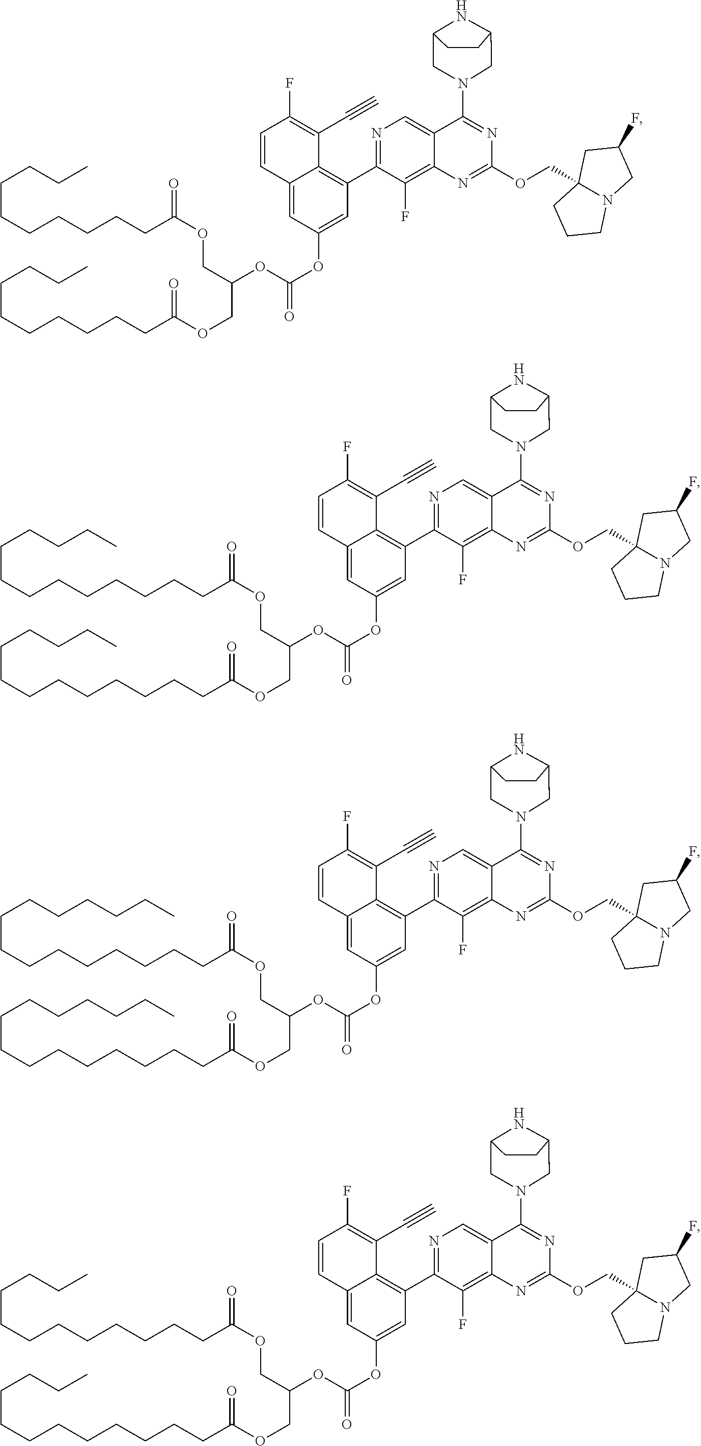 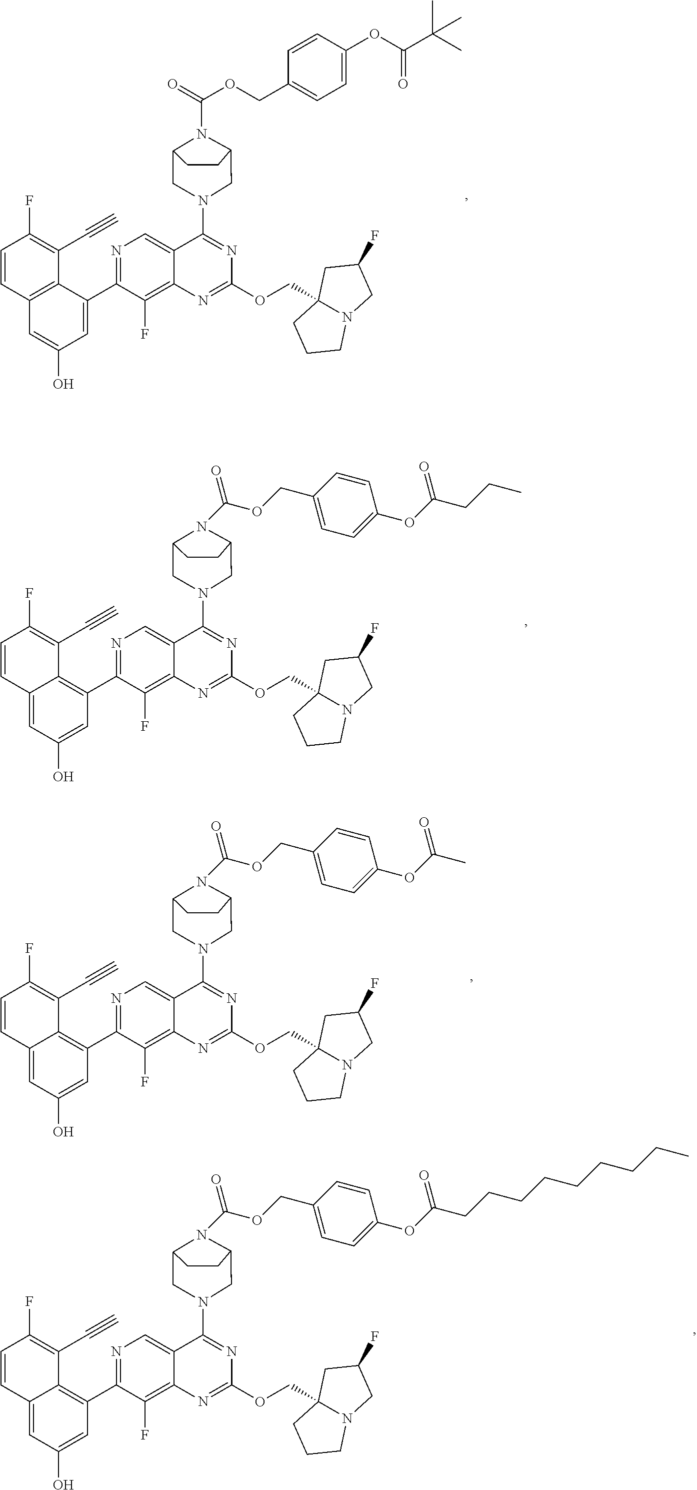 [SMILES: C#Cc1c(F)ccc2cc(O)cc(-c3ncc4c(N5CC6CCC(C5)N6C(=O)OCc5ccc(OC(=O)C(C)(C)C)cc5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)OCc5ccc(OC(=O)CCC)cc5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)OCc5ccc(OC(C)=O)cc5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)OCc5ccc(OC(=O)CCCCCCCCC)cc5)nc(OC[C@@]56CCCN5C[C@H](F)C6)nc4c3F)c12] 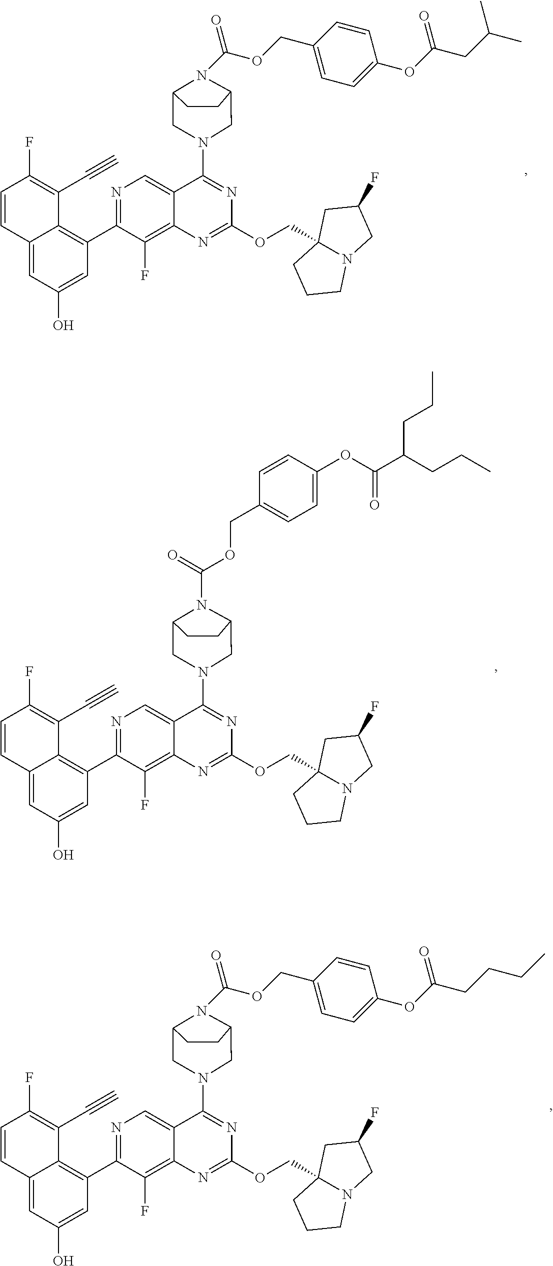 [SMILES: C#Cc1c(F)ccc2cc(O)cc(-c3ncc4c(N5CC6CCC(C5)N6C(=O)OCc5ccc(OC(=O)CC(C)C)cc5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)OCc5ccc(OC(=O)C(CCC)CCC)cc5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)OCc5ccc(OC(=O)CCCC)cc5)nc(OC[C@@]56CCCN5C[C@H](F)C6)nc4c3F)c12] 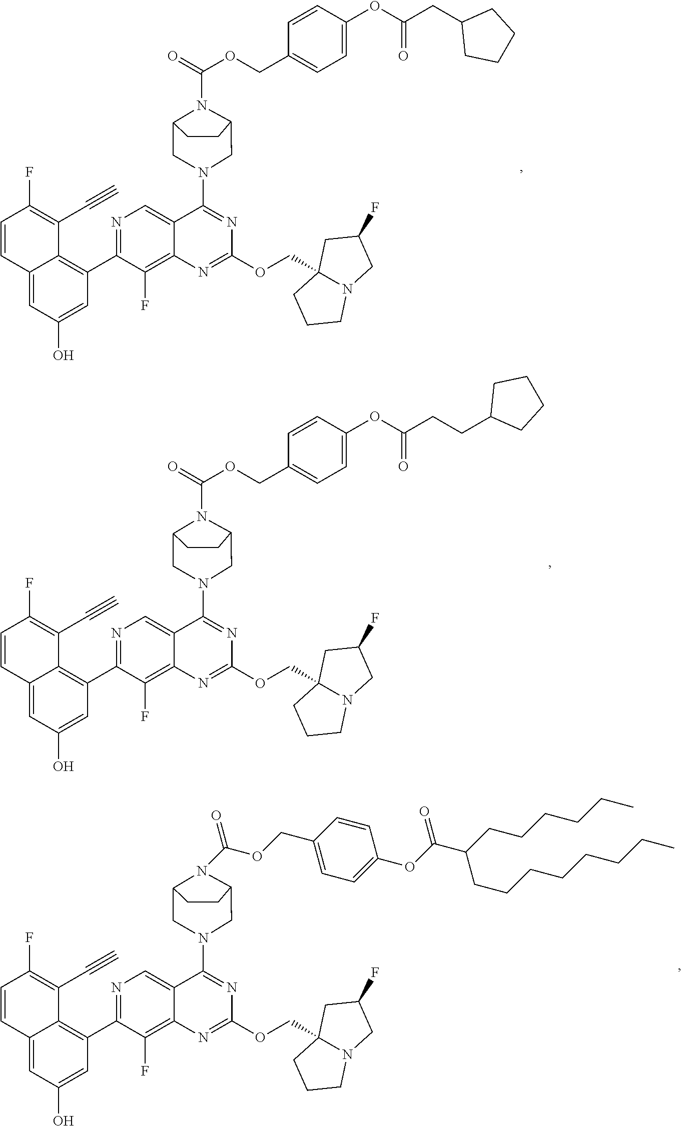 [SMILES: C#Cc1c(F)ccc2cc(O)cc(-c3ncc4c(N5CC6CCC(C5)N6C(=O)OCc5ccc(OC(=O)CC6CCCC6)cc5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)OCc5ccc(OC(=O)CCC6CCCC6)cc5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)OCc5ccc(OC(=O)C(CCCCCC)CCCCCCCC)cc5)nc(OC[C@@]56CCCN5C[C@H](F)C6)nc4c3F)c12] 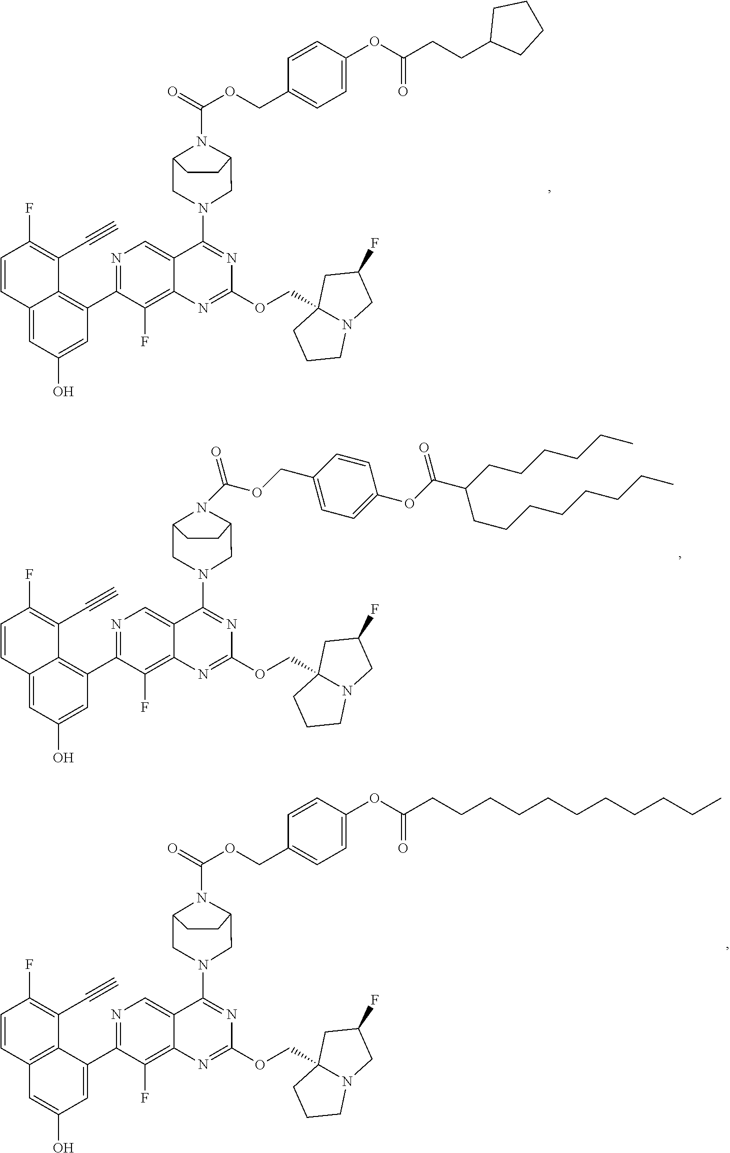 [SMILES: C#Cc1c(F)ccc2cc(O)cc(-c3ncc4c(N5CC6CCC(C5)N6C(=O)OCc5ccc(OC(=O)CCC6CCCC6)cc5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)OCc5ccc(OC(=O)C(CCCCCC)CCCCCCCC)cc5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)OCc5ccc(OC(=O)CCCCCCCCCCC)cc5)nc(OC[C@@]56CCCN5C[C@H](F)C6)nc4c3F)c12] 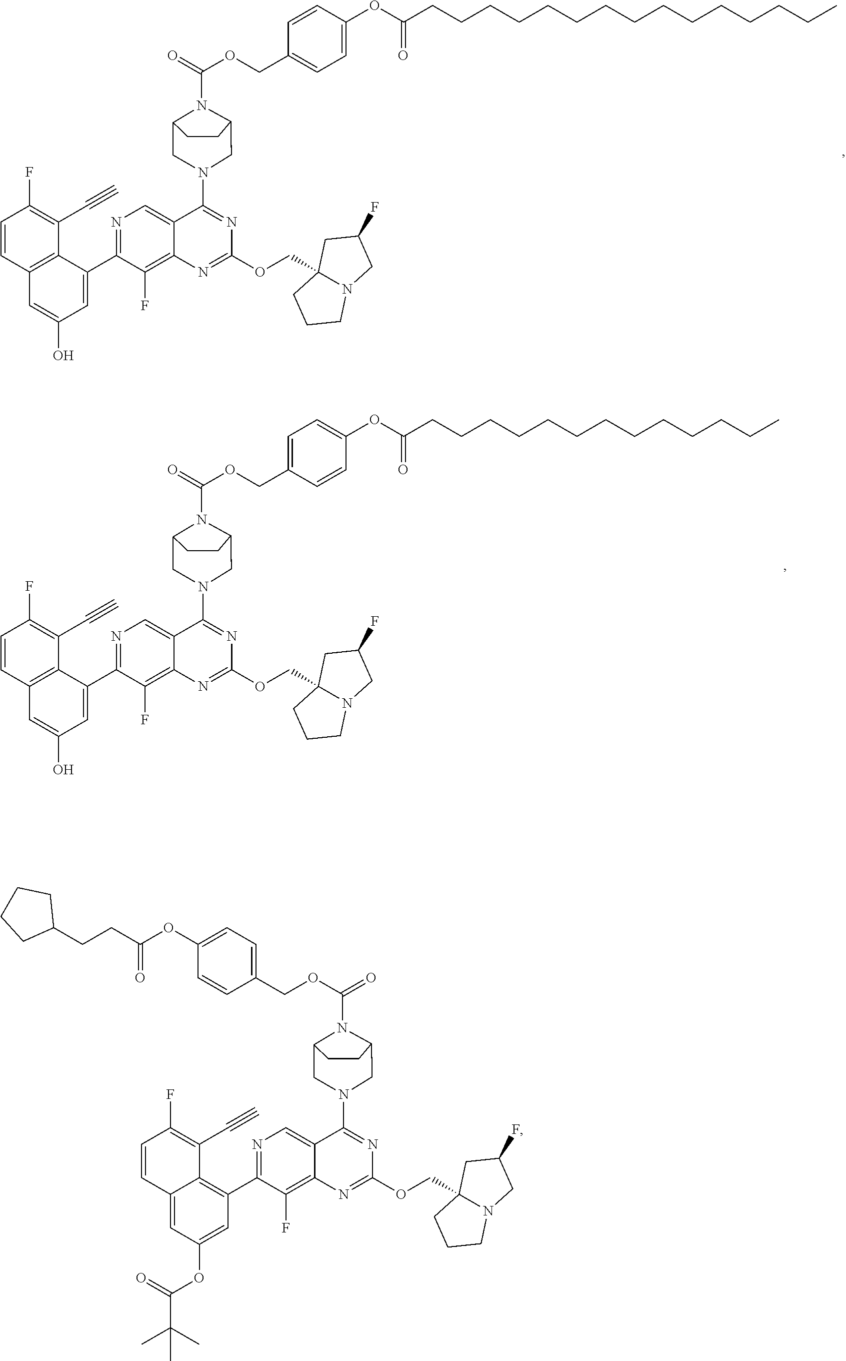 [SMILES: C#Cc1c(F)ccc2cc(O)cc(-c3ncc4c(N5CC6CCC(C5)N6C(=O)OCc5ccc(OC(=O)CCCCCCCCCCCCCCC)cc5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)OCc5ccc(OC(=O)CCCCCCCCCCCCC)cc5)nc(OC[C@@]56CCCN5C[C@H](F)C6)nc4c3F)c12 ; C#Cc1c(F)ccc2cc(OC(=O)C(C)(C)C)cc(-c3ncc4c(N5CC6CCC(C5)N6C(=O)OCc5ccc(OC(=O)CCC6CCCC6)cc5)nc(OC[C@@]56CCCN5C[C@H](F)C6)nc4c3F)c12] 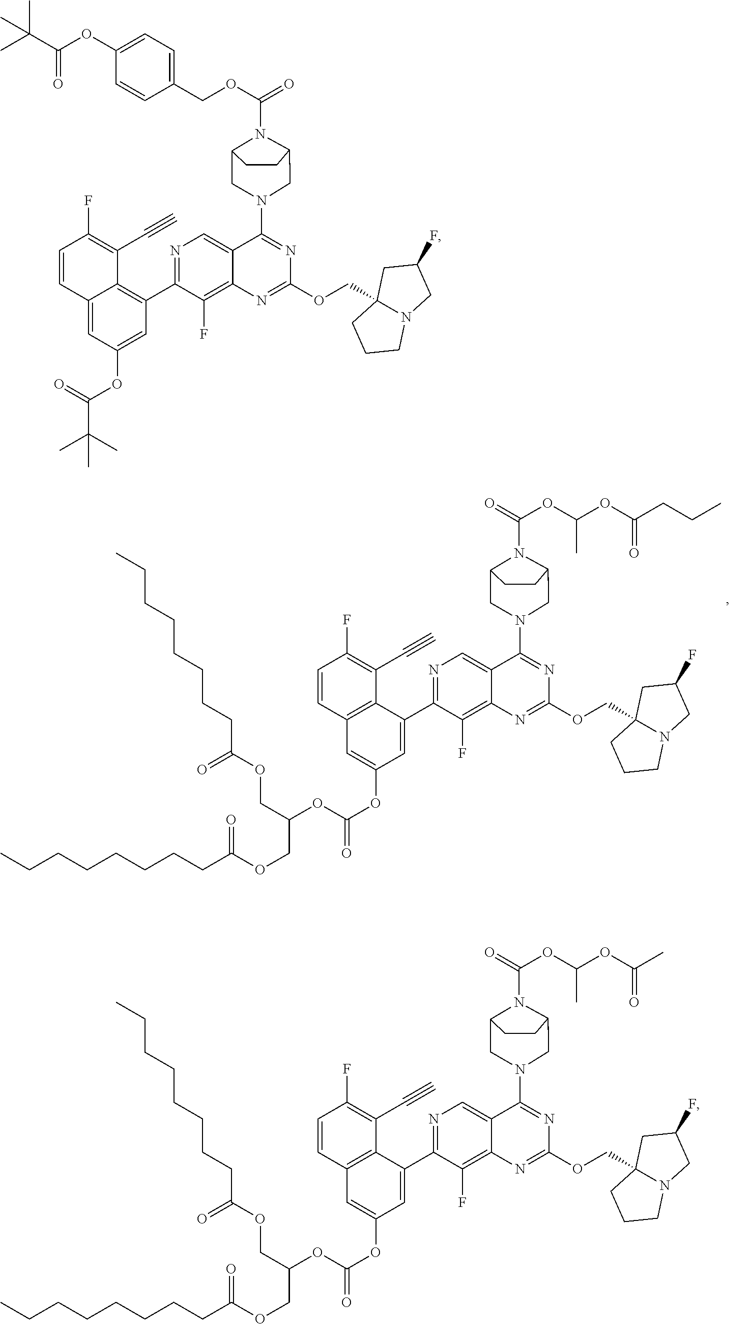 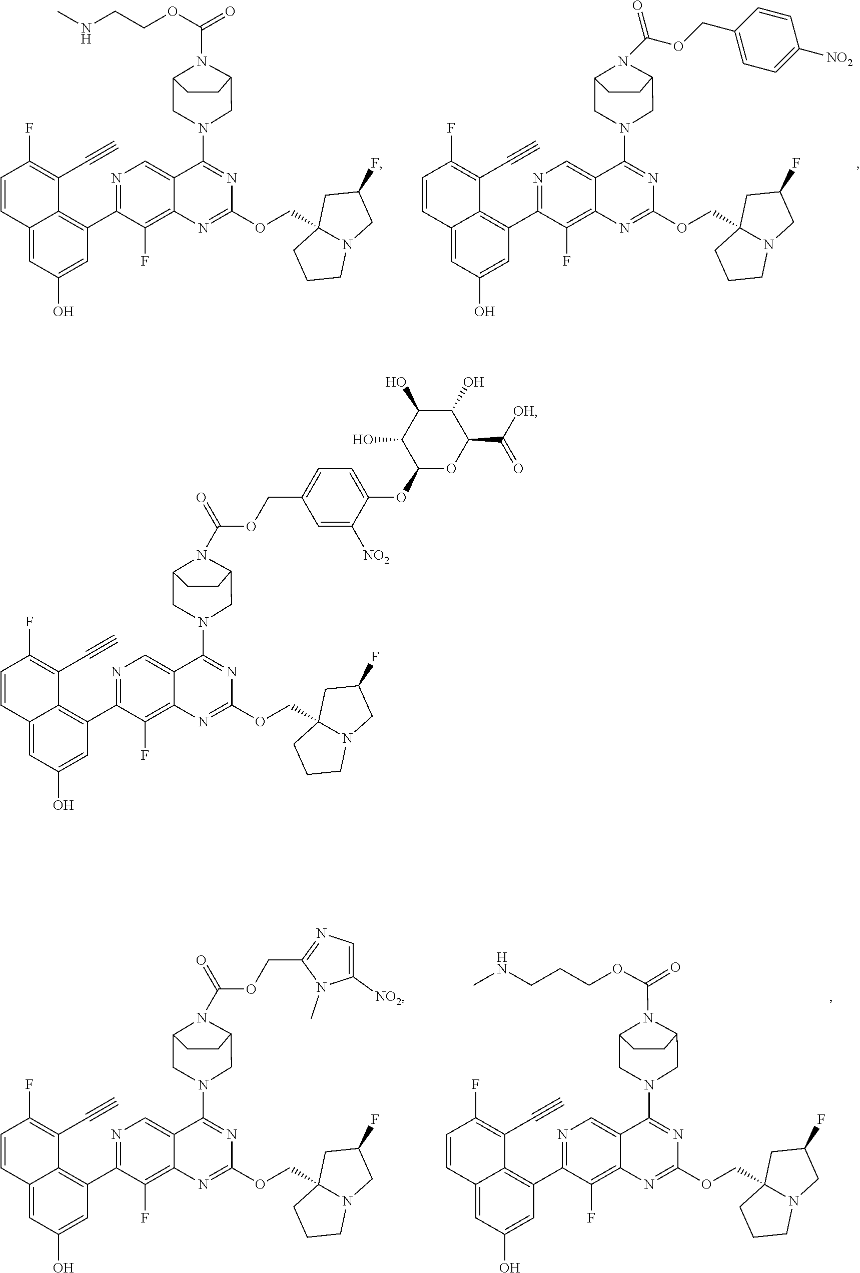 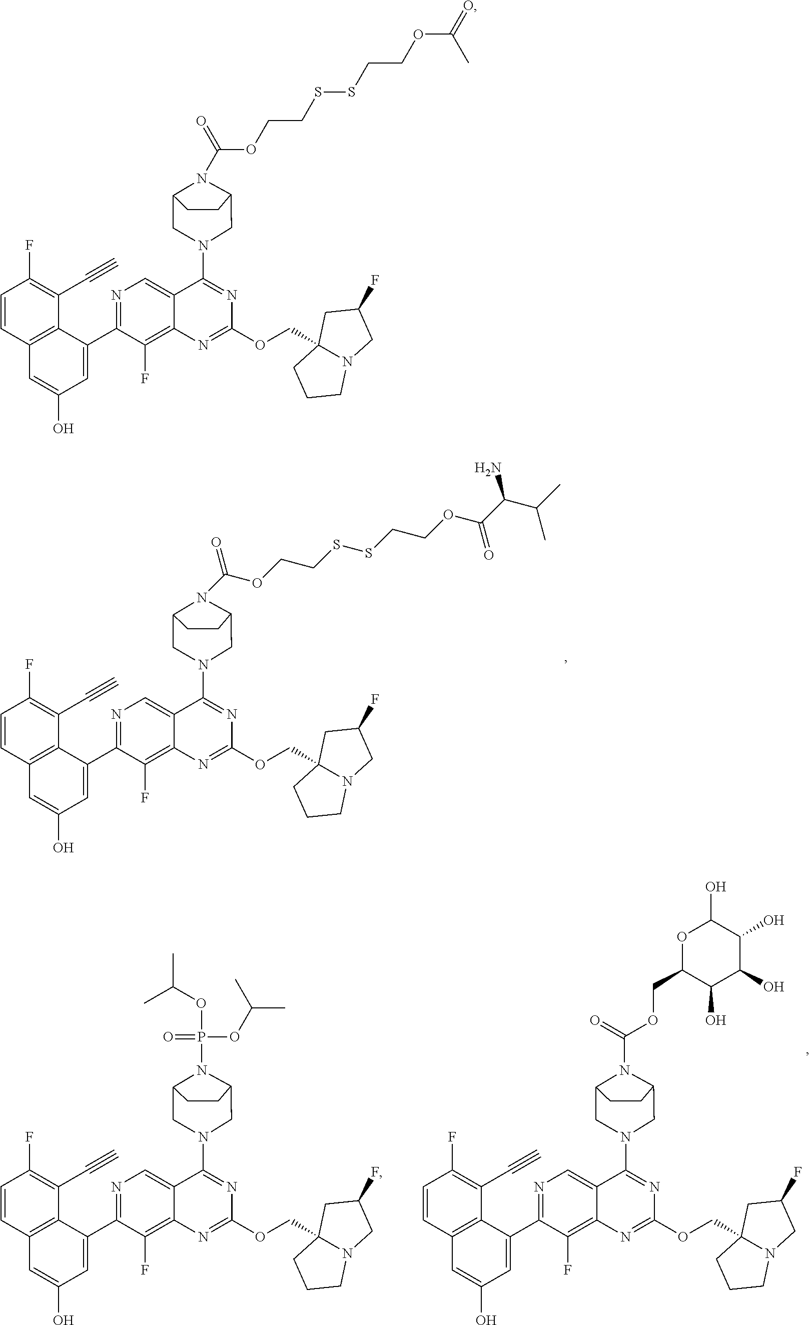 [SMILES: C#Cc1c(F)ccc2cc(O)cc(-c3ncc4c(N5CC6CCC(C5)N6C(=O)OCCSSCCOC(C)=O)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)OCCSSCCOC(=O)[C@@H](N)C(C)C)nc(OC[C@@]56CCCN5C[C@H](F)C6)nc4c3F)c12 ; C#Cc1c(F)ccc2cc(O)cc(-c3ncc4c(N5CC6CCC(C5)N6P(=O)(OC(C)C)OC(C)C)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)OC[C@H]5OC(O)[C@H](O)[C@@H](O)[C@H]5O)nc(OC[C@@]56CCCN5C[C@H](F)C6)nc4c3F)c12] 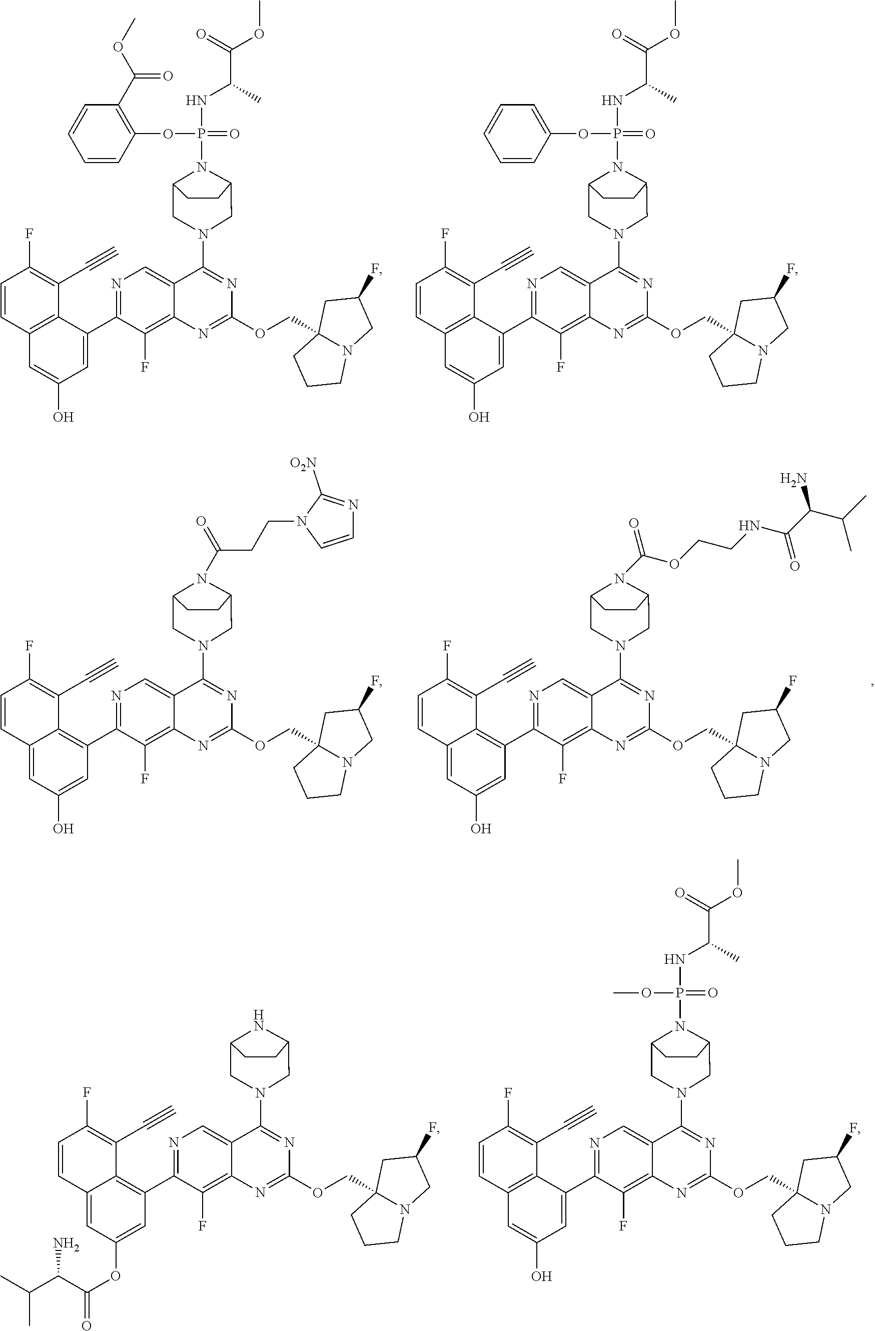 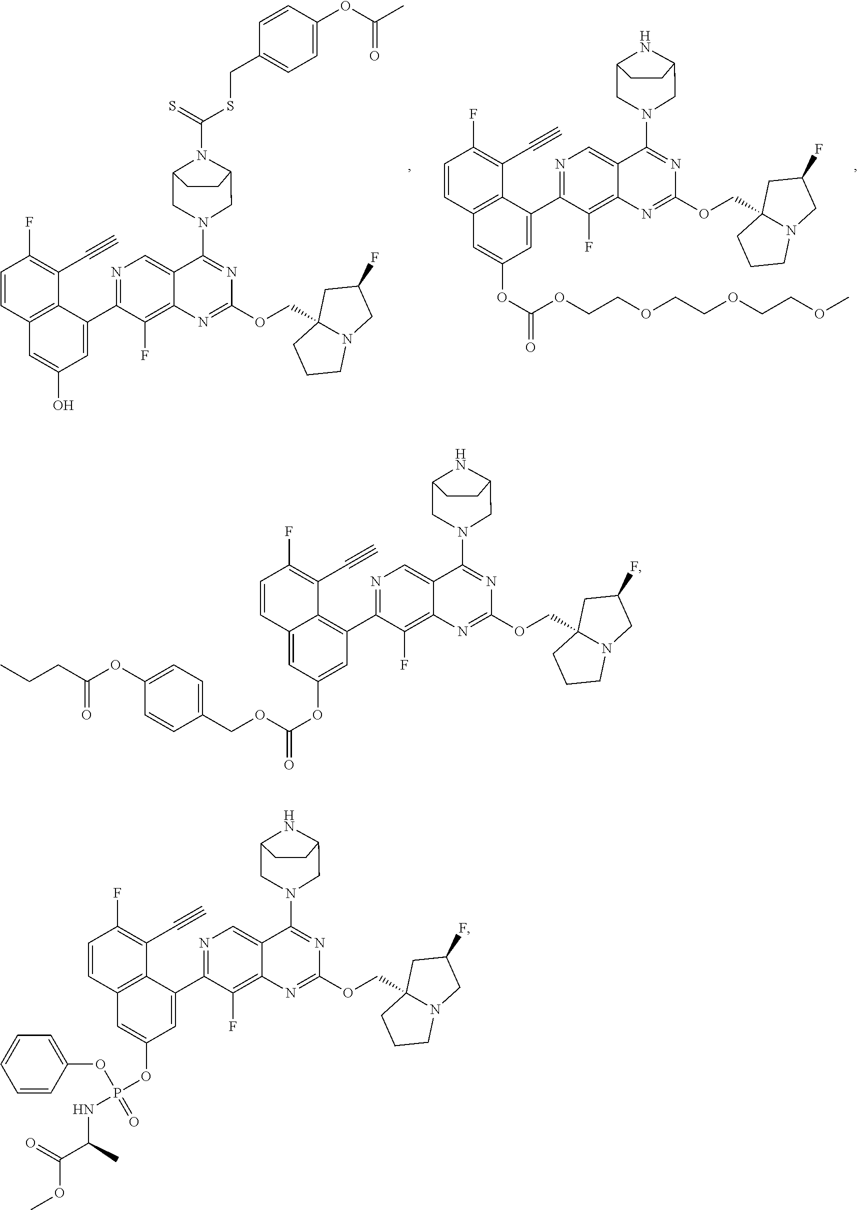 [SMILES: C#Cc1c(F)ccc2cc(O)cc(-c3ncc4c(N5CC6CCC(C5)N6C(=S)SCc5ccc(OC(C)=O)cc5)nc(OC[C@@]56CCCN5C[C@H](F)C6)nc4c3F)c12 ; C#Cc1c(F)ccc2cc(OC(=O)OCCOCCOCCOC)cc(-c3ncc4c(N5CC6CCC(C5)N6)nc(OC[C@@]56CCCN5C[C@H](F)C6)nc4c3F)c12 ; C#Cc1c(F)ccc2cc(OC(=O)OCc3ccc(OC(=O)CCC)cc3)cc(-c3ncc4c(N5CC6CCC(C5)N6)nc(OC[C@@]56CCCN5C[C@H](F)C6)nc4c3F)c12 ; C#Cc1c(F)ccc2cc(OP(=O)(N[C@@H](C)C(=O)OC)Oc3ccccc3)cc(-c3ncc4c(N5CC6CCC(C5)N6)nc(OC[C@@]56CCCN5C[C@H](F)C6)nc4c3F)c12] 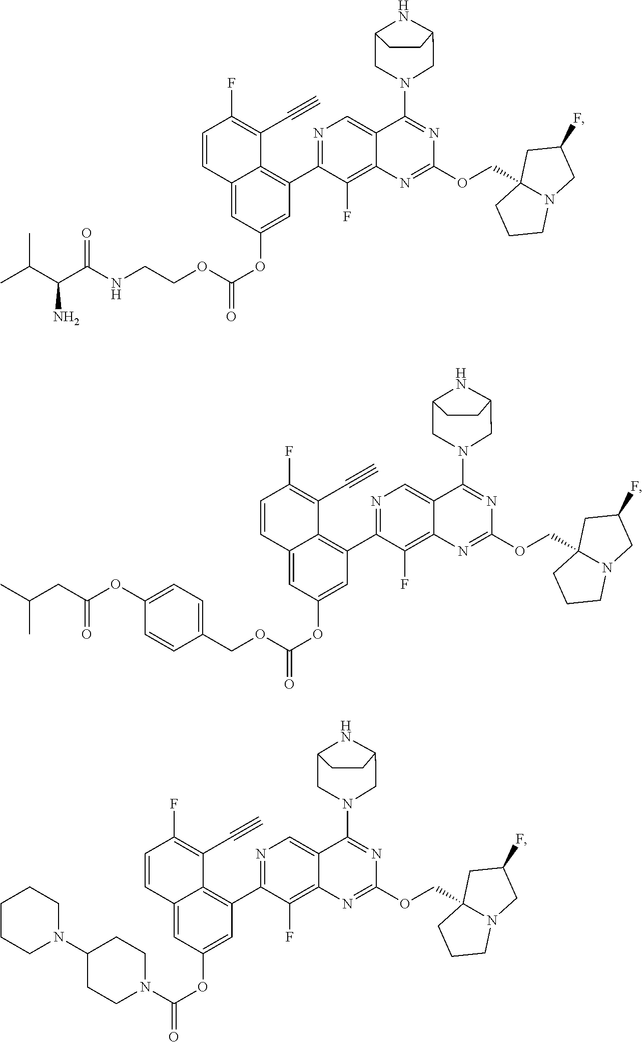 [SMILES: C#Cc1c(F)ccc2cc(OC(=O)OCCNC(=O)[C@@H](N)C(C)C)cc(-c3ncc4c(N5CC6CCC(C5)N6)nc(OC[C@@]56CCCN5C[C@H](F)C6)nc4c3F)c12 ; C#Cc1c(F)ccc2cc(OC(=O)OCc3ccc(OC(=O)CC(C)C)cc3)cc(-c3ncc4c(N5CC6CCC(C5)N6)nc(OC[C@@]56CCCN5C[C@H](F)C6)nc4c3F)c12 ; C#Cc1c(F)ccc2cc(OC(=O)N3CCC(N4CCCCC4)CC3)cc(-c3ncc4c(N5CC6CCC(C5)N6)nc(OC[C@@]56CCCN5C[C@H](F)C6)nc4c3F)c12] 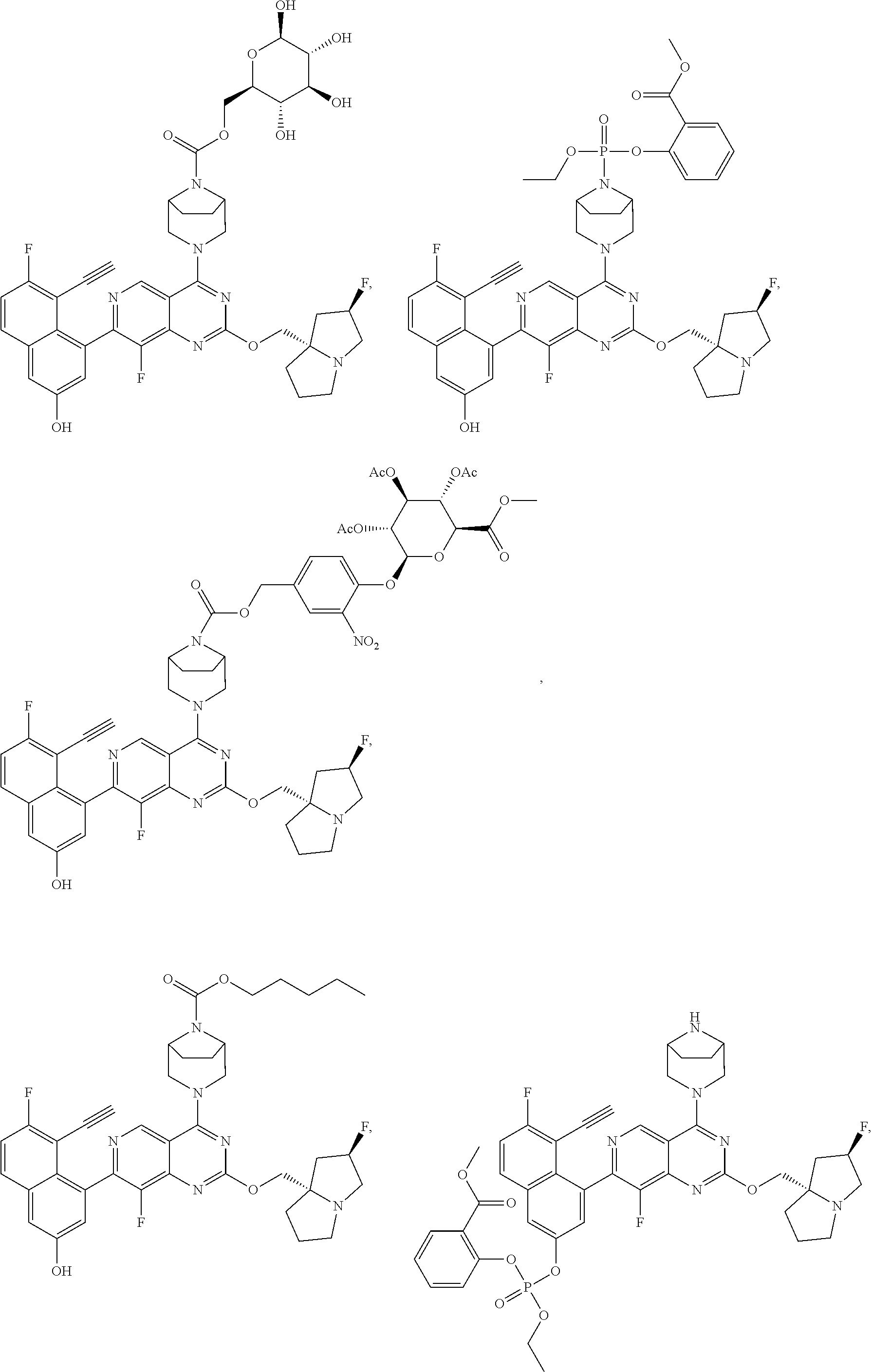 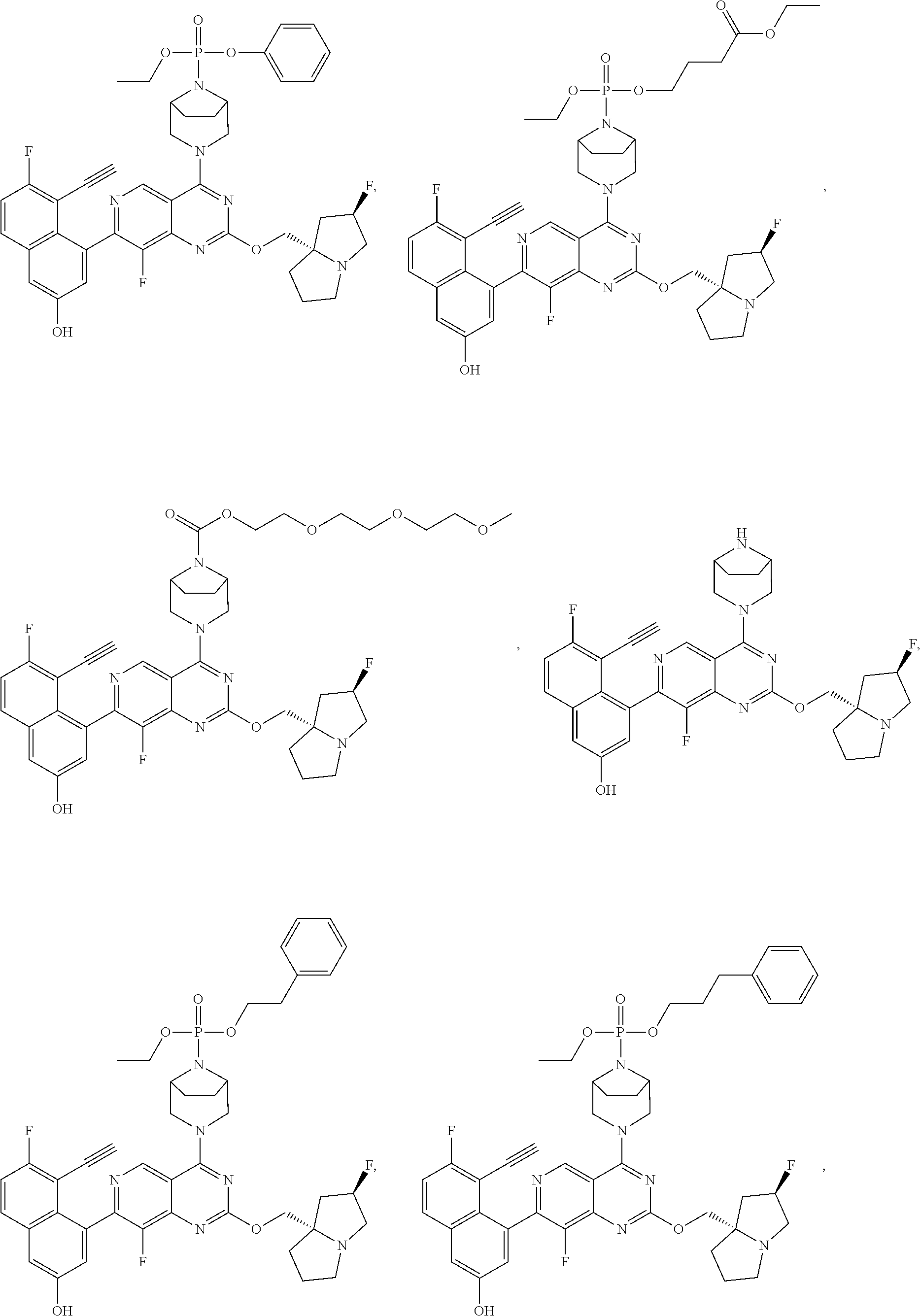 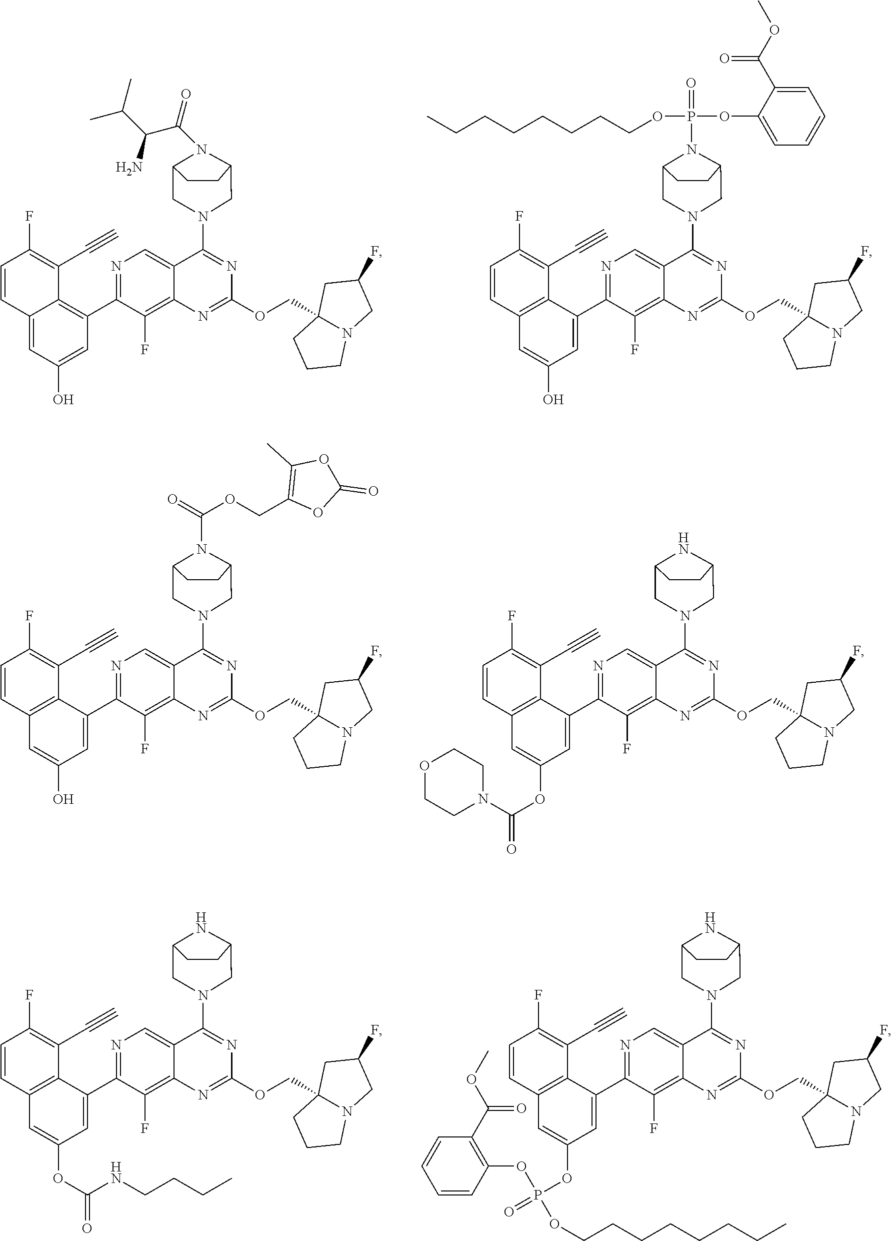 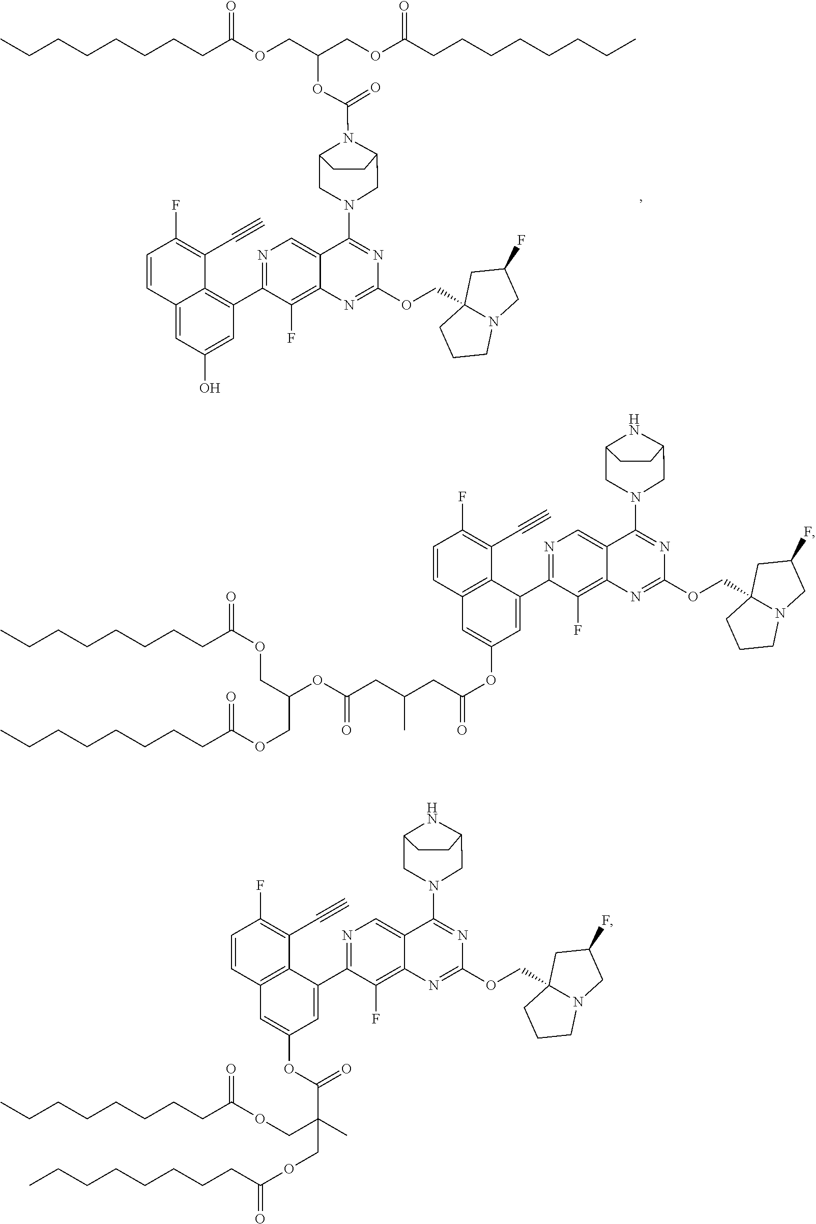 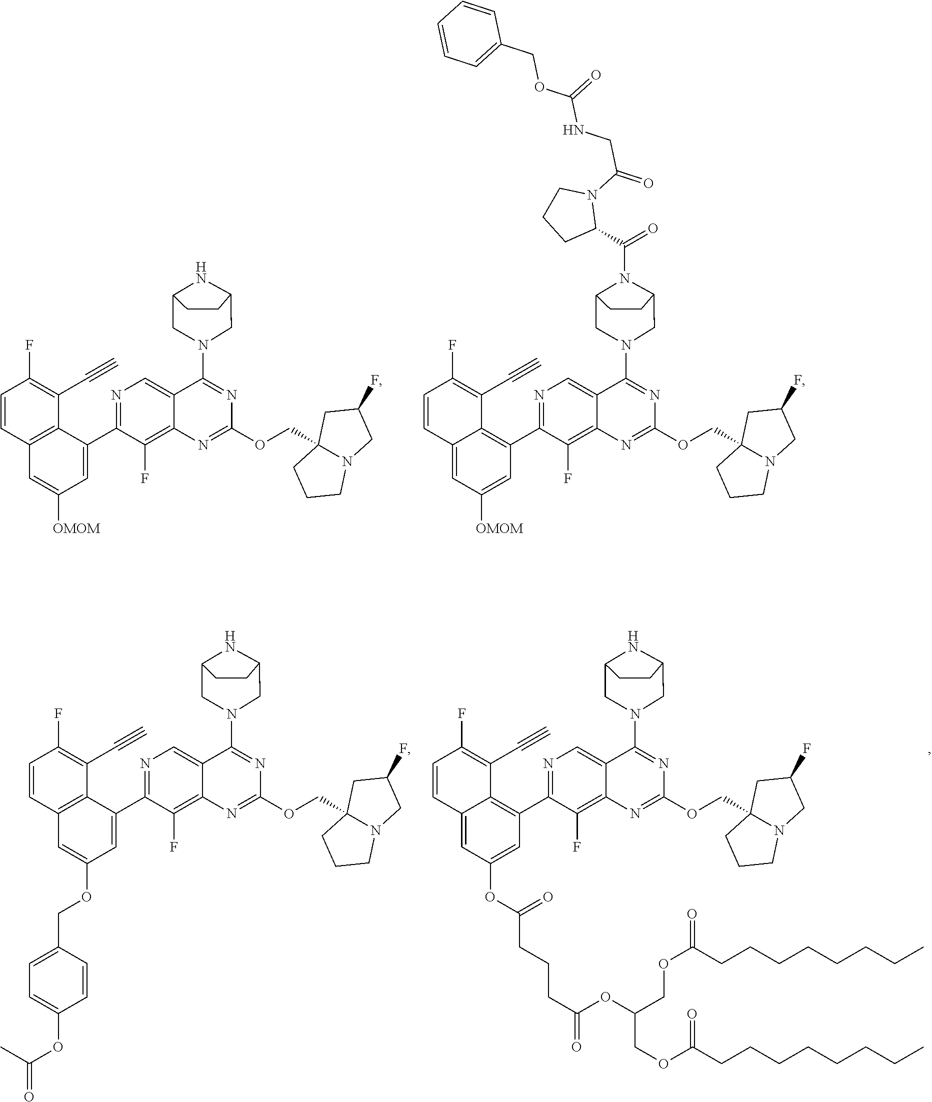 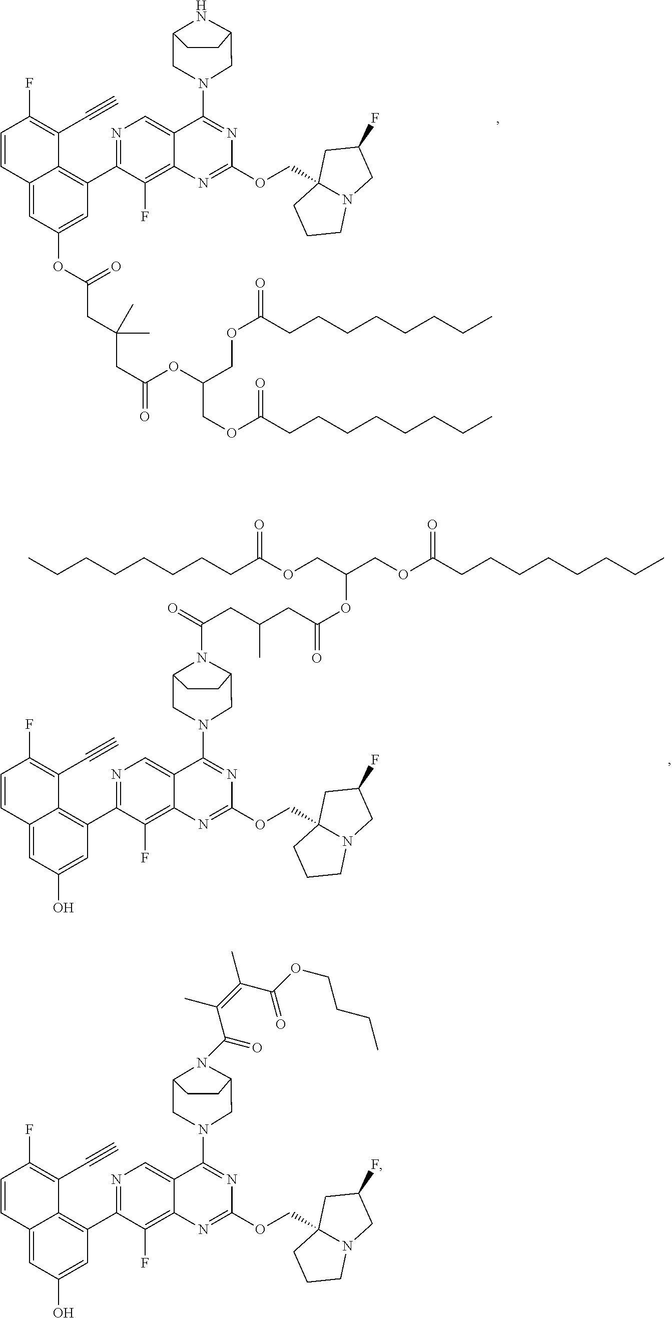 [SMILES: C#Cc1c(F)ccc2cc(OC(=O)CC(C)(C)CC(=O)OC(COC(=O)CCCCCCCC)COC(=O)CCCCCCCC)cc(-c3ncc4c(N5CC6CCC(C5)N6)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)CC(C)CC(=O)OC(COC(=O)CCCCCCCC)COC(=O)CCCCCCCC)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)/C(C)=C(/C)C(=O)OCCCC)nc(OC[C@@]56CCCN5C[C@H](F)C6)nc4c3F)c12] 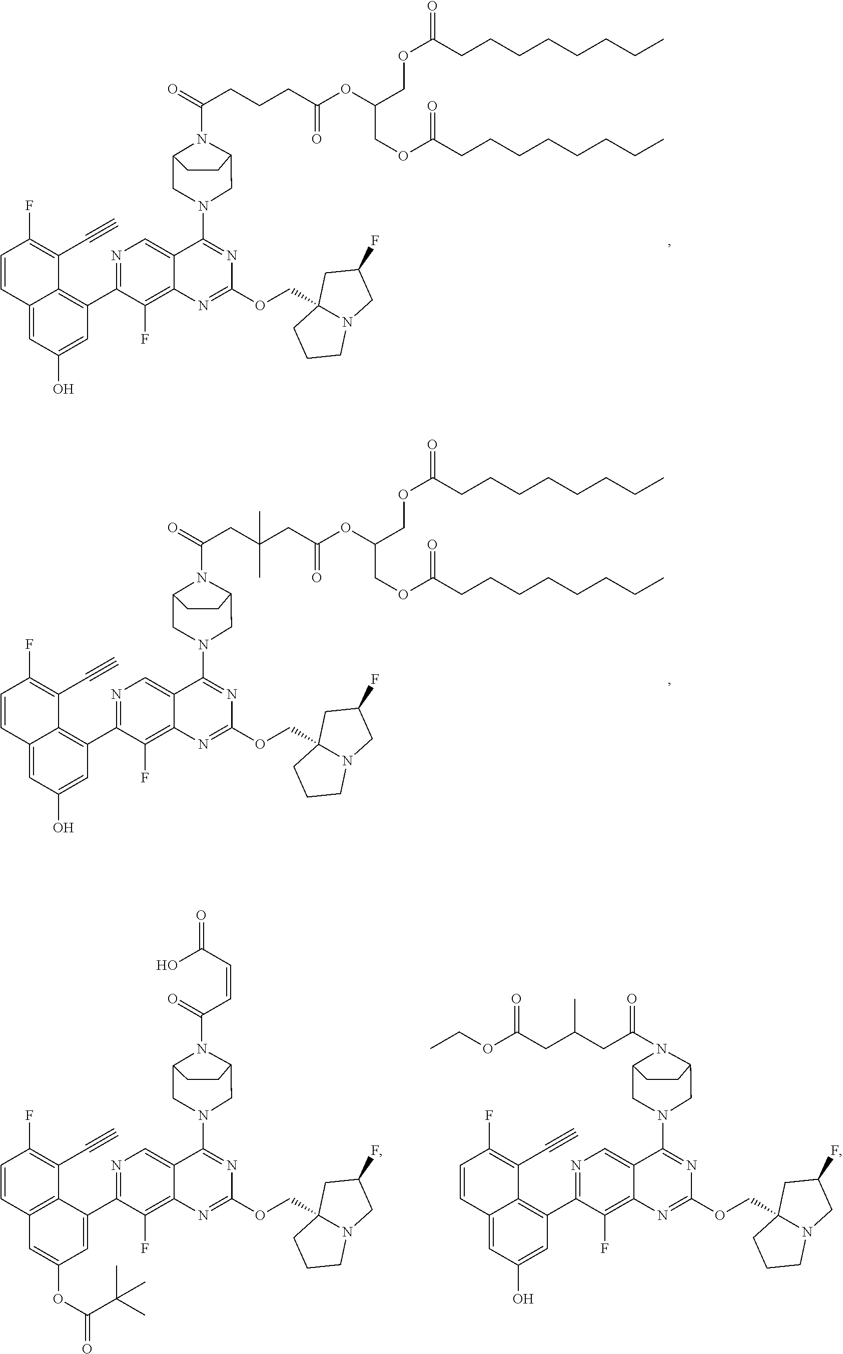 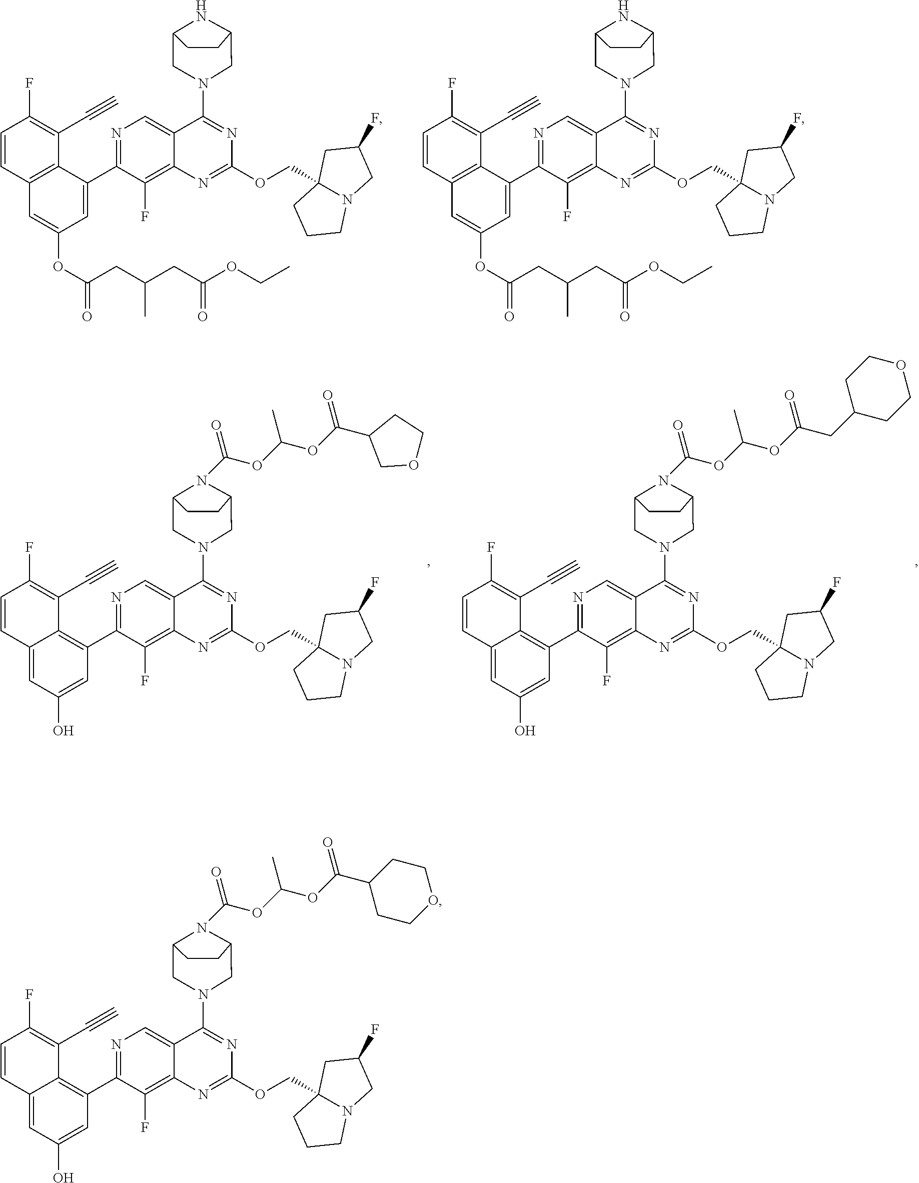 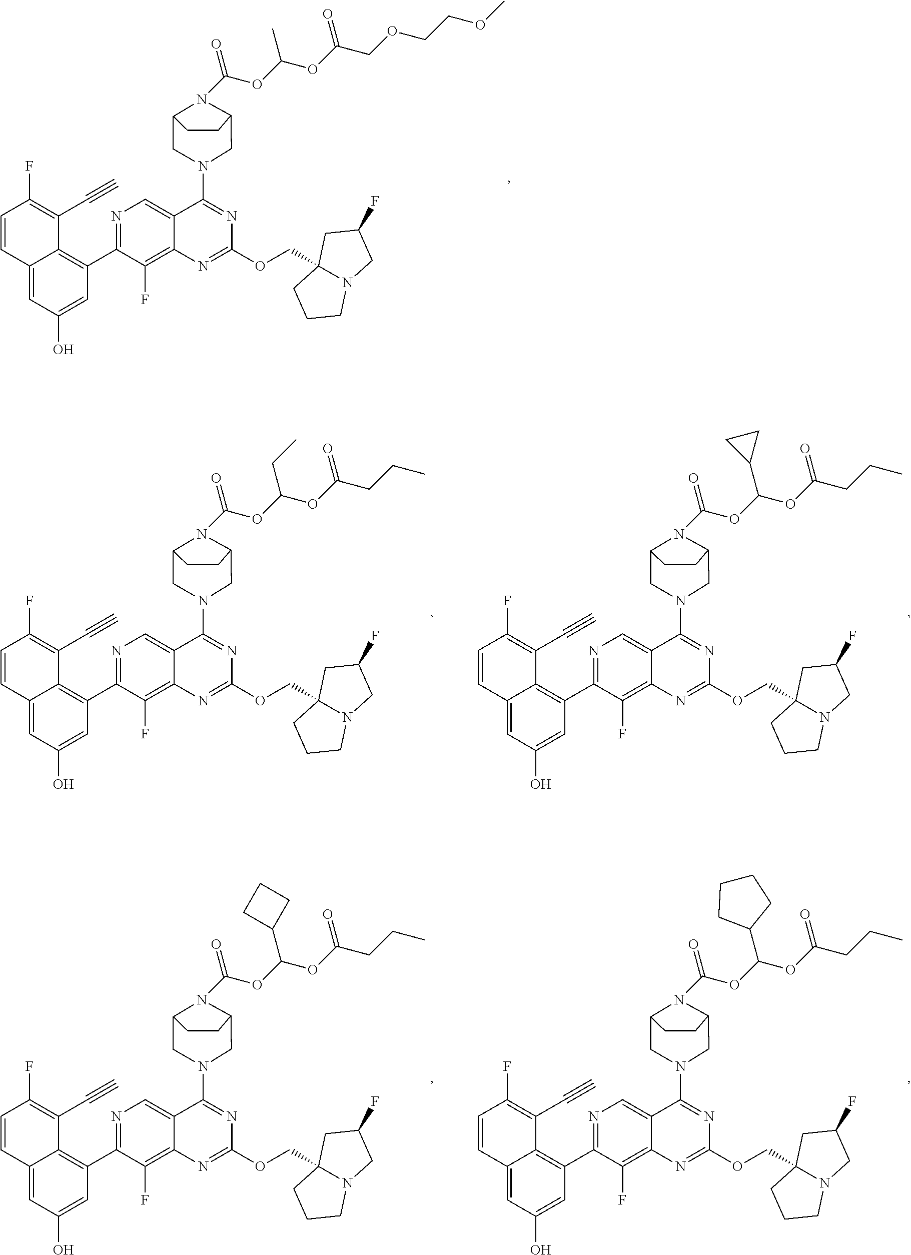 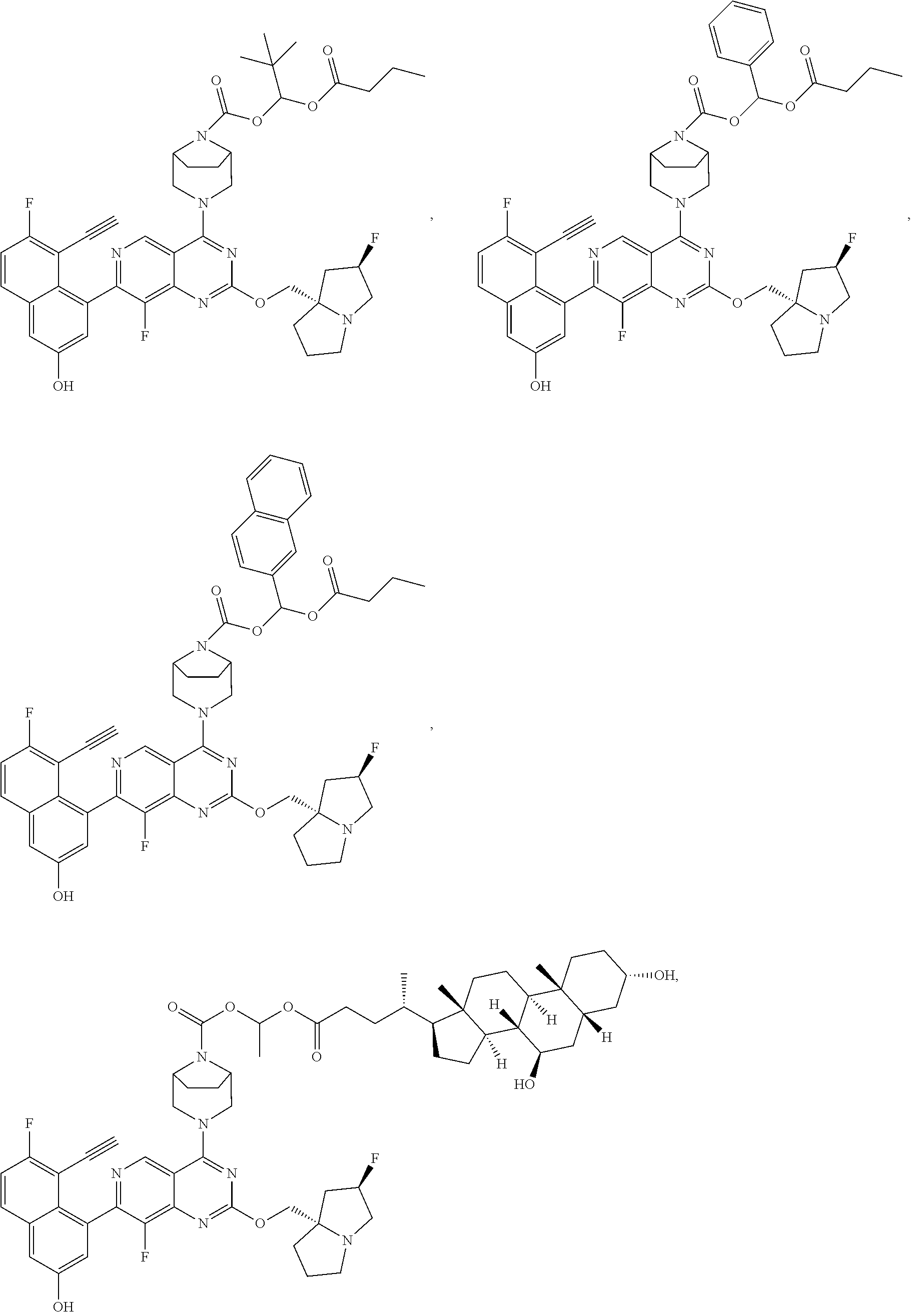 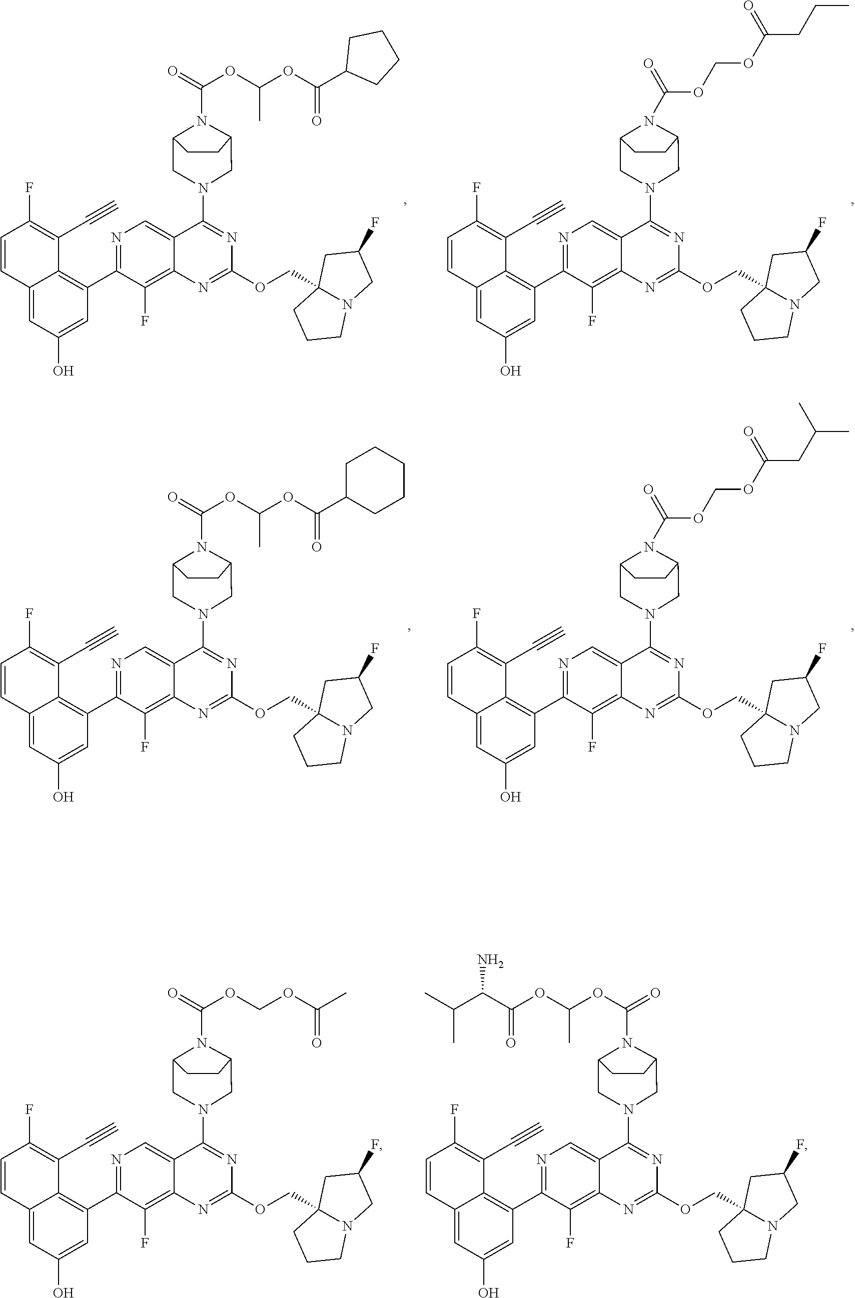 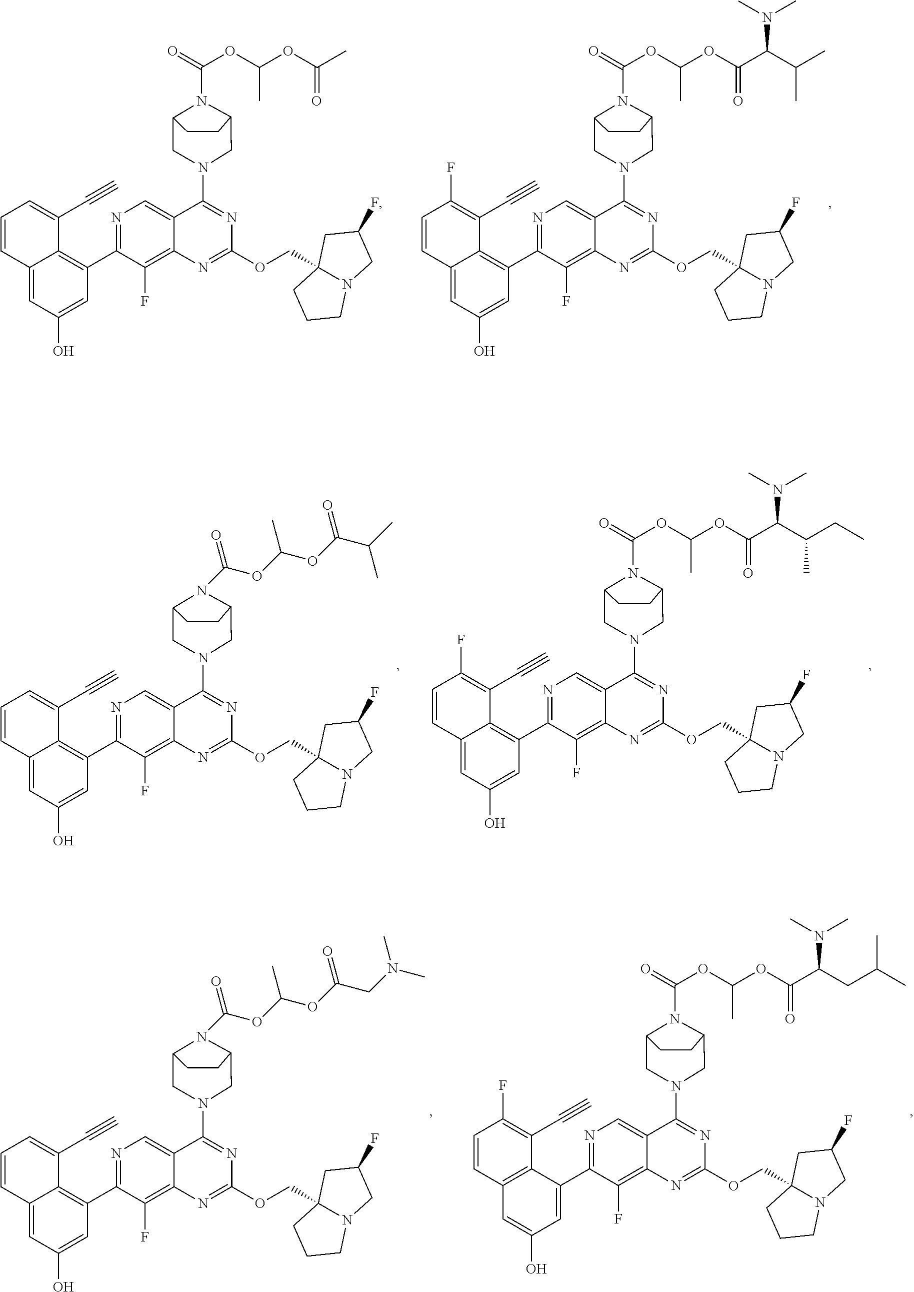 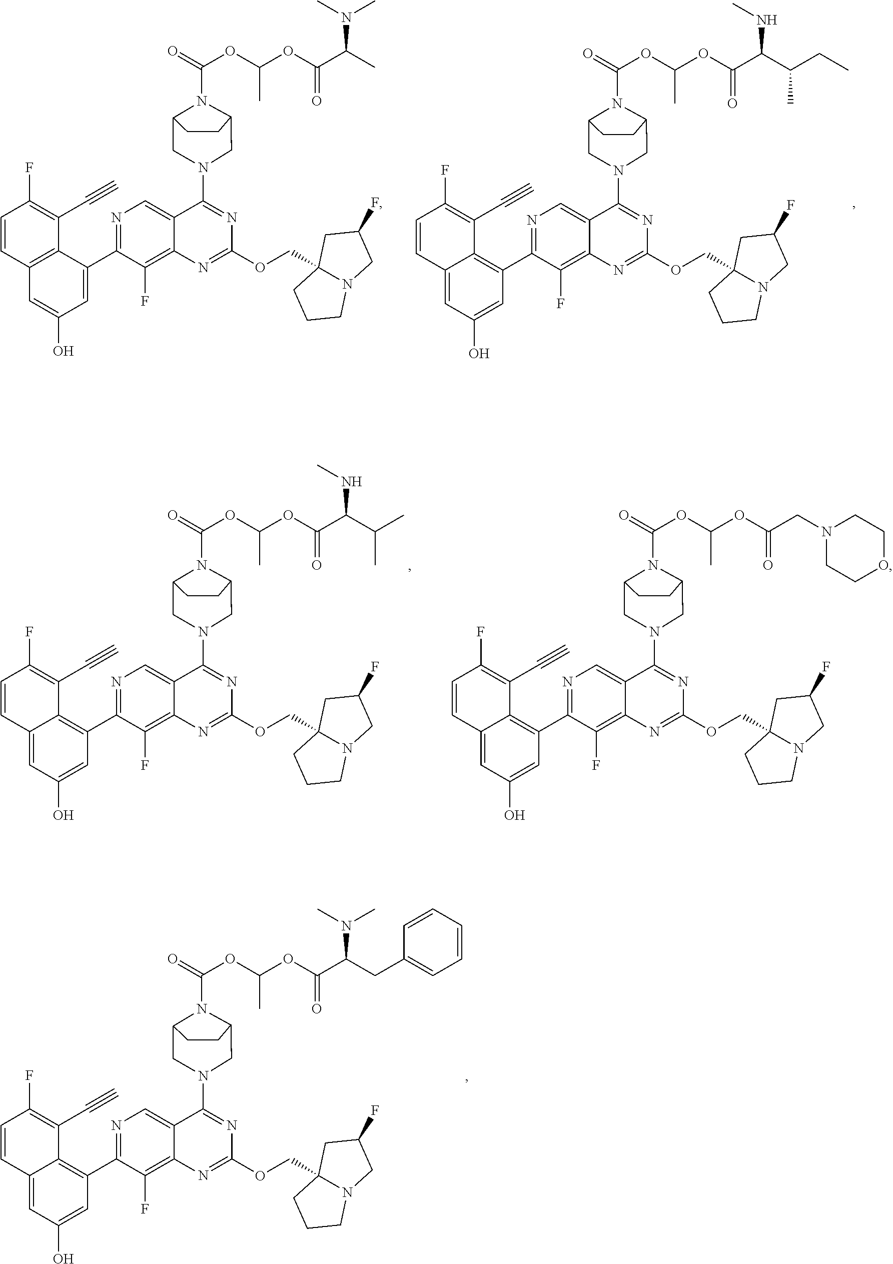 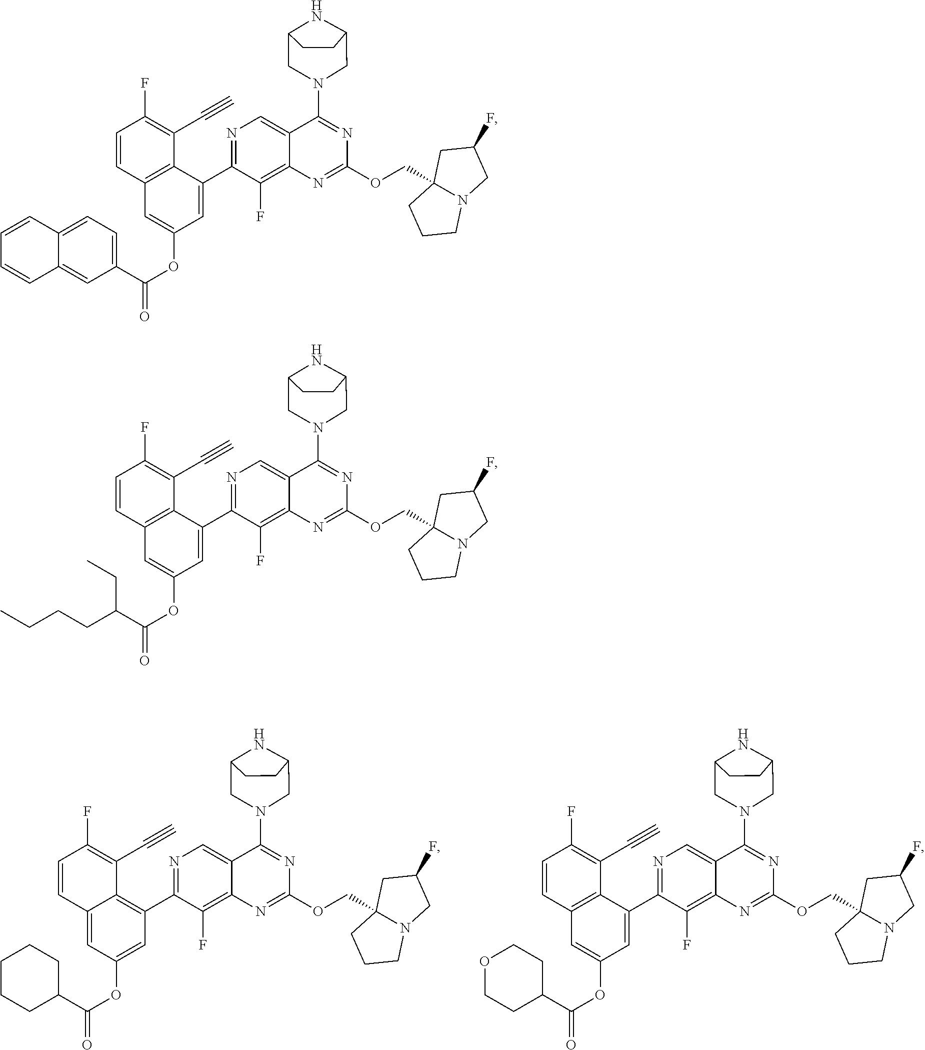 [SMILES: C#Cc1c(F)ccc2cc(OC(=O)c3ccc4ccccc4c3)cc(-c3ncc4c(N5CC6CCC(C5)N6)nc(OC[C@@]56CCCN5C[C@H](F)C6)nc4c3F)c12 ; C#Cc1c(F)ccc2cc(OC(=O)C(CC)CCCC)cc(-c3ncc4c(N5CC6CCC(C5)N6)nc(OC[C@@]56CCCN5C[C@H](F)C6)nc4c3F)c12 ; C#Cc1c(F)ccc2cc(OC(=O)C3CCCCC3)cc(-c3ncc4c(N5CC6CCC(C5)N6)nc(OC[C@@]56CCCN5C[C@H](F)C6)nc4c3F)c12 ; C#Cc1c(F)ccc2cc(OC(=O)C3CCOCC3)cc(-c3ncc4c(N5CC6CCC(C5)N6)nc(OC[C@@]56CCCN5C[C@H](F)C6)nc4c3F)c12] 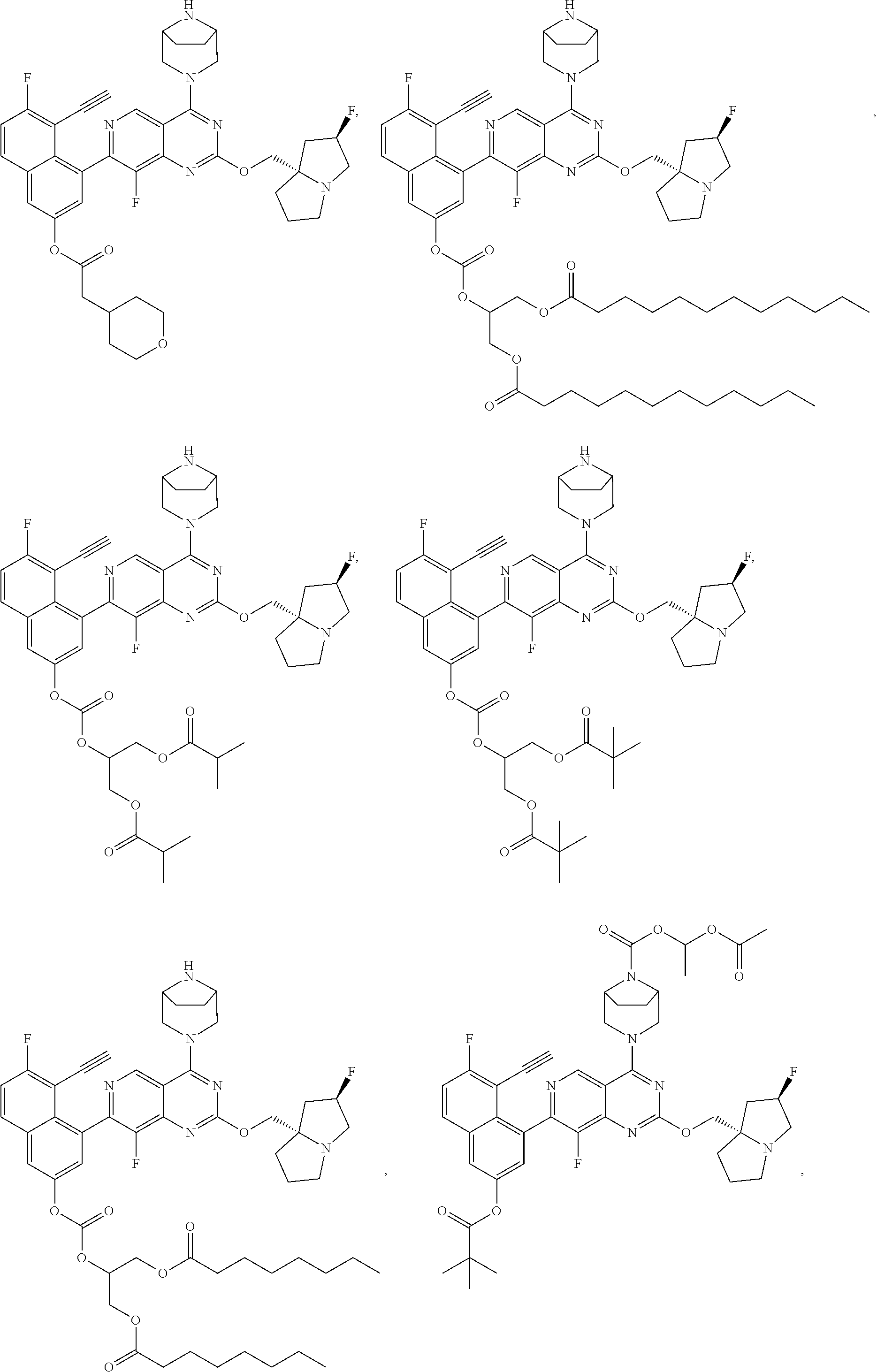 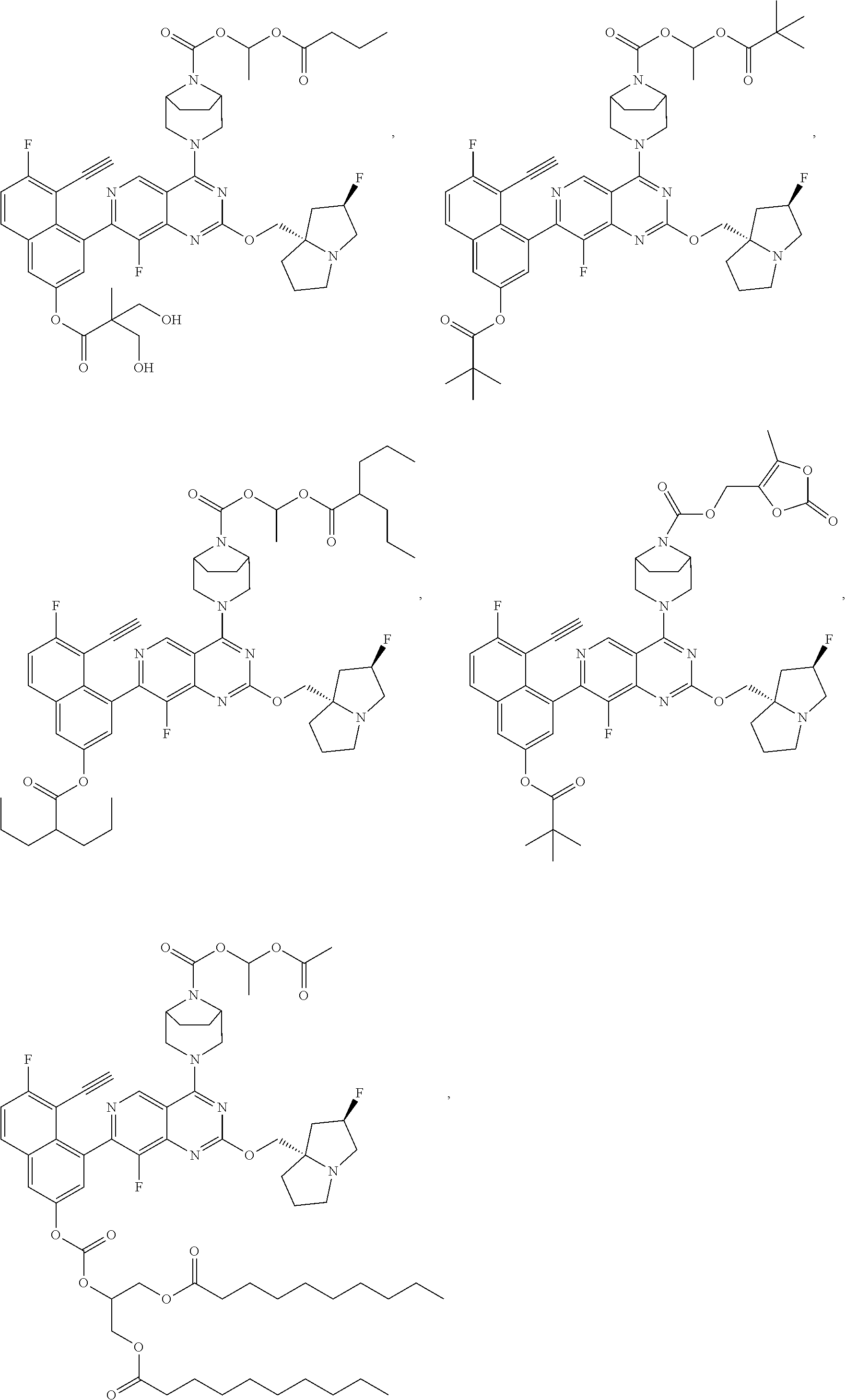 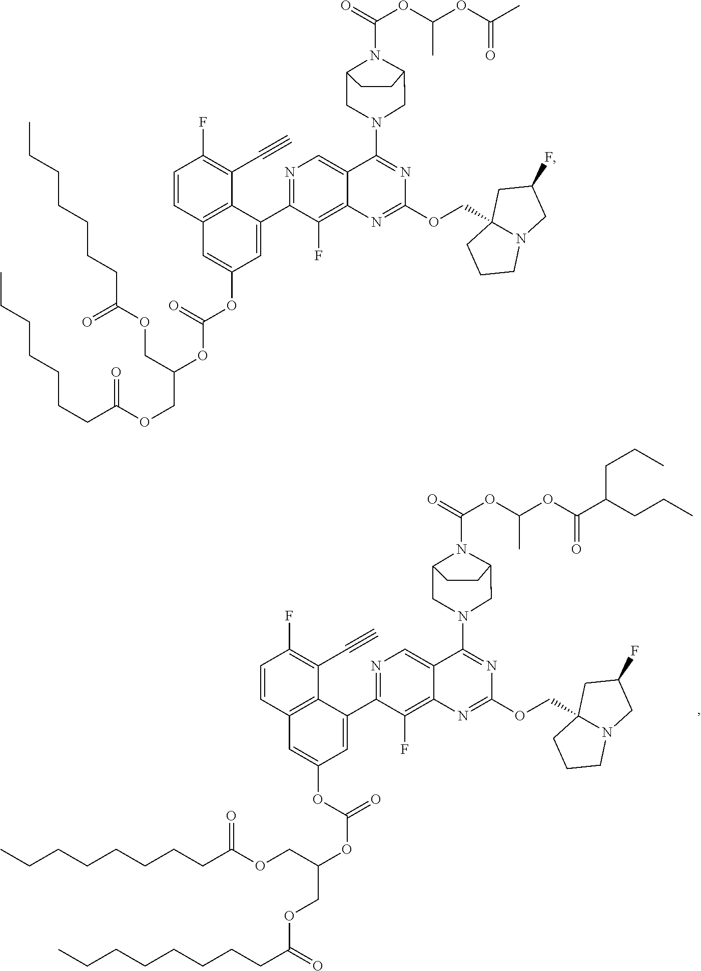 [SMILES: C#Cc1c(F)ccc2cc(OC(=O)OC(COC(=O)CCCCCCC)COC(=O)CCCCCCC)cc(-c3ncc4c(N5CC6CCC(C5)N6C(=O)OC(C)OC(C)=O)nc(OC[C@@]56CCCN5C[C@H](F)C6)nc4c3F)c12 ; C#Cc1c(F)ccc2cc(OC(=O)OC(COC(=O)CCCCCCCC)COC(=O)CCCCCCCC)cc(-c3ncc4c(N5CC6CCC(C5)N6C(=O)OC(C)OC(=O)C(CCC)CCC)nc(OC[C@@]56CCCN5C[C@H](F)C6)nc4c3F)c12] 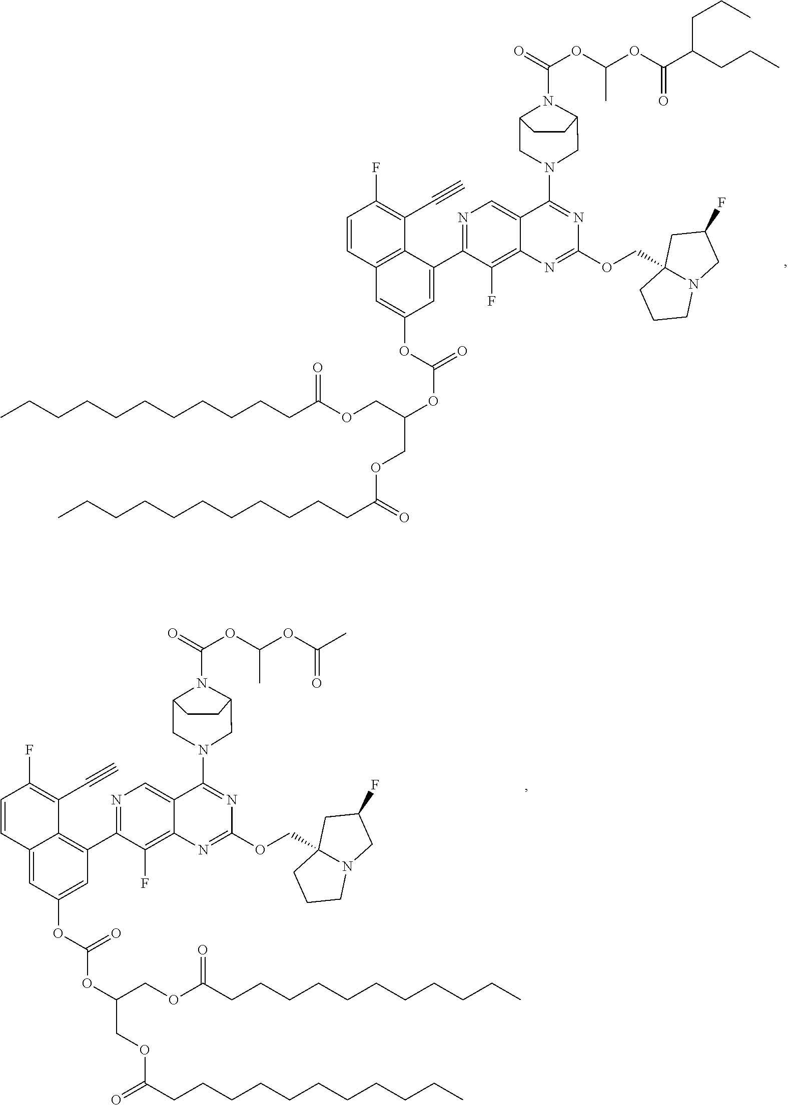 [SMILES: C#Cc1c(F)ccc2cc(OC(=O)OC(COC(=O)CCCCCCCCCCC)COC(=O)CCCCCCCCCCC)cc(-c3ncc4c(N5CC6CCC(C5)N6C(=O)OC(C)OC(=O)C(CCC)CCC)nc(OC[C@@]56CCCN5C[C@H](F)C6)nc4c3F)c12 ; C#Cc1c(F)ccc2cc(OC(=O)OC(COC(=O)CCCCCCCCCCC)COC(=O)CCCCCCCCCCC)cc(-c3ncc4c(N5CC6CCC(C5)N6C(=O)OC(C)OC(C)=O)nc(OC[C@@]56CCCN5C[C@H](F)C6)nc4c3F)c12] 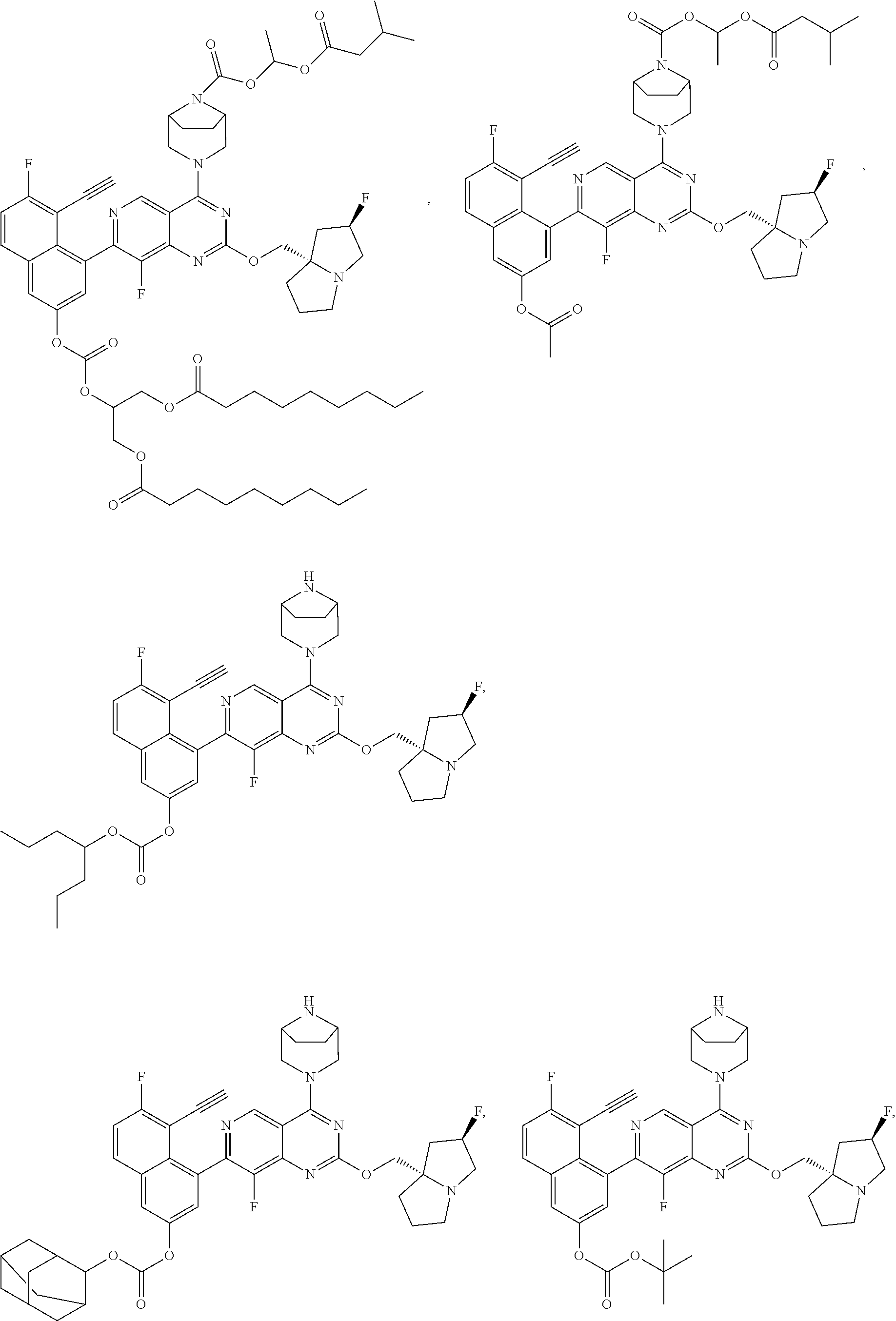 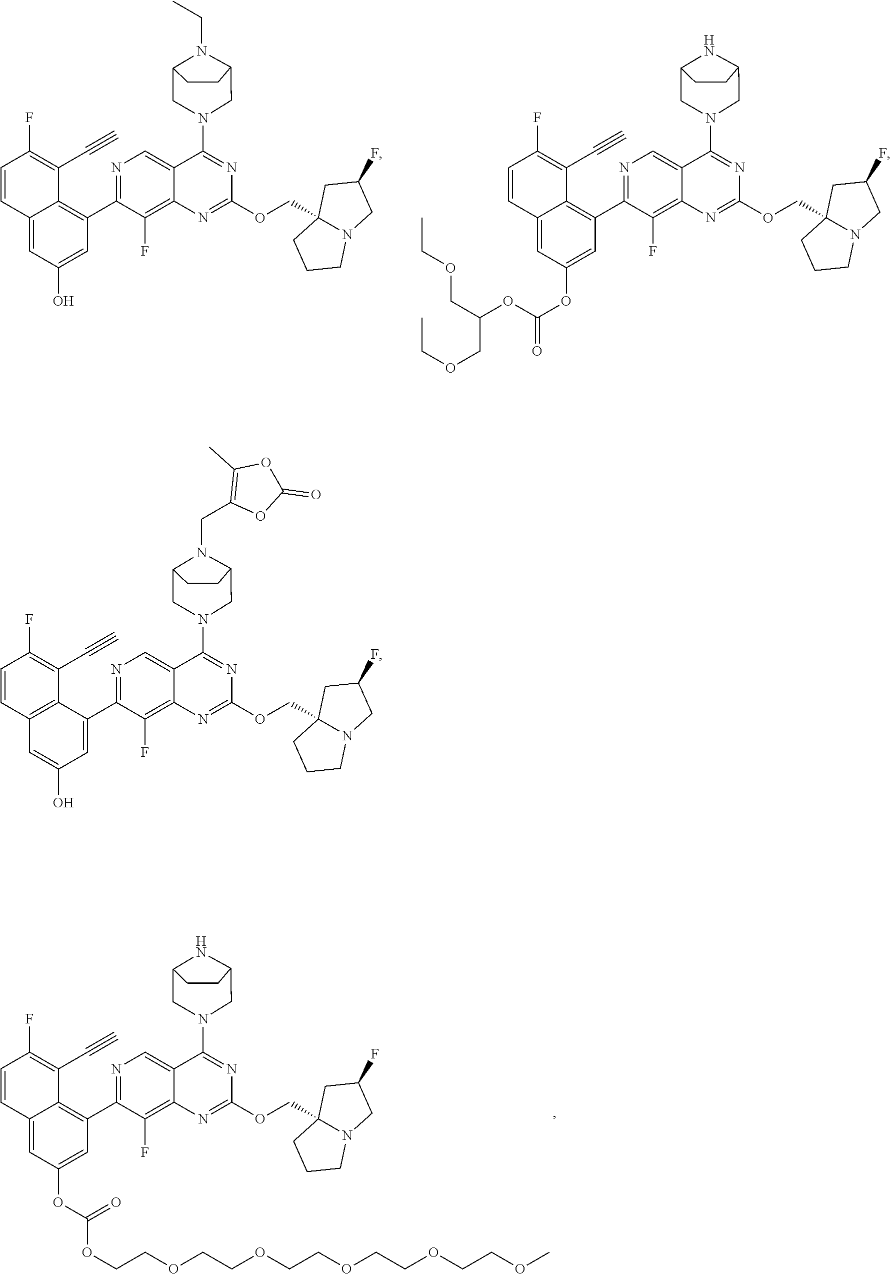 [SMILES: C#Cc1c(F)ccc2cc(O)cc(-c3ncc4c(N5CC6CCC(C5)N6CC)nc(OC[C@@]56CCCN5C[C@H](F)C6)nc4c3F)c12 ; C#Cc1c(F)ccc2cc(OC(=O)OC(COCC)COCC)cc(-c3ncc4c(N5CC6CCC(C5)N6)nc(OC[C@@]56CCCN5C[C@H](F)C6)nc4c3F)c12 ; C#Cc1c(F)ccc2cc(O)cc(-c3ncc4c(N5CC6CCC(C5)N6Cc5oc(=O)oc5C)nc(OC[C@@]56CCCN5C[C@H](F)C6)nc4c3F)c12 ; C#Cc1c(F)ccc2cc(OC(=O)OCCOCCOCCOCCOCCOC)cc(-c3ncc4c(N5CC6CCC(C5)N6)nc(OC[C@@]56CCCN5C[C@H](F)C6)nc4c3F)c12] 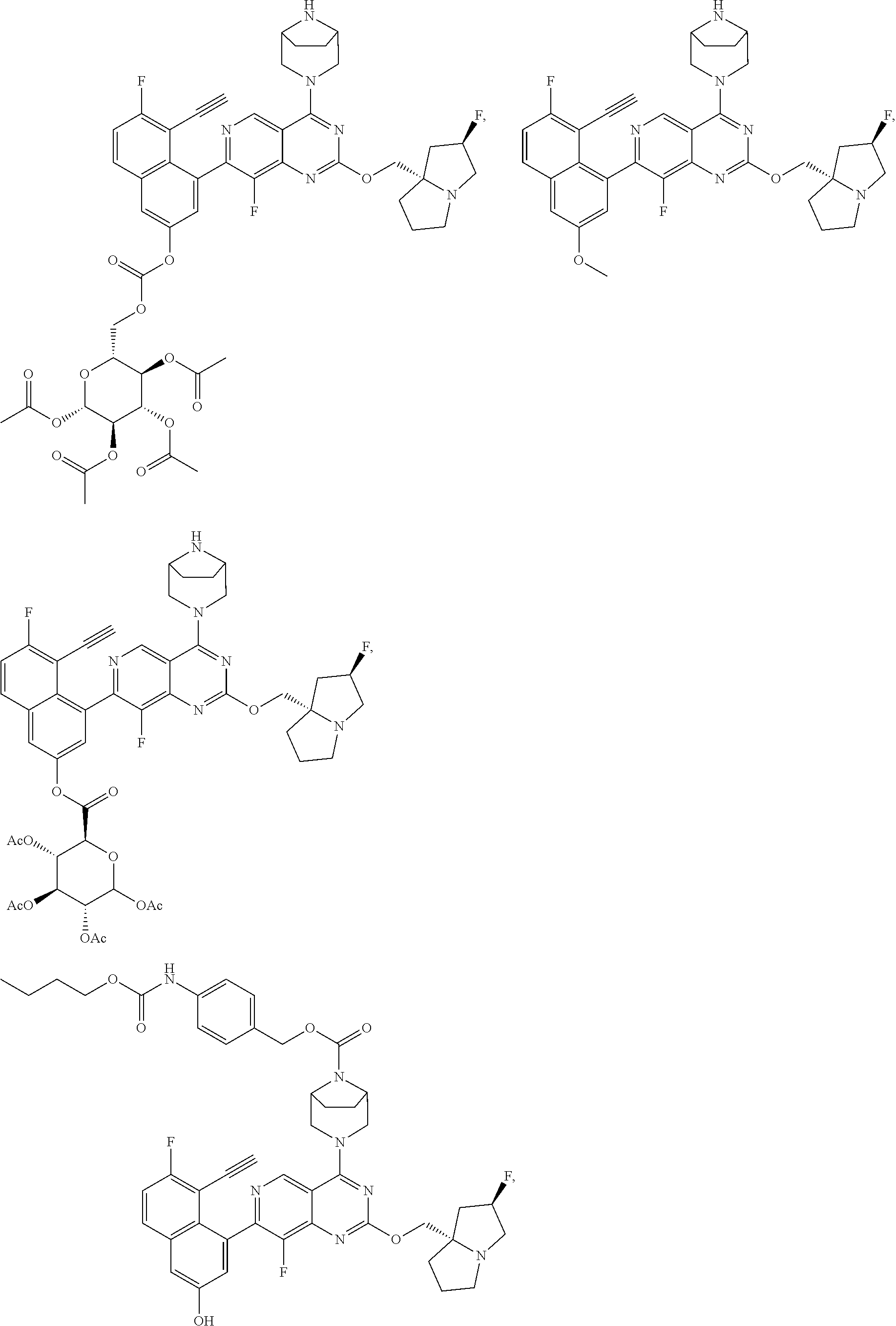 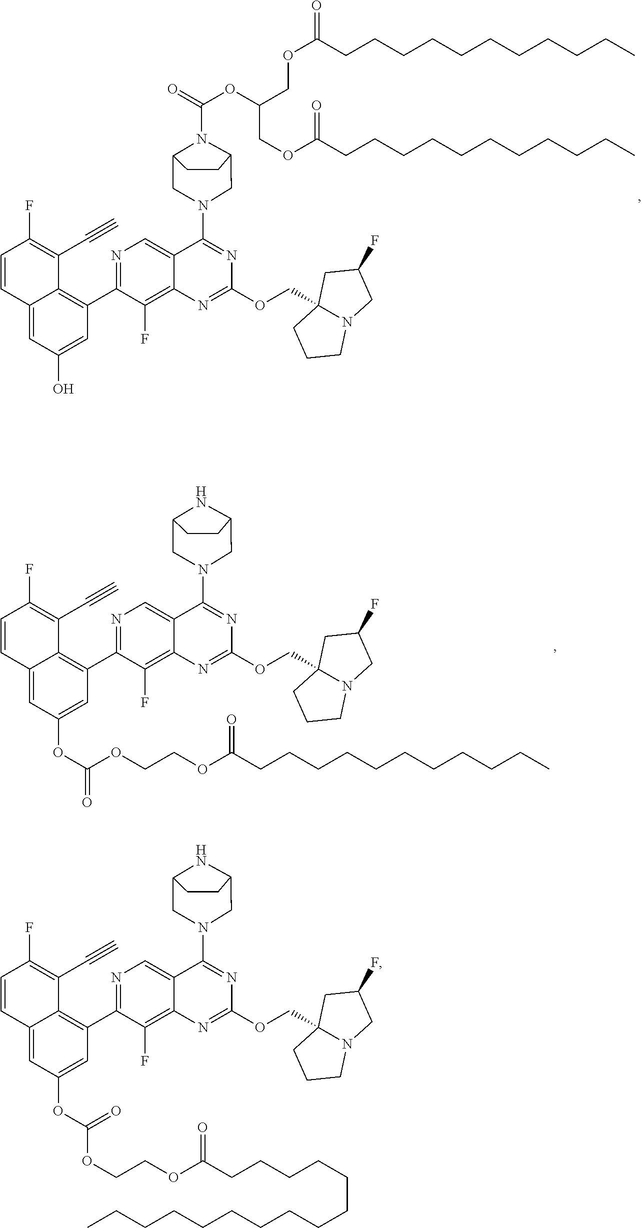 [SMILES: C#Cc1c(F)ccc2cc(O)cc(-c3ncc4c(N5CC6CCC(C5)N6C(=O)OC(COC(=O)CCCCCCCCCCC)COC(=O)CCCCCCCCCCC)nc(OC[C@@]56CCCN5C[C@H](F)C6)nc4c3F)c12 ; C#Cc1c(F)ccc2cc(OC(=O)OCCOC(=O)CCCCCCCCCCC)cc(-c3ncc4c(N5CC6CCC(C5)N6)nc(OC[C@@]56CCCN5C[C@H](F)C6)nc4c3F)c12 ; C#Cc1c(F)ccc2cc(OC(=O)OCCOC(=O)CCCCCCCCCCCCCCC)cc(-c3ncc4c(N5CC6CCC(C5)N6)nc(OC[C@@]56CCCN5C[C@H](F)C6)nc4c3F)c12]
or a pharmaceutically acceptable salt, ester, hydrate, solvate, or stereoisomer thereof.

49. The compound of claim 48, wherein the compound is: 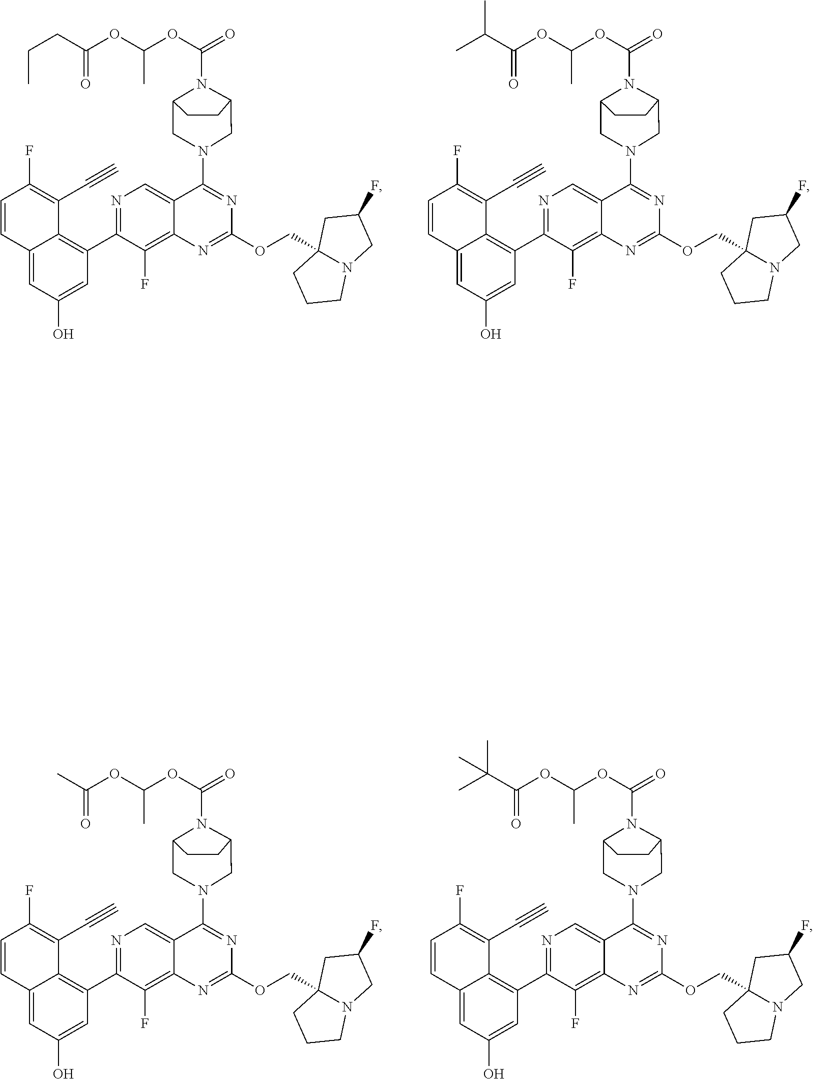 [SMILES: C#Cc1c(F)ccc2cc(O)cc(-c3ncc4c(N5CC6CCC(C5)N6C(=O)OC(C)OC(=O)CCC)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)OC(C)OC(=O)C(C)C)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)OC(C)OC(C)=O)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)OC(C)OC(=O)C(C)(C)C)nc(OC[C@@]56CCCN5C[C@H](F)C6)nc4c3F)c12] 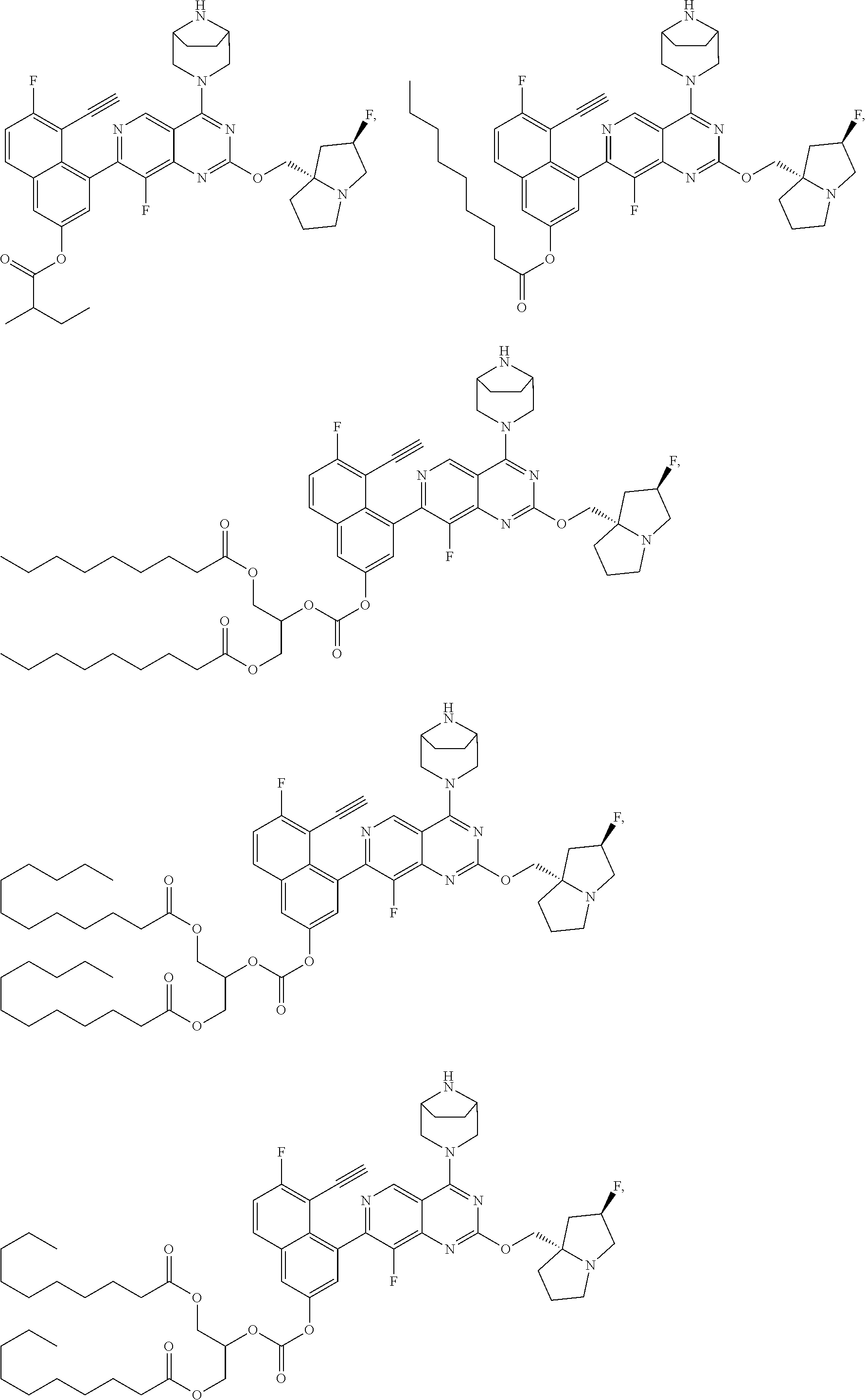
or a pharmaceutically acceptable salt, ester, hydrate, solvate, or stereoisomer thereof.

50. A pharmaceutical composition comprising the compound or the pharmaceutically acceptable salt, ester, hydrate, solvate, or stereoisomer thereof of claim 1 and a pharmaceutically acceptable excipient, carrier or diluent.

51.-54. (canceled)

55. The pharmaceutical composition of claim 50, wherein the composition is suitable for administration by injection, e.g., subcutaneous, intravenous, intramuscular, or intraperitoneal administration.

56.-57. (canceled)

58. A method for inhibiting, treating and/or preventing a hyperproliferative disorder in a subject comprising administering an effective amount of the compound or the pharmaceutically acceptable salt, ester, hydrate, solvate, or stereoisomer thereof of claim 1 to the subject.

59. The method of claim 58, wherein the hyperproliferative disorder is a KRAS^{G12D}-associated cancer or tumor.

60. The method of claim 59, wherein the cancer or tumor is a cardiac, lung, gastrointestinal, genitourinary tract, liver, biliary tract, small intestine, large intestine, bone, nervous system, gynecological, hematologic, skin, or adrenal gland cancer or tumor.

61.-80. (canceled)

81. The method of claim 59, wherein the cancer or tumor is non-small cell lung cancer (NSCLC), small cell lung cancer, pancreatic cancer, colorectal cancer, colon cancer, bile duct cancer, cervical cancer, bladder cancer, liver cancer or breast cancer.

82.-84. (canceled)

85. The method of claim 58, wherein the method further comprises administration of at least one additional therapeutic agent to the subject.

86.-105. (canceled)
